(19) United States
(12) Patent Application Publication
(10) Pub. No.: US 2026/0191922 A1
(43) Pub. Date: Jul. 9, 2026

(54) DISPOSABLE PERSONAL CARE PRODUCTS COMPOSED OF NONWOVEN FABRICS COATED WITH BACTERIOPHAGES TO PREVENT AND REDUCE BACTERIALLY CAUSED SKIN PROBLEMS

(71) Applicant: PARALLEL HEALTH, INC., San Francisco, CA (US)

(72) Inventors: Nathan BROWN, Venice, CA (US); Natalise Kalea ROBINSON, Venice, CA (US)

(21) Appl. No.: 19/134,106

(22) PCT Filed: Nov. 29, 2023
(86) PCT No.: PCT/US2023/081698

§ 371 Date: May 29, 2025

Related U.S. Application Data
(60) Provisional application No. 63/385,398, filed on Nov. 29, 2022.

(51) Int. Cl. A61K 35/76; A61K 9/00; A61K 9/70; D04H 1/435

(52) U.S. Cl. CPC A61K 35/76; A61K 9/0014; A61K 9/70; D04H 1/435; D10B 2331/04; D10B 2401/13; D10B 2509/00

(57) ABSTRACT

Provided herein are compositions of phage combinations attached to surface-active polymer fabrics. The polymer fabrics may be integrated into personal care products. Provided herein are also methods for attaching the phage combinations onto the surface-active polymer fabrics. Methods for selecting the phage combinations are also described. Also provided are methods for using the composition of phage combinations for treating one or more skin conditions.

### 1. CROSS REFERENCE TO RELATED APPLICATIONS

This application claims the benefit of U.S. Provisional Application No. 63/385,398, filed Nov. 29, 2022, which is hereby incorporated by reference in its entirety.

### 2. SEQUENCE LISTING

The instant application contains a Sequence Listing which has been submitted electronically in XML format and is hereby incorporated by reference in its entirety. Said XML copy, created on Nov. 28, 2023, is named 54391WO_CRF_sequencelisting, and is 1,895,212 bytes in size.

### 3. BACKGROUND

Commonplace skin problems such as rashes, sensitive skin, itching, and discoloration are often associated with the use of disposable personal care products made of nonwoven fabrics that occlude the skin. These problems are usually associated with contact dermatitis and manifest in common consumer complaints such as diaper rash, maskne, sanitary pad rash, and incontinence-associated rash. The personal care products associated with these complaints are fitted against the skin for hours at a time and can elevate the pH, temperature, and moisture of the skin. The design of the personal care product itself and the texture of the nonwoven fabric worn closest to the skin have been optimized to wick moisture away from the skin, absorb excess bodily fluids distal from the skin, and reduce irritation from friction. Nonetheless, these products induce changes in skin physiology and the skin microbiome that lead to contact dermatitis and related problems. Occluding the skin increases the viable bacterial count on the skin. It was also found to skew the composition of the bacterial community growing on the skin. Only recently has high-throughput DNA sequencing enabled precise, quantitative measurement of the skin microbiome.

High-throughput DNA sequencing methods such as shotgun metagenomics allow precise identification of microbial strains and their quantities on the skin. These sequencing methods have been automated and costs have been reduced sufficiently in recent years to allow sequencing of the skin microbiome for large consumer populations. Quantitative, precise population-level data from consumer skin microbiome tests clarifies which microbes are associated with certain kinds of skin problems such as contact dermatitis. The common detrimental microbial species can also be targeted for removal. In this way, large-scale skin microbiome data enables precision engineering of the skin microbiome for a large consumer population.

Antimicrobials have been added to consumer products for decades, but some of these have recently been banned by the FDA for certain consumer applications. Triclosan and triclocarban were especially common broad-spectrum antimicrobials added to consumer products such as plastic toys, cutting boards, and hygiene products like antibacterial hand soaps. Some of those products included disposable personal care products composed of nonwoven fabrics, such as sanitary pads. However, the antimicrobials were found to be absorbed through the skin, persist in the environment, and exacerbate the spread of antibiotic resistance in bacteria found in the community. In 2016, the FDA banned triclosan and triclocarban from being marketed in over-the-counter consumer antimicrobial hand and body washes. Broad-spectrum small-molecule antimicrobials fell out of favor in consumer markets largely due to their negative side effects on the skin and gut microbiome, human endocrine system, and antibiotic stewardship. Although there is clear consumer demand for effective antimicrobials in consumer products, including disposable consumer products composed of nonwoven fabrics, acceptable options have become limited.

New, precision antimicrobials with minimal side effects are needed to address contact dermatitis and other conditions caused by disposable personal care products composed of nonwoven fabrics.

### 4. SUMMARY

Personal products, such as disposable personal care products composed of nonwoven fabrics or materials-such as sanitary pads, diapers, and face masks-occlude the skin, increase total bacterial counts, promote contact dermatitis, and give rise to consumer complaints such as diaper rash, maskne, sanitary pad rash, and incontinence-associated rash. Broad-spectrum, small-molecule antimicrobials previously used to reduce the problem have been banned in some cases of consumer use by the FDA or fallen out of favor with consumers.

In one aspect, disclosed is a composition comprising a phage combination attached to a polymeric fabric, wherein the phage combination comprises one or more sets of phages that infect one or more bacterial species, wherein the phages are selected from Myoviridae, Podoviridae and Siphoviridae, and wherein the polymeric fabric is selected from at least one of a polyhydroxybutyrate (PHB) or a polyethylene terephthalate (PET) nonwoven fabric.

A composition comprising a phage combination attached to a polymeric fabric, wherein the phage combination comprises one or more sets of Caudoviricetes phages, and wherein the polymeric fabric is a polyethylene terephthalate (PET) nonwoven fabric.

In one aspect, disclosed is a composition comprising a phage combination attached to a polymeric fabric, wherein the phage combination comprises at least Staphylococcus aureus infecting phages, and wherein the polymeric fabric is selected from at least one of a polyhydroxybutyrate (PHB) or a polyethylene terephthalate (PET) nonwoven fabric.

In another aspect, disclosed is a composition comprising a phage combination attached to a polymeric fabric, wherein the phage combination comprises at least Escherichia coli infecting phages, and wherein the polymeric fabric is selected from at least one of a polyhydroxybutyrate (PHB) or a polyethylene terephthalate (PET) nonwoven fabric.

In another aspect, disclosed is a composition comprising a phage combination attached to a polymeric fabric, wherein the phage combination comprises one or more sets of Caudoviricetes phages, and wherein the polymeric fabric is selected from the group consisting of a polylactic acid (PLA), polyester (PE), polycaprolactone (PCL), acrylics, olefins, polyhydroxyalkanoates (PHA), polyhydroxybutyrate (PHB), polyethylene terephthalate (PET), polypropylene (PP), and polypropylene carbonate (PPC) nonwoven fabric.

In one aspect, disclosed is a composition comprising a phage combination attached to a polymeric fabric, wherein the phage combination comprises one or more sets of phages that infect one or more bacterial species.

In accordance with any one of the embodiments, the phages are of the genus Pahexavirus or Rosenblumvirus. The phages can be tailed viruses. In some embodiments, the phages are at least one of Myoviridae or Podoviridae. In some embodiments, the phages are T4 myoviruses. In some embodiments, the phages are podoviruses.

In accordance with any one of the embodiments, the one or more sets of phages infect at least one common target bacterial species.

In accordance with any one of the embodiments, each set of phages comprises at least two, three, four, five, or more phages selected from one or more phage libraries, wherein the one or more phage libraries comprise phages from one or more genera.

In accordance with any one of the embodiments, the phage combination is noncovalently or covalently attached to the polymeric fabric. The polymeric fabric can be synthetic nonwoven fabric.

In accordance with any one of the embodiments, the phage combination is deposited onto at least one material that forms at least a portion of a personal care product. In some embodiments, the personal care product is selected from a group consisting of facemasks, sanitary pads, tampons, baby diapers, adult incontinence products, or cosmetic facial pads or wipes, fingernail wipes, or patient bathing hygiene textiles.

In accordance with any one of the embodiments, the bacterial species is commonly associated with contact dermatitis. In some embodiments, the bacterial species is selected from a skin microbiome biobank. In some embodiments, the bacterial species is from a skin microbiome isolated from a biological sample of a subject.

In accordance with any one of the embodiments, the one or more bacterial species are selected from a population of microorganisms comprising Staphylococcus aureus, Staphylococcus argenteus, Streptococcus pyogenes, Streptococcus pneumoniae, Escherichia coli, Enterococcus faecalis, Cutibacterium acnes, Corynebacterium tuberculostearicum, or combinations thereof.

In accordance with any one of the embodiments, the phage is an Escherichia coli infecting phage.

In accordance with any one of the embodiments, the phages are Staphylococcus aureus infecting phages.

In accordance with any one of the embodiments, the polymeric fabric further comprises a fabric is selected from the group consisting of a polylactic acid (PLA), polyester (PE), polycaprolactone (PCL), acrylics, olefins, polyhydroxyalkanoates (PHA), polyhydroxybutyrate (PHB), polyethylene terephthalate (PET), polypropylene (PP), and polypropylene carbonate (PPC) nonwoven fabric.

In one aspect, disclosed are methods of selecting the phage combination in accordance with any one of the embodiments.

In one aspect, disclosed are methods of attaching the phage combination in accordance with any one of the embodiments to a nonwoven polymeric fabric. In some embodiments, attaching the phage combination is achieved by permeating the phage capsid onto the surface of the nonwoven polymeric fabric. The phage capsid can be absorbed to the surface of the polymeric fabric via electrostatic charge. In some embodiments, the tail is exposed away from the fabric.

In some embodiments, attaching the phage combination is achieved by misting, chemical bonding, physical adsorption, bulk mixing, plasma-treated surface bonding, or a combination thereof.

In some embodiments, attaching the phage combination is achieved by plasma-treated surface bonding.

In one aspect, disclosed are methods of preparing a personal care product, the method comprising contacting a nonwoven polymeric fabric with a composition comprising a phage combination, wherein the phage combination comprises one or more sets of phages of Caudoviricetes phages that infect one or more bacterial species, and wherein the nonwoven polymeric fabric is selected from at least one of a polyhydroxybutyrate (PHB) or a polyethylene terephthalate (PET) nonwoven fabric.

In one aspect, disclosed are methods of preparing a personal care product, the method comprising (a) contacting a nonwoven polymeric fabric with a plasma composition to generate a plasma-treated nonwoven fabric, wherein the nonwoven polymeric fabric is selected from at least one of a polyhydroxybutyrate (PHB) or a polyethylene terephthalate (PET) nonwoven fabric; (b) immobilizing a combination of phages comprising at least one or more sets of Caudoviricetes phages onto the nonwoven polymeric fabric, wherein the phage combination comprises one or more sets of phages that infect one or more bacterial species; and (c) incorporating the nonwoven polymeric fabric into the personal care product.

In one aspect, disclosed is a nonwoven polymeric fabric comprising a phage combination attached to the polymeric fabric, wherein the phage combination comprises one or more sets of Caudoviricetes phages that infect one or more bacterial species, and wherein the polymeric fabric is selected from a polylactic acid (PLA), polyester (PE), acrylics, olefins, polyhydroxyalkanoates (PHA), polyhydroxybutyrate (PHB), polyethylene terephthalate (PET), polypropylene (PP) and polypropylene carbonate (PPC) nonwoven fabric.

In one aspect, disclosed are methods for the manufacturing of a personal care product for reducing contact dermatitis, reducing skin infection and/or reducing body odor, the personal care product comprising a nonwoven polymeric fabric, wherein the nonwoven polymeric fabric comprises a phage combination attached to the nonwoven fabric selected from at least one of a polyhydroxybutyrate (PHB) or a polyethylene terephthalate (PET) nonwoven fabric, and wherein the phage combination comprises one or more sets of Caudoviricetes phages that infect one or more bacterial species. In some embodiments, the personal care product is selected from a group consisting of facemasks, sanitary pads, tampons, baby diapers, adult incontinence products, or cosmetic facial pads or wipes, fingernail wipes, or patient bathing hygiene textiles.

In accordance with any one of the embodiments, the phage of the method, or the phage of the nonwoven polymeric fabric, or the phage of the personal care product, wherein the phage comprises a nucleic acid sequence having at least 80%, 85%, 90%, 95%, 96%, 97%, 98%, or 99% identity with any one of SEQ ID NOs.: 1-132.

In accordance with any one of the embodiments, the phage of the method, or the phage of the nonwoven polymeric fabric, or the phage of the personal care product, wherein the phage comprises a nucleic acid sequence having at least 95% identity with any one of SEQ ID NOs.: 1-132.

In accordance with any one of the embodiments, the phage of the method, or the phage of the nonwoven polymeric fabric, or the phage of the personal care product, wherein the phage comprises a nucleic acid sequence having a sequence of any one of SEQ ID NOs.: 1-132.

Described herein are compositions of bacteriophage mixture and methods of using bacteriophages (phages) as antimicrobial for consumer products, such as disposable consumer products composed of one or more nonwoven fabrics.

Accordingly, in one aspect, described herein is a composition of one or more phages that infect bacteria on the surface of the skin that are associated with contact dermatitis. In one aspect, described herein is a method for selecting one or more phage combinations to reduce the concentration of bacteria commonly associated with contact dermatitis. In one aspect, described herein is a method for attaching one or more phages combinations to nonwoven fabrics that are incorporated into disposable personal care products.

These phages infect bacterial species including members of Gram-positive and Gram-negative bacteria. In certain embodiments, the phages infect one or more species including, but are not limited to, members of the Bacillota, Proteobacteria, and Actinobacteria phyla. In certain embodiments, the phages infect one or more species including, but are not limited to, members of the Staphylococcaceae, Streptococcaceae, Enterobacteriaceae, Enterococcaceae, Propionibacteriaceae, and Corynebacteriaceae families. In some embodiments, the phages infect one or more species including, but are not limited to, members of the Staphylococcus, Streptococcus, Escherichia, Enterococcus, Cutibacterium, and Corynebacterium genera. For instance, the phages can infect bacterial species such as Staphylococcus aureus, Staphylococcus argenteus, Streptococcus pyogenes, Streptococcus pneumoniae, Escherichia coli, Enterococcus faecalis, Cutibacterium acnes, and/or Corynebacterium tuberculostearicum. In certain embodiments, the phage infects one or more fungi or viruses (including dsDNA virus). In some embodiments, the phage infects Malassezia yeast. In certain embodiments, the phage infects mites (e.g., Demodex mites). In some embodiments, the phage infects a population of microorganisms including bacteria, fungi, viruses, and/or mites.

In the composition or method of the preceding embodiment, the phages are noncovalently attached to surface-active nonwoven fabrics integrated into personal care products, though these can be covalently attached, as well. In some embodiments, the personal care product is disposable, non-disposable, or biodegradable. The phages are attached so that the capsid adsorbs to the surface of the nonwoven fabric via electrostatic charge and the tail is exposed away from the surface of the fabric. When the nonwoven fabric of the personal care product comes into contact with the skin, then the exposed phage tail can initiate infection of any susceptible bacteria on the skin.

In the composition or method of any proceeding embodiment, the combination of multiple phages are attached to plasma surface-treated synthetic nonwoven polymer fabrics including polylactic acid (PLA), polyester (PE), acrylics, olefins, polyhydroxyalkanoate (PHA), polyethylene terephthalate (PET), polypropylene (PP) and polypropylene carbonate (PPC). The surface-treated nonwoven fabric is incorporated in a disposable personal care product that is in close contact with the skin, such as a facemask, sanitary pad, tampon, baby diaper, or adult incontinence products.

In the composition or method of any preceding embodiment, the phage combination is attached to natural polymer fabric. Non-limiting examples of natural polymer fabrics are silk, wool, DNA, cellulose, and proteins.

In the composition or method of any preceding embodiment, target bacterial strains from the species Staphylococcus aureus, Staphylococcus argenteus, Streptococcus pyogenes, Streptococcus pneumoniae, Escherichia coli, Enterococcus faecalis, Cutibacterium acnes, and/or Corynebacterium tuberculostearicum are identified from a large shotgun metagenomic dataset of skin microbiome samples collected from contact dermatitis patients. In some embodiments, phages infecting those strains are isolated from environmental samples. In some embodiments, phage isolates are amplified on the target bacterial strain. DNA extractions from the isolates are sequenced to screen the phage genomes for genes related to antibiotic resistance, toxicity, lysogeny, or transduction. Screened phages are tested for host range and combined to maximize host range. In certain embodiments, sets of phages that infect the same host species are tested in pairs for preventing bacterial regrowth in broth culture. In certain embodiments, pairs that prevent bacterial regrowth are selected. In some embodiments, more than two phages or two pairs of phages that prevent bacterial regrowth are selected.

In the composition or method of any preceding embodiment, phages are concentrated in a low-salt phage buffer before attaching to the nonwoven fabric. In some embodiments, plasma surface treatment is applied to the nonwoven fabric. In some embodiments, the phage buffer is misted onto the surface-treated nonwoven fabric and dried at room temperature. In some embodiments, the nonwoven fabric is incorporated into the disposable personal care product.

### 5. BRIEF DESCRIPTION OF THE SEVERAL VIEWS OF THE DRAWINGS

These and other features, aspects, and advantages of the present invention will become better understood with regard to the following description, and accompanying drawings.

FIG. 1 depicts a phage attached to a surface-activated polymer fabric via the negatively charged capsid and the tail is free for binding to a phage receptor presented on a target host bacterial species, which lives in or on the skin surface of a subject. Legend: (1) skin surface; (2) bacterial species targeted for removal; (3) phage receptor on target host bacterial species, including but not limited to Staphylococcus aureus, a coliform, or Cutibacterium acnes; (4) untargeted skin microbial commensal; (5) phage; (6) negative dipole moment of bacteriophage capsid; (7) positive charge of activated surface; (8) nonwoven-fiber activated surface functionalized with immobilized phage; (9) podovirus tailed phage, a distinct phage morphotype from a myovirus tailed phage.

FIGS. 2A-2C show phages can be attached to plasma-treated PET nonwoven fabric and inhibit bacterial growth. Shown are phage inhibition of Escherichia coli strain 11303 growth on agar plates.

FIGS. 3A-3C show phages can be attached to plasma-treated PET nonwoven fabric and inhibit bacterial growth. Shown are phage inhibition of Escherichia coli strain 11303 growth on agar plates.

FIGS. 4A-4C show phages can be attached to plasma-treated PET nonwoven fabric and inhibit bacterial growth. Shown are phage inhibition of Staphylococcus aureus strain 100311 growth on agar plates.

FIGS. 5A-5C show phages can be attached to plasma-treated PHB nonwoven fabric and inhibit bacterial growth. Shown are phage inhibition of Staphylococcus aureus strain 100311 growth on agar plates.

FIG. 6 shows effect of T4 myovirus phage attached to plasma-treated PET and PHB nonwoven fibers on Escherichia coli strain 11303.

FIG. 7 shows effect of S. aureus podovirus Rosenblumvirus P00474 phages attached to plasma-treated PET and PHB nonwoven fibers on Staphylococcus aureus strain 100311.

FIG. 8 illustrates enablement of inline phage treatment of nonwoven fabrics during manufacturing.

### 6. DETAILED DESCRIPTION

### 6.1. Bacteriophages

Phages are viruses that infect bacteria. As used herein, the terms “phage” or “bacteriophage” are used interchangeably. Phages were independently discovered in 1915 and 1917 and were used early on in bacteriophage therapy to treat acute bacterial infections. Phages are ubiquitous in the environment, and are a significant component of the skin microbiome. Although they are effective antimicrobials under specific conditions, they are precise in which bacterial species and strains they kill. Broad-spectrum, small-molecule antibiotics have been preferred over phages partly because the data for targeted use of phages has been absent. Only broad-spectrum antimicrobials were practical. Precise, quantitative and population-scale skin microbiome data recast phages as a candidate for precision antimicrobials.

In various embodiments, the phages described throughout are of the class Caudoviricetes. In some embodiments, the phages belong to genus Pahexavirus. In some embodiments, the phages belong to genus Rosenblumvirus. In some embodiments, the phages are selected from Myoviridae, Siphoviridae, Podovirdae, or Rountreeviridae. In some embodiments, the phages are Myoviridae having long tails that are contractile. In some embodiments, the phages are Podoviridae having short noncontractile tails. In some embodiments, the phages are Siphoviridae having long noncontractile tails. In some embodiments, the phages have a nucleic acid sequence with at least 80%, 85%, 90%, 95%, 96%, 97%, 98%, or 99% identity with any one of SEQ ID NOs.: 1-132. In some embodiments, the phages has a nucleic acid sequence having at least 95% identity with any one of SEQ ID NOs.: 1-132. In some embodiments, the phages have a nucleic acid of any one of SEQ ID NOs.: 1-132.

In various embodiments, the phage infects a population of microorganisms including bacteria, fungi, viruses, and/or mites. In some embodiments, the population of microorganisms commonly found living on or in the skin sample obtained from a subject. In some embodiments, the population of microorganisms comprises one or more microorganisms associated with acne. In some embodiments, the population of microorganisms comprises one or more microorganisms associated with contact dermatitis, diaper rash, maskne, sanitary pad rash, incontinence-associated rash, inflammation, redness, eczema, rosacea, enlarged hair follicle pore size, rough skin texture, increased trans-epidermal water loss, skin discoloration, or disproportionate elasticity of a stratum corneum and underlying dermis. In some embodiments, the population of microorganisms comprises one or more microorganisms associated with aged skin. In some embodiments, the population of microorganisms comprises a Cutibacterium acnes bacterium. In some embodiments, the population of microorganisms comprises a Staphylococcus aureus bacterium. In some embodiments, the population of microorganisms comprises a Corynebacterium bacterium. In some embodiments, the population of microorganisms comprises a Malassezia yeast. In some embodiments, the population of microorganisms comprises Staphylococcus aureus, Staphylococcus argenteus, Streptococcus pyogenes, Streptococcus pneumoniae, Escherichia coli, Enterococcus faecalis, Cutibacterium acnes, and/or Corynebacterium tuberculostearicum.

Phages can be attached to nonwoven fabrics. They are attached by activating the surface of the fabric with corona or plasma treatment. Surface activation imparts a charge to the nonwoven fabric, to which phages can be attached noncovalently by electrostatic adsorption. Phages are composed of a capsid that carries the genetic material necessary for infection and a tail that interacts with the bacterial host and facilitates infection. The phage capsid is charged and can be attached to the nonwoven fabric so that the phage tail is exposed, facing away from the surface of the fabric to facilitate infection. Nonwoven fabrics are integrated in disposable personal care products where the products come into contact with the skin. In this orientation, phage tails can infect bacteria on the skin when the nonwoven fabric on the surface of disposable consumer products comes into contact with the skin.

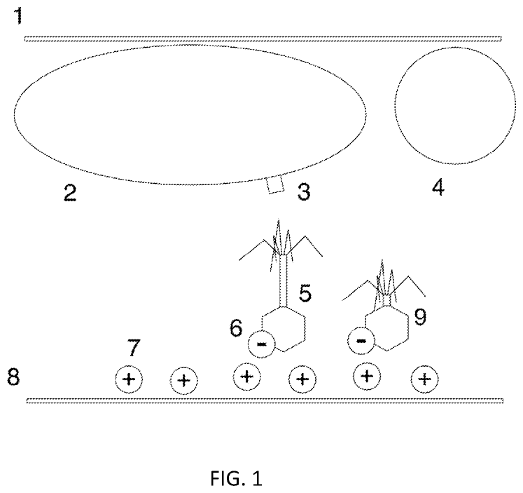

FIG. 1 illustrates the use of a composition comprising phages as described herein for use in a personal care product. A subject's skin harbors a variety of bacterial species living in or on the skin surface (1) commensally. Phage targets a specific type of bacteria (2) by recognizing the phage receptor (3) presented on a target host bacterial species. It is noted that the nontargeted skin microbial commensal (4) remains uninterrupted. The phage (5) presents a negatively charged capsid (6), which is capable of binding to the positively charged (7) and activated polymer fabric surface (8). The polymer fabric is a nonwoven fiber. The phage (5) is a myovirus tailed phage. The phage (9) is a podovirus tailed phage, a distinct phage morphotype from a myovirus tailed phage. Upon phage attachment, the polymer fabric (e.g., surface activated nonwoven-fiber) (8) is functionalized with the immobilized phage (5) or (9). This particular method of attachment or deposition of the phages allows the phages tails to be presented outward on the fabric since the charged capsid binds the activated polymer fabric surface, allowing the phages to be more effective in bacterial infection.

In some embodiments, the composition comprises a bacteriophage for fungal virus formulation comprising a set of nucleic acid sequence reads extracted from the genomes of a plurality (e.g., 1, 2, 3, 4, 5, or more) of bacteriophages or fungal viruses and at least one virus is capable of lysing a microorganism of the population of microorganisms identified as living in or on the skin of a subject. Methods and compositions for identifying such microorganisms and bacteriophage targeting the microorganisms are described in WO2022198091A1, which is incorporated by reference herein in its entirety.

### 6.2. Composition

Described herein are compositions comprising phages attached to fabrics. In certain embodiments, the phages are attached to fabrics, such as polymer fabrics, and incorporated into personal care products. The polymer fabrics can be surface-active nonwoven fabrics. The surface-active nonwoven fabrics can be plasma-treated. The fabrics can be synthetic, artificial, or natural.

In some embodiments, the polymer fabric comprises a degradable or non-degradable polymer. In some embodiments, the polymer fabric comprises polylactic acid, polyhydroxyalkanoate or polycaprolactone. In some embodiments, the polymer fabric is nonwoven fabric.

In some embodiments, the composition comprises phages noncovalently attached to surface-active nonwoven fabrics integrated into disposable personal care products. In some embodiments, the composition comprises one or more sets of phage or fungal virus DNA sequences isolated from the skin sample of a subject, and optionally incorporated onto the surface of a personal care product. In some embodiments, the composition comprises one or more sets of phage DNA isolated from a first bacteriophage and a second bacteriophage. In some embodiments, the first bacteriophage and the second bacteriophage are capable of lysing a microorganism of the population of microorganisms. In some embodiments, the first bacteriophage and second bacteriophage individually or collectively prevent resistance or regrowth of the microorganism in vitro.

In some embodiments, the polymer surface is modified to facilitate phage attachment using plasma treatment alone, plasma treatment followed by activation by 1-ethyl-3-(3-(dimethylamino) propyl) carbodiimide hydrochloride (EDC) and N-hydroxysulfosuccinimide (sulfo-NHS), plasma-initiated acrylic acid grafting, or plasma-initiated acrylic acid grafting with activation by EDC and sulfo-NHS.

In some embodiments, the first bacteriophage and the second bacteriophage are capable of killing, preventing resistance or regrowth of a population of microorganisms including, but is not limited to, Staphylococcus aureus, Staphylococcus argenteus, Streptococcus pyogenes, Streptococcus pneumoniae, Escherichia coli, Enterococcus faecalis, Cutibacterium acnes, and/or Corynebacterium tuberculostearicum.

In some embodiments, the first bacteriophage and the second bacteriophage have a different host range. The different host range may comprise at least two C. acnes strains, or the different host range comprise at least one C. acnes strain and at least one C. namnatense strain. In some embodiments, the different host range does not comprise a C. granulosum strain. The first bacteriophage and the second bacteriophage may be lytic bacteriophage. In some embodiments, the bacteriophage or fungal virus formulation is part of a cosmetic formulation. The cosmetic formulation may also comprise one or more liposomes comprising the first bacteriophage and the second bacteriophage. The cosmetic formulation may further comprise an anti-aging component.

Phage isolates used in formulating phage-functional activated polymer fabric (e.g., nonwoven fabric) surfaces are provided in Table 1.

TABLE 1
Phage isolates for use in formulating phage-functional activated nonwoven fabric surfaces

| Phage | Host | Phage | Host |
|---|---|---|---|
| P00031 | Cutibacterium acnes sp. 100085 | P00060 | Cutibacterium acnes sp. 100119 |
| P00032 | Cutibacterium acnes sp. 100085 | P00061 | Cutibacterium acnes sp. 100119 |
| P00033 | Cutibacterium acnes sp. 100085 | P00062 | Cutibacterium acnes sp. 100124 |
| P00034 | Cutibacterium acnes sp. 100085 | P00063 | Cutibacterium acnes sp. 100124 |
| P00035 | Cutibacterium acnes sp. 100088 | P00064 | Cutibacterium acnes sp. 100124 |
| P00036 | Cutibacterium acnes sp. 100088 | P00065 | Cutibacterium acnes sp. 100124 |
| P00037 | Cutibacterium acnes sp. 100088 | P00066 | Cutibacterium acnes sp. 100126 |
| P00038 | Cutibacterium acnes sp. 100088 | P00067 | Cutibacterium acnes sp. 100126 |
| P00039 | Cutibacterium acnes sp. 100088 | P00068 | Cutibacterium acnes sp. 100126 |
| P00040 | Cutibacterium acnes sp. 100088 | P00069 | Cutibacterium acnes sp. 100126 |
| P00041 | Cutibacterium acnes sp. 100111 | P00070 | Cutibacterium acnes sp. 100132 |
| P00042 | Cutibacterium acnes sp. 100111 | P00071 | Cutibacterium acnes sp. 100132 |
| P00043 | Cutibacterium acnes sp. 100111 | P00072 | Cutibacterium acnes sp. 100132 |
| P00044 | Cutibacterium acnes sp. 100111 | P00073 | Cutibacterium acnes sp. 100132 |
| P00045 | Cutibacterium acnes sp. 100111 | P00074 | Cutibacterium acnes sp. 100132 |
| P00046 | Cutibacterium acnes sp. 100111 | P00075 | Cutibacterium acnes sp. 100144 |
| P00047 | Cutibacterium acnes sp. 100111 | P00076 | Cutibacterium acnes sp. 100144 |
| P00048 | Cutibacterium acnes sp. 100111 | P00077 | Cutibacterium acnes sp. 100144 |
| P00049 | Cutibacterium acnes sp. 100111 | P00078 | Cutibacterium acnes sp. 100144 |
| P00050 | Cutibacterium acnes sp. 100111 | P00079 | Cutibacterium acnes sp. 100144 |
| P00051 | Cutibacterium acnes sp. 100111 | P00080 | Cutibacterium acnes sp. 100144 |
| P00052 | Cutibacterium acnes sp. 100111 | P00081 | Cutibacterium acnes sp. 100144 |
| P00053 | Cutibacterium acnes sp. 100111 | P00082 | Cutibacterium acnes sp. 100144 |
| P00054 | Cutibacterium acnes sp. 100111 | P00083 | Cutibacterium acnes sp. 100144 |
| P00055 | Cutibacterium acnes sp. 100111 | P00084 | Cutibacterium acnes sp. 100144 |
| P00056 | Cutibacterium acnes sp. 100111 | P00085 | Cutibacterium acnes sp. 100146 |
| P00057 | Cutibacterium acnes sp. 100111 | P00086 | Cutibacterium acnes sp. 100146 |
| P00058 | Cutibacterium acnes sp. 100111 | P00087 | Cutibacterium acnes sp. 100146 |
| P00059 | Cutibacterium acnes sp. 100119 | P00088 | Cutibacterium acnes sp. 100146 |
| P00089 | Cutibacterium acnes sp. 100146 | P00202 | Enterococcus faecalis sp. PH100667 |
| P00090 | Cutibacterium acnes sp. 100146 | P00223 | Enterococcus faecalis sp. PH100667 |
| P00091 | Cutibacterium acnes sp. 100146 | P00224 | Enterococcus faecalis sp. PH100667 |
| P00092 | Cutibacterium acnes sp. 100146 | P00235 | Enterococcus faecalis sp. PH100667 |
| P00093 | Cutibacterium acnes sp. 100146 | P00391 | Staphylococcus argenteus sp. PH100253 |
| P00094 | Cutibacterium acnes sp. 100146 | P00392 | Staphylococcus argenteus sp. PH100253 |
| P00095 | Cutibacterium acnes sp. 100146 | P00393 | Staphylococcus argenteus sp. PH100253 |
| P00096 | Cutibacterium acnes sp. 100146 | P00394 | Staphylococcus argenteus sp. PH100253 |
| P00001 | Cutibacterium namnetense sp. 100047 | P00395 | Staphylococcus argenteus sp. PH100253 |
| P00362 | Enterococcus faecalis sp. PH100347 | P00396 | Staphylococcus argenteus sp. PH100253 |
| P00363 | Enterococcus faecalis sp. PH100347 | P00406 | Staphylococcus argenteus sp. PH100286 |
| P00364 | Enterococcus faecalis sp. PH100347 | P00409 | Staphylococcus argenteus sp. PH100286 |
| P00365 | Enterococcus faecalis sp. PH100347 | P00411 | Staphylococcus argenteus sp. PH100286 |
| P00218 | Enterococcus faecalis sp. PH100665 | P00248 | Staphylococcus argenteus sp. PH100315 |
| P00220 | Enterococcus faecalis sp. PH100665 | P00249 | Staphylococcus argenteus sp. PH100315 |
| P00221 | Enterococcus faecalis sp. PH100665 | P00250 | Staphylococcus argenteus sp. PH100315 |
| P00222 | Enterococcus faecalis sp. PH100665 | P00251 | Staphylococcus argenteus sp. PH100315 |
| P00242 | Enterococcus faecalis sp. PH100665 | P00252 | Staphylococcus argenteus sp. PH100315 |
| P00245 | Enterococcus faecalis sp. PH100665 | P00253 | Staphylococcus argenteus sp. PH100315 |
| P00246 | Enterococcus faecalis sp. PH100665 | P00254 | Staphylococcus argenteus sp. PH100315 |
| P00260 | Enterococcus faecalis sp. PH100665 | P00371 | Staphylococcus argenteus sp. PH100351 |
| P00261 | Enterococcus faecalis sp. PH100665 | P00372 | Staphylococcus argenteus sp. PH100351 |
| P00262 | Enterococcus faecalis sp. PH100665 | P00373 | Staphylococcus argenteus sp. PH100351 |
| P00263 | Enterococcus faecalis sp. PH100665 | P00374 | Staphylococcus argenteus sp. PH100351 |
| P00264 | Enterococcus faecalis sp. PH100665 | P00375 | Staphylococcus argenteus sp. PH100351 |
| P00198 | Enterococcus faecalis sp. PH100667 | P00371 | Staphylococcus argenteus sp. PH100352 |
| P00199 | Enterococcus faecalis sp. PH100667 | P00372 | Staphylococcus argenteus sp. PH100352 |
| P00200 | Enterococcus faecalis sp. PH100667 | P00373 | Staphylococcus argenteus sp. PH100352 |
| P00201 | Enterococcus faecalis sp. PH100667 | P00374 | Staphylococcus argenteus sp. PH100352 |
| P00375 | Staphylococcus argenteus sp. PH100352 | P00316 | Staphylococcus aureus sp. PH100342 |
| P00283 | Staphylococcus argenteus sp. PH100533 | P00317 | Staphylococcus aureus sp. PH100342 |
| P00285 | Staphylococcus argenteus sp. PH100533 | P00318 | Staphylococcus aureus sp. PH100342 |
| P00286 | Staphylococcus argenteus sp. PH100533 | P00319 | Staphylococcus aureus sp. PH100342 |
| P00287 | Staphylococcus argenteus sp. PH100533 | P00320 | Staphylococcus aureus sp. PH100342 |
| P00289 | Staphylococcus argenteus sp. PH100533 | P00321 | Staphylococcus aureus sp. PH100342 |
| P00291 | Staphylococcus argenteus sp. PH100533 | P00275 | Staphylococcus aureus sp. PH100669 |
| P00322 | Staphylococcus argenteus sp. PH100533 | P00276 | Staphylococcus aureus sp. PH100669 |
| P00323 | Staphylococcus argenteus sp. PH100533 | P00277 | Staphylococcus aureus sp. PH100669 |
| P00324 | Staphylococcus argenteus sp. PH100533 | P00278 | Staphylococcus aureus sp. PH100669 |
| P00325 | Staphylococcus argenteus sp. PH100533 | P00279 | Staphylococcus aureus sp. PH100669 |
| P00366 | Staphylococcus argenteus sp. PH100533 | P00386 | Staphylococcus aureus sp. PH100669 |
| P00367 | Staphylococcus argenteus sp. PH100533 | P00387 | Staphylococcus aureus sp. PH100669 |
| P00369 | Staphylococcus argenteus sp. PH100533 | | |
| P00370 | Staphylococcus argenteus sp. PH100533 | | |
| P00312 | Staphylococcus aureus sp. PH100339 | | |
| P00313 | Staphylococcus aureus sp. PH100339 | | |
| P00314 | Staphylococcus aureus sp. PH100339 | | |
| P00315 | Staphylococcus aureus sp. PH100339 | | |
| P00316 | Staphylococcus aureus sp. PH100339 | | |
| P00317 | Staphylococcus aureus sp. PH100339 | | |
| P00318 | Staphylococcus aureus sp. PH100339 | | |
| P00319 | Staphylococcus aureus sp. PH100339 | | |
| P00320 | Staphylococcus aureus sp. PH100339 | | |
| P00321 | Staphylococcus aureus sp. PH100339 | | |
| P00312 | Staphylococcus aureus sp. PH100342 | | |
| P00313 | Staphylococcus aureus sp. PH100342 | | |
| P00314 | Staphylococcus aureus sp. PH100342 | | |
| P00315 | Staphylococcus aureus sp. PH100342 | | |

In some embodiments, phage isolates used in formulating phage-functional activated polymer fabric (e.g., nonwoven fabric) surface have a nucleic acid sequence of at least 30%, 40%, 50%, 60%, 70%, 80%, 90%, 95%, or 100% similarity with any one of the sequences listed in Table 2 or any one of SEQ ID NOs.: 1-132.

TABLE 2
Informal Sequence Listing

| Phage | Contigs |
|---|---|
| P00001 | SEQ ID NO.: 1; >353 len: 12656 cycle: O |
| | CATCGGCGCCCACAAAGACTCCTTTACTTTCGCCTACGGTGCCACCACCAAGCAACCCGGAGCATCCAAGGCACAATTGAAA |
| | GTCTTCTGCCAAGTCTCTGGCACCGAAGACAAGCCGCAGGTAGAGGACATGTCCATCAACCCTTCACGCTAACCCGAAAGAC |
| | AACACACCCATTATGCCTCTCATGTCCACCTACATGGCTAATGCGGGCCTGTCCGACATCATGCACATCCTCCATACCGCCGG |
| | GGGAGCCTGCCGGACAGGCACCGACATCGCCGCACGAATCAACACACTCCACCCAGAATACAATCTCCAAGCCACCACCGA |
| | CCAGCTCATCGACATCGAAAACCGCATCGACACTATTCGCAAAAACATTCAAAAAGAAATCAAGAAACACCCGTGAACATC |
| | ACCGACCACACCACCCCAGACCTGCACTCCTACGAAACGTTCTTCACCAGCCTCGCATGGATCCAAGGCGGCATCATCACAT |
| | GGATGTACGCAACCGGCACCCCACACAAGGCAGCCCTCGCCATTATTGCCGCATGCGCCCTCGCCACCCTCCTGGGTGCATC |
| | AACACTCACATACAAGCCCAAAGACAGCAAATGATCACAACACCCATACTGATCGCTGAAACCCTCGCCATAATTATTCTCG |
| | CAGTAGCACTCGCCCACAACAACAACCAGTAACCCACCCTTAAGGAGCACACATCTCATGGATGAGCCCACCCGCATGTACA |
| | CCGACCCCAACACCGGTGCCCGAAAAGAATTGAAACTCTGCAGGCTCTCCCTCATCGACCCCGCATCCCTTCACGCTCTCGGC |
| | GAAGTAGCAGGCTACGGTGCCACCAAATACGGCGACAACAACTGGACCGGAGGATACCCGTGGAGCCACAGTGTCGACGCC |
| | CTCTACCGCCACCTACTATCATGGCAGCAAGGCAACAACCTCGACCGCGAATCCGGGCTACCCCATTTGGCACACGCCGCCT |
| | GGCACTGCCTCGCACTCCTCGCATACCAGCAACACAATGCCGGGGTAGACACCCGCAACCCATGGAACACCCACAAAGGCG |
| | ACCAGTAATGCCTCTAGCACAATACCCGAAAACCATCCACCATCCAGGCCACATCTCCTACTCATCACTGTCACAGTGGGCCG |
| | AATGCGGAGAAAAATGGCGCCTATCCCACGGCTACCACACCCAACACCACACCTGGTACGCCACCATCGCCGGAAGCGCCAT |
| | ACACCACATCACCGAACAATACGACCTACACCTGTACAATCCCGCCGAATACCCTGCACTGCCAGACAAACTCTCATCCTTCA |
| | AAAATGTTTTCGACACCCAAGTCGCCCTCGCCGAATCCGAAGGCACAGAAATCAAACCCTCCGGCAGAATATGCAAAAACAT |
| | GTGCGAGTCGGGCGGGCCACACAAGAAGGACTACGACTGGTGGATGGTTTACGGCCCCACCTTTGTGGACCGCTGGAAAACA |
| | TGGAGGCGCAACCACCCAGAATACATCACCGCAATCCTGGGCGGTAAACCAGGCATCGAATACCCGGTAGAAACCATCCTCG |
| | ACGATGACACAAAAATAGTCGGATACATCGACCGGGTATTCACAGATACAAATACGGGTGAAACCTTCATTTTGGACCTCAA |
| | AACCGGCCGTCTACCCGCCGACAGTATGCAGCTGCACACATACCGGTACATGCTCAACCAACACGGCAACGATGTGACAAAA |
| | GGCATGTTTTGGACACCCGCCACCACTAAGGACGATAAGCTATCCTCTGAGCATGGCACCGCTACAGACTTGATTGATCTTGA |
| | CAACAACACCTACCGGCATGTATCATCCATGTACAGTCAAGCAATGAAAGGAATCAGCGAAGGCATCTTCGTCCCACACGTC |
| | ACAACACTCTGTAAAGGATGCCCCGTACGAGACGCCTGCTGGGCCGTCAATGGCAAAAACTCGTACAGATACCCGGTAGAAT |
| | CCACCGTACAGCCACCCCAAACAGACAATAAAGAAAAGGACACCTAATGACCGATAAAGACAAGATCGACAATGATCGACT |
| | CACGATCACACTCAAATACGGTGGCGACTATGCTGCCCCATGGACCGTCATTAGGGGAGACACAGCAGAGCAGACAAAACA |
| | GGCCATCATCGACCTGCTAGGAGGATTGAAAGACGAAACCGTATCGAAAGACTGGGACCTTGCAACACTCGTAGCGAGCGC |
| | ATCCATCATCCTCCAAGACCGATACAATCAGGCCACCAAAGACTACGTAAACAAAATCGCCAATCAGGAAAACGACATCATA |
| | ATTGACCGGATCAACAAAGCCACCAGCAAGGCAGAGCTCGCCGACCTTCTGAAACAGTACAAGAAGATCATCACCAGTAAC |
| | AGGGACGTGTCTGAGGCTTTCCGCAGCAAACGCAACAGCCTCACCCGATAAAAACCGACACAAACCAACAAAACCAAACAT |
| | AAACAGTAAAAGGAAAACAGTTATGGGACTCGCAAACTACCGCAACAACAGCAACAGCACCTTCTTCAACCCCTCCCGAAA |
| | CCAGGACGCCACCGCCATCGCCTTCAAAGTCCACGATGTAGAACACAACACTGAAGGCTACGGTGGACAAACAGCAGACCG |
| | CATCTACGCTGATGTCACAATCTTCCACACCCTAGGCGATCTCAACAACGGCACCCCAGAAACCATCCCCAACGCTATTATTG |
| | AGAAAGCGCGCGGCAACAACGACCGCCCACACTCCATGATCCGCGATCTAGAAGCCTATCTTGGCGAGGAGCAGGCCTTCAA |
| | ACTCGCCACCGTACGCACCAAAAACGGCTTCAACGCGGTAATCCTCAAACCCCTTGATGACGCCATCTACGATAAGGTTGCC |
| | GCATACGTAGACAAGCGTGATAACGGCCAGCTAGACGACACCACAGCCTCAACTGATGCTGATATCGACATCGACTCCATCT |
| | GACCACCAAAACACAACCAACCAATAGATAGATAAGGTCCCGATGCTCTCTCTCCAACGATCCTTCGAGAGAGCCTCCCAAA |
| | CCGCAGCCGAGCTGCCCCGCATACCCCAGCTAGAACCCCTCTACCGCAACCAGGACATGCACATCCACAAAGGGGATCTCAC |
| | CATGATTGCGGGGCGCTCCGGCAGCCAAAAATCCGGGCTAGCCATGTTCATCACAGCGATGCTCAACCAGCCCGCCCTCTAC |
| | ATATCAGGGGACATGACACCCTGGGAGGCCTCCACACGAATCATCTCACTCAACACCCAACACACCACCACACAGATACAAC |
| | AAAACATCGACGACTACGGGCCAGAATACTATCGAGACAGCATCCACCACGGCCAACACATCACATTCTCATTCCAGTCACC |
| | CATCACATGGACCGACATCACCACGGAATTGCAAGCCTACATGGAAATGTGGAACACCTTCCCACCCATCATTGTTATCGAC |
| | AACCTGATGGACATCCAAGACTGCGAGAGTGACTATCAGGCACAGCAAGAAGCCATGCAATGGATCACAGCATTAGGTAGG |
| | GATACTGGCTCCACCATCATCGTCACACACCATGCAACCGACAAAACCGGCTCCGACATCGAACACCCGCCAGCACGGCGAG |
| | AAATCAAAAACGGACTCTCAGAAAAACCACAACTCATCCTCGGAGTCTCATTGTATGGTGGCGAGGATAATGGCAACGGCCT |
| | CACGATACCGGCAGAGGCACGCATCGCCGTACTCAAACAGCGCACCGGCAAATCCGGTCCTGACGGAACCCGATACGAACG |
| | ACTCAAAGCCTACCCTGAATACACATTCTTCGGGCCCCTCGCCGAAAAACAGCCCTGGAACATGACCACAACACACAAAGGA |
| | CTCTAAAAAACTATGGCCACACAGCAGGCACGCAACCGGCGGGCCGGAGCCGAATGGGAAACACGACTCCTCCACCAGCTA |
| | CGCGACACCGGCCACGATATAGAACGCCTCCACCTCAACGGCCGCGAAGACGAAGGAGACCTCATCCTTCAAACCGGAAAC |
| | AAAACCTACGTTATCGAAGCGAAAGCCGGGCAGCCCCATCTCGCCGAATTCGTGAAACAAGCCAGCCGGGAGGCACGCAAC |
| | TACGAAACCCACCGAAACCGAGAAAGCCAGTCCACCATCGGACTCGTCGTCATGAAACAGCGCAACAAACCCTGGAGCGAA |
| | GCCTATGTGGTATCAACCCTCAACGAGCTCCTCCCACACCTCTGACACCTGCCGCCTCCTCGACACCTACCGGATACGGTACA |
| | ACCCATCCAAGAACGAGCAACACATCCTCTGCCCGTTCCACGACGACCACCAGCCCTCCATGAGCATCAACCTCGACAAGGG |
| | TGTCTGGTACTGCCACACATGCGGTGTCGGAGGCGGACTTGCCAAGCTACAACAACGATTAGAAGAAGAAAACCCGAATGTA |
| | CGACAGTATACGCCCATACAACATTGCGGAACGCCGCCGAATCCAGAAAGCCTCAGCCCGCTACGAAACCCACCTCGAAAAC |
| | ATACTCGACCTGCTCTCAGCAAGAGGCATCAGCGAAGAAACAGCCCGGCTCCACCACCTTGGATACATCGACAATGACCCCA |
| | TACCAGGCCACGAAGACTACAACCAGTGCATCACCATCCCCTACATGTACCCCGTCTGGGGCGGCCCAGCCGAAATACGAAA |
| | AATGCGTTTCCGCTGCTCACTCCAGCATGATTGCAAAACCCACAACCACCCCAAATATCTAACCCCGGCAGGGGACACAGGC |
| | TCCATCTACAACATGGCCGCCATGGCCAACCCGGCAGCCGAAATGCACATTTGCGAAGGCGAATCCGACTCCATGATCCTCG |
| | AACAATGCGGATGGTCGGCCGTAGCCCTACCCGGCGCAACCTCGTGGCAAAACTTTTGGACCAAATTCTTCGAAGGCTACGA |
| | CCACATCTACATCTGGTCAGACCCAGACCCCGCAGGCGACAAGATGGCCCAAACCCTAACCCAAGCATTACCGCAAGCCACC |
| | CATGTGCCCCTCACCCTGGGGGATGTCACAGACACCTACCTGCAGGCCGGAAAAACAGGGTTGGCACAAGCACTAGACACTG |
| | TGCTACAGTAAAACACGTCAACAACACGAAACCAGGAAGGTAAACTAAAACATCATGGATCCCCTCGACACGTGCCCCATCC |
| | CCAACCGGCGCAACACCAGCCAAACAGTCAGGAGGCGTATCCGCCTCGCCATCTGTGCAGAAAAATGGGCTGATGGTGAAG |
| | ACCCCACCTACATCATGCACACCTGGGGCACCACCTATGATGGGATGCGATCCATGATCCGCGCCAACCCCGACATTAAACT |
| | ACCCGACGACATGGCCAGACGGTTACACAAAATCTGCCGGAAAGCATACCCCAAAAACCAGCCCAACAGGCACCGAAGCGG |
| | ATGGGACGCCTACGAGAAAAACTACTACACCCACGAAATCCTCTTCCTCGACCAGTTCAACATCCCAGCCCTCGAAATCCTC |
| | AACCGGCTCGACGTGTCATGGGCCATGTGGAAACAAATCATCACCGAAAACCATCTCACACGGCTACAAGACGAGACCTACA |
| | ATGCGTGCCACTGGCACTATCTGAAACAGCAACACCCAGACTGGACCGACCAGCAAATCACACAAGCCCAACACGCCGGGA |
| | ACAACACCTTCAGCCAGTTCATGCAAGACGAACAGCCGGCACTATCGTGAGCATCTCGTTCAAACCCACAAAACGCAACCTT |
| | CAAACCATCCGAGACATCCTCACCCAGGCCACCAACCTCAACCCAGATGATCTGCCAGATGACACCCTCAACAACATCATCC |
| | AATACTGTTGGAACACCTTCACCGACAACAACCGGTACGCCGTCGCGGCACAATACTGGCGAGGCCAAAACCCGCCCGACCA |
| | GCAACATCAGCGCATACTAGTCGGCTACTACAAAACCGTTAAACAAGCCATGAACGCGGCCAAACAATTCCACTGGAACACC |
| | CGGCTACAACAACAATGGAAAACATGGATACTCCCAGTCCACAACGGCACCGTATCAGAGCATTTCACCAACCAGAAAACA |
| | CTCTTCGACACACAAACCAGCAACCAGGGTGACGCCGCACTGCCGGAGCATCTACAAAACGTCCTGTGCGGCAAAACACTCA |
| | ACCACACAGACGGAACCGTATCGTGGTGCACACGCAAACCAGGACACGACGGCGACTGCCGCACAGGATGGCAGCCCACCA |
| | CACAACCCCTAGGACATCATGGCAGCCAAAACTGAAACCCTCATCCAACGCTACGGCAACAAAGCCGCAGACGTCCTCGCCG |
| | ACAAAACCATCCCCGCCACACAGCTAGCCCAAATGCTCACTGAAGCCGGATACCCCATCTCCGCCACCGTCATTAAAGACTA |
| | TCGCCGCAAACAAGCCAACACCACCCCACAAACAGAAGAGGAAAACCAGTGATAGACAACATTGACCGGCTACTCACACAG |
| | CTAGCCAACTACGACAACGCCATCGACACCATCGACGACAATCTAGCCAATGGTACTGTACGACGCACACGAATCTCCGAAT |
| | GGACACTCCCCAACGGAGAAACAGGCCGATCCATACAAAAAATAATCGACCACCAACCCGCAACAAACCCCTACCCTGTAG |
| | ACGAACTCGTCGATAAACTAGCCGAATGGACACCCCCAAAACCAGCCGACAACACCCACACCGACTACAGCACTGCGGCCTT |
| | CGTCATCGGGGCGGGAGACTTCCAAATCGGCAAAGGCATCCCCGGCGGAGAAACAGCACACTTCGCCGACGACTACCTACA |
| | CTCCCTCGCCTCAGCTAAACACTACTGGCAGCAAGCAGGCCAGCCGCAACGAGTCCACATAGCCTTCCTCGGCGACATGATC |
| | GAAGGATACGTGTCACAAGGAGGCAACAACGCCTGGCGCACACAAACCCCCTTGACGGAACAAATCCGGCTCACCCGCATG |
| | GCCATGATGCAACTCGTCCACATGTTCGACCACTGCCAAAACGTGACAGTCACATCCATCCCCGGCAACCACGGAGAAGCCG |
| | TACGCTTCGGCAAAGGCGTCACCACCTACGACGACTCCTTCGACGTAGACTGCTGCCGCGCCATCGCAGAAGCCTACCAACT |
| | CACCAACAACTACCCCAACCTCCACTTTTATTTTCCTGACAGGGACGAAATGACCACCACCGTTGATGTGGCCGGAACACGG |
| | ATACTGCACGCCCACGGACACCAATGGCGCAACAACCAACACTACGAATGGTGGCGCGGCCAAGAATTCCACAACGGCACC |
| | GTATCCCACATTCTCATGGCCGGACACAGGCACCACCTCGAAATATCCGAGCAAGGACAACGCACATTCATACAATGCCCAT |
| | CCATGGAAGGAGAATCCGTCTGGTACCGGCACAAGACAGGCACCACCGGCAACCCCGGACTCGTGTGCTACACTATCAACCA |
| | CAAAACACCAGACAACTACCAAATAGCCAGATAAAAGAGATGCCATGAGTAGACGACCAACCAAAGTCCAACAAGCAACCA |
| | CAGCCAACTGGGAATGGGCCACCCCCAACCACCAACACCAGCTACACAAAGCCTGCACCAACATTGCCCGCCACTACCCGGC |
| | CGTCAACCCCGACGACCTGTACCAAGACTCTCTACTATATATTGCGGTACGCAACCAATACCACCAACTCACAGGCAACCAG |
| | TGGACCCAAATGTGCTACCGTGTCGCCCAACGACTAGCAAACAAAACAGTCCACCACCTAGACCTACCCAAACCCGTCCAAG |
| | AAATCACAGCCATAGCCGACAACCAAACCAGCAACTAAGGAGAACCACAATGGTCACAACCATCCTCGACGACGGCACCCA |
| | AACCACCAGGCTACAAACAGTAGGCGCCACCACCACCGCCATCATCACCAACACAGAAACACCCGAAACCATCACCGCCAA |
| | ATACACCATCAGTAAAGACGGCACAGCCACCTACAGCATCAGCGGAAACACCTATTTGGGAGACCACCAACACATCATCAA |
| | ACTCATGTACGACTACCGCCACTGCGTCGGACGCTTCGACACCACCAACCCAGACAACCCAGACAACCTCGACAACATGTTA |
| | AGGGGATGACACATGAACCGAACCTACACCACCGCCGACATCATTCAAGCCGCCCAATGGATCTGGAACGGAGGCCCATGG |
| | AAACCCTCCGTGGAGCCAGGCATGCCACCCCCACCAACCGCGCCACAGCATCACGGCAACAACATTGTCACCATGATCGATC |
| | TACAGCTAGCCATCGACGACTACACACTCGCCTGCCAGCCATCCAAACAGCGAAAACATTTGGCACGGCTGGCAGCATTCCG |
| | TGAAGTATACGGATACGATCAAACCTATTCGGTGGCAGCCCAGCGACTTGGGGTGACCAGGCAAACCGTGAAACAGTGGGC |
| | AGACCAAACACTCATCACCCTAACAGGCTACGCAAACAGCAGATACTATCCAGACGATAACGACGACAGCACAGGGATGGG |
| | ATAAAAACATGAACAACACACACAATATCCCCTACACTGCCCTCAAAACAGCGGTACACCGTATCATCCAACAACAGCCCAC |
| | CAACATGCAACAGTTGGAAAACATTGTTGGCGGTGTCGAAAACCAGTACCGTGTACCAATCTCACTCGACAACGTGAACCTT |
| | ACCGTCAACGAAGTCAGCCTCGACGATCTTGCTATCGACCAGGACACGCTAGACGAATGCAGTGAAATCCTATGGGACTGCG |
| | ACAGTGCAGGACACCCCACAAACAACAGCAACACCCGTGACAACAGCGAGGACCAGAGCCCCTATGCAAGCCAGGAAGCAC |
| | TAGACTGGCTCGCCGGAATCGCATACCAGGCAAAACTACTGCAAGCGGAGGCCGACGATATCATGCAGTCCATCCTCTACCA |
| | CCGCGACAACCACGAAGGCGTTATCGGCCAGGATGTTCTAGACCAGGCCAACGATACTATCTCAGCCTGCCTCCACCTGGAC |
| | CAGCTCATCGAAGACACACTAGACAACAACCTGTAAAACCCCTATAGACACAAAAAGAGTGCCCCAGCAGCAGCCACCACC |
| | CAACCGTGGCAGCACCGCTGGGGCACACATCTATATTCACTTATGCAACAGTAGACTCTACCGTGCCAACCTCAGACTCGGCT |
| | GCACGCCGAGGCACATAGCCGGCAAAACCATCCACATCATCTGCAGGCTCAATCATGCCAGGATCCGACACATCCACCGAGT |
| | GCGGCTCAACCAAGCCCCCATCGTCGGGGGGAACAAGCCCAGCATCCACAACCGTGGTTTTAGGTTTGCCGGCCACAAACGA |
| | CGGGCTACCAAACGATGTAGCAACCGACAACACCGCAGCCACCGTTGCTGTTATCAGGGCAGACTCCCACGGCAAACCGCGA |
| | AACGACTCCGCAGTATACGTGACACCCGCCGTCACACCCAACACTGCAACAAACGTTTGCACAAAAGTCTTAGCCGCCCGCT |
| | CCAGTAAACCTAACCAAAACTGTTTACCCACAACAAACCACCATCACTTTTTCAAACCGTTGACAGTCGACTCAAGCCTGTCA |
| | ATACGGCTACGACACGCCAGCACGTAATACCAGACACTCCACAAAGCATCCTTAGTGCGCCACAGCTTCCCCGTCACAGGAT |
| | TCTTCACCCACGACAAAGCCTCGACACGCTTACGCAAGTCACCATTCTGCACCTGAACCACACCAACATCGTGATGCAGCTTA |
| | TTCACCGAACCAGTAAGCTGAGCAGACAATTGTTTAATCTGATCACGCAAGGCTTTCACATCAGCCACCGTTAACTCCTCACT |
| | CTCTCCACTGCCGCCGTTCACGACGGCCATAAACCTGTCCCACGGAAACCACGGCCCCGGATCGTCATGATCCGACTGATGCC |
| | ACGCATCCGTCACATCAGTGTGCCCGCAGATGCCCCGCCTGCCAGCCTTCAAATCAGCCACAGACAGTTTCCTCTTCGGAACA |
| | CCATGCTTGTCACACAACTGCCGGCACAGCACCGCCGCTCTTTCCACCGCGGGCCACACCCGAGGATCCAGCCACTGCTCAC |
| | GAGTGTAAGCATGCCCTGGCACCCGGAACGAGGCGTGCGAACCGCCATCTGCGCAAACCTCTATACCCAAACTATGCGGATT |
| | CGGCGGGGCATGCCACCCAATCGTAGACTCCGACAAGCACTGCACCGTCTCCCCAATATCGCAAACATAATGCGCCGAACCA |
| | CCAGACGATGGGGACACGAAATAGTTTGCCGTGGACACCGCCCGCCCTTTACGGGAGGCAGACGGAAACCCCACATCCGGG |
| | CACGTCGCATGAATCACAACCCTGTTCACCGGACTATTCGAGCCGGCCGAGTGATGTGCTGCAGGAATAAACCTCACCACAC |
| | ACCACCACCAAACACTACCATCACAGCCACTCCTTTCTATTTGTGGGATGATATAGTCACTATAGGCGACGGTTTCACACCCT |
| | GGCAGGCCACCGAGCCCGATATTGTAGAAGCCACACCGTCACTATATTTCACAACAAGGCGGCCCCCGGAACAGTACACAGA |
| | CGCAACAGAACGGCCATCCTTACCATCTTTACCATCGGATCCGTTCGCACCGGCGGGGCCGCGCTCACCCCGTTCACCCTGTG |
| | CACCTTGCGGGCCGGCAGGACCTGAAGGGCCCACATCACCGCGCTCACCGGCCGAACCATCCCGACCAGCAGTGCCATCAAC |
| | GCCGTTCACACCGTCAGCACCTGCACGGCCTGGAACACCATCACGGCCATCCGACCCATTCACACCAGGCAACCCGTCAGGA |
| | CCTTTCACACCATTCAAACCAGGAGAACCCTGCGGACCAACAGGGCCAACCAGCCCAGCCGAACCATTAACACCATCACGCC |
| | CATCAGCACCTGCAGGCCCTTGCGGGCCGCGCTCACCGGCAGGACCCGGCACACCCTGCACGCTACGCTCAACACGCACAGC |
| | ATCCACACACAAACCAGACTGGTGAAGACGCACCGACTCCCGCCCACCCGAAGCACACACCCGCTTCACACTGCTAGCCAAC |
| | CCTTTAGCCGCTGTACCATTAGACTGGGCCCTCGCCTGCTCCGAATCCCGCTCAGAGGATACAGCCCCGAAACGCAAAGCAC |
| | CCCCGGCAACCACCGCCAACAACACAAGCGACAAAAACAGCAACACCAGTGAAAACTTCTCAAAATTGCGGCGCTGCCGCT |
| | TCTCCTCCTCCAACTCCCTCATCATTCACCCCCCAACGTGTCCGACAGGAAACTAGGCAACTCAGGCAAAACAATCGGATCAA |
| | CCCTGTCAGGAAGCCGGGCGTTAAACTGGCGCACCTCGCGCCGCACACACCACGTATACTCTTCCATCGCATCTACCTGAGCC |
| | GACAGCCGCCGCAAACGCTTCCGAGATTTAGATGTGGCAGCCTGAACCGAACCCAAAACCGTGGCCAATGCGGTACAGATA |
| | GAGGCCACCAGTGCAGGAGTAAACCACGACACCACAGCCCCCCGACATCACACCATCCGCCACAACAACAGCCCGGTCACA |
| | CGCCGACAGCAATCCAGTTAGCTATCGCAGGCACACCAGAAGGCTTAGACCCGTCATTCGTAATAAAAGCCAAACTAAAATT |
| | ACTATTAGTAATATTGTAGGCTTTCACATCAATCTGTGTCGTACCCCCAGCCGCCGTAGCCATAGACGCCACCACAACCGGCG |
| | GAGCACCGAACGGCCGCTCAAACGGGATCGTGTAAGCATACACAGCAGACCCGCCAAACATGATCTGCTTAGTGCCCGTCTC |
| | AATCCTCGGAGACAGTAGCATCCACTCGCCCGAATGGTTAGCCCACACAGCCCCAGAAGGAACCATCACACGGTCACCCTCC |
| | ACAGGGGTCGGGTCACACGCAGCAGACTCCCCAAACGCCACACGGGCAGCTATAGCACGCCTGTCAAGCTGCTGCTGCAACC |
| | CGTTAGACGACACCACCAGGGTAGCCAACAGTTGCTGATGGAACACGCCAGGCTCGGCCCTCAACACGTCACGGGCACGCTC |
| | CGCACGGCCACCCTGAACAATCTCCAACTTGGCTGTGTTCTGCTCCCAATCCCGAGACAACACCACATAGTCGTATCGGGTCT |
| | CACCGGGGCCAGGCAGCTGCCCTGTCACCGTCTCAACACTACTAGAAGTGCACATCACCCCGTGAGCCCAAGCCTGCCCCGG |
| | AAAAACCTCACACAACACCGTGGCACCCTGAACAGTAGTACCCACACGAAAATCGTCCGGCCCCTTCACAGACGGCATATTA |
| | CCCATCAGACCAGACATTTGAGCCCAATCATACTCGGTCAACACACCATCAAACCCTTTACACACAATACCCACAACAAACC |
| | CCTAATCTTTTTAGAATTTTTGCAAATCCCGCACACCCGCAGCCAAACCAGCAACACGCCGTGCTAGCAACGCCGACGGATTA |
| | TCCTCATAATCCCCCGCCACAGGAGTCACCTTCGTCCAACCATCACCAGGCGATACACACTCCACATCAATCTGCCGCACAAT |
| | CTCCGCAATAGGGCCAGAACCCACATCAACATAGATCAAATCCCCAGGCATCAGCTTGCCCGGCCCAANACGCAACACATCC |
| | NACTCCGCCAACTCGNTCTTAAACCCCGANGAAGCCTGCAACTCCCC |
| | SEQ ID NO.: 2; >2765 len: 15103 cycle: O |
| | GGTTGGTGGTCGATGATTTTTTGTATGGATCGGCCTGTTTCTCCGTTGGGTAAGGTCCATTCGGAGATGCGTGTGCGGCGTAC |
| | GGTTCCGTTGGCGAGATCATCGCGGATGGTGTCTGCTTCGTTGTCGTGGTTGGCTAGCTGGGTGAGGAGCCGGTCTATGTTGT |
| | CTATCATCGGATATCCTCTTCTTTTTTCTGTGTGGTGTTGGCTTGTTTGCGGCGATAGTCTTTAATAACAGTGGCGGAGATAG |
| | GGTATCCTGCCTGGGTGAGCTGTTTTGCTAGCCATGAGGGGGGATGGTTTTGTCTGCGAGCACGTCGGCAGCCTTATTGCCGT |
| | AGCGTTGAATAAGGGTTTCAGTTTTGGTTGCCATGATGTCCTATCGGTTGTGTGGTGGGCTGCCACCCTGTGCGGCAGTCGCC |
| | GTCGTGTCCTGGTTTGCGTGTGCACCGTGTTGTGGTTCCGTCTGTGTGGTGGAGTGTTTTACCGCACATGACGTTTTGTAGAT |
| | CTCCGGCAGTGCGGCGTCACCCTGGTTGCTGGTTTGTGTGTCGAAGAGTGTTTTCTGGTTGGTGAAATGCTCTGACACGGTGC |
| | GCGTTGTGGACTGGGAGTATCCATGTTTTCCATTGTTGTTGTAGTCGGGTGTTCCAGTGGAATTGTTTGGCGGCGTTTTCTGC |
| | CTGTTTTATGGTTTTGTAGTAGCCGACGAGTATGCGCTGGTGTTGCTGGTCGGGCGGGTTTTGGCCTCGCCAGTATTGTGCCG |
| | CGACGGCGTACCGGTTGTTGTCGGTGAAGGTGTTCCAGCAGTATTGGATGATGTTGTTGATGGTGTCATCTGGCAGATCATCT |
| | GGGTTGAGGTTGGTGGCCTGGTTGAGGATGTCTCGGATGGTTTGAAGGTTGCGTTTTGTGGGTTTGAACGAGATGCTCACGAT |
| | AGTGCCGGCTGTTCGTCTTGCATGAACTGGTTGAAGGTGTTGTTCCCGGCGTGTTGGGCTTGTGTGATTTGCTGGTCGGTCCA |
| | GTCGGGGTGTTGCTGTTTCAGATAATACCAGTGGCACGCATCGTAGGTTTCGTTTTGTAGCCGTGTGAGATGGTTTTCTTCGA |
| | TGATTTGTTTCCACATGGTCCATGACACGTCGAGCCGGTTGAGGATTTCGAGGGCTGGGATGTTAAACTGGTCGAGGAAGAGG |
| | ATTTCGTGGGTGTAGTAGTTTTTCTCGTATTGGTCCCATCCGCTTCGGTGCCTGTTGGGCTGGTTTTTGGGGTAGGCTTCCCG |
| | GCAGATTTTGTGTAACCGTTTGGCCATGTCGTCGGGTAGTTTAATGTCGGGGTTGGCGCGGATCATGGATCGCATCCCATCAT |
| | AGGTGGTGCCCCAGGTGCGCATGATGTAGGTGGGGTCTTCGCCATCAGCCCATTTTTCTGCTCAGATGGCGAGGCGGATGCGC |
| | CTCCTGGCAGCTTGGCTGGTGTTGCGCCGGTTGGGGATTGGGCACGTGTCGAGGGGATCCATGATGTTTTAGTGTACCTTTCT |
| | GGTTTCGTGTTGTTGACGTGTTTTACTGTAGCACAGTGTCTAGTGCTTGTGTCAACCCTGTTTTTCCGGCCTGCAGGTAGGTG |
| | TCTGTGACATCCCCCAGGGTGAGGGGCACATGGGTGGCTTGCGGTAATGCTTGGGTTAGGGTTTGCGCCATCTGGTGGCCCGC |
| | CTTGTCTGGGTCGGACCAGATGTAGATGTGGTCATAGCCTTCAAAAAATTTGGTCCAAAAAGTTTGCCACGAGGTTGCGCCGG |
| | GTAGGGCAACGGCCGACCATCCGCATTGTTCGAGGATCATGGAGTCGAATTCGCCTTCGCAAATGTGCATTTCGGATGCCGGG |
| | TTGGCCATGGCGGCCATGTTGTAGATGGAGCCTGTGTCCCCTGCCGGTGTCAAATATTTGGGGTGGTTGTGGGTTTTGCAGTC |
| | GTGCGGGAGTGAGCAGCGGAAACGCATTTTTCGTATTTCGGCTGGGCCGCCCCAAACGGGGTACATGTATGGGATGGTGATGC |
| | ACTGGTTGTAGTTTTCGTGGCCGGGTATGGGGTCATTGTCGATGTATCCAAGGTGGTGGTAGCGGGCTGTTTCTTCGCTGATG |
| | CCTCTTGCTGAGAGCAGGTCGAGTATGTTTTCGAGGTGGGTTTCGTAGAGGGCCGAGGCTTTCTGGATTCGGTGGCGTTCCGC |
| | AATGTTGTAGGGGCGTATGCTGTCGTACATTCGGGTTTTCTTTCTCTAGTCGTTGCTGTAGCTTGTGGAGTCCGCCTCCGATA |
| | CCGCATGTGTGGCAGTACCAGACGCCCTTGTCGAGGTTGATGCTCATGGAGGGCTGGTGGTCGTCGTGGAGCGGGCAGAGGAT |
| | GTGTTGCTCGTTTTTGGACGGGTTGTAGCGTATCTGGTAGTGGTCGAGGAGGCGGCAGGTGTCAGAGGTGTGGGAGGAGCTCG |
| | TTGAGGGTTGATACCACATAGGCTTCGCTCCAGGGTTTGTTGCGCTGTTTCATGATGACGAGTCCGATGGTGGACTGGTTTTC |
| | GCGGTTTCGGTGGGTTTCGTAGTTGCGTGCCTCCCGGCTGGCTTGTTTCACGAATTGGGCTGGGTGTGGCTGGCCCGCTTTGG |
| | CTTCGATCACATAGGTTTTGTGGCCGGTTGTGAGGATGAGGTCGCCTTCGTCTTCGCGGCCGTTGAGGTGGAGGCGTTCTATA |
| | TCGTGGCCGGTGTCGCGTAGCTGGTGGAGGAGTCGTGTTTCCCATTCGGCTCCGGCCCGCCGGTTGCGTGCCTGCTGTGTGGC |
| | CATAGTTTTTTAGAGTCCTTTGTGTGTTGTGGTCATGTTCCAGGGCTGTTTTTCGGCGAGGGGCCCGAAGAATGTGTATTCAG |
| | GGTAGGCTCTGAGTCGTTCGTATCGGGTTCCGTCAGGACCGGATTTGCCGGTGCGCTGTTTCAACACTGCGATGCGGGCTTCG |
| | GCTGGGATCGTGAGCCCGTTGCCGTTGTCTTCGCCACCATACAATGAGACTCCGAGGATGAGTTGTGGTTTTTCGGAGAGGCC |
| | GTTTTTGATTTCGCGGCGTGCCGGGGGGTGTTCGATGTCGGTTCCTGTTTTGTCGGTTGCGTGGTGGGTGACGATGATGGTGG |
| | AGCCAGTATCTCTGCCCAATGCTGTGATCCATTGCATGGCTTCTTGCTGTGCCTGGTAGTCACTCTCGCAGTCTTGGATGTCC |
| | ATCAGGTTGTCGATGACAATGAGTGGTGGGAAGGTGTTCCACATTTCCATGTAGGCTTGTAATTCCATGGTGATGTCTGTCCA |
| | TGTGATGGGTGACTGGAATGAGAATGTGATGTGTTGGCCGTGGTGGATGCTGTCTCGATAGTATTCTGGCCCGTAGTCGTCGA |
| | TGTTTTGTTGTATCTGTGTGGTGGTGTGTTGGGTGTTGAGTGAGATGATTCGTGTGGAGGCCTCCCAGGGTGTCATGTCCCCT |
| | GATATGTAGAGGGCGGGCTGGTTGAGCATGGCGGTGATGAACATGGCTAGCCCTGATTTTTGGCTGCCGGAGCGCCCCGCAAT |
| | CATGACCAAATCCCCTTTGTGGATGTGCATGTCCTGGTTGCGGTAGAGGGGTTCTAGCTGGGGTATGCGGGGCAGCTCGGCGG |
| | CTGTTTGGGAGGCCCTCTCGAAGGATCTTTGGAGAGAGAGCATCGGGACCTTATCTATCTATCGGTTGGATGTGTATTGGTGG |
| | TCAGATGGAGTCGATATCGATATCAGCATCAGTTGAGGCTGTGGTGTCGTCTAGCTAGCCGTTATCGCGCTTGCCTACGTATTC |
| | GGCAACCTTATCGTAGATGGCGTCATCAAGGGGTTCGAGGATTACCGCGTTGAAGCCGTTTTTGGTGCGCACGGTGGCGAGTTT |
| | GAAAGCCTGCTCCTCGCCAAGGTATGCTTCTAGATCGCGGATCATGGAGTGTGGGCGGTCGTTGTTACCGCGTGCTTTCTCAA |
| | TAATAGCGTTGGGGATGGTTTCTGGGGTGCCGTTGTTGAGATCGTCTAGAGTGTGGAAGATTGTGACATCAGCGTAGATGCG |
| | GTCTGCGACCTGTCCGCCGTAGCCTTCAGTGTTGTGCTGCACGTCGCGGATTTTGAAGGCGATGGCGGTGCCGTCCTGCTTTC |
| | GGGAGGGGTTGAAGAAGGTGCTGTTGCTGTTGTTGCGGTAGTTTGCGAGTCCCATAACTGTTTTCCTTTTACTGTTTATGTTTG |
| | GTTTTGTTGGTTTGTGTCGGTTTTTATCGGGTGAGGCTGTTGCGTTTGCTGCGGAAAGCCTCAGACACGTCCCTGTTACTGGTG |
| | ATGATCTTCTTGTACTGTTTCAGAAGGTCGGCGAGCTGTGCCTTGCTGGTGGCTTTGTTGATCCGGTCGATGATGATGTCGCTT |
| | TCCTTGTTGGCGATCTTGTTGACGTAGTCTTTGGCGGCTTTATCGTATCGGTCTTGAAGCAGGATTGCTGCGCTAGCGATGAG |
| | GGTTGCGAGGTCCCAGTCTTTGGATACGGTTTCGTCTTTCAATCCTCCTAGCAGATCAATAATGGATTGTTTGATGTCTTCTGC |
| | GGTGTCTCCGCGGATGACGGCCCATGGGGCAGCATAGTCGCCACCGTATTTGGGAGTGATCGTGAGTCGATCATTGTCGATCT |
| | TGTCTTTATCGGTCATTTGGTGTCCTTTTCTTTATTGTCTGTTTGGGGTGGCTGTACGGTGGATTCTACCGGGTATCTGTACGA |
| | GTTTTTGCCATTGACGGCCCAGCAGGCGTCTCGTACGGGGCATCCTTTACAGAGTGTTGTGACGTGTGGGACGAAGATGCCTTC |
| | ACTGATTCCTTTCATTGCTTGACTGTACATGGATGATACATGCCGGTAGGTGTTGTTGTCAAGATTGTAGAGTTCGGTGGATG |
| | TGCCCTGTGCGGGGGATTTATCGTCGTTGCGGCTGGTGGCCGGTGTCCAAAACATGCCTTTTGTCACATGTATGTCGTGTTGTT |
| | CGAGCATGTACCGGTATGTGTGCAGCTGCATACTATCGGCTGGTAGGCGTCCTGTTTTGAGGTCGAGGATGAATGTTTCACCC |
| | GTATCTGTGTCAGTGAAAACGCGGTCAATATATCCGACTATTTTTGTGTCATCGTCGAGGATTGTTTCTACCGGGTATTCGATG |
| | CCTGGCTGGCCGTCTAGGACTGCGGTGATGTATTCTGGGTGGTTGCGCCTCCATGTTTTCCACCGGTCCACGAAGGTTTGGCC |
| | GTAAACCATCCACCAGTCGTAGTCTTTCTTGTGTGGCCCGCCCGACTCGCACATGTTCTTGCATATTCTGCCGGAGGGTTTGAT |
| | TTCTGTGCCTTCGGATTCGGCGAGGGCGACTTGGGTGTCGAAAATGTTTTTGAAGGATGAGAGTTTGTCTGGTAGTGCAGAGT |
| | ATTCGGTGGGGTTGTACAGGTGTAGGTCGTATTGTTCGGTGATGTGGTGTATGGCGCTTCCGGCGATGGTGGCGTACCAGGTG |
| | TGGGAGGTGGCTTTGTATCCGTGTTGGAGGCGCCATTTTTCTCCGCATTCGGCCCACTGGGTGAGTGAACTGTAGGAGATGTG |
| | GCCTGGGTGGTTGATGGTTTTCGGGTGTTGTGCTAGAGGCATGTTAGTTGTTTTTGTTCCATGGGTTGCGGGTGTCTTGGCCGG |
| | CCTGGTGTAGCTGGTAGGCAAGGAGTGCGAGGCAGTGCCAGGCAGCATGGGCTAGATGCGGTAGCCCGGATTCGCGGTCGA |
| | GGTTGTTGCCTTGCTGCCATGATAGTAGATGCCTGTAGAGGGCGTCAATGCTGTGGCTCCACGGATAGCCGCCGGTCCAGTTG |
| | TTGTCGCCGTATTTAGTAGCACCGTATCCGGCTACTTCGCCTAGGGCGTGCAAGGCTGCGGGGTCGATGAGTGAGAGCCTGC |
| | AGAGTTTTAATTCTTTTCGGGCTCCGGTGTTGGGGTCGGTGTACATGCGGGTGGGCTCATCCATGGGGTGTGTGCTCCTTAAG |
| | TGTGGGTTACTGGTTGTTGTTGTGGGCTAGTGCTACGGCGAGAATAATGATGGCGAGGGTTTCAGCGATGAGGATGGGTGTT |
| | GTGATCATTTGGTGTCTCGGGGATTGTTGGTGAGGGTTGAGGCACCCAGGAGAGTGGCGAGGGCACATGCGGCGATGATGGC |
| | GAGGGCTGCCTTGTGTGTGGTGCCGGTCGCGTACATCCATGTGATGATGCCGCCTTGGATCCAGGCTAGGCTGGTGAAGAAC |
| | GTTTCGTAACTGTGTAGCTCAATGTTATTGTTGGGTGTGTTCATGCTTGCTCCTGAAGAATGGTGTTGATGGTTTTATAAATGT |
| | TGTACAGGTCGGTTTCGATAGATAACAGTTGGTTGATTTGGTGGTCGAGATCAATGTCTGGGTTGAGGGTGTTGATGCGGGAG |
| | GCGATGTCGGTGGCTGTGCGTAGTGTGCCGCCGGTGTGGTGAATGATGTGTGCCGTGTCGGCGAGTCCGGTGGTGACAGCGT |
| | AGTGGGAGAGGAGAGGCATAGCTGGGGGGTGCTCCTTGGCGGGTTACTGTTGCGGGTTGATGTTGAGGTCGGTGACGTGCGG |
| | GTGGTCTTCTGTTCCGGTGACGAGGCAGTGGACTGTGACGGGTAGTTTGGATGCTCCCGGCTGTTTCGCGGTTGCGCCGTAGA |
| | CGATGGAGAAGGTGTCTTTACCAATAATTTTGTGGAGTTGTAGGTCGATGTCGGGGTTGCCGTTCCATTTGACACCGTTTTCT |
| | GCGGCTGCCTGGGTGGCTTTCTGGTTGCAGGCGTGTGCGGCGGTGATCATGGTTAGCCCCTTGCTGGTTTCTTCACCCCTTGCT |
| | TGGGCTTGCCGGTGGGCGCGCTCCTGTTCTGCTTGTAGGGAGCGGACTGCTGCGGCCTGGCGGGCTTTCTTCTCAGTCTTGCG |
| | CTGCTGGACGGTTTTGGGTGTCCATTCGGCGTTTGCTGTGGTGGCTTGTGGGGGGGGCTGTGAGGCGAGTGGCGGGTTGTCGT |
| | CTGGGGCTGGCAGGAAGGAGGCTGCGGCGATGATGGCGGCTGTGATGCCTGCGATGGTGTAGCCTTTTTTCTTGTTCATGACT |
| | GTTGTCCCCTTTCCGGGGTGTTGTTCGTTGCTGACATGATTAATTATGGTGTGGGCGGTGGCCCATGTCAAGGCTGCGCTCAA |
| | CGATTATGAGCGATCCTTGTATGGCTAGGGGTTATATCGGGCGCACAGGGTGAGTAGGTGGCCAACATTGATGCGGCTCACA |
| | TTCCAGTAGAGTTGCGTGGCTTCCCCACCGGTGAGTGGCTTCCACTCGTCATGGCTGTAGACGGTGCCGTCGGTGGCGATGAA |
| | CGTGTTGGGGCGCAGCTTGTGAAGTTCAGTCTCTACATGCTGCCGGTAGGCTTCGGCGAGGCCCTCGAAATCGAGGTGGTCG |
| | CAGGAGAGGTTTTCGAGGCGTGTCAAGTCGAAAGGCTCAGGGCAGTCCAGGGCGGGGGTGTAGAGCTGGGTGAAGTGGTCG |
| | GCGATCTTCTGCATGACGGGTTCCTTTCTGGTGTGTGGATGGTTTTTATCGTGTGGTTTCTTGGAGGTTTGTGGTGGTGTCGAT |
| | GGTGTCGATCTCCCAGGCGAGTTCCTCGGCCTCATCCGCGGTGAGTGGCTGCCAGTTGCGTGGCCCATACACTGCACCGTCGA |
| | GAGTGATGGTCCAGTCGGGGCGGAAGAGGTTTAAGGCTTCTTGCACAGCATCCCGATAGACATGTTTGGCGGCGTCCAGGTT |
| | GATGTGGTTGGCGTGTCGGCCGGCTAGCTGGGTGAGGTCGAGGGGGTCGATTTCTGTCTGCCCGTAGAGGGAGGTGATGGAT |
| | GGGGTGATGAGTGTGGTGGCCATTTTTGGTGTGTTCCTTTCGGTGGTGTATGGGTTGTTGTGGTTTCTAGAGTGTGTAGGTTGC |
| | AACCGGGAGTCAAGGCTGCGCTCAACCGGATTGAGCGTTTCATGCTGGAGTGTCGGGTGTGACAGATGCCACTTAAGCCTTT |
| | ATGGCATCTCTCAGCGTCTCAAATCTTCTGGGGGTAGGATGATGCAGGGTTAGTCCTGCTGATCGATTCTAGGCCCCATACAG |
| | GGCGTCTCAGTGGTATGTCTGGGTGATAGCGGGTGTGGCAGATGATCTAGCGGGTCAAGGTGCCGGGCTTAGACATAAGATC |
| | TATCATCTAGATGTGTGAGATGTATCACACTCTCCTGGCTTGGTGTGCCCCCTCAAGGCCACCCAGCCGATCTGGCGTGAAGG |
| | GTGTAGCCCAGAAATACCGTTTAAAGCTGTGACACGGCGCCTAGGAGCGCCTTACAGGGTGGGGGCTAGGTATTTATACCCC |
| | CAAGCAATTCTGATCGATTCTAGACGTCCCCCAGAGCCTGATACACGATCAACCATCCCGGCATAGATCATCAGCCCCTATCC |
| | TGGTTAGCTCAGCCTCAACTATGTGGACAGTGTGGGATACTGTGGGGGGAGAAGGACGCGGTAAAAGAAAGAGGGGGGCAT |
| | CAGCCTTAGGGTCTTAGCACTGATGGACTTAGCACCGAGCCCCTCAGGGGCTCGACATCAGCCCGAGCAGGCTCAGCTCATC |
| | AGGCACAGCCCTGAAAGAGGTACATGCCATCAGGGAAGGCTTGAGAGTACGAGGAGCCTTAGCGACGAGTACTCGAAAGCC |
| | TGAGGGAACACCCTCAGCACTGATGGGCCTAGCGTGTTCGGAAAAGACACGGGTCAAGGTGTGACAGCTGTCCGGGAGTGA |
| | AACCCGTTCTGACTAGGGGTTTCAGCCTTAACCACCTGTAAAGGTTACAAGACTCTAAGAAAATTTAAGGAAAAGTTTAGGT |
| | TTAATTTTTGGACCTTTACTACCAAAAACACCCGTTTCCACCCCTCAAACCCGCCTATAGAGCCAAATCCACCAGTTTGACTC |
| | ATCCGAGGTGGGGTGGGATAGGCTGGACAGGTAGCCAGCTGGACGCAAGGCCGGAAAGTGCTAACGCACTTCCCAACCTCG |
| | CTTAGCATCAGTCTACCAAACACTTAAAGACCTAAGGGCTTAGCGCTAAGGTGCTGATAGCTTAGCACCGAGCCCCTCAAGG |
| | GCTCGGCATCAGTCTTAGGTACTTAAGGATCTAAGTTACTATAAAAGCTTTAAAGTCTTAAAGTAACTATAAAACCTTAAAGG |
| | CTAAGCACTTAAGGATATAAAATTAACATCAGTGTGTAAGACTTAAAGAGTTAAAATAACCATAAAGCTTTAAACACTTAAA |
| | GTAACTATAAAACATTAAAGACCTTAAGTACTTAAAGTTAACCATCAGTCTTAAACTTTAATATTATAACCTATAAGTCTTAA |
| | AGCTTATAGGTATAATAATATAATATAACTATATAAGTATTAAAGCTTATAANTTATAAAAGTTTTAGNAGAGCTAAGGGGTT |
| | AACTTCTTTACTTCTCTTCTCTCTTTGGTTCTTTCTCTCTTCTCTTCTTTTCTTCATCAGGGGAGAAGAGGAACCTTTTACCAT |
| | CAGCGTCGATGGGCTTTTCACCGTGTGACTCGTGTACCACCGGTCGCACGCTCCCGGTGACACACTCCCCACACTCTGACACCC |
| | GTGTTCCTTTCAGGCTTAGCGTGCTTAGCTGAAGGCGTACGGCGTGTCACGCCAACATCCTTAACACTAGGTAAGACTTAAAGT |
| | GTATATTATATGTAGAAGACTTTAAAAACTTTGAAGGTGTTCCCGCTTAGCCTGTGTTCTACACCGCTAGGCGCTAAGCGCTA |
| | AGTCTTGAAACGCGAACACACACCCACCCCCTTTTTCTTTCGTGTCCTTCTTCTTTTGACACCGCTGGGGGGCGATGTGATCTT |
| | TCTCACATGCCAGGGGGTAGTGGAGAAAACAACCACCCCACCACACACAGAACACCCCCTCAAACAAACAAAACGCCCCCC |
| | CAGAATCGAACAGCAGGGCAAGGGCAAGGTATTCATACCCCCAACAGTTCCCAGGCCGTTAGAGAGGCAAATAAGACCCAT |
| | ACAGGGCTAGGCGAGGAACAGACACATCATGGCACGCACCAACCGCACAGCATCCTCCGCCCACCGGCGCTGGCGGGCAAG |
| | ACTCATCACCCAAGCCCGACAACAAGGCCAAACCGAATGCCCACTCTGCGGAGCAACCATCACCTGGGACACACACCAGCTA |
| | CCAACAAGCCCCGAAGCCGACCACATCACACCCGTCAGCCGGGGAGGACTCAACACCCTCGACAACGGGCAAATCATCTGC |
| | AGAACATGCAACAGAAGCAAAGGCAATCGCAGTGAACCAAACATTAGTTTCCAACAACAAACCACAAAAACGTTGATTCCA |
| | TGGTGAAAAACCCGCCAACCCCCACCGGGGACACCCCCTGCACACCCGTGCAAAACCTCGTACGGCTTAGTGAAATACCTCC |
| | CTTTTGTGGTTTTGTCTGTTTGTCGACTTTTTGTGTTGGTGGTGAGTGTTGTGCAGCCTGAGCTTCCTGATAGTCGTGAGTGGT |
| | GTGGGGAGACGCGTCGTTGGTGGCGTGTGTGGGGTGAGGATAGTCGCGCGCAGTATGTGTCTGATGAGGAGTGGCTGTTTCT |
| | CATGGATGCTGCGGTGATTCATGATGTGGTGTGGCGTGAGGGTCGCGCTGATTTGGTGGCTTCGCTTCGTGCTCATGTGAAGG |
| | CGTTTATGGGCATGTTGGATCGTTATTCGGTTGATGTGGTGTCTGGTGGCCGTGGTGGGGGTTCTGCGGTGGCGATGATTGAC |
| | CGGTATCGGAAGCGTAAAGGGGCCTAATGTCGAGCGTTGTTGGTTCTCAGGTTCCTCGTCATCGTGTGGCTGCAGCGTATTCG |
| | GTGTCTGCGGGCGGGGATGCTGGGGAGCTTGGTAGGGAGTATGGGCTGACGCCTGATCCGTGGCAGCAGCAGGTGTTGGATG |
| | ATTGGCTTGCGGTGGGTGCAGGGGGTCGTCTTGCTTCGGGTGTGTGTGGGGTGTTTGTGCCTCGCCAGAATGGCAAGAATGCT |
| | ATTTTGGAGATTGTGGAGTTGTTTAAGGCGACTATTCAGGGTCGCAGGATTTTGCATACTGCTCACGAGTTGAAGTCGGCTCG |
| | TAAGGCGTTTATGCGGTTGAGGTCGTTTTTTGAGAATGAGCGGCAGTTTCCTGACTTGTATCGTGTGGTGAAGTCGATTCGTG |
| | CGACGAATGGTCAGGAGGCTATTGTGTTGCATCACCCGGATTGTGCCACGTTTGAGCGTAAGTGTGGTTGTCCGGGTTGGGGT |
| | TCGGTGGAGTTTGTGGCCCGCAGTCGGGGTTCTGCTCGCGGGTTTACGGTTGATGATTTGGTGTGTGATGAGGCTCAGGAGCT |
| | GAGCGATGAGCAGTTGGAGGCTTTGCTTCCTACGGTAAGTGCTGCCCCGTCGGGTGATCCGCAGCAGATTTTTCTTGGTACCC |
| | CGCCGGGGCCGTTGGCGGACGGTAGCGTGGTGTTGCGTCTTCGCGGGCAGGCTTTGTCGGGTGGTAAAAGGATTGCGTGGAC |
| | GGAGTTTTCGATTCCTGACGAGTCTAACCCGGACGATTTAGCTCGACAATGGCGGAAATTGGCTGGGAATACTAATCCAGCC |
| | TTGGGTCGCCGCCTGAATTTTGGGACAGTCTCGGATGAGCATGAGTCGATGTCTGCTGCCGGGTTTGCTCGGGAGCGGCTTGG |
| | CTGGTGGGATCGTGGCCAGTCTGCTTCGTCAGTGATTCCGGCGGATAAGTGGGTCCAGTCGGCTGTGGATGAGGCTAGTCTG |
| | GTTGGCGGGAAAGTGTTTGGTGTCTCGTTTTCTCGTTCTGGGGATCGGGTTGCTTTGGCGGGTGCTGGCCGGACTGATGCTGG |
| | GGTTCATGTTGAGGTTATTGATGGGCTGTCGGGAACGATTGTTGATGGTGTGGGCCGGTTGGCTGACTGGTTGGCGGTTCGTT |
| | GGGGTGACACGGAGCGGATCATGGTTGCCGGGTCTGGTGCGGTGTTGTTGCAGAAGGCGTTGACGGATCGTGGCATTCCGGG |
| | CCGTGGCGTGGTGGTTGCTGATACTGGGGTGTATGTGGAGGCGTGTCAGGCGTTTTTGGAGGGTGTCAGGTCGGGTGTGATC |
| | AGTCATCCTCGTGCTGATTCTCGCCGTGACATGTTGGATATTGCTGTGAGGTCGGCTGTGCAGAAGAAGAAAGGCTCTGCGTG |
| | GGGTTGGGGTTCCTCGTTTAAGGACGGCAGTGAGGTTCCTTTGGAGGCTGTGTCGTTGGCGTTTTTGGGGGCTAAACGTGTTC |
| | GTCGTGGCCGTCGGGAGCGTAGTGGTAGGAAGCGGGTGTCTGTGGTATGAACGTGGATGAGTTGGCTCTGATTGAGGGCATG |
| | TTTGATCGTATCCAAGGGTTGTCTTCGTGGCATTGCCGCATTGAGGGCTACTATGAGGGCTCTAGCCGGGTGCGTGATTTGGG |
| | GGTGGCTATTCCTCCGGAGTTGCAGCGTGTGCAGACGGTGGTGTCGTGGCCTGGTATAGCTGTGGATGCTTTGGAGGAGCGTC |
| | TGGATTGGCTTGGCTGGACTAACGGTGACGGCTACGGCCTGGATGGTGTGTATGCGGCGAATCGGCTTGCTACGGCGTCGTG |
| | TGATGTGCATTTGGATGCGCTGATTTTTGGGTTGTCGTTTGTGGCTGTTATTCCCCAAGGCGATGGGTCGGTGTTGGTTCGTCC |
| | GCAGTCACCCAAGAACTGTACAGGCCGGTTTTCGGCTGACGGGTCTCGTCTGGATGCTGGCCTTGTGGAGCAGCAGACGTGT |
| | GATCCTGAGGTTGTTGAGGCTGAGTTGTTGTTGCCTGATGTGATTGTTCAGGTGGAGCGGCGTGGGTCTCGTGAGTGGGTTGA |
| | GACGGGCCGTATTGTGAATGTTTTGGGTGCGGTTCCGTTGGTGCCTGTTGTGAATCGTCGCCGTACGTCGAGGATTGATGGCC |
| | GTTCGGAGATTACGAGGTCTATTAGGGCTTACACGGATGAGGCTGTTCGCACACTGTTGGGGCAGTCTGTGAATCGTGACTTC |
| | TATGCGTATCCTCAGCGTTGGGTGACTGGCGTGAGCGCGGATGAGTTTTCGCAGCCTGGCTGGGTCCTGTCGATGGCTTCTGT |
| | GTGGGCTGTGGATAAGGATGATGACGGTGACACCCCGAATGTGGGGTCGTTTCCTGTGAATTCTCCTACACCGTATTCGGATC |
| | AGATGCGGCTGTTGGCGCAGTTGACTGCGGGTGAGGCTGCGGTTCCTGAACGCTATTTCGGGTTTATCACGTCTAACCCGCCT |
| | AGTGGGGAGGCTTTGGCTGCTGAGGAGTCACGGCTTGTGAAGCGTGCTGAGCGGCGTCAGACGTCGTTTGGTCAGGGCTGGC |
| | TGTCGGTTGGTTTCCTGGCTGCCAAGTCGCTTGATTCTTCAGTTAACGAAGCTGATTTTTTTGGTGATGTTGGTTTGCGGTGGC |
| | GTGATGCATCAACACCTACTCGGGCGGCTACAGCGGATGCTGTGACGAAGCTTGTGGGTGCCGGTATTCTTCCGGCGGATTCT |
| | CGGACGGTGTTGGAGATGTTGGGTTTGGATGATGTGCAGGTTGAGGCTGTGATGCGGCATCGTGCCGAGTCTTCGGATCCGTT |
| | GGCGGCACTGGCTGGGGCGATATCGCGGCAAACTAACGAGGTTTGATAGGTGATGGCTTCGGGTGTTGCGTCGCGGATGGCT |
| | GCTGCCGGGTATCAGCGTGAGGCGGTCAGGTTTGCTGGGAAGTATGCGGGCTATTATGCCGAGCTTGGTCGTTTGTGGCATTC |
| | CGGGAAGATGACAGATGCGCAGTATGTGCGTTTGTGTGTGGAGTTGGAGCGTGCCGGCCATGACGGTTCCGCGGCGCTGGCG |
| | GGCAAATTCGTGTCCGATTTTCGGAAGCTTAACGGTGTCGATCCTGGTTTGATTGTGTATGACGAGTTTGATGCTGCCGCCGC |
| | GTTGGCTAGGTCGTTTTCGACTATGAAGATGATGAATAGTGACCCGGATAGGGTGAATGATACGATTGATGTGATGGCTGCG |
| | GGTGTTGATCGGGCTGTTATGAATGCTGGCCGTGACACGGTTGAGTGGTCTGCTGGTGCGCAGGGTAGGTCGTGGCGCAGGG |
| | TGACGGATGGTGATCCGTGTGCTTTTTGTGCCATGTTGGCTACGAGGTCGGATTATACGACTAAGGAAAGGGCACTCACTACT |
| | GGCCATACGCGGCGTCATAGGCGTGGTGGTAAGCGCCCGTTTGGTTCGAAGTATCATGATCATTGTGGGTGTACGGTGGTTG |
| | AGGTTGTTGGGCGTTGGGAGCCCTCCTCTGCGGACACCGCATATCAGCGGGTTTATGAGAAGGCTCGTGAGTGGGTTGATGA |
| | TCATGGGTTGCAGCAGTCGCCTGGCAATATTTTGAAGGCTATGCGTACTGTTGGCGACATGAGATGATGGTTTCCGGTTGTGT |
| | GCCGCCGGTTATCGGTGCACAGGGTTGTCACCCGCACGGGGGTCAACAATGTTGTGTTGTTTTCCGCAAGGAGTGTAGGGTTA |
| | GGCTATGGCCGATCAGAGTGTTGAGGAACAGAATGTTGACAATGATGTTGTGGAGTCCGGAAAGGATAACGGCATTGTTGAT |
| | ACGATAAAAGACGATGGCGGGCAGGAGGTAGCCGACAATCAGTTGAAGAATGAAGGCGGTGATAAGCCGTCTGGGACTGAT |
| | TGGAAGGCGGAGGCTCGCAAATGGGAGTCTCGTGCTAAAAGTAATTTTGCTGAGCTAGAGAAGCTTCGCTCCACCGACGATA |
| | ATTCGGAGTCTACTATTGATGGTCTTCGTCGTAAAAACGAGGAACTTGAGAGTAGGCTGAATGAGCTGGTTTTGGAGGGTGT |
| | GAAGCGTGAGGTGGCCACCGAGCATGGCCTCTCGGCTGATGCTGTAGCTTTCTTGTCTGGTGGTGATCGTGAGTCGCTTGCCG |
| | AGTCTGCTAAAGCTCTTAAGGGGCTGATTGACCATAGTGGTAGTGGTGGCGCGGGTGTGCGCCGTCTTGCGGGGAGTGCCCC |
| | CGTGACTGATGAGAAGCATCGCGAGGGTGTCGCTTTCGTGGATGCTTTAGTAAACAATTCTAGGAGACGATTTATCATGGCTG |
| | ACGATTTTCTTTCTGCAGGGAAGCTTGAGCTTCCTGGTTCTATGATTGGTGCGGTGCGTGACCGTGCTATCGATTCTGGTGTGC |
| | TTGCTAAGCTGTCGCCGGAGCAGCCGACGATTTTCGGGCCTGTTAAGGGTGCCGTGTTTTCGGGTGTTCCTCGCGCCAAGATT |
| | GTTGGTGAGGGTGAGGTCTGTCTCTTATACACATCTC |
| | SEQ ID NO.: 3; >3432 len: 10869 cycle: O |
| | GTCGGGTGATCCGCAGCAGATTTTCCTTGGTACGCCGCCTGGGCCGTTGGCTGACGGTAGCGTGGTGTTGCGTCTTCGTGGGC |
| | AGGCTTTGTCGGGTGGTAAAAGGTTTGCGTGGACGGAGTTTTCGATTCCTGACGAGTCTGATCCGGATGATTTGGGGCGGCA |
| | GTGGCGGAAGTTGGCTGGTGACACTAATCCGGCGTTGGGTAGGCGTCTGAATTTTGGGACTGTGTCGGATGAGCATGAGTCG |
| | ATGTCTGCTGCCGGGTTTGCTCGGGAGCGGCTTGGCTGGTGGGATCGTGGCCAGTCTGCTTCGTCAGTGATTCCGGCGGATAA |
| | GTGGGTCCAGTCGGCTGTGGATGAGGCTAGTCTGGTTGGCGGGAAAGTGTTTGGTGTCTCGTTTTCTCGTTCTGGGGATCGGG |
| | TTGCTTTGGTGGGTGCCGGCCGGACTGATGCTGGGGTTCATGTTGAGGTTATTGATGGGCTGTCGGGAACGATTGTTGATGGT |
| | GTGGGCCAGCTGGCTGACTGGCTGGCGTTGCGTTGGGGTGACACGGAAAAGATCATGGTTGCCGGTTCGGGTGCTGTGCTTTT |
| | GCAAAAGGCTTTAAGTGATCGTGGTGTCCCGGGCCGTGGCGTGGTGGTTGCCGATACTGGGGTGTATGTGGAGGCGTGTCAG |
| | GCGTTTTTGGAGGGTGTCAGGTCGGGTGTGATCAGTCATCCTCGTGCTGATTCTCGCCGTGACATGTTGGATATTGCTGTGAG |
| | GTCGGCTGTGCAGAAGAAGAAAGGTTCTGCGTGGGGTTGGGGTTCCTTGTTTAAGGATGGTTCTGAGGTTCCTTTGGAGGCTG |
| | TGTCTTTGGCGTATCTTGGTGCGAAGATGGCGAAAGCGAAGCGGCGTGAGAGGTCTGGTAGGAAGCGGGTGTCTGTGGTATG |
| | AACTCGGATGAGTTGGCTCTGATTGAGTGCATGTACGATCGTATCCAAAGGTTGTCTTCATGGCATTGTCGTATTGAGGGCTA |
| | CTATGAGGGCTCGAGCCGGGTGCGTGATTTGGGTGTGGCTATTCCGCCGGAGTTGCAGCGGGTGCAGACTGTGGTGTCGTGG |
| | CCTGGTATAGCCGTGGATGCTTTGGAGGAGCGTCTGGGTTGGCTTGGCTGGACTAACGGTGACGGCTACGGCCTGGATGGTG |
| | TGTATGCGGCGAATCGGCTTGCTACGGCGTCGTGTGATGTTCATTTGGATGCGCTGATTTTTGGGTTGTCGTTTGTGGCTGTTA |
| | TTCCCCAAGGCGATGGGTCGGTGTTGGTTCGTCCGCAGTCACCCAAGAACTGTACAGGCCGGTTTTCGGCTGACGGGTCTCGT |
| | CTGGATGCTGGCTTTGTGGTGCAGCAGACGTGTGATCCTGAGGTTGTTGAGGCGGAGTTGTTGTTGCCTGATGTGATTGTTCA |
| | GGTGGAGCGGCGAGGTAGCCGTGAGTGGGTTGAGGTGGATCGTATACCGAATGTGCTTGGGGCGGTTCCGTTGGTGCCGATT |
| | GTGAATCGGCGTAGGACGTCGCGTATTGATGGGCGTTCGGAGATTACGAGGTCTATTAGGGCTTACACGGATGAGGCTGTGC |
| | GCACACTGTTGGGGCAGTCTGTGAATCGTGACTTCTATGCCTACCCTCAGCGTTGGGTGACTGGCGTGAGCGGGGATGAGTTT |
| | TCGCAGCCTGGCTGGGTCCTGTCGATGGCTTCTGTGTGGGCTGTGGATAAGGATGATGACGGTGACACTCCGAATGTGGGAT |
| | CGTTTCCTGTCAATAGTCCTACACCGTATTCGGATCAGATGCGGCTGTTGGCGCAGTTGACGGCTGGGGAGGCTGCGGTTCCG |
| | GAACGCTATTTCGGGTTTATCACATCGAATCCGCCATCTGGGGAGGCGCTTGCGGCTGAGGAGTCTCGGCTTGTGAAGCGTGC |
| | TGAGCGGCGTCAGACGTCGTTTGGTCAGGGCTGGCTGTCGGTTGGTTTCCTGGCTGCCAAGTCGCTTGATTAATACGGTAACG |
| | AAGCTGATTTTTTTGTTGATGTTGGTTTGCGGTGGCGTGATGCATCAACACCTACTCGGGCGGCTACAGCGGATGCTGTGACG |
| | AAGCTTGTTGGTGCCGGTATTCTTCCTGCGGATTCTCGGACGGTGTTGGGGATGTTGGGTTTGGATGATGTGCAGGTTGAGGC |
| | TGTGATGCGTCATCGTGCCGAGTCGTCTGATCCGTTGGCGGCGCTGGCTGGGGCTATTTCTCGTCAAACTAACGAGGTTTGAT |
| | GAATGGCTTCGGGTGTTGCGTCGAGGCTGGCTGCTGCCGGGTATCAGCGTGAGGCGATTCGTTTTGCCGGGAAGTATGCGGG |
| | CTATTATGCTGAGCTTGGTCGTTTGTGGCATTCCGGGAAGATGACAGATGCGCAGTATGTGCGTTTGTGTGTGGAGTTGGAGC |
| | GTGCCGGCCATGACGGTTCCGCGGCGCTGGCGGGCAAATTCGTTTCAGATTTTCGGAAGCTTAACGGTGTGGATCCTGGTTTG |
| | ATTGTGTATGACGAGTTTGATGCTGCCGCCGCGTTGGCTAGGTCGTTTTCGACTATGAAGATGATGAATAGTGACCCGGATAG |
| | GGTGAATGATACGATTGATGCGATGGCTGCGGGTGTTGATCGGGCTGTTATGAATGCTGGCCGTGACACGGTTGAGTGGTCT |
| | GCTGGTGCGCAGGGTAGGTCGTGGCGCAGGGTGACTGATGGTGATCCGTGCGCGTTTTGTGCCATGTTGGCTACGAGGTCGG |
| | ATTATACGACTCGGGAAAGGGCGCTTACTACTGGTCATACTCGGCGTCATAAGCGTGCCGGTAAGCGTCCGTTTGGTTCGAA |
| | GTATCATGATCATTGTGGGTGTACGGTGGTTGAGGTTGTGGGGCGTTGGGAGCCCTCCTCTGCGGACACCGCATATCAGCGG |
| | GTTTATGAGAAGGCTCGTGAGTGGGTTGATGATCATGGGTTGCAGCAGTCGCCTGGCAATATTTTGAAGGCTATGCGTACTGT |
| | TGGCGACATGAGATGATGGTTTCCGGTTGTGTACCGCCGGTTATCGGTGCACAGGTTTGTCTCCCGCACGGGGGTCAACAATG |
| | TTGTGTTGTTTTCCGCAAGGAGTGTTAGGTTAGGCTATGGCCGATCAGAGTGTTGAGGAACAGAATGTCGACAATGATGTTGT |
| | TGAGCCCGGAAAGGGTGGAGACATTGTTGATGTTGTGAAGGATGATCAAAGCGGGCAGGCAGGCGATCAGTCGAAGCATGA |
| | TGGTGTAGATAAGCCGTCTGGCACGGATTGGAAGGCTGAGGCCCGTAAGTGGGAGTCTCGTGCTAAAAGTAATTTCGCCGAG |
| | TTGGAGAAGCTTCGCGCCTCGGATGGTGATGCGGGGTCTGTGATTGATGATCTTCGCCGCAAGAATGAGGAACTCGAAGACC |
| | GGATCAATGGGTTTGTTCTTAAGGGTGTGAAGCGTGCTGTTGCGGCCGAGTGTGGCTTGTCGGGTGATGCGGTCGCTTTCTTG |
| | CACGGTAGCGACCGTGAAGCGCTGGTGGAGTCTGCTAAGGCTTTGAAGGGTTTGATCGACCATAGTAGTGGTGGCGCGGGTG |
| | TGCGCCGTCTTGCGGGGAGTGCCCCCGTGGATGATGTTAAACGACGTGAGGGTGTCGCTTTTGTTGATGCTCTTGTCAATAAT |
| | TCTAGGAGATGATTTGTGATGGCTGATGATTTTCTTTCTGCAGGGAAGCTTGAGCTTCCTGGTTCTATGATTGGTGCGGTTCGT |
| | GATCGTGCTATCGATTCTGGTGTTCTTGCTAAGCTGTCGCCTGAGCAGCCGACTATTTTTGGTCCAGTGAAGGGTGCCGTGTTT |
| | TCGGGTGTTCCTCGCGCCAAGATTGTTGGTGAGGGTGAGGTTAAGCCTTCCGCTTCGGTTGGTGTTTCGGCGTTTACTGCGCA |
| | GCCTATCAAGGTTGTGACTCAGCAGCGTGTCTCGGACGAGTTTATGTGGGCTGATGCTGATTACCGTCTGGGTGTTTTGCAGG |
| | ATCTGATTTCGCCTGCTTTGGGTGCTTCGATTGGTCGCGCCGTGGATCTGATTGCTTTCCATGGTGTTGATCCTGCCACTGGTA |
| | AGCCTGCCGCGGCTGTCAAGACTTCGCTGGATAAGACGAAGCATATTGTTGATGCTACGGATTCTGCTACGACTGATCTTGTT |
| | AAGGCTGTCGGCCTGATCGCTGGGGCCGGTTTGCAGGTTCCTAACGGTGTTGCTTTGGATCCGGCGTTTGCTTTTGCTCTGTCG |
| | ACTGAGGTGTATCCGAAGGGGTCGCCGCTTGCCGGTCAGCCGATGTATCCTGCCGCCGGTTTCGCTGGTTTGGATAATTGGCG |
| | TGGCCTTAATGTTGGTGCTTCTTCGACTGTTTCGGGTGCCCCGGAGATGTCGCCTGCCTCGGGTGTTAAGGCTATTGTTGGTGA |
| | TTTCTCTCGTGTTCATTGGGGTTTCCAGCGTAATTTCCCGATCGAGCTTATCGAGTATGGTGACCCGGATCAGACTGGCCGTG |
| | ACCTGAAGGGCCATAATGAGGTTATGGTTCGTGCCGAGGCTGTGCTGTATGTTGCGATTGAGTCGCTTGATTCGTTTGCCGTG |
| | GTGAAGGAGAAGGCTGCCCCGAAGCCTAATCCTCTGGCTGGTAACTGATTCATTTGTTGCGATAATGTTTATGCTGTGTGCAG |
| | GGGGTGGTGTTGTTGGGTATCATTTTGAAGCCTGAGGATATTGAGCCTTTCGCCGATATTCCTAGAGAGAAGCTTGAGGCGAT |
| | GATTGCCGATGTGGAGGCTGTGGCTGTCAGTGTCGCCCCCTGTATCGCTAAACCGGATTTCAAATATAGGGATGCCGCTAAG |
| | GCTATTCTGCGCAGGGCTTTGTTGCGCTGGAATGATACCGGGGTTTCGGGTCAGGTGCAGTATGAGTCTGCGGGCCCGTTTGC |
| | TCAGACTACACGGTCTAATACTCCCACGAATTTGTTGTGGCCTTCTGAGATTGCTGCGTTGAAGAAGTTGTGTGAGGGTGATG |
| | GTGGGGCTGGTAAAGCGTTCACTATCATACCCACTATTAATGGTCGATATGTACATTCTGAGGTGTGTTCCACGGTGTGGGGT |
| | GAGGGTTGCTCGTGCGGATCTGATATTAACGGCTACGCTGGTCCTTTGTGGGAGATATGATATGACCAGTTTTCCTTACGGTG |
| | AAACGGTTGTGATGCTTCAGCCGACTGTTCGCGTCGATGATCTTGGCGACAAGGTGGAAGACTGGTCTAAGCCTGTCGAGAC |
| | TGTGTACCATAACGTGGCCATCTATGCTTCGTTGTCGCAGGAGGATGAGGCCGCGGGGCGTGACTCGGATTATGAGCATTGG |
| | TCGATGCTGTTCAAGTCCCCTGTTGTGGGTGCTGATTATCGTTGCAGGTGGCGTATCCGGGGTGTTGTGTGGGAGGCTGACGG |
| | GTCTCCTATCGTGTGGCATCATCCAATGTCTGGCTGGGATGCGGGCACGCAGATCAATGTGAAGCGTAAGAAGGGCTGATGG |
| | GTAGTGGCTCAGGATGTGAATGTGAAGCTGAACTTGCCGGGTATTCGTGAGGTGTTGAAGTCTTCTGGGGTGCAGGGCATGT |
| | TGGCTGAGCGTGGCGAGCGTGTCAAGCGTGCGGCCTCGGCGAATGTGGGCGGTAACGCTTTCGATAAGGCCCAATACCGTAA |
| | TGGTTTGTCGTCGGAGGTGCAGGTTCACCGTGTGGAGGCTGTGGCCCGTATTGGCACCACCTATAAGGGTGGGAAGCGTATT |
| | GAGGCGAAGCATGGCACGCTGGCTAGGTCGATTGGGGCGGCGTCGTGATCGTCTACGATGACCCCAGGAAGTGGGCTAAAC |
| | GCGTGCTCAAGGATGATGGCTGGCTGTCTGGGATACCATGCACCGGGACAGTGCCGGATAGCTTTGAGGGTGACCTTGTTTG |
| | GTTGGCTCTTGATGGTGGCCCGCAGTTGCATGTGCGTGAGCAGGTTTTTTTGCGGGTGAATGTGTTTTCTGATACGCCGGATC |
| | GGGCTATGTCTTTGGCGCGTCGCGTTGAGGCTGTCCTTGTTGACGGGGTTGATGGTGGCCCGGTGGTGTACTGTAAACGGTCT |
| | ACTGGCCCTGATTTGCTGGTTGATGGTGCACGTTTTGATGTGTATTCGCTTTTTGAGCTGGTGTGTCGGCCTGTCGAATCCGAG |
| | TAAGCGTATCGTTGTTTTTGTTGATTGATTTTTAGTTTGATTGTTTTTGGGGGTTATGATGGCTGCAACACGTAAAGCGTCTAA |
| | TGTTCGCTCTGCTGTTACGGGTGACGTCTATATTGGTAAAGCTCATGCCGGTGACGCTATTGATGGTGTGAAGACGGTTCCTG |
| | AGGGGTTGACTGCTTTAGGGTATCTGTCTGATGACGGGTTTAAGATTAAGCCTGAGCGTAAAACGGATGATTTGAAGGCTTG |
| | GCAGAATGCGGATGTTGTTCGCACGGTGGCTACAGAGTCGTCTATCGGGATTTCTTTCCAGCTGATCGAGTCTAAGAAGGAG |
| | GTTATCGAGCTGTTTTGGCAGTCGAAGGTTACTGCCGGATCCGATTCGGGTTCGTTCGATATTTCTCCTGGTGCTACCACTGGC |
| | GTGCACGCTTTACTGATGGATATTGTGGATGGCGATCAGGTTATTCGCTACTATTTCCCTGAGGTTGAGTTGATCGATCGTGA |
| | CGAGATTAAGGGTAAGAATGGCGAGGTGTATGGGTATGGTGTGACGTTGAAGGCGTATCCTGCCCAGATTAATAAGAAGGGT |
| | GATGCGGTGTCTGGTCGGGGGTGGATGACGGCTTTAAAAGCTGATACCCCTCCGGTTCCTCCGAAGCCTCAGCCGGATCCGA |
| | ATCCGCCGTCCGATAACTGATACACGATTTTAGGGATTGTTGATAGATGAGTGATACTGGTTTCACGTTGAAGATTGGTGACC |
| | GTAGCTGGGTGTTGGCGGATGCGGAGGAGACGGCTCAGGCTGTTCCTGCTCGCGTGTTTCGCCGTGCCGCTAAGATTGCCCA |
| | GTCGGGTGAGTCTGCGGATTTCGCCCAGGTTGAGGTGATGTTTTCTATGTTGGAGGCTGCCGCCCCGGCTGACGCGGTGGAGG |
| | CCCTGAGGGGCTTCCTATGGTTCGTGTGGCGGAGGTTTTCCGTGAGTGGATGGAATACAAGCCTGAAGGTAAGGGTGCCTCG |
| | CTGGGGGAATAGTTTGGCTCCACGGCCTGATTGATGATTATCGTGGGGCCATCGAATACGATTTCCGCACCAAGTTTGGTGTT |
| | TCTGTTTATAGTGTTGGTGGCCCGCAGATGTGTTGGGGTGAGGCTGTCCGGCTGGCTGGCGTGTGGTGTACCGATACGTCTAG |
| | CCAGTTGGCGGCCCACCTGAATGGTTGGCAGCGCCCGTTTGAGTGGTGCGAGTGGGCTGTGCTGGACATGTTGGATCATTAC |
| | AGGTNTGCTAATAGTGAGGGGCAGCCGGAGCCTGTGGCGAGGCCGACGGATGAGCGTAGGGCCCGGTTTACGTCTGGGCAG |
| | GTGGACGATATTTTGGCGCGTGTTCGTGCTGGTGGCNGGGTGTCTCGCGAGATTAATATTATGGGGTGAATAGTGTATGTCTG |
| | GTGAGATTGCTTCCGCATATGTGTCGTTGTATACGAAGATGCCGGGTTTGAAGTCTGATGTTGGTAAACAGTTGTCGGGTGTG |
| | ATGCCTGCTGAGGGTCAGCGTTCGGGTAGCCTGTTTGCTAAGGGTATGAAGTTGGCTTTGGGTGGCGCGGCGATGATGGGTG |
| | CCATTAATGTTGCTAAGAAGGGCCTCAAGTCTATCTATGATGTGACTATTGGTGGCGGTATTGCTAGGGCGATGGCTATTGAT |
| | GAGGCTCAGGCTAAACTGACTGGTTTGGGTCACACGTCGTCTGACACGTCGTCGATTACGAATTCGGCTATTGAGGCTGTGAC |
| | GGGTACGTCGTATGCGTTGGGGGATGCGGCGTCTACGGCTGCGGCGTTGTCTGCTTCGGGTGTGAAGTCTGGCGGGCAGATG |
| | ACGGATGTGTTGAAGACTGTCGCCGATGTGTCTTATATTTCTGGTAAGTCGTTTCAGGATACGGGCGCTATTTTTACGTCCGTA |
| | ATGGCTCGCGGTAAGTTGCAGGGCGATGACATGTTGCAGCTTACTATGGCTGGTGTTCCTGTGCTGTCTTTGCTTGCCAGGCA |
| | GACGGGTAAAACCTCGGCTGAGGTGTCGCAGATGGTGTCGAAGGGACAGATTGATTTTAACACGTTTGCGGCTGCGATGAAG |
| | CTTGGCATGGGTGGTGCTGCGCAGGCGTCTGGTAAGACGTTTGAGGGCGCTATGAAGAATGTTAAGAGCGCCCTGGGTTATC |
| | TTGGTGCTACGGCTATGGCGCCGTTTCTTAACGGGTTGCGGCAGATTTTTGTTGCGTTGAGTCCGGTTATCAAGTCGGTGACG |
| | GATTCTGTGAAGCCGATGTTTGCGTCGGTGGATCAGGGTATTCAGCGGATGATGCCGTGTATTTTGGCGTGGATTAACCGTAT |
| | GCCGGCCATGATTACGAGAATGAATGCACAGATGCGCGCCAAGGTGGAGCAGTTGAAGGGCAGTTTTGCGAGAATGCATTTG |
| | CCTGTCCCTAAAGTGAATTTGGGTGCCATGTTTGCTGGCGGCACCGCAGTGTTTGGTATTGTTGCTGCCGGTGTGGGGAAGCT |
| | TGTTGCAGGGTTTGCCCCGTTGGCGGTGTCGTTGAAGAATCTCCTGCCGTCATTTGGTGCTTTGAAGGGTGCCGCTGGCGGGC |
| | TTGGCGGCGTGTTTCGCGCCTTAGGTGGCCCTGTCGGTATTGTGATCGGGCTGTTTGCGGCAATGTTTGCCACGAACGCCCAG |
| | TTCCGTGCCGCGGTGATGCAGCTTGTGGCTGTGGTTGGCCAGGCGTTGGGCCAGATCATGGCTGCTGTGCAGCCGCTGTTCGG |
| | GATTGTTGCTGGTTTGGTGGCACGGTTGGCTCCCGTTTTTGGCCAGATTATCGGCATGGTTGCCGGTTTGGCTGCCCAGTTGAT |
| | GCCTGTGATTGGTATGCTGGTGGCCCGGCTGGTGCCTGTGATCACGCAGATTATTGGTATGGTGACGCAGGTTGCGGCCATGT |
| | TGCTGCCTACGTTGATGCCGGTTATTCAGGCTGTTGTTGCTGTGATACGGCAGGTTGTTGGTCTTATCCGGTCGCTGATACCAG |
| | TCATCATGTCGATTATGCGTGTGGTGGTGCAGGTTGTTGCGGTTGTGATACAGGTGGTGGCCCGTATTCTTGCGGTTGTGGCT |
| | CCGATGGTGGCTGCTGTGGTGGGGTTTGTTGCCCGTATTGTTGGTGCTGTCGTGTCGGCTGTGGCCCGTGTGATTGCTGCTGTT |
| | GCCCGTGTCGTCTCATGGGTTGTGGCCCATTTGGTGTCTGGTGTGGCGCATATGGGTTCGGCTATACTGAATGGCTGGAAACG |
| | TATTAGGGCGTTTACGTCTGCGTTTATGAGCGGGTTTAAGTCGCTCATTTCTGCCGGTGTGGGCGCTGTTGTCGGGTTTTTTGC |
| | GCGGCTTGGTTTGTCGGTTGCTTCCCATGTTCGGTCTGGGTTTGGCGCGGCTCGTGGTGCTGTTTCGGCTGCGATGAATGGGAT |
| | TCGGAGTGTTGTGCCTTCGGTGGTGTCCGCGGTGGGCTCGTTTTTTGGTTCGATGGCTTCTAGGGTTCGTGGTGCTGCCTCGTC |
| | TGGGTTTAACGGGGCCAGGAGTGTGGCTTCTTCTGCTATGCATGCTATGGGGTCTGCGGTGTCTAGTGGTGTGCATGGTGTGC |
| | TGGGTTTTTTCCGGAATTTGCCTGGCAATATTCGGCGTGCGCTTGGTAATATGGGGACCCTGTTGGTGTCTGCTGGCCGGGAT |
| | GTGGTGTCTGGTTTGGGTAATGGTATCCGGAATGCTATGAGTGGCCTGTTGGATACGGTGCGTAATATGGGTTCTCAGGTTGC |
| | TAATGCGGCGAAGTCGGTGTTGGGTATTCATTCCCCGTCTAGGGTGTTTCGTGAACAGGTTGGCCGCCAGGTTGTTGCCGGTT |
| | TGGCTGAGGGGATCACCGGGAATGCCGGTTTGGCGTTGGATGCGATGTCGGGTGTGGCTGGTCGGTTGCCTGATGCGGTTGA |
| | TGCCCGGTTTGGTGTGCGATCGTCGGTGGGCTCGTTACACCGTATGGCAGGTATCAGCGCATGAATGATAAGAGTGTTGTGGT |
| | GAATGTTAACGGGCCAACCTATGGGGATCCTAACGAGTTTGCGAAGCGGATTGAGCGGCAGCAGCGTGACGCGTTGAACGC |
| | GTTGGCTTACGTGTGATTGCGGGGTGTGGTTCATGTTTATTCCTAACCCGTCTGATCGTGCCGGTTTGACGGTGGATTGGACG |
| | ATGTTTCCGTTGATCGGTAATAATCCGGAGCGTGTTCTTCATTTGACGGATTATACGGGTGCGTCCCCGGTCATGTTGTTGAAT |
| | GATTCGTTGCGCGGTTTGGGTGTTCCTGAGGTGGAGCATTTTTCTCAAACTCATGTTGGGGTGCACGGCTCGGAGTGGCGCGG |
| | GTTTAATGTGAAGCCTCGCGAGGTGACATTACCTGTGTTGGTGTCGGGTGTTGACCCGGATCCGGTGGGCGGGTTTCGTGACG |
| | GTTTTTTGAAAGCCTATGACGAGTTGTGGTCTGCTTTTCCTCCGGGCGAGGAGGGGGAGTTGTCGGTGCGGACCCCGGCGGGT |
| | GTTGAGCGTGTGTTGAAGTGCCGGTTTGATTCGGCGGATGACACGTTTACGGTTGATCCGGTTAATCGTGGCTATGCGCGCTA |
| | TCTGTTGCATTTGACAGCCTATGACCCGTTTTGGTATGGGGATGAGCAGAAGTTTCGTTTCAGTAACGCGAAGTTGCAGGATT |
| | GGTTGGGTGGCGGCCCTGTCGGCAAGGATGGTACGGCGTTTCCTGTGGTGTTGACGCCTGGTGTGGGCTCTGGTTGGGATAAT |
| | CTGTCTAATAAGGCTCATGTGCCTG |
| | SEQ ID NO.: 4; >3530 len: 1909 cycle: O |
| | CCCTAATACGATTCCAGCCAGCCTGAACCACCGAACCCATACGCGCCAAACCAGACACAAAATGGGCCACAACCCACCCAAC |
| | AACACGGGCAACAGCAGCAAAAACACGGGCCACAGCCGACACAACAGCACCAACAATACGGGCAACAAACCCCACCACAG |
| | CAGCCACCATCGGAGCCACAACCGCAAGAATACGGGCCACCACCTGTATCACAACCGCAACAACCTGCACCACCACACGCAT |
| | AATCGACATGATGACTGGTATCAGCGACCGGATAAGACCAACAACCTGCCGTATCACAGCAACAACAGCCTGAATAACCGG |
| | CATCAACGTAGGCAGCAACATGGCCGCAACCTGCGTCACCATGCCAATAATCTGCGTGATCACAGGCACCAGCCGGGCCACC |
| | AGCATACCAATCACAGGCATCAACTGGGCAGCCAAACCGGCAACCATGCCGATAATCTGGGCAAACACTGGCGCCAACCGT |
| | GCCACCAGCCCCGCAACCAAACCAAACAGCGGCTGAAGAGCGGCCATAATCTGGCCCAGGGCTTGGCCAACCACAGCCACA |
| | AGCTGCATCACAGCGGCACGGAACTGGGCGTTCGTAGCAAACATGGCAGCAAACAGCCCGATCACAACACCGACAGGGCCA |
| | CCCAGGGCACGGAACACGCCGCCAAGACCCCCGGCGGCACCCTTCAAAGCACCAAACGATGGTAGCAGATTCTTCAACGAC |
| | ACCGCCAACGGGGCAAACCCCGCAACAAGCTTCCCAACACCTGCAGCAACAACACCGAACACGGCGGTGCCGCCAGCAAAC |
| | ATGGCAGCCAAATTCACTTTAGGGACAGGCAAATGCAGCCTCGCAAAAATGCCCTTCAACTGCTCCGCCTTGGCGCGCATCT |
| | GTGCATTCATTCGTGTGATCATAGCCGGCATCCGGTTAATCCACGCCAAAATAGACGGCATCATCCGCTGAATACCCTGATCC |
| | ACCGACGCAAACAGGGGCTTCACAGAATCCGTGACAGACTTAATAACCGGATTCAACGCAACAAAAATCTGCCGCAGACCG |
| | TTAAGAAACGGCGCCATAGCCGTAGCACCAAGATAACCCAGGGCACCCTTAACATTCTTCATAGCGCCCTCAAACGTCTTAC |
| | CAGACGCCTGCGCAGCACCACCCATGCCAAGCTTCATCGCAGCCGCAAACGTGGCAAAATCAATCTGCCCCTTCGACACCAT |
| | CTGCGACACCTCGGCCGACGTTTTACCCGTCTGCCTGGCAAGCAAAGACAGCACAGGAACACCCGCCATCGTAAGCTGCAAC |
| | ATGTCATCGCCCTGCAACTTACCGCGAGCCATCACAGACGTAAAAATAGCGCCCGTATCCTGAAACGACTTACCAGAAATAT |
| | AAGACACATCGGCGACAGTCTTCAACACATCCGTCATCTGCCCGCCAGACTTCACACCCGAAGCAGACAACGCCGCAGCCGT |
| | AGACGCCGCATCCCCCAACGCATACGAAGTACCAGTCACAGCCTCAATAGCCAAATTCATAATCGAAGACGTGTCAGAAGAC |
| | GTGTGACCCAAACCAGTCAACTTCGCTTGGGCCTCATCAATAGCCATAGCGCGAGCTATACCGCCACCAATAGTCACATCAT |
| | AAATCGACTTGAGGCCCTTCTTAGCAACGTTGACGGCACCCACCATTGCGGCGCCACCCAAAGCCAACTTCATACCCTTAGCA |
| | AACAGGCTACCCGAACGCTGACCCACAGCAGGCATCACACCCGACAACTGATTACCAACATCAGACTTCAAACCCGGCATCT |
| | TCGTATACAACGACACATACGCGGAAGCAATCTCACCAGACATACACTATTCACCCCATAATATTAATCTCGCGAGACACCC |
| | CGCCACCGGCACGAACACGCGCCAAAATAT |
| | SEQ ID NO.: 5; >3531 len:2184 cycle: O |
| | GGGTATGAAGTTGGCTTTGGGTGGCGCCGCAATGGTGGGTGCCATCAACGTGGCTAAGAAGGGTCTCAAGTCTATCTATGAT |
| | GTGACTATTGGTGGCGGTATTGCTAGGGCGATGGCTATTGATGAGGCTCAGGCTAAGTTGACTGGTTTGGGTCACACGTCTTC |
| | TGACACGTCTTCGATTATGAATTCGGCTATTGAGGCTGTGACTGGTACGTCGTATGCGTTGGGTGATGCGGCTTCTACTGCGG |
| | CGGCGTTGTCTGCTTCGGGTGTGAAGTCTGGCGGGCAGATGACGGATGTGTTGAAGACTGTCGCGGATGTGTCTTATATTTCG |
| | GGTAAGTCGTTTCAGGATACGGGCGCTATTTTTACGTCTGTGATGGCTCGCGGTAAGTTGCAGGGCGATGACATGTTGCAGCT |
| | TACGGTGGCGGGTGTTCCTGTGCTGTCTTTGCTTGCCAGGCAGACGGGTAAAACGTCTGCTGAGGTGTCGCAGATGGTGTCGA |
| | AGGGGCAGATTGATTTTGCCACGTTTGCGGCTGCGATGAAGCTTGGTATGGGTGGTGCTGCTCAGGCGTCTGGTAAGACGTCT |
| | GAGGGCGCTATGAAGAATGTTAAGGGCGCTTTGGGCTATCTTGGCGCTACTGCTATGGCCCCGTTTCTTAACGGGTTGCGGCA |
| | GATTTTTGTTGCGTTGAATCCGGTTATCAAATCGGTTACGGATTCTGTGAAGCCTTTGTTTGCGTCGGTGGATCAGGGTATTCA |
| | GCGGATGATGCCGTCTATTTTGGCGTGGATTAACCGTATGCCGGGCATGATCACGAGAATGAATGCACAGATGCGCGCCAAG |
| | GTGGAGCAGCTGAAGGGCATTTTTGCGAGAACGCATTTGCCTGTCCCTAAAGTGAATTTGGGTGCCATGTTTGCTGGCGGCAC |
| | CGCAGTGTTCGGTATTGTGGCTGCCGGGGTGGGGAAGCTTGTTGCAGGGTTTGCCCCGTTGGCGGTTGCGTTGAAGAATCTAC |
| | TGCCGTCGTTTGGTGCTTTGAAGGGTGCCGCTGGCGGGCTTGGCGGCGTGTTTCGCGCCCTGGGTGGCCCTGTCGGGATTGTG |
| | ATCGGCTTGTTTGCTGCCATGTTTGCCACTAACGCCCAGTTCCGTGCCGCTGTTATGCAGCTTGTTGGTGTGGTTGGCCAGGCT |
| | TTGGGGCAGATCATGGCTGCTGTGCAGCCGCTGTTCGGGATTGTTGCTGGTCTGGTGGCACGGTTGGCTCCCGTTTTTGGCCA |
| | GATTATTGGTTTGGTTGCCGGGTTGGCTGCCCAGTTGATGCCTGTGATTGGTATGTTGGTGGCCCGTCTTGTTCCTGTGATCAC |
| | GCAGGTTATTGGTATGGTGACGCAGGTTGCGGCCATGTTGCTGCCTACGTTGATGCCGGTTATTCAGGCTGTTGTTGCTGTGA |
| | TACGGCAGGTTGTTGGTCTTATCCGGGCGCTGATACCAGTCATCATGTCGATTATGCGTGTGGTGGTGCAGGTTGTTGCGGTT |
| | GTGATACAGGTGGTGGCCCGTATTCTTGCGGTTGTGGCTCCGATGGTGGCTGCTGTGGTGGGGTTTGTTGCACGTATTCTTGG |
| | CGCTATTGTGTCTGCTGCAGCCCGGATTATTGGGACTGTCACCCGTGTCATTTCATGGGTTGTGAATCATTTAGTGTCTGGCGT |
| | GAGGTCTATGGGCACGGCCATCTTGAATGGCTGGAATCATATTAGAGCGTTTACGTCTGCGTTTATTAACGGTTTCAAGTCGG |
| | TGATTTCTGGCGGCGTGAACGCGGTTGTGGGGTTTTTTGCCCGGCTGGGTTCTTCGGTTGCTTCTCATGTGAGGTCTGGTTTTA |
| | GCGCGGCTCGTGGCGCTGTTTCTTCTGCGATGAATGCTATCCGGAGTGTTGTGTATTCGGTGGTGTCCGCGGTGGGCTCGTTTT |
| | TTGGTTCGATGGCTTCTAGGGTTCGTGGTGCTGCCTCGTCTGGGTTTAACGGGGCCAGGAGTGTGGCTTCTTCTGCTATGCATG |
| | CTATGGGGTCTGCGGTGTCTAGTGGTGTGCATGGTGTGCTGGGTTTTTTCCGGAATTTGCCTGGCAATATTCGGCGTGCGCTTG |
| | GTAATATGGGGTCCCTGTTGGTGTCTGCTGGCCGTGATGTGGTGTCTGGTTTGGGTAATGGTATCCGGAATGCTATGAGTCTG |
| | TCTCTTATACACATCTGACG |
| | SEQ ID NO.: 6; >3532 len: 1031 cycle: O |
| | CCCCTCAAAAACCTGTACTGCACCGACACAACAATGGCACCCAAACCAACCATCTCAATATTCACACTCTTAGAACCGCCAG |
| | GCGGGATAGGGGCAAACTCCCACTCCTTCAAACGATCCATCACATCCTCAAACCCGTTCAACAATGCAGACTGTTTGCGAGG |
| | ATCCGTATCAATAGTGATCCAATCATACTCCTCGACAGGATAGTCCGAAGACACACGCAAACCATCAATCTGCACAGACCAC |
| | GACTCCAAAGGCCCCTCAACACGAATCACAGGCCACGCAGGCACATCACCCTTATTAGACAGGTTATCCCAACCAGAACCAA |
| | CACCAGGCGTCAACACCACCGGAAACGCAGTACCCTTCTTATTAACAGGGCCGCCACCCAACCAATCCTGCAACTTCGCGTT |
| | ACTGAAACGAAACTTCTGCTCATCCCCATACCAAAACGGGTCATAGGCTGTCAAATGCAACAGATAGCGCGCATAGCCACGA |
| | TTAACCGGATCCACCGTAAACGTGTCATCCACCGAATCAAACCGGCATTTTAGCGCACGCTCAACACCGGCAGGAGTCTTCA |
| | CCGACAACTCCCCCTCCTCGCCCGGAGGAAAAGCAGACCACAACTCGTCATAGGCTTTCAAAAAACCGTCACGAAACCCGCC |
| | CACCGGATCCGGGTCAACACCCGACACCAACACCGGCAGCGTCACCTCGCGAGGCTTCACATTAAACCCGCGCCACTCCGAG |
| | CCGTGCACCCCAACATGTGTTTGAGAAAAATGCTCCACCTCAGGAACACCCAAACCGCGCAACGAATCATTCAACAACATCA |
| | CCGGAGACGCACCCGTATAATCCGTCAAATGAAGCACACGCTCGTCGCCAGACAGCGGATCCATAAACCATGTCACAGTCAA |
| | ACCCGAACGATCAGACGGGTCAGGAATAAACATGCACAACACCCCAATCACACGTAAGCCAACGCGTTCAAAGCGTCACGC |
| | TGCTGCCGCTCAATCCGCTTCGCAAACTCGTTAGGATCACCATAAGTA |
| | SEQ ID NO.: 7; >3533 len: 1357 cycle: O |
| | GAAGCATAGATGGCCACGTTATGGAACCCAGTCTCGACAGGCTTAGACCAGTCTTCCACCTTGTCGCCAAGATCATCGACAC |
| | GAACAGTCGGTTGAAGCATCACAACCGTTTCACCATAAGGAAAACTGGTCATATCATATCTCCCACAACGGGCCATCATGGC |
| | CGTTAATGTTAGACCCGCACGAGCAGCCCTCACCCCACACCGTGGAACACACCTCAGAATGAGCATATCCACCACGAATCGT |
| | GGGTGTGATCGTGAACGCTTTACCAGCCCCACCATCACCCTCACACAACTTCTTCAACGCAGCAATCTCGGAAGGCCACAAC |
| | AAATTCGTGGGCGTATTAGACCGTGTAGTCTGGGCAAAAGGACCCGCAGACTCATACTGCACCTGCCCCGAAACCCCGGTAT |
| | CATTCCAGCGCAGCAAAGCCCTACGCAGAATAGCCTTAGCGGCATCCTTATATTTGAAATCCGGTTTAGCGATACAGGGGGC |
| | GACACTGACAGCCACAGCCTCCACATCGGCAATCATCGCCTCAAGCTTCTCTCTAGGAATATCGGCGAAAGGCTCAATATCCT |
| | CAGGCTTCAAAATGATACCCATCAACACCACCCCCTGCACACAGCATAAACATTATCGCAACAAATGAATCAGTTACCAGCC |
| | GGAGGATTAGGCTTCGGGGCAGCCTTCTCCTTCACCACGGCAAACAAATCAAGCGACTCAATCGCAACATACAGCACAGCCT |
| | CGGCACGAACCATAACCTCATTATGGCCCTTCAGGTCACGGCCAGTCTGATCCGGGTCACCATACTCGATAAGCTCGATCGG |
| | GAAGTTACGCTGGAACCCCCAATGAACACGAGAGAAATCACCAACAATAGCCTTAACACCAGAGGCAGGCGACATCTCCGG |
| | GGCGCCAGAAACAGTCGAAGAAGCACCAACATTCAGGCCGCGCCAATTATCCAAACCGGCAAACCCGGCGGCAGGATACAT |
| | CGGCTGGCCGGCAAGCGGAGACCCCTTCGGATACACCTCAGTCGACAGAGCAAACGAGAACGCCGGATCCAAAGCAACACC |
| | GTTAGGAACCTGCAAACCAGCACCAGCGATAAGGCCGACAGCCTTAACAAGATCGGTCGTAGCAGAATCCGTGGCATCAAC |
| | AACATGCGTCGTCTTATCCAGCGAAGACTTGACAGCCGCAGCAGGCTTACCCGTAGCCGGATCAACACCATGGAAAGCAATC |
| | AGATCAACAGCGCGACCAATCGAAGCACCAAGAGCCGGGGAAATCAGATCCTGAAGCACACCCAGACGGTAATCAGCATCA |
| | GCCCACATAAACTCGTCCGAGACACGCTGCTGAGTCACAACCTTAATAG |
| | SEQ ID NO.: 8; >3534 len: 1122 cycle: O |
| | GGCGACATCTGTGGTTGTGGGTGGCCGCGGCGAGGGCCGGGATCGGCTGTTTTGTGAGGATGTTGATTCGATGGCCGAGGAT |
| | GACTGGTTTGATCGTGTCGAGGTGTTTAAGGATGCCCGTAACACGGATTCTGAGCATGTGCATCTCATCGATGAGGCTGAGCA |
| | GGTGCTGTCCGAGTTGGGGGCCACGTCGGGGTTTAAGATTGAATTGGCTGAGTCGGAGGTGTTGCGTTTTGGGCCAGGCAAT |
| | CTGATGCCGGGTGATTTGATCTATGTGGATGTGGGTTCGGGCCCTATTGCGGAGATTGTTCAGCAGATTGATGTGGAGTGTGT |
| | ATCGCCTGGTGATGGTTGGACGAAGGTGACTCCGGTTGCGGGGGATTATGAGGATAATCCGTCGGCCCTGTTGGCTCGCCGT |
| | GTGGCTGGTTTGGCTGCGGGTGTGCGGGATTTGCAAAAATTTTAGAAAAGAATTGGTGGTTTGTTGTGGGTATTGTGTGTAAA |
| | GGGTTTGATGGTGTGTTGACCGAGTATGATTGGGCTCAAATGTCTGGTCTGATGGGTAACATGCCTTCGGTTAAGGGGCCGGA |
| | GGATTTTCGTGTCGGCACTACTGTTCAGGGTGCCACGGTGTTGTGTGAGGTTTTGCCGGGGCAGGCTTGGGCTCACGGGGTGA |
| | TGTGCACGTCGAATAGTGTTGAGACGGTGACGGGCCAGCTGCCGGGCCCTGGTGAGACCCGCTACGACTATGTGGTGTTGTC |
| | TCGGGATTGGGAGCAGAACACAGCCAAGTTGGAGATTGTTCAGGGTGGCCGTGCGGAGCGTGCCCGTGACGTGTTGCGTGCC |
| | GAGCCTGGCGTGTACCATCAGCAGCTACTGGCTACTTTGGTGGTGTCGTCTAACGGGTTGCAGCAGCAGTTGGATAGGCGTG |
| | CTGTGGCGGCTAGGGTTGCGTTTGGGGAGTCTGCTGCGTGTGATCCTACCCCTGTGGAGGGTGACCGTNTGATGGTTCCTTCT |
| | GGGGCTGTGTGGGCTAACCATTCGGGCGAGTGGATGCTACTGTCTCCGAGGATTGAGACGGGTTCGAAGTCTATCATGTTTG |
| | GTGGATCGAACGTGTATGCTTACACGATCCCGTTCGAGCGGCCGTTTACT |
| | SEQ ID NO.: 9; >3535 len: 1324 cycle: O |
| | CTATTACACCGACCATGAGGAGTAGTGTGAATCATTCTGAGGTGTGTTCCACGGTGTGGGGTGAGGGTTGCTCGTGCGGGTC |
| | GAATATTAACGGCTACGCTGGCCCTTTGTGGGAGATATGATATGACCAGTTTTCCTTATGGTGAAACGGTTGTGATGCTTCAA |
| | CCGACTGTTCGCGTCGATGATCTTGGTGACAAGGTTGAGGATTGGGGGCATCCTGTAGAAACCGTGTACCATAACGTGGCCA |
| | TCTATGCTTCCGTTTCACAGGAGGATGAGGCCGCGGGGCGTGACTCTGACTATGAGCATTGGTCGATGCTTTTCAAGCAGTCT |
| | GTTGTGGGTGCTGATTATCGTTGCCGGTGGCGTATTCGGGGTGTTGTGTGGGAGGCTGACGGGTCTCCTATGATGTGGCATCA |
| | CCTAATGTCCGGTTGGGATGCGGGCACGCAGGTTAATGTGAAGCGTAAGAAGGGCTGATAGATTGTGGCTCAGGATGTGAAT |
| | GTGAAGCTGAACTTGCCGGGTATTCGTGAGGTGTTGAAGTCTTCTGGGGTGCAGGGCATGTTGGCTGAGCGTGGTGAGCGTG |
| | TCAAGCGTGCAGCCTCTGCGAATGTGGGCGGTAACGCTTTCGATAAGGCCCAATACCGTAATGGTTTGTCGTCAGAGGTGCA |
| | GGTTCACCGTGTTGAGGCTGTCGCTCGTATAGGCACCACATATAAGGGTGGGAAGCGTATTGAGGCGAAGCATGGCACGCTG |
| | GCTCGTTCGATTGGGGCTGCGTCGTGATCGTCTACGATGACCCCAGGAAGTGGGCTAAACGCGTGCTCAAGGATGATGGCTG |
| | GCTGTCTGGGATACCTTGTACTGGGACAGTGCCGGATAGTTTTGAGGGTGACCTTGTTTGGTTGGCTCTTGATGGTGGCCCGC |
| | AGTTGCATGTGCGTGAGCAGGTTTTTTTGCGGGTGAATGTGTTTTCTGATACGCCGGATCGGGCTATGTCTTTGGCGCGTCGC |
| | GTTGAGGCTGTCCTTGTTGACGGGGTTGATGGTGGCCCGGTGGTGTACTGTAAACGGTCTACTGGCCCTGATTTGCTGGTTGA |
| | TGGTTCACGTTTTGATGTGTATTCGCTGTTCGAGCTGATATGTAGGCCTGCGGAGTCCGAATAAGCTTATTGTTTTTGTTTTAA |
| | TGTAATTGTTTGATATTTAATGGGGGTTATGATGGCTGCAACACGTAAAGCGTCTAATGTTCGCTCTGCTGTTACGGGTGACG |
| | TCTATATTGGTAAAGCTCATGCCGGTGACACTATTGATGGTGTGAAGACGGTTCCTGAGGGGTTGACTGCTTTAGGGTATCTG |
| | TC |
| | SEQ ID NO.: 10; >3536 len: 1069 cycle: O |
| | GTGTCGAATCGTCCGACGCAGTGGCAGTAGTCGTACATGAGTTTAATAATGTGTTGGTGGTCGCCAAGGTAGGGGTTTGCGCT |
| | GATGCTGTAGGTGGCTGTGCCGTCTTTGCTAATAGTGTATTTGGCGGTGATGGTTTGGGGGTTTTCTGTGTTGGTGATAATTGC |
| | TGTGGTGGTGGCGCCTACGGTTTGGAGGACGGTGGTTTGGGTTCCGTCGTCTAGTGTTGTGGTAACCATAATTGGGGTTCTCC |
| | TTTAGATGCTTGTCTGGTGGTCGGCTAGATGAATAATATCGGATAAAGGTTTCGGTTGGTCTAGGTGTTGTATGGTTTTGTTGG |
| | CTAGCCGTTTGGCTGCCCTGTAACACATTTTGGTGTAGTGTTTGTTGTCTAGGTTGTGGTATTGTTCACGTACCGCAATATATA |
| | GTAGAGAGTCTTGGTACAGGTCGTCGGGGTTGACGGCCGGGTAGTGGCGGGCAATGTTGGTGCAAGCTTTGTGTAGCTGGTG |
| | TTGGTGGTGGTGGGTTGCCCACCCCCAGTTGGCGGTGGTGGCTTGTTGGACTTTGGTTGGTCGTCTACTCATGGTTTTTATCTC |
| | GCTATCTGATAGTTGTTTGGTGTTTTGTTGTTGATAGTGTAGCACACTAGTCCGGGGTTTCCGGTGGTGCCTGTGCGGTGCCGG |
| | TACCAGACTGATTCTCCTTCCATGGATGGGCATTGGATGAAGGTGCGTTGTCCTTGCTCGGAGATTTCTAGGTGGTGCCGGTG |
| | GCCTGCCATCAGGATGTGTGATGTGGTGCCGTTGTGGAATTCTTGGCCGCGCCACCATTCGTAGTGTTTGCCGGTGCGCCATT |
| | GGTGTCCGTGGGCGTGCAGGATGGTAGCCCCGGCTACGTTGACGGTGGTGGTCATTTCGTCGCGTTGGGGGAAATAATAGTG |
| | GAGGTTGGGGTAGTTGTTGGTGAGTTGGTAGGCTTCTGCGATGGCGCGGCAGCAGTCCACGTCGAATGAATCATCGTAGGTC |
| | GTGACGCCTTTGCCGAATCTAACGGCTTCTCCGTGGTTGCCGGGCATGGAGGTGACCGTAAGGTAGGACCA |
| | SEQ ID NO.: 11; >3537 len: 1224 cycle: O |
| | CGTGTTTGGGCGCAGCTTGTGGAGTTCGGCTTCCACGCTCTGCCGGTAGGCTTCGGCGAGGCCCTCAAAATCCATGTGGTCGC |
| | AGGAGAGGTTTTCGAGGCGTGTCAGGTCGAAGGGTGTGGGGCAGTCGTAGCTGGCGGGGGTGTAGAGCTGGGTGAAGTGGT |
| | TGGCGATCTTCTGCATGACTGGTTCCTTTCTGGTGTGTGGATGGTTTTTATCGTGTGGCTTCGGCGATGATGGCGTCCACATCG |
| | ATTGTGTCGATCATGTCGTGGAGTTCCTCAGTCTCATCTGGGGTGAGTGGCTGCCAGTTGCGTGGCCCATACACTGCACCGTC |
| | GAGAGTGATGGTCCACAGGGGTCGGATGAGTCGTACGGCTTCTTCGACTTTGGCACGGTATAGGCGGCAGATGATAGACGTG |
| | TGGGTGTTACCTATGTCACATCCTGCCAGGTGTGTGGGGTGGAGTGGGTTGATTTCTGTTTGCCCGTAGAGGCTGGTGAAGGA |
| | TGGGGTGATGAGTGTGGTGGCCATTTTTGGTGTGTTCCTTTCGGTGGTGTATGGGTTGTTGTGGTTTCTAGAGTGTGTAGGTTG |
| | CAACCGGGAGTCAAGGCTGCGCTCAACCGGATTGAGCGTTTCATGCTGGAGTGTCGGGTGTGACAGATGCCACTTAAGCCTT |
| | TATGGCATCCCTCAGCGTCTCAAATCTTCTGGGGGTAGGATTATGCAGGGTTGGCCCTGCTGATCGATTCTAGGGCCTTCTAG |
| | GGCGTCTCAGGGGTATGTCTGGGTGATAGCAGGTCCGGTAGATGACCCGGCAGATCTACCTTGGCTTTCATCACGGGGGTCG |
| | AGATGCCATATCTGGACATGGAATCTAAACCCTCATACTGTGTGAGATGTATCACACTCGCCTAGTATGGTGTGCACCCTCGA |
| | GGCTACTCTGTCGATCTTGCGTGGAGGGTGTAGCCCAGAAATGCCGTTTAAAGCTTCAGGGGTACGCCTAGGAGCGCCTTAC |
| | AGGATGGTGGCTAGGTATTTATACCACCAAGCAATTCTGATCGATTCTAGACGCCTCCAGGAGACTGATACGCGATCCGCTAT |
| | CCAGACGCCGACCACCAGCCCCTATCCTGGTTAGCTAAGCCTCAACTATGTGGACAGTGTGGGATACTGTGGGGGGAGAAGG |
| | ACGCGGTAAAAGAAAGAGGGGGGCATCAGCCTTAGGGTCTTAGCACTGATGGACTTAGCACCGAGCC |
| | SEQ ID NO.: 12; >3538 len: 1266 cycle: O |
| | CATCGGCGCCCACAAAGACTCCTTTACTTTCGCCTACGGTGCCACCACCAAGCAACCCGGAGCATCCAAGGCACAATTGAAA |
| | GTCTTCTGCCAAGTCTCTGGCACCGAAGACAAGCCGCAGGTAGAGGACATGTCCATCAACCCTTCACGCTAACCCGAAAGAC |
| | AACACACCCATTATGCCTCTCATGTCCACCTACATGGCTAATGCGGGCCTGTCCGACATCATGCACATCCTCCATACCGCCGG |
| | GGGAGCCTGCCGGACAGGCACCGACATCGCCGCACGAATCAACACACTCCACCCAGAATACAATCTCCAAGCCACCACCGA |
| | CCAGCTCATCGACATCGAAAACCGCATCGACACTATTCGCAAAAACATTCAAAAAGAAATCAAGAAACACCCGTGAACATC |
| | ACCGACCACACCACCCCAGACCTGCACTCCTACGAAACGTTCTTCACCAGCCTCGCATGGATCCAAGGCGGCATCATCACAT |
| | GGATGTACGCAACCGGCACCCCACACAAGGCAGCCCTCGCCATTATTGCCGCATGCGCCCTCGCCACCCTCCTGGGTGCATC |
| | AACACTCACATACAAGCCCAAAGACAGCAAATGATCACAACACCCATACTGATCGCTGAAACCCTCGCCATAATTATTCTCG |
| | CAGTAGCACTCGCCCACAACAACAACCAGTAACCCACCCTTAAGGAGCACACATCTCATGGATGAGCCCACCCGCATGTACA |
| | CCGACCCCAACACCGGTGCCCGAAAAGAATTGAAACTCTGCAGGCTCTCCCTCATCGACCCCGCATCCCTTCACGCTCTCGGC |
| | GAAGTAGCAGGCTACGGTGCCACCAAATACGGCGACAACAACTGGACCGGAGGATACCCGTGGAGCCACAGTGTCGACGCC |
| | CTCTACCGCCACCTACTATCATGGCAGCAAGGCAACAACCTCGACCGCGAATCCGGGCTACCCCATTTGGCACACGCCGCCT |
| | GGCACTGCCTCGCACTCCTCGCATACCAGCAACACAATGCCGGGGTAGACACCCGCAACCCATGGAACACCCACAAAGGCG |
| | ACCAGTAATGCCTCTAGCACAATACCCGAAAACCATCCACCATCCAGGCCACATCTCCTACTCATCACTGTCACAGTGGGCCG |
| | AATGCGGAGAAAAATGGCGCCTATCCCACGGCTACCACACCCAACACCACACCTGGTACGCCACCATCGCCGGAAGCGCCAT |
| | ACACCACATCACCGAACAATACGACCTACACCTGTAC |
| | SEQ ID NO.: 13; >3539 len: 1481 cycle: O |
| | GTCTGGGGCCACGTCGGGGTTTAAGATCGAGTTGGCGGAGTCGGATGTGTTGCGGTTTGGGCCCGGCAATCTGATGCCAGGG |
| | GATTTGATCTATGTGGATGTGGGCTCGGGTCCTATTGCGGAGATTGTTCGGCAGATTGATGTTGAGTGTGTATCGCCTGGGGA |
| | TGGGTGGACGAAGGTGACACCGGTTGCCGGTGATTATGAGGATAATCCGTCGGCTTTGCTGGCTCGCCGTGTGGCTGGTTTGG |
| | CTGCCGGTGTGCGGGATTTGCAAAAATTCTAGAATGATGGGGGTTTGTTGTGGCTATTGTGTGTAAAGGGTTTGGTGGTGTGT |
| | TGACCGAGTATGATTGGGCTCAAATGTCTGGTCTGATGGGTAATATGCCGTCCGTGAAAGGGCCGGACGATTTTCGTGTCGGC |
| | ACGACGATTCAGGGTGCCACAGTGTTGTGCGATGTCCTGCCGGGGCAGGCTTGGGCTCACGGGGTGATGTGCACTTCTAGTA |
| | GTGTTGAGACGGTGACAGGGCAGCTGCCTGGCCCTGGTGAGACCCGATACGACTATGTGGTGTTGTCTCGGGATTGGGAGCA |
| | GAACACAGCCAAGTTGGAGATTGTTCAGGGTGGCCGTGCGGAGCGTGCCCGTGACGTGTTGCGTGCCGAGCCTGGCGTGTAC |
| | CATCAGCAGCTACTGGCTACTTTGGTGGTGTCGTCTAACGGGTTGCAGCAGCAGTTGGATAGGCGTGCTGTGGCGGCTAGGG |
| | TTGCGTTTGGCGAGTCTGCTGCGTGTGATCCTACCCCTGTGGAGGGTGATCGTGTGATGGTGCCTTCGGGGGCTGTGTGGGCT |
| | AACCATGCCGGGGATTGGATGTTGTTGTCTCCCAGGGTTGAGACGGGTTCGAAGTCTATCATGTTTGGCGGGTCTGCTGTGTA |
| | TGCTTACACGATCCCGTTCGGGCGGCCGTTTACTAGTCCGCCTATCGTGGTGGCGTCTATGGCTACGGCTGCTGGGGGCACGC |
| | AGCAGATCGATGTGAAAGCCTACAATGTGACTGCCCAAAATTTTGGTTTGGCGTTTATTACGAATGATGGTTCGAAGCCGAAT |
| | GGTGTGCCTGGGGTGGCTAACTGGATTGCTGTCGGCGTGTGACCGGGCTGTTGTTGTGGCGGATGTTGTGATATTTGGGGGCT |
| | GTGGTGTCGTGGTTTACTCCTGCACTGGTGGCCTCTATCTGTACCGCATTGGCCACGGTTTTGGGTTCGGTTCAGGCTGCCACA |
| | TCTAAATCTCAGAAGCGTTTGCGGCGGCTGTCGGCTCAGGTAGATGCGATGGAAGAGTATACGTGGGGTGTGCGCCGTGAGG |
| | TGCAAAGGTTTAACGCCGGGCTTCCTGATGATGTGGAGCCGATTCATCTCCCTGATGTGCCCGAGTTTTTGAAAGATACTGTT |
| | GATGGTGGAGGTGAGTAGGGTTGAGGGAGTTGGAGGAGGAGAAGCGGCAGCGCCGCAATTTTGAGAAGTTTTCAC |
| | SEQ ID NO.: 14; >3558 len: 11424 cycle: O |
| | GGATCCGGTGAGTCGTGGCTATGCGCGCTATCTGTTGCATTTGACAGCCTATGACCCGTTTTGGTATGGGGATGAGCAGAAGT |
| | TTCGTTTCAGTAACGCGAAGTTGCAGGATTGGTTGGGTGGCGGCCCTGTTAATAAGAAGGGTACAGCGTTTCCGGTGGTGTTG |
| | ACGCCTGGTGTTGGTTCGGGCTGGGATAACCTGTCTAATAAGGGTGACGTGCCGGCGTGGCCTGTGATTCGTGTTGAGGGGC |
| | GGTTGTCATCGTGGTCTGTGCAGATTGATGGTTTGCGTGTGTCTTCGGACTATCCGGTTGAGGAGTATGATTGGATTACTATTG |
| | ATACGGATCCTCGGCAGCAGTCTGCGTTGTTGAACGGGTTTGAGGATGTGATGGATCGTTTGACAGAGTGGGAGTTTGCGCCT |
| | ATTCCGCCTGGCGGTTCTCGGAGTGTGAGTATTGAGATGGTTGGTTTGGGTGCCATTGTTGTGTCGGTGCAGTACAGGTTTTT |
| | GAGGGCTAGGTGAATAGTTGATGGCTGGTCTTGTTCCGCATGTAACATTGTTTACGCCGGATTATCGCCGTGTGGCGCCTATC |
| | AATTTTTTTGAGTCGTTGAAATTGTCGTTGAAGTGGAATGGTTTGTCGACGCTGGAGTTGGTGGTGTCGGGGGATCATTCTAG |
| | GCTGGACGGGTTGACGAGGCCTGGTGCACGGCTGGTTGTTGATTATGGTGGTGGCCAGATTTTTTCGGGGCCTGTGCGCAAG |
| | GTGCACGGTGTGGGGCCGTGGCAGTCCTCGAGGGTGACTGTGACGTGTGAGGATGATATTCGGCTGTTGTGGCGCATGTTGA |
| | TGTGGCCTATCAATTATCTTCCCAGTTTTGTGGGTTCGGAGTGGCGTGCGGACAGGGATTATGCCCATTATTCGGGTCCGGCG |
| | GAGTCGGTGGCGAAGAAGGTGTTGCGGGATAATGCTTGGCGGTTTCCGCCGGATGTGTTTCTGACTGATGATGAGAAGCGTG |
| | GACGGTTTATTAAGGATTTTCAGGTGCGGTTTCACGTGTTTGCCGATAAACTGTTGCCGGTGTTGTCGTGGGCTCGGATGACT |
| | GTTTCGGTGAAGCAGTTTGAGGATGTGAAGCTGGATCGGCGTGGTTTGGTGTTTGATTGTGTGCCGGCTGTGACCCGTAGTCA |
| | TGTGTTGACTGCCGAGTCTGGTTCGATTGTGTCGTGGGAGTATGTGCGTGACGCCCCGAAGGCTACTTCGGTGGTGGTTGGTG |
| | GCCGCGGCGAGGGTAAGGATAGGCTGTTTTGTGAGGATGTTGATAGCTTGTCTGAGGGGGAGTGGTTTGATCGTGTCGAGGT |
| | GTTTAAGGATGCCCGTAACACGGATTCTGAACATGTGCATCTTATTGATGAGGCTGAGCGGGTGTTAGGGGAGTCTGGGGCC |
| | ACGTCGGGGTTTAAGATCGAGTTGGCGGAGTCGGATGTGTTGCGGTTTGGGCCCGGCAATCTGATGCCAGGGGATTTGATCT |
| | ATGTGGATGTGGGCTCGGGTCCTATTGCGGAGATTGTTCGGCAGATTGATGTGGAGTGTGTATCGCCTGGTGACGGGTGGAC |
| | GAAAGTAACACCGATTGCGGGGGATTATGAGGATAATCCGTCAGCTTTGCTGGCGCGGCGTGTGGCTGGTTTGTCTGCGGGT |
| | GTGCGGGATTTGCAAAAATTCTAGAATGATGGGGGTTTGTTGTGGCTATTGTGTGTAAAGGGTTTGATGGTGTGTTGACCGAG |
| | TATGATTGGGCTCAATTGTCTGGTCTGATGGGTAATATGCCGTCTGTGAAAGGGCCGGACGATTTTCGTGTCGGCACTACTGT |
| | TCAGGGTTCCACGGTGTTGTGTGAGGTTTTGCCGGGGCAGGCTTGGGCTCACGAGGTGATGTGCACGTCGAATAGTGTTGAG |
| | ACGGTGACAGGGCAGCTGCCTGGCCCGGGTGAGACCCGATACGACTATGTGGTGTTGTCTCGGGATTGGGAGCAGAACACAG |
| | CCAAGTTGGAGATTGTTCAGGGTGGCCGTGCGGAGCGTGCCAGTGACGTGTTGCGTGCCGAGCCTGGCGTGTACCATCAGCA |
| | GCTACTGGCTACTTTGGTGGTGTCGTCTAACGGGTTGCAGCAGCAGTTGGATAGGCGTGCTGTGGCGGCTAGGGTTGCGTTTG |
| | GGGAGTCTGCTGCGTGTGATCCTACCCCTGTGGAGGGTGACCGTGTGATGGTGCCTTCGGGGGCTGTGTGGGCTCACCATGCC |
| | GGCGAGTGGATGTTGTTGTCCCCCAGGATTGAGACGGGCACTAAGCAAATCCAGTTTGGCGGGTCTGCTGTGTATGCTTACAC |
| | GATCCCGTTTGAGCGGCCGTTTAGTAGTGCGCCCGTTGTGGTGGCGTCTATGGCTACGGCGGCTGGGGGCACGGCACAGATT |
| | GATGTGAAAGCCTACAATATTACTAATAAGGATTTTAGTTTAGCGTTTATTACGAATGATGGTTCGAAGCCGAATGGTGTGCC |
| | TGCGGTGGCTAACTGGATTGCTGTCGGCGTGTGACCGGGCTGTTGTTGTGGCGGATGTTGTGATATTGGGGGGCTGTGGTGTC |
| | GTGGTTTACTCCTGCACTGGTGGCCCCTATCTGTACCGCATTGGCCACGGTTTTGGGTTCGGTTCAGGCTGCCACATCTAAATC |
| | TCGGAAGCGTTTGCGGCGGCTGTCGGCTCAGGTAGATGCGATGGAAGAGTATACGTGGGGTGTGCGCCGTGAGGTGCGAAG |
| | GTTTAACGCCGGGCTTCCTGATGATGTGGAGCCGATGCATCTCCCTGATGTGCCCGAGTTTTTGAAAGATACTGTTGATGGTG |
| | GAGGTGAGTAGGGTTGAGGGAGTTGGAGGAAGAAAAAAGGCAGCGCCGCTCGTTTGAGAAGGCTTCCCTGATACTGTTGTTT |
| | TTGTCGCTTGTGCTGTTGGCGGTGGTTGCCGGGGGTGCTTTACGGTACGGGTCTGTGGCTTCGCAAAGGGATTCGGAGCAGGC |
| | GAGGGCCCAGTCTAATGGTACGGCGGCTCGGGGTTTAGCTGCCCGTGTGAAGCAGGCGTGTACTTCGGGTGGGGTGGAGTCT |
| | GCGCGGCTTCACCAGTCTGGCTTGTGTGTGGATGCTGTGCGTGTTGAGCGTAGCGTGCAGGGTGTGCCGGGTCCTGCCGGTGA |
| | GCGCGGCCCGCAAGGGCCTGCAGGTGCTGATGGGCGTGATGGTGTTAATGGTTCGGCTGGGCTGGTTGGCCCTGTTGGTCCG |
| | CAGGGTTCTCCTGGTTTGAATGGTGTGAAAGGTCCTGACGGGTTGCCTGGTGTGAATGGATCGGATGGCCATGATGGTGTTCC |
| | GGGCCGTGCAGGTGCTGATGGTGTGAACGGTGTTGACGGTCGGGATGGTTCGGCCGGTGAGCGCGGTGATGTGGGCCCTTCA |
| | GGTCCTGCCGGCCCGCAAGGTGCACAGGGTGAACGGGGTGAGCGTGGCCCCGCCGATGCGAACGGATCCGATGGTAAAGAT |
| | GGTAAGGATGGCCGTTCTGTTGTGTCTGTGTACTGTTCCGGGGGCCGCCTGGTTGTGAAATATAGTGACGGTACGGCCTCTAC |
| | CATAGCGGGTTCTGCAGCCTGCGAGAGTGTGAAACCATCACCTGTGGTTACCGTATCATCCCATAGGTGAACAAGAAGAGGG |
| | AAGGGTGTTACTAGTGTTGGTCGTGTTTGGTGGTGGTGTGTGGTGAGATACATTCCAGCGGCGCACCATTCGGCCGGTTCGAA |
| | TAGTCCGGTGAATAGGGTTGTGATTCACGCGACATGCCCGGATGTGGGGTTTCCGTCTGCCTCCCGTAAAGGGCGGGCGGTG |
| | TCCACGGCAAACTATTTCGCGTCCCCATCGTCTGGTGGTTCGGCGCATTATGTTTGCGATATTGGGGAGACGGTGCAGTGCTT |
| | GTCGGAGTCTACGATTGGGTGGCATGCCCCGCCTAACCCGCATTCTTTGGGTATAGAGATTTGCGCGGATGGGGGTTCGCACG |
| | CCTCGTTCCGGGTGCCAGGGCATGCTTACACTCGTGAGCAGTGGCTTGATCCTCGCGTGTGGCCTGCGGTGGAGAAGGCTGCC |
| | ATGCTGTGTAGACGTTTGTGTGACAAATATAATGTTCCGAAAAGGAAACTGTCGGCTGCCGATTTGAAGGCTGGCAGGCGTG |
| | GTGTTTGCGGGCATGTGGATGTGACGGATGCGTGGCATCAGTCGGATCATGACGATCCGGGGCCGTGGTTTCCGTGGGACAG |
| | GTTTATGGCCGCCGTCAACGGTGGTAGTGGTAGTGAGGAGTTAACTGTGGCTGATGTGAAAGCCTTGCATGATCAGATTAAA |
| | CAGTTGTCTGGGCAGGTGGCCCAGTCGGTGAACCGGCTGCATCACGATGTTGGTGTGGTTCAGGTGCAGAATGGTGACTTGC |
| | GTAAGCGTGTCGAGGCTTTGTCGTGGGTGAAGAATCCTGTGACGGGGAAGCTGTGGCGCACTAAGGATGCTTTGTGGAGCAT |
| | CTGGTATTACGTGCTGGAGTGTCGCAGCCGTATTGACAGGCTCGAGTCTGCTGTCAGCGGTTTGAAAAAGTGATGGTGGTTTG |
| | TTGTGGGTAAACAGTTTTGGTTAGGTTTACTGGAGCGGGCGGCTAAGACTTTTGTGCAAACGTTTGTTGCGGTGTTGGGTGTG |
| | ACGGCGGGTGTTACTTATACTGCGGAGTCGTTTCGTGGTTTGCCGTGGGAGTCTGCCCTGATCACAGCAACGGTTGCTGCGGT |
| | GTTGTCGGTTGCTACCTCGTTTGGTAGCCCGTCGTTTGTGGCCGGTAAGCCGAAGGTGACGCCTGTTGTGGATGCTGGGCTTG |
| | TTCCACCTGATGACGGGGGCTTGGTTGAGCCGCACTCGGTGGATGTGTCGGATCCTGGCATGATCGAGCCTGTAGACGATGCT |
| | GATCTTGGTGGCTATGTGCCGAGGCGTGCCGCCGAGTCGGAGGTTGGCACGGTAGAGTCTACTGTTGCATAATTGAATATGT |
| | GTGTGCCCCAGCGGTGCTGCCACGATTGTGTGGTGGTTGCCGCTGGGGCACTATTTTTGTGTCTATAGGGGTTTTACAGGTTG |
| | TTGTCTAGTGTGTCTTCGATGAGCTGGTCCAGGTGGAGGCAGGCTGAGATAGTATCGTTGGCCTGGTCTAGAACATCCTGGCC |
| | GATAACGCCTTTGTGGTTGTCGCGGTGGCGGATGATAGCCCACATGATCTCGTCGGCTGCCGCCTGTAATAGTTTTGCCTGGT |
| | ATGCGATTCCGGCGAGCCAGTCTAGTGCTTCCTGGCTTGCATAGGGGCTCTGGTTCTCGCTGTTGCCGCGGGTGTTGCTGTTGT |
| | TTGTGGGGTGTCCTGCACTGTCGCAGAACCATAGGATTTCGCTGCACTCGTCTAGCGTGTCTTGGTCGATAGCGAGATCGTCG |
| | AGGCTGACATTGTTGACGGTAAGGTTAACGTTGTCGAGGGAGATGGGTACACCGTACTGGTTTTCGACACTGTCAACAATGTT |
| | TTGTAGCTGTTGCATGTTGGTGGGCTGTAGTTGGACGATACGATGTATCGCTGTGTTGAGGGTGGTGTAGGTGATATTGTGTG |
| | TGTTGTTCATGGCTGTTGTCCCATCCCTGTGCTGTCGTCGTTGTCGTCTTGGTAGTATCGACTGTTTGCGTATCCTGTGAGGGT |
| | GATGAGTGTTTGGTCTGCCCACTGTTTCACGGTTTGCCGGGTGACTCCGAGACGTTGGGCTGCCACCGAATAGGTTTGGTCAT |
| | ACCCGTATACTTCCCTGAATGCTGCCAACCTAGCTAGCCGTATCCTTTGTTTGGGTGGCTGGCAGGTGAGGGTGTAGTCGTCG |
| | ATGGCTAGCTGCAAATCGATCATGGAGACGATGTTGTTGCCGTGGTGTTGGGGGGCGGTTGGTGGTGGTGGCATTCCTGGTTC |
| | GACGGAGGGTTTCCATGGGCCTCCGTTCCAGATCCATTGGGCGGCTTGGATGATGTCGGCGGTGGTGTAGGTTCGGGTCATGT |
| | GTCACCCCTGGAACAGGTTGTCGAGGTTGTCTGGCTGGCTGGTGGTGTCGAATCGTCCCACACAGTGGCAGTAGTCGTACATG |
| | AGTTTGATAATGTGTTGGTGGTCTCCCAAATAGGTGTTTCCGCTGATGCTGTAGGTGGCTGTGCCGTCTTTACTGATGGTGTAT |
| | TTGGCGGTGATGGTTTCGGGGGTTTCTGTGTTGGTGATGATGGCGGTGGTGGTGGCGCCTACTGTTTGTAGCCTGGTGGTTTG |
| | GGTGCCGTCGTCGAGGATGGTTGTGACCATTGTGGTTCTCCTTAGTTGCTGGTTTGGTTGTCGGCTATGGCTGTGATTTCTTGG |
| | ACGGGTTTGGGTAGGTATAGGTGGTGGAGTGTTTTGTTTGCTAGTCGTTGGGCGACACGGTAGCACATTTGGGTCCACTGGTT |
| | GCCTGTGAGTTGGTGGTATTGGTTGCGTACCGCAATATATAGTAGAGAGTCTTGGTACAGGTCGTCTGGGTTGACAGCCGGGT |
| | AGCGGCGGGCAATATTGGTGCAGGCTTTGTGTAGCTGGTGTTGGTGGGCTGGTGTGGTCCATCCCCAATCGGCTGTGGTTGCT |
| | AGGTCTGCTTTGGTTGGTCGTCTACTCATGGCATCTATTCTATCGGGCTATCTGGTAGTTGTTTGGTGTTTTGTTGTTGATAGT |
| | GTAGCACACGAGTCCGGGGTTGCCGGTGGCGCCTGTCCTGTGTCGGTACCAGACTGATTCTCCTTCCATGGATGGGCATTGGAT |
| | GAATGTGCGTTGTCCTTGCTCGGAGATTTCGAGGTGGTGCCTGTGTCCGGCCATGAGAATGTGGGATACGGTGCCGTTGTGGA |
| | ATTCTTGGCCGCGCCACCATTCGTAGAGTTGGTTGTTGCGCCATTGGTGTCAGTGGGCGTGCAGGATGGTAGCCCCGGCTACG |
| | TTGACGGTGGTGGTCATTTCGTCGCGTTGGGGGAAATAAAAGTGGAGGTTGGGGTAGTTGTTGGTGAGTTGGTAGGCTTCTGC |
| | GATGGCGCGGCAGCAGTCCACGTCGAATGAATCATCGTAGGTGGTGACGCCTTTGCCGAATCGTACGGCTTCTCCGTGGTTGC |
| | CGGGGATGGAGGTGACCGTAAGGTTGGTGCAGTGGTCGAACATGTGGATGAGTTGCATCATGGCCATGCGGGTGAGCCGGAT |
| | TTGTTCCGTCAAGGGGGTTTGTGTGCGCCAGGCGTTGTTGCCTCCTTGTGACACGTATCCTTCGATCATGTCGCCGAGGAAGG |
| | CTATGTGGACTCGTTGCGGCTGGCCTGCTTGCTGCCAGTAGTGTTTGGCGGCTGTGAGTGAGTGCAAATAGTCGTCGGCGAAT |
| | CGGCTGGTTTCTCCGCCGGGGATGCCTTTGCCGATTTGGAAGTCTCCTGCCCCGATGACGAAGGCCGCGGTGCTGTAGTCGGT |
| | GCGGGTGTCCTGTTCGGGTTTTGGGGGTGCCCATTCGGCTAGTTTATCGACGAGTTCGTCTACAGGGTAGGGGTCGGTTGCGG |
| | GTTGGTGGGCGATGATTTTTTGTATGGATCGGCCTGTTTCTCCGTTGGGTAGGGTCCATTCGGAGATGCGTGTGCGGCGTACG |
| | GTTCCGTTGGCGAGATCATCGCGGATGGTGTCTGCTTCGTTGTCGTGGTTGGCTAGCTGGGTGAGGAGCCGGTCTATGTTGTC |
| | TATCATCGGGTATTCTCTTCCTTTTGTGGAGTGGTGTTGGCTTGTTTGCGGCGGTAGTCTTTGATGACGGTGGCGGAGATGGG |
| | GTATCCGGCTTGGGTGAGTTGTTTTGCTAGCCAGGATGCGGGTATAGACCTGTCGGCGAGGACGTTTGCGGCTTTGCGGCCGT |
| | AGCGTTGGATCAACGTTTCAGTTTTGGTTGCCATGGTGTCCTATCGGTTGTGTGGTGGGCTGCCATCCTGTGCGGCAGTCTCCG |
| | TCGTGTCCTGGTTTGCGTGTGTACGATGTGATGGTGCCGTCTGTGTGGTGGAGTGTTTTGCCGCACATGACGTTTTGTAGATGC |
| | TCCGGCAGCTCGCTATTGCTATCGTCTTGCTCATCTAGCAAAGTTTTTTGTTGGGTGAAAAACTCGGACACGGTGCCGTTGTG |
| | GACTGGGAGTATCCATGTTTTCCATTGTTGTTGTAGTCGGGTGTTCCAGTGGAATTGTTTGGCGGCGTCTTCTGCCTGTTTTAT |
| | GGTTTTGTAGTAGCCGACGAGTATGCGCTGGTGTTGCTGGTCGGGCGGGTTTTGGCCTCGCCAGTATTGTGCCGCGACGGCGT |
| | ACCGGTTGTTGTCGGTGAAGGTGTTCCGGCAGTATTGGATGATGTTGTTGAGGGTGTCATCTGGCAGATCATCTGGGTTGAGG |
| | TTGGTGGCCTGGGTGAGGATGTCTCGGATGGTTTGAAGGTTGCGTTTTGTGGGTTTGAACGAGATGCTCACGATAGTGCCGGC |
| | TGTTCGTCTTGCATGAACTGGTTGAAGGTGTTGTTCCCGGCGTGTTGGGCTTGTGTGATTTGCTGGTCGGTCCAGTCGGGGTGT |
| | TGCTGTTTCAGATAGTGCCAGTGGCACGCATTGTAGGTCTCGTCTTGTAGCCGTGTGAGATGGTTTTCGGTGATGATTTGTTTC |
| | CACATGGCCCATGACACGTCGAGCCGGTTGAGGATTTCGAGGGCTGGGATGTTNAACTGGTCGAGGAAGAGGATTTCGTGGG |
| | TGTAGTAGTTTTTCTCGTAGGCGTCCCATCCGCTTCGGTGCCTGTTGGGCTGGTTTTTGGGGTAGGCTTCCCGGCATACTTTGT |
| | GTAAACGCTTGGCCATGTCGTCGGGTAGTTTAATGTCGGGGTTGGCGCGGATCATGGATCGCATCCCATCATAGGTGGTGCCC |
| | CAGGTGTGCATGATGTAGGTGGGGTCTTCACCATCAGCCCATTTTTCTGCACAGACGGCGAGGCGGATACGCCTCCTGACTGT |
| | TTGGCTGGTGTTGCGCCGGTTGGGGATGGGGCACGTGTCGGGGGGATCCATGATGTTTTAGTTTACCTTTCTGGTTTCGTGTTG |
| | TTGACGTGTTTTACTGTAGCACAGTGTCTAGTGCTTGTGTCAACCCTGTTTTTCCGGCCTGCAGGTAGGTGTCTGTGACATCCC |
| | CGACAGTGAGGGGCACATGTACGGCTTGGGGGAGTGCTGCCTGGATGGTTTGTGCCATCTGGTGGCCTGCGGGGTCTGGGTC |
| | GGACCAGATGTAGATGTGGTCATAGCCTTCAAAAAATTTGGTCCAAAAAGTTTGCCACGAGGTTGCGCCGGGTAGGGCAACG |
| | GCCGACCATCCGCATTGTTCGAGGATCATGGAGTCGAATTCGCCTTCGCAAATGTGCATTTCGGATGCCGGGTTGGCCATGGC |
| | GGCCATGTTGTAGATGGGGCCTGTGTCTCCTGCCGGGGTTAGGTATTTGGGGTGGTTGTGGGTTTTGCAGTCGTGCGGGAGTG |
| | AGCAGCGGAAACGCATTTTCTTATTTCTGCTGGGCCGCCCCAAACGGGATACATGTATGGGATGGTGATGCACTGGTTGTAGT |
| | TTTCGTGGCCGGGTATGGGGTCATTGTCGATGTATCCAAGGTGGTGGTAGCGGGCTGTTTCTTCGCTGATGCCTCTTGCTGAG |
| | AGCAGGTCGAGTATGTTTTCGAGGTGGGTTTCGTAGAGGGCCGGGGCTTTCTGGATTCGGCGGCGTTCCGCAATGTTGTATGG |
| | GTGTATGCTGTCGTACATTCGGGTTTCTTTCTTCGAGTTGTTGTTGTAGTTTGGCGAGTCCGCCTCCGACACCGCATGTGTGGC |
| | AGTACCAGACGCCCTTGTCGAGGTTGATGCTCATGGAGGGCTGGTGGTCGTCGTGGAACGGGCAGAGGATGTGTTGCTCGTT |
| | CTTGGATGGGTTGTACCGTATCCGGTAGGTGTCGAGGAGGCGGCAGGTGTCAGAGGTGTGGGAGGAGCTCGTTGAGGGTTGA |
| | TACCACATTGGCTTCCCTCCATGGCTTGTTGCGCTGTTTCATCACTACTAGTCCGATGGTGGATTGTGGCTGCCGGTTTCGGTG |
| | TGTTTCGTAGTTTCGTGCCTCTTGGCTGGCTTGTTTCACGAATTCGGCGAGATGGGGCTGCCCGGCTTTCGCTTCGATAACGTA |
| | GGTTTTGTTTCCGGTTTGAAGGATGAGGTCTCCTTCGTCTTCGCGGCCGTTGAGGTGGAGGCGTTCTATGTCGTGTCCGGTGTC |
| | GCGTAGCTGGTGCAATAATCGTGTTTCCCATTCTGCGCCTGCCCCGCGGTTGCGTGCCTGCTGTGAAGCCATCATAGTCCTTTG |
| | TGTGTTGTGGTCATGTTCCATGGCTGTTTTTCGACGAGGGGTCCGAAGAATGTGTATTCGGGGTAGGCTCGTAGCCGCTCGTA |
| | TCGGGTTCCGTCAGGACCGGATTTGCCGGTGCGCTGTTTGAGTACGGCGATGCGTGCCTCTGCCGGTATCGTGAGGCCGTTGC |
| | CATTATCCTCGCCACCATACAATGAGACTCCGAGGATGAGTTGTGGTTTTTCTGAGAGTCCGTTTTTGATTTCTCGCCGTGCTG |
| | GCGGGTGTTCGATGTCGGAGCCGGTTTTGTCGGTTGCATGGTGTGTGACGATGATGGTGGAGCCAGTATCCCTACCTAATGCT |
| | GTGATCCATTGCATGGCTTCTTGCTGGGCCTGGTAGTCGGATTCGCAGTCTTGAATGTCCATCAGGTTGTCGATAACAATGAG |
| | TGGTGGGAAAGTGTTCCACATTTCCATGTAGGCTTGCAGTTCCATGGTGATGTCTGTCCATGTGATGGGTGACTGGAATGAGA |
| | ATGTGATGTGTTGGCCGTGGTGGATGCTGTCTCGATAGTATTCTGGCCCGTAGTCGTCGATGTTTTGTTGTATCTGTGTGGTGG |
| | TGTGTTGGGTGTTGAGTGAGATGATTCGTGTGGAGGCCTCCCAGGGTGTCATGTCCCCTGATATGTAGAGGGCGGGCTGGTTG |
| | AGCATGGCGGTGATGAACATGGCTAGCCCGGATTTTTGGCTGCCGGACCGCCCCGCGATCATGACGAGATCCCCTTTGTGGA |
| | TGTGCATGTCCAGGTTGCGGTAGAGGGGTTCTAGTTGTGGTATGCGGGGCAGCTCGGCGGCTGTTTGGGAGGCTCTCTCGAA |
| | GGATCGTTGTAGAGAGAGCATCGGAGCCTTAATCTATCTGTCTATCGGTTGGATGATGTTTTGGTGGTCAGATGGAGTCGATG |
| | TCGATATCAGCATCTGTAGGGGCTGTGGTGTCGTCTAGCTGGCCGTTATCGCGCTTGTCTACGTATTCGGCAACCTTATCGTA |
| | GATGGCGTCGTCTAATGGTTTGAGCACGACCGCGTTGAACCCGTTTTTGGTGCGTACGGTGGCGAGTTTGAAGGCCTGTTCTT |
| | CTCCAAGGTAGGCTTCGAGGTCGCGGATCATGGAGTGTGGGCGGTCGTTGTTGCCGCGCGCTTTCTCAATAATAGCGTTGGGG |
| | ATGGTTTCTGGGGTGCCGTTGTTGAGATCGTCTAGGGTGTGGAAGATTGTGACATCAGCGTAGATGC |
| | SEQ ID NO.: 15; >4617 len: 12408 cycle: O |
| | GGGGGGGTTCCTGGCCTGGGGGGCCCTGTCGGTATTGTGATCGGCTTGTTTGCTGCCATGTTTGCCACTAACGCCCAGTTCCG |
| | TGCCGCGGTGATGCAGCTTGTGGGTGTGGTTGGTCAAGCCCTGGGGCAGATTATGGCGGCTATTCAGCCGCTGTTTGGTTTGG |
| | TTGCGGGGCTGGTGGCCCGGTTGGCGCCAGTGTTTGCCCAGATTATTGGTTTGGTTGCCGGTTTGGCTGCCCAGTTGATGCCT |
| | GTGATTGGTATGCTGGTTGCCCGGCTGGTGCCTGTGATCACGCAGATTATTGGTGCGGTGACGCAGGTAGCTGCCATGTTGTT |
| | GCCTGCGCTTATGCCGGTGTTGTAGGCTGTTGTGGCTGTGATACGTCAGGTTGTTGGCGTGGTTATGCAGTTGGTGCCGGTGT |
| | TGATGCCGGTGATTCAACAGATTTTGGGTGCTGTCATGTCTGTGCTGCCACCGGTTATTGGCCTGATCCGGTCGCTGATACCA |
| | GTCATCATGTCTGTTATGCGTGTGGTGGTGCAGGTTGTTGCGGTTGTGATACAGGTGGTGGCCCGTATTCTTGCGGTTGTGGCT |
| | CCGATGGTGGCTGCTGTGGTGGGGTTTGTTGCCCGTATTGTTGGTGCTGTTGTGTCGGCTGTGGCCCGTGTTATTGCTGCTGTT |
| | GCCCGTGTTGTTGGGTGGGTTGTGGCCCATTTTGTGTCTGGTTTGGCGCGTATGGGTTCGGTGGTTCAGGCTGGCTGGAATCG |
| | TATTAGGGCGTTTACGTCTGCGTTTATGAGCGGGTTTAAGTCGATCATTTCTGCCGGTGTGGGCGCTGTTGTCGGGTTTTTTGC |
| | GCGGCTTGGTTTGTCGGTTGCTTCCCATGTTCGGTCTGGGTTTGGCGCGGCTCGTGGTGCTGTTTCGGCTGCGATGAATGGGAT |
| | CCGGAGTATTGTGTCTTCGGTGGCGTCTGCTGTTGGCGGGTTTTTCAGTTCGATGGCGTCTAGGGTTCGTAGTGGTGCTGTGCG |
| | CGGGTTTAATGGTGCCCGGAGTGCGGCATCTTCTGCTATGCATGCTATGAGGTCCGCTGTTTCCAATGGTGTGCATGGTGTGC |
| | TAGGGTTTTTCCGGAATCTGCCCGGTAATATTAGGCGCGCTCTCGGTAATATGGGGTCCCTGTTGGTGTCTGCGGGCCGTGAA |
| | GTGGTTTCTGGTATGGGTAATGGTATCCGGAATGCTATGAGTGGCCTGTTGGATACGGTGCGTAATATGGGTTCTCAGGTTGC |
| | TAATGCGGCGAAGTCGGTGTTGGGTATTCATTCCCCGTCGAGGGTGTTTCGTGACCAGGTTGGCCGGCAGGTTGTTGCCGGTT |
| | TGGCTGAGGGGATCACCGGGAATGCCGGTTTGGCTTTGGATGCGATGTCGGGTGTGGCGGGTCGGCTGCCTGATGCGGTTGA |
| | TGCCCGGTTTGGTGTGCGATCGTCGGTGGGCTCGTTTACCCCGTATGGCAGGTATCAGCGTATGAGCGAGAAGAGTGTTGTGG |
| | TGAATGTGAATGGGCCTACTTATGGGGATCCTAACGAGTTTGCGAAGCGGATTGAGCGGCAGCAGCGTGACGCTTTGAATGC |
| | GTTGGCTTACGTGTGATTGCGGGGTGTGGTTCATGTTTATTCCTAACCCGTCTGATCGTGCCGGTTTGACGGTGGATTGGACG |
| | ATGTTTCCGTCGATTGGTAATAATCCGGAGCGTGTTCTTCATTTGACGGATTATACGGGTGCGTCTCCTGTCATGTTGTTGAAT |
| | GATTCGTTGCGCGGTTTGGGTGTTCCCGAGGTGGAGCATTTTTCTCAAACTCATGTTGGGGTGCACGGCTCGGAGTGGCGCGG |
| | GTTTAATGTGAAGCCTCGCGAGGTGACATTACCTGTGTTGGTGTCGGGTGTTGACCCGGATCCGGTGGGCGGGTTTCGTGACG |
| | GTTTTTTGAAAGCCTATGACGAGTTGTGGTCTGCTTTTCCTCCGGGCGAGCAGGGGGAGTTGTCGGTGAAGACTCCTGCCGGT |
| | GTTGAGCGTGTGTTGAAGTGCAGGTTTGATTCGGCTGATGACACGTTTACGGTGGATCTGGTGAATCGTGGCTATGCGCGTTA |
| | TGTGCTTCATTTGACAGCTTATGACCCGTTTTGGTATGGGGATGAGCAAAAGTTTCGTTTTAGTAACGCGAAGTTGCAGGATT |
| | GGCTGGGTGGCGGTCCTGTCGGCAAGAAGGGTACAGCGTTTCCTGTGGTGTTGACGCCTGGTGTGGGCTCTGGTTGGGATAA |
| | TCTGTCTAATAAGGGTGATGTGCCTGCGTGGCCTGTGATTCGTGTTGAGGGGCCTTTGGAGTCGTGGTCTGTGCAGATTGATG |
| | GTTTGCGTGTGTCTTCGGACTATCCTGTCGAGGAGTATGATTGGATCACTATTGATACGGATCCTCGCAAACAGTCTGCATTG |
| | TTGAACGGGTTTGAGGATGTGATGGATCGTTTGAAGGAGTGNGAGTTTGCCCCGATTCCGCCTGGCGGTTCTAAGAGTGTGA |
| | ATATTGAGATGGTTGGTTTGGGTGCCATTGTTGTGTCGGTGCAGTACAGGTTTTTGAGGGCTTGGTGAATAGTTGATGGCTGG |
| | TCTTGTTCCGCATGTAACATTGTTTACGCCGGATTATCGTCGTGTGGCGCCTATCAATTTTTTTGAGTCGTTGAAATTGTCGTT |
| | GAAGTGGAATGGTTTGTCGACGCTGGAGTTGGTTGTGTCGGGGGATCATTCAAGGCTTGAAGGGTTGACGAAGCCGGGTGCA |
| | CGGCTGGTTGTTGATTATGGTGATGGCCAGATTTTTTCTGAGCCTGTGCGTAAGGTGCACGGTGTGGGTCCTTGGCGTTCTTCG |
| | CGGGTGACTATCACGTGTGAGGATGATATCCGCCTGTTGTGGCGTATGCTGATGTGGCCTGTGAATTATCGTCCCGGTTTGGT |
| | GGGTTCGGAGTGGCGTGCCGACAGGGATTATGCCCACTATTCTGGTGCTGCGGAGTCGGTGGCTAAGCAGGTGTTGGGGGAT |
| | AATGCTTGGCGTTTTCCGCCTGGTTTGTTTATGACCGATGATGAGCGTCGTGGCCGCTATATTAAGGATTTTCAGGTGCGGTTT |
| | CACGTGTTTGCCGATAAACTGTTGCCGGTGTTGTCGTGGGCTCGGATGACTGTCACGGTGAAACAGTTTGAGAATGCGAAGTT |
| | TGATCAGCGTGGTTTGCTGTTTGATTGTGTGCCTGCGGTGACCCGGAAGCATGTGTTGACTGCCGAGTCGGGTTCGATTGTGT |
| | CGTGGGAGTATGTGCGTGACGCCCCGAAGGCGACATCTGTGGTTGTGGGTGGCCGCGGCGAGGGTAAGGATAGGCTGTTTTG |
| | TGAGGATGTTGATAGCTTGTCTGAGGGGGAGTGGTTTGATCGTGTCGAGGTGTTTAAGGATGCCCGTAACACGGATTCCGAG |
| | AAGGTGCATCTTATTGATGAGGCTGAGCAGGTGTTGTCCGAGTCTGGGGCCACGTCGGGGTTTAAGATCGAGTTGGCTGAGT |
| | CGGATGTGTTGCGGTTTGGGCCCGGCAATCTGATGCCAGGGGATTTGATCTATGTGGATGTGGGCTCGGGTCCTATTGCGGAG |
| | ATTGTTCGGCAGATTGATGTGGAGTGTGTATCGCCTGGTGATGGTTGGACGAAGGTGACTCCTGTGGCGGGGGATTATGAGG |
| | ATAATCCGTCGGCGTTGCTAGCACGGCGTGTTGCTGGTTTGGCTGCGGGTGTGCGGGATTTGCAAAAATTCTAAAAAGATTAG |
| | GGGTTTGTTGTGGGTATTGTGTGTAAAGGGTTTGATGGTGTGTTGACCGAGTATGATTGGGCTCAAGTGTCTGGTCTGATGGG |
| | TAATATGCCGTCTGTGAAGGGCCCGGACGATTTTCGTGTCGGCACGACGGTTCAGGGTGCCACAGTGTTGTGTAGTGTTTTGC |
| | CGGGGCAGGCGTGGGCTCACGGGGTGATGTGCACGTCGAATAGTGTTGAGACGGTGACGGGGCAGCTTCCGGGTCCGGGTG |
| | AGACCCGATACGACTATGTTGTCCTGTCTCGGGATTGGGAGCAGAACACAGCCAAGTTGGAGATTGTTCCCGGTGGCCGTGC |
| | GGAGCGTGCCCGTGACGTGTTGCGTGCCGAGCCTGGCGTGTATCATCAGCAGTTGCTAGCGACTTTGGTGGTGTCGTCTAACG |
| | GGTTGCAGCAGCAGTTGGATAGGCGAGCTGTGGCGGCTAGGGTTGCGTTTGGGGAGTCTGCTGCGTGTGATCCTACCCCTGT |
| | GGAGGGTGACCGTGTGATGGTTCCTTCTGGGGCTGTGTGGGCTAACCATGCCGGCGAGTGGATGCTACTGTCTCCGAGGATT |
| | GAGACGGGCACTAAGCAGATCCAGTTTGGTAGTTCGAATGTGTATGCTTACACGATCCCGTTTGATCGCCCGTTTAGTAGTCC |
| | GCCGGTTGTGGTGGCGTCTATGGCCACGGCTGCTGGGGGCACGCAGCAGATCGATGTGAAAGCCTACAATGTGACTGCCCAA |
| | AATTTTGGTTTGGCGTTTATTACGAATGATGGTTCGAAGCCGAATGGTGTGCCTGCGGTGGCTAACTGGATTGCTGTCGGCGT |
| | GTGACCGGGCTGTTGTTGAGGCGGATGTTGTGATATTGGGGGGCTGTGGTGTCGTGGTTTACTCCTGCACTGGTGGCCTCTAT |
| | CTGTACCGCATTGGCCACGGTTTTGGGTTCGGTTCAGGCTGCCACATCTAAATCTCGGAAGCGTTTGCGGCGGCTGTCGGCTC |
| | AGGTAGATGCGATGGAAGAGTATACGTGGGGTGTGCGCCGTGAGGTGCGAAGGTTTAACGCCGGGCTTCCTGATGATGTGGA |
| | GCCGATGCATCTCCCTGATGTGCCCGAGTTTTTGAAAGATACTGTTGATGGTGGAGGTGAGTAGGGTTGAGGGAGTTGGAGG |
| | AAGAGAAGCGGCAGCGCCGCAATTTTGAGAAGGCTTCACTGGTGTTGTTGTTTTTGTCGCTTGTACTGTTGGCGGTGGTTGCC |
| | GGGGGTGCTTTACGTTTCGGGGCTGTATCCTCTGAGCGGGATTCGGAGCAGGCTAAAGCCCAGTCTAATGGTACAGCCGCTA |
| | AAGGGTTGGCTGCCCGTGTGCGGCAGGTGTGTGCTTCTGGCGGGCAGGAGTCGGTGCGTCTTCACCGGTCTGGCTTGTGTGTG |
| | GATGCTCAGCGTGTTGAGCGTAGCGTGCAGGGTGTGCCGGGTCCTGCCGGTGAGCGCGGCCCGCAAGGGCCTGCAGGTGCTG |
| | ATGGGCGTGATGGTGTTAATGGTTCGGCTGGGCTGGTTGGCCCTGTTGGTCCGCAGGGTTCTCCTGGTTTGAATGGTGTGAAA |
| | GGTCCTGACGGGTTGCCTGGTGTGAATGGATCGGATGGCCATGATGGTGTTCCGGGCCGTGCAGGTGCTGATGGTGTGAACG |
| | GTGTTGACGGTTGGGATGGTTCGGCCGGTGAGCGCGGTGATGTGGGCCCTTCAGGTCCTGCCGGCCCGCAAGGTGCACAGGG |
| | TGAACGGGGTGAGCGCGGCCCCGCCGGTGCGAACGGATCCGATGGTAAAGATGGTAAGGATGGCCGTTCTGTTGTGTCTGTG |
| | TACTGTTCCGGGGGCCGCCTGTTTGTGAAATATAGTGACGGTGTGGCGTCCACGATATCGGGTTCGGTGGCCTGCGAGAGTGT |
| | GAAACCATCACCTGTGGTTACCGTGTCATCCCCGCAAGTGAGAGAGGAAGGGTGTTACTGATGTTGGTAGTGTTTGGTGGTG |
| | GTGTGTGGTGAGGTTTATTCCTGCGGCGCATCACTCGGCTGGTTCTAATAGTCCGGTGAATAGGGTTGTGATTCATGCGACGT |
| | GCCCGGATGTGGGTTTTCCGTCTGCCTCGCGTAAAGGGCGGGCGGTGTCGACGGCGAACTATTTCGCGTCCCCATCGTCTGGT |
| | GGTTCGGCGCATTATGTTTGCGATATTGGGGAGACGGTGCAGTGCTTGTCGGAGTCTACGATTGGGTGGCATGCCCCGCCGA |
| | ATCCGCATAGTTTAGGTATCGAGATTTGCGCGGATGGGGGTTCGCACGCCTCGTTTAGGGTGCCGGGGCACGCTTACACTCGG |
| | GAGCAGTGGCTTGATCCTCGCGTGTGGCCTGCGGTGGAGAAGGCTGTCATGCTGTGTAGACGTTTGTGTGACAAATATAATGT |
| | TCCGAAAAGGAAACTGTCGGCTGCCGATTTGAAGGCTGGCAGGCGTGGTGTTTGCGGGCATGTGGATGTGACGGATGCGTGG |
| | CATCAGTCGGATCATGACGATCCGGGGCCGTGGTTTCCGTGGGACAGGTTTATGGCCGTCGTGAACGGCGGCAGTGGAGAGA |
| | GTGAGGAGTTAACGGTGGCTGATGTGAAAGCCTTGCATGATCAGATTAAACAATTGTCTGCCCAGCTTACTGGTTCGGTGAA |
| | AAAGCTGCATCACGATGTTGGTGTGGTTCAGGTACAGAATGGTGATTTGGGTAAACGTGTTGATGCCCTGTCGTGGGTGAAG |
| | AATCCGGTGACGGGGAAGCTGTGGCGCACCAAGGATGCTTTGTGGAGTGTCTGGTATTACGTGCTGGAGTGTCGTAGCCGTA |
| | TTGACAGGCTTGAGTCGACTGTCAACGGTTTGAAAAAGTGGTGGTGGTTTGTTGTGGGTAAACAGTTTTGGTTGGGCTTGTTT |
| | GAGCGTGCACTGAAAACTTTTATTCAAACGTTTGTTGCTGTGTTGGGTGTGACGGCGGGTGTCACGTATACTGCGGAGTCGTT |
| | TCGCGGTTTGCCGTGGGAATCTGCCTTGATAACAGCAACGGTGGCTGCGGTGTTGTCGGTTGCTACCTCGTTTGGTAACCCGT |
| | CGTTTGTTGCCGGTAAACCGAAAACGACGCCTGTTGTGGATGCTGGGCTTGTTCCACCGGATGATCCTGGAATAGTGGAGTCT |
| | CACATGGTTGACGAAACGGATCCTGGCATGATAGAGCCTATCGACGATGCCGATGCTGGAGGCTATGTGCCGAGGCGTGCTG |
| | CTGAGTCGGAAGTTGGCACGGTAGAGTCTACTGTTGCATAAGTGAATATAGATGTGTGCCCCAGCGGTGCTGCCACGGTTGT |
| | GTGGTGGCTGCTGCTGGGGCACTCTTTTTGTGTCTATAGGGGTTTTACAGGTTGTTGTCTAGTGTGTCTTCGATGAGCTGGTCC |
| | AGGTGGAGGCAGGCTGAGATAGTATCGTTGGCCTGGTCTAGAACATCCTGGCCGATAACGCCTTTGTGGTTGTCGCGGTGGT |
| | AGAGGATGGACTGCATGATATCGTCGGCCTCCGCTTGCAGTAGTTTTGCCTGGTATGCGTTTCTGGCGAGCCAGTCTAGTGCT |
| | TCCTGGCTTGCATAGGGGTTCTGGTCCTCGCTGTTGTCACGGGTGTTGCCGTTGTTTGTGGGGTGTCCTGCACTGTCGCAGTCC |
| | CACAGGATTTCGCTGCACTCGTCTAGCGTGTCCTGGTCGATGTCGAGATCGTCGAGGCTGACTTCTTTGACGGTGAGGTTCAC |
| | ATCATCGAGGGAGATGGGTACATGGTACTGGTTTTCGATACCGGCAACAATGTTTTCCAACTGTTGCATGTTGGTGGGTTGTT |
| | GTTGGATGATTCGGTGTACTGCTGTTTTTAGGGCAGTGTAGGGGATATTGGTTGTGTTGTTCATGGTTTTATCCCATCCCTGTG |
| | CTGTCGTCGTTGCCGTCTGGATAGTATTTACTGTTTGCGTATCCTGTGAGGGTGATGAGTGTTTGGTCTGCCCACTGTTTCACG |
| | GTTTGTCTTGTCACACCCAATCGTTGGGCGGCCGACGCATATGTTTGGTCATACCCGTATACTTCCCGGAATGCTGCCAACCG |
| | TGCCAAATGTTTTCGCTGTATGGATGGCTGGCAGGTGAGGGTGTAGTCGTCGATGGCTAGCTGCAAATCGATCATGGAGACG |
| | ATGTTGTTGCCGTGGTGTTGGGGGGGGGTTGGTGGTGGTGGCATTCCTGGTTCGACGGAGGGTTTCCATGGGCCTCCGTTCCA |
| | GATCCATTGGGCGGCTTGGATGATGTCGGCGGTGGTGTAGGTTCGGTTCACTGGTCACCCCTGGAATAGGTTGTCGAGGTTGT |
| | CTGGGTTGCTGGTGTTGGTGGTGTCGAATCGTCCGACGCAGTGGCAGTAGTCGTACATGAGTTTGATGATGTGTTGGTGGTCT |
| | CCCAAATAGGTGTTTCCGCTGATGCTGTAGGTGGCTGTGCCGTCTTTGCTGATGGTGTATTTGGTGGTGATGGTTTCGGGTGTT |
| | TCGGTGTTGGTGATGATGGCTGTGGTGGTGGAGCCTACGGTTTGTAGCCTGGTGGTTTGGGTGCCGTCGTCGAGGATGGTTGT |
| | GACCATTGTGGTTCTCCTTAGTTGCTGGTTTGGTTGTCGGCTATGGCTGTGATTTCTTGGACGGGTTTGGGTAGGTCTAGGTGG |
| | TGGATTGTTTTGTTTGCTAGTCGTTGGGCGACACGGTAGCACATTTGGGTCCACTGGTTGCCTGTGAGTTGGTGGTATTGGTTG |
| | CGTACCGCAATATATAGTAGAGAGTCTTGGTACAGGTCGTCGGGGTTGACGGCCGGGTAGTGGCGAGCAATGTTGGTGCAAG |
| | CTTTGTGTAGCTGGTGTTGGTGGTGGTGGGTTGCCCACCCCCAGTTGGCGGTGGTGGCTTGTTGGACTTTGGTTGGTCGTCTAC |
| | TCATGGTTTTTATCTGGCTATCTGGTAGTTGTTTGGTGTTTTATGGTTGATAGTGTGGCACACGAGTCAGGGGTTGCCGGTGGT |
| | GCCTGTCTTGTGCCGGTACCAGACGGATTCTCCTTCCATGGATGGGCATTGTATGAATGTGCGTTGTCCTTGCTCGGATATTTC |
| | GAGGTGGTGCCTGTGTCCGGCCATGAGGATGTGGGATGTGGTGCCGTTGTGGAATTCTTGGCCGCGCCACCATTCGTACTGTT |
| | TGCCGGTTTTCCATTGGTGCCCGTGGGCGTGCAGGATTTGTGTGCCGGCCACATCAACGGTGGTGGTCATTTCGTCCCGGCTG |
| | GGGAAGTGGAAGTGAAGGTTGGGATATTGGTTGTTGAGCTGGTAGGCTTCTGCGATGGCCCGGCAGCAGTCCACGTCGAATG |
| | AATCATCGTAGGTGGTGACGCCTTTGCCGAATCGTACGGCTTCTCCGTGGTTGCCGGGGATGGATGTGATGGTCACGTTTTGG |
| | CAGTGGTCGAATTGGTGGATGAGTTGCATCATGGCCATGCGGGTGAGCCTGATTTGTTCCGTCAGGGGTGTTTGTGTGCGCCA |
| | GGCGTTGTTGCCTCCTTGTGACACGTATCCTTCGATCATGTCGCCTAGGAATGCGATGTGGACTCGTTGCGGTTTACCGGCTTG |
| | TTGCCAGTAGTGTTTTGCGGCTGTGAGGAAGTGTAGGTAGTCGTCGGCGAAGTGTGCTGTTTCCCCGCCGGGGATGCCTTTGC |
| | CGATTTGGAAGTCTCCCGCCCCGATGACGAAGGCCGCAGTGCCGTAGTCGGTGTGGGTGTCCTGTTCGGGTTTTGGTGGCTGC |
| | CATTCGGCTAGTTTATCGACGAGTTCGTCTACAGGGTAGGGGTTGGTTGCTGGTTGGTGGTCGATGATTTTTTGTATGGATCG |
| | GCCTGTTTCTCCGTTGGGTAGGGTCCATTCGGAGATGCGTGTGCGGCGTACGGTTCCGTTGGCGAGATCATCGCGGATGGTGT |
| | CTGCTTCGTTGTCGTGGTTGGCTAGCTGGGTGAGGAGCCGGTCTATGTTGTCTATCATCGGGTATCCTCTTCCTTTTGTGGAGT |
| | GGTGTTGGCTTGTTTGCGGCGGTAGTCTTTGATGACGGTGGCGGAGATGGGGTATCCTGCCTGGGTGAGCATTCGGGCTAGCT |
| | GTGTGGCGGGGACGGTTTTATCGGCGAGCACGTCGGCGGCTTTGTTGCCGTAGCGTTGGATGAGGGTTTCAGTTTTGGTTGCC |
| | ATGATGTCCTAGGGGTTGTGTGGTTGGTTGCCATCCTGTGCGGCAGTCGCCGTCGTGTCCTGGTTTGCGTGTGCACCATGTTGT |
| | GGTGCCGTCTGTGTGGTGGAGTGTTTTGCCGCACATGACTTCTTGGAGATGCTGTGGTAGCTGCTCCGGGTTGTTATCGTTTTG |
| | TGTGTCGAAGAGTGTTTTTTGTTTGGTGAAATGTTCGGAGACGGTTCCGTTGTGTACGGGTAGTATCCATGTTTTCCATTGTTG |
| | TTGTAGTCGGGTGTTCCAGTGGAATTGTTTGGCGGCGTTTTCTGCCTGTTTTATGGTTTTGTAGTAGCCGACGAGTATGCGCTG |
| | GTGTTGCTGGTCGGGCGGGTTTTGGCCTCGCCAGTATTGTGCCGCGACGGCGTACCGGTTGCTGGCTGTGAAGGCGTCCCAGC |
| | AGTATTCGATAATGTGCTGGAGCACATTATCGGGCATGTCTCGTACTTGGTTTTCGTCGAGCCACTCGTCGACAATAATGTTG |
| | CGTATGGCTTGCTTGTCTTTGGTGGTGGATTTGAACGAGATGCTCACGATAGTACCGGCTGGTCGTCTTGCATGAACTGGTTG |
| | AAGGTGTTGTTCCAGGCGTGTTGGGCTTGTGTGATTTGCTGGTCGGTCCAGTCGGGGTGTTGCTGTTTCAGATAATACCAGTG |
| | GCACGCATTGTAGGTTTCGTTTTGTAGCCGTGTGAGATGGTTTTCTTCGATGATTTGTTTCCACATGGTCCATGACACGTCGAG |
| | CCGGTTGAGGATTTCGAGGGCTGGGATGTTGAACTGGTCGAGGAACAGGATTTCGTGGGTGTAGTAGTTTTTCTCGTAGGCGT |
| | CCCATCCGCTTTGGTGCCTGTTGGGCTGGTTTTTGGGGTAGGCTTCCCGGCAGATTTTGTGTAAACGCTTGGCCATGTCGTCGG |
| | GTAGTTTAATGTCGGGGTTGGCGCGGATCATGGATCGCATCCCATCATAGGTGGTGCCCCAGGTGTGCATGATATGCAGTGG |
| | GTCTTCACCGTCTGCCCATTTTTCTGCACAGATGGCGAGGCGGATACGCCTCCTGACTGTTTGGCTGGTGTTGCGCCGGTTGG |
| | GGATGGGGCACGTGTCGAGGGGATCCATGATGTTTTAGTTTACCTTTCTTGTTTCGTGTTGTTGACGTGTTTTACTGTAGCACA |
| | GTGTCTAGTGCTTGTGTCAACCCTGTTTTTCCGGCCNGCNTGTAGGTGTCTGTGAAGGCACCGATAGTGAGGGGCACATGGGN |
| | GGNTTGNNGTNNCNCGGTTTGGNGGGTTTGGGNCATCTTGTCTCCCGCTTTGTCTGGGTCTGACCAGATGTAGATCCGGTCGT |
| | AGCCTTCGAAGAATTTGGTCCAGAAGGTTTGCCACGAGGTTGCGCCGGGTAGGGCGACGGCCGACCATCCGCATTGTTCGAG |
| | GATCATGGAGTCGAATTCGCCTTCGCAGATGTGTATTTCGGCTGCCGGGTTGGCCATGGCGGCCATGTTGTAGATGGAGCCTG |
| | TGTCTCCGGCCGGGGTCAAATATTTGGGGTGATTATGTCCCTTGCAATCATGCTGGAGTGAGCAGCGAAACCGGATTTTGCGT |
| | ACCTCGGCTGGGCCGTCCCATGTGGGGTACATGTAGGGGATGGTGATGCACTGGTTGTAGTTTTCGTGGCCTGGGATGGGGTC |
| | ATTGTCGATGTATCCAAGGTGGTGGTTGCGGGCTGTTTCTTCGCTGATGCCTCTTGCTGAGAGCAGGTCGAGTATGTTTTCGA |
| | GGTGGGTTTCGTAGAGGGCCGAGGCTTTCTGGATTCGGCGGCGTTCCGCAATGTTGTATGGGCGTATACTGTCGTACATTCGG |
| | GTTTTCTTCTTCTAATCGTTGTTGTAGCTTGGCAAGTCCGCCTCCGACACCGCATGTGTGGCAGTACCAGACGCCCTTGTCGAG |
| | GTTGATGCTCATGGAGGGCTGGTGGTCGTCGTGGAACGGGCAGAGGATGTGTTGCTCGTTCTTGGATGGGTTGTACCGCATCC |
| | GGTAGGTGTCGAGGAGGCGGCAGGTGTCAGAGGTGTGGGAGGAGCTCGTTGAGGGTTGATTCCACATAGGCTTCGCTCCATG |
| | GCTTGTTGCGCTGTTTCATCACTACGAGTCCGATGGTGGAATTGTTTTCTCGGTTTCGGTGGGTTTCGTAGTTGCGTGCCTCCC |
| | GGCTGGCTTGTTTCACGAATTCGGCAAGGTGTGCCTGTCCTGCTTTGGCTTCGATCACATAGGTTTTGTGGCTGGTTGTGAGG |
| | ATGAGGTCGCCTTCATCCTCTTTACCGTTGAGGTGGAGGCGTTCTATATCATGGCCGGTGTCGCGTAGTTGGTGGAGGAGTCG |
| | TGTTTCCCATTCGGCTCCGGCCCGGCGGTTGCGTGCCTGTTGTGTTGACATGATAGTCCTTTGTGGTGTTCGGTCATGTTCCAT |
| | GGCTGTTTTTCGGCGAGTGGCCCGAAGGATGTGTATTCGGGGTAGGCTCTGAGTCTTTCGTATCGGGTTCCGTCTGGGCTGGA |
| | TTTGCCTGTGCGCTGTTTGAGTACAGCGATGCGTGCCTCAGCGGGGATG |
| | SEQ ID NO.: 16; >4912 len:9543 cycle: O |
| | GGCTTAAGTGGCATCTGTCACACCCGACACTCCAGCATGAAACGCTCAATCCGGTTGAGCGCAGCCTTGACTCCCGGTTGCA |
| | ACCTACACACTCTAGAAACCACAACAACCAACCCAAACAGTTCAGAAGGGAGCACACTCGTGGATGGCACACTCGTCACACC |
| | ATCCTTCACCAGCCTCTACGGGCCCACAGACATCAACCCACTCCACCCCGCACACCTGGCAGGATGTGACATAGGCAACACC |
| | CACACGTCTATCATCTGCCGCCTGCACCGTGCCAAAGTCGAGGAAGCCATCCGACTCATCCGGCCCCTGTGGACTGTCACCCT |
| | CGACGGAGCCGTATACGGGCCACCCGACTGGCAGTCACTCACCGAAAACGAAGCCGAAGAGTTGCACGACATGATCGACAT |
| | GATCGACGTGGACGCCATCATCGCCGAAGCCACACACAACGAGCCTAGAATCGCCTAGAAATACTCGACCTATATAGTTGTA |
| | CCCCCAAGGGCTGAAAGTCGCTCTAATCCTCAGAGTACAAGCTTAAAACACGAAAGAAGGACCAATCATGCAATGGACATG |
| | CCACAAGTGCGATAGCACAATCATCGGATACCGACCAGAGCACTGCACAGTCTGCCACGAGACATTTAATGGCACTCATGCC |
| | GGGGACATGCACCGGACAGGCGATCACGGGGTCAAGGAAGGCCCCAGTCGCCGCAGGTGCCTCACTGTGGACGAGATGGAG |
| | GCTAAGGGATGGCTCGTAGCCGCCATGGATACTGGACGCAAGGTAGTGACATGTCACGCTTAAATGACAAAACCCCAAGCCA |
| | CACAGGAAACGCTCACACAAACTGAGCGACACCTTGACAGTGGTAGTACAGTCTGGAAATATTAACCATGTCAGCAACGAAC |
| | AACACCCCAGAAAGGGGAACACAGCCATGAACAAGAAAAAAGGCTACACCATCGCCGGAATCACAGCTGCCATCATCGCCG |
| | CATGCTCATTTATGCCAGCCCCAGACGACAATCCGCCACTCGCCTCACAGCCAGCCCCACAGGCCACCACAGCCAACACCGA |
| | ATGGACCCCCAAGACTACCCAACAGCGCAAAGCCGAGAAAGCGGCACGGCAGGCAGCCGCCACCCGCTCCATACAAGCCGA |
| | GCAGGCCAAAGCCCACAAGCAAGCCCAAGCAAGGGGGGAAGAAACCGCCACCGGACTCACCATGATCACGGCAGCACACA |
| | CCTGCAACCGCAAAGCCGAACAACAGGCCGCCGCACACGGTGTCAACTGGAACGGCAACCCCGACATCGACCTCCAACTCC |
| | ACAAAACCATCGGTAAAGACACATTCAGCATCGTCTACGGCGCAACCGCGAAACAGCCCGGCGCATCCAAACTACCCGTCAC |
| | CGTCCACTGCCTCGTCACCGGAACAGAAGACCACCAGCACGTCACCGACCTCAACATCAACCCGCAACAGTAACCCGCCAAG |
| | GAGCATCCCCGCTATGCCTCTCCTCTCCCACTACGCTGTCACCACCGGACTCGCCGACACGGCACACATTATTCACCACACCG |
| | GCGGCACACTACGCACAGCCACCGATATTGCCTCCCGCATCAACACCCTCAACCCAGACATTGATCTCGACCACGAAATCCG |
| | ACAACTGTTATCTATCGAAACCGACCTGTACAACATTTATAAAACCATCAACACCATTCTTCAGGAGCAAGCATGAACACAC |
| | CCAACAACAACATTGAGCTACACAGCTACGAAACGTTCTTCACCAGCCTCGCCTGGATCCAAGGCGGCATCATCACATGGAT |
| | GTACGCAACCGGCACCCCACACAAGGCAGCCCTCGCCATCATCGCCGCATGCGCCCTCGCCACCCTCCTGGGTGCCTCAACC |
| | CTCACCAACAATCCCCGAGACACCAAATGATCACAACACCCATCCTCATCGCTGAAACCCTCGCCATCATTATTCTCGCCGTA |
| | GCACTAGCCCACAACAACAACCAGTAACCCACACTTAAGGAGCACACACCCCATGGATGAGCCCACCCGCATGTACACCGAC |
| | CCCAACACCGGAGCCCGAAGAGAATTAAAACTCTGCAGGCTCTCACTCATCGACCCCGCAGCCTTGCACGCCCTAGGCGAAG |
| | TAGCCGGATACGGTGCTACTAAATACGGCGACAACAACTGGACCGGCGGCTATCCGTGGAGCCACAGCATTGACGCCCTCTA |
| | CAGGCACCTGCTATCATGGCAGCAAGGAAACAACCTCGATTATGAATCCGGGCTACCCCACCTGGCACACGCAGCCTGGCAC |
| | TGCCTCGCACTCCTCGCATACCAGCAACACAATGCCGGCCGAGACACCCGCAACCCATGGAACAAAAGCGACAAGTAATGCC |
| | TCTAGCACAATACCAGAAAACCATCAGCCATCCAGGCCACATCTCCTACAGTTCACTCACCCAGTGGGCCGAATGTGGAGAA |
| | AAATGGCGCCTATCCCACGGCTACCACACCCAACACCACACCTGGTACGCCACCATCGCCGGAAGCGCCATACACCACATCA |
| | CCGAACAATACGACCTACACCTGTACAACCCCACCGAATACTCTGCACTACCAGACAAACTCTCATCCTTCAAAAACATTTTC |
| | GACACCCAAGTCGCCCTCGCCGAATCCGAAGGCACAGAAATCAAACCCTCCGGCAGAATATGCAAAAACATGTGCGAGTCG |
| | GGCGGGCCACACAAGAAAGACTACGACTGGTGGATGGTTTACGGCCCCACCTTTGTGGACCGCTGGAAAACATGGAGGCGC |
| | AACCACCCAGGATACATCACCGCAATCCTGGACGGTAAACCAGGCATCGAATACCCGGTAGAAACCATCCTCGACGATGACA |
| | CAAAAATAGTCGGCTACATCGACCGCGTTTTCACCGACACCGACACCGGCGAAACCTTCATCCTCGACCTCAAAACCGGACG |
| | CCTACCCGCCGACAGTATGCAGCTGCACACATACCGATACATGCTCAACCAACACGGCAACGATGTGACAAAAGGCATGTTT |
| | TGGGCGCCAGCCACCGGCTGCAACGACGACAAGTCCCCAACACAAGGCACATCCACCGAACTCTACGACCTTGACAACAAC |
| | ACCTACCGGCATGTATCATCCATGTACAGTCAAGCAATGAAAGGAATCAGCGAAGGCATCTTCGTCCCACACGTCACAACAC |
| | TCTGTAAAGGCTGCCCCGTCAAGGACGCCTGCTGGGCTGTCAACGGGAAAGACGCCTACAGGTACCCTATAGAAACCACCAT |
| | CACAGCCCCAACAAAAGAAGACAAGGAGCACCAGTGACCGACAACACAGACGACGACCGATTCACCGTCACACTCAAATAC |
| | GGAGGCGACTACGCCGCCCCATGGACCGTCATCCGCGGAGACACCGCCGACCAGGTAAAGAAGGCTATCATCGACCTGCTA |
| | GGCGGACTCAAAAACAGCTCCGCGGCAAGGAACTGGGACCTGGCAACACTGATCGCCACAGCATCCATCATCCTCCAAGACC |
| | GATACAACCAGGCCGCCAAAGACTACGTAGACAACATCGCCTCAGAAGGAAACAACACCATCATCGACAAAATCAACAATG |
| | CAACAAGCAAAGCACAGCTAGCCGACCTCCTCAAACAGTACAAAAAGATCATCACCAGTAACAGGGACGTGTCTGAGGCTTT |
| | CCGCAGCAAACGCAACAGCCTCACCCGATAAAAACCGACACAAACCAACAAAACCAAACATAAACAGTAAAAGGAAAACA |
| | GTTATGGGACTCGCCAACTACCGAAACAACAGCAACAGCACCTTCTTCAACCCCTCCCGAAACCAGGACGCCACCGCCATCG |
| | CCTTCAAAATCCACGACGTCCAGCACAACACCGAAGGCTACGGCGGACAGACCGCAGACCGCATCTACGCCGATGTCACAAT |
| | CTTCCACACCCTAGACGATCTCAACAACGGCACCCCAGAAACCATCCCCAACGCCATTATCGAGAAAGCGCGCGGCAACAAC |
| | GACCGCCCACACTCCATGATCCGCGATCTAGAAGCCTATCTTGGCGAGGAGCAGGCCTTCAAACTCGCCACCGTACGCACCA |
| | AAAACGGCTTCAACGCGGTAATCCTCAAACCCCTTGATGACGCCATCTACGATAAGGTTGCCGAATACGTAGACAAGCGCGA |
| | TAACGGCCAGCTAGACGACACCACAGCCTCAACTGATGCTGATATCGATATCGACTCCATCTGACCACCAATACACATCCAA |
| | CCGATAGATAGATAAGGTCCCGATGCTCTCTCTCCAACGATCCTTCGAGAGAGCCTCCCAAACCGCAGCCGAGCTGCCCCGC |
| | ATACCCCAGCTAGAACCCCTCTACCGCAACCAGGACATGCACATCCACAAAGGGGATTTGGTCATGATCGCGGGGCGGTCCG |
| | GCAGCCAAAAATCAGGGCTAGCCATGTTCATCACAGCGATGCTCAACCAGCCAGCCCTCTACATATCAGGGGACATGACACC |
| | CTGGGAGGCCTCCACACGAATCATCTCACTCAACACCCAACACACCACCACACAGATACAACAAAACATCGACGACTACGGG |
| | CCAGAATACTATCGAGACAGTATCCACCACGGCCAACACATCACATTCTCATTCCAGTCACCCATCACATGGACAGACATCA |
| | CCATGGAATTGCAAGCCTACATGGAAATGTGGAACACCTTCCCACCACTCATTGTTATCGACAACCTGATGGACATCCAAGA |
| | CTGCGAGAGTGACTATCAGGCACNGCAAGAAGCNATGCAATGNANNACNNCATNAGNCAGGGATACTGGCTCCACCATCAT |
| | CGTCACCCACCACGCAACNGANAAAACCGGAACCGACATAGAACACCCGCCAGCTAGACGGGAAATCAAAAATGGCCTCTC |
| | CGAAAAACCACAACTCATCCTCGGGGTCTCATTGTATGGTGGCGAGGATAATGGCAACGGCCTCACGATACCGGCAGAGGCA |
| | CGCATCGCCGTACTCAAACAGCGCACCGGCAAATCCGGTCCTGACGGAACCCGATACGAACGACTCAGAGCCTATCCTGAAT |
| | ACACATTCTTCGGGCCCCTCGCCGAAAAACAGCCCTGGAACATGACCACAACACACAAAGGACTATCATGTCGACACAACAG |
| | TCACGCAACCGCCGGGCCGGCGCAGAATGGGAAACACGACTCCTCCACCAGCTCCGTCACATCGGCCATGATATAGAACGCC |
| | TCCACCTCAACGGTAAAGAGGACGAAGGCGACCTCATCCTCACCACCAGCCACAAAACCTATGTGATAGAAGCCAAAGCAG |
| | GACAGCAGCATCTAGCCCAATTCGTCAAACAAGCCAGCCGAGAAGCACGCAACTACGAAACACACCGAAACAAACAAAACA |
| | ATTCCACCATCGGACTCGTAGTGATGAAACAGCGCAACAAACCATGGAGCGAAGCCTATGTGGTATCAACCCTCAACGAGCT |
| | CCTCCCACACCTCTGACACCTGCCGCCTCCTCGACACCTACCGGATACGGTACAACCCATCCAAGAACGAGCAACACATCCTC |
| | TGCCCGTTCCACGACGACCACCAGCCCTCCATGAGCATCAACCTCGACAAGGGCGTCTGGTACTGCCACACATGCGGTGTCG |
| | GAGGCGGACTTGCCAAGCTACAACAACGATTAGAAGAAGAAAACCCGAATGTACGACAGTATACGCCCATACAACATTGCG |
| | GAACGCCGCCGAATCCAGAAAGCCTCGGCCCTCTACGAAACCCACCTCGAAAACATACTCGACCTGCTCTCAGCAAGAGGCA |
| | TCAGCGAAGAAACAGCCCGCTACCACCACCTTGGATACATCGACAATGACCCCATCCCAGGCCACGAAAACTACAACCAGTG |
| | CATCACCATCCCATACATGTACCCCGTCTGGGGGCGGCCAGCCGAAATACGAAAAATGCGTTTCCGCTGCTCACTCCCGCAC |
| | GACTGCAAAACCCACAACCACCCCAAATATTTAACCCCCGCCGGGGACACAGGCTCCATCTACAACATGGCCGCCATGGCCA |
| | ACCCGGCAGCCGAAATGCGCATTTGCGAAGGCGAATTCGACTCCATGATCCTCGAACAATGCGGATGGTCGGCCGTAGCCCT |
| | ACCCGGGGCCACCTCGTGGCAAACTTTCTGGACCAAATTCTTCGAAGGCTACGACCGCATCTACATCTGGTCCGACCCCGACA |
| | AAGCAGGCGACCAGATGGCCCAAACCCTCCAAACCGCACTCCCCCAAGCCATCCACGTGCCCCTCACTGTCGGAGATGTCAC |
| | AGACACCTACCTGCAGGCCGGCAAAACAGGGTTGACACAAGCCCTCAACACAGTGCTACAATAAAACCAGACAAACAACCC |
| | AAACCAAGAAAGGCATATAAAGCATCATGGATCCCCTCGACACCTGCCCCATCCCCGGCCGCCGCGACACCTCCAAGGCCGC |
| | CAGGAGACGCATCCGCCTCGCCATCTGTGCAGAAAAATGGGCCGACGGCGTGGACCCACTCCACATCATGCACACCTGGGGC |
| | ACCACCTATGATGGGATGCGATCCATGATCCGCGCCAACCCCGACATCAAACTACCCGACGACATGGCCAAACGTTTACACA |
| | AAAGCTGCCGGGATGCCTACCCCAAAAACCAGCCCAACAGGCACCGAAGCGGATGGGACGCCTACGAGAAAAACTACTACA |
| | CCCACGAAATCCTCTTCCTCGACCAGTTTAACATCCAGCCCTCGAAATCCTCAACCGGCTCGACGTGTCATGGACCATGTGGA |
| | AACAAATCATCGAAGAAAACCATCTCACATGGCTACAAAACGAAACCTACAATGCGTGCCACTGGTATTATCTGAAACAGCA |
| | ACACCCCGACTGCACCGACCAGCAAATCACACAAGCCCAACACGCCGGGAACAACACCTTCAACCAGTTCATGCAAGACGA |
| | ACAGCCGGCACTATCGTGAGCATCTCGTTCAAACCCACAAAACGCAACCTTCAAACCATCCGAGACATCCTCACCCAGGCCA |
| | CCAACCTCAACCCAGATGATCTGCCAGATGACACCCTCAACAACATCATCCAATACTGCTGGAACACCTTCACTGACAACAA |
| | CCGGTACGCCGTCGCGGCACAATACTGGCGAGGCCAAAACCCGCCCGACCAGCAACACCAGCGCATACTCGTCGGCTACTAC |
| | AAAACCATAAAACAGGCAGAAAACGCCGCCAAACAATTCCACTGGAACACCCGACTACAACAACAATGGAAAACATGGATA |
| | CTACCCGTACACAACGGAACCGTCTCCGAACATTTCACCAAACAAAAAACACTCTTCGACACACAAAACGATAACAACCCGG |
| | AGCAGCTACCACAGCATCTCCAAGAAGTCATGTGCCGCAAAACACTCCACCACACAGACGGCACCACAACATGGTGCACAC |
| | GCAAACCAGGACACGACGGCGACTGCCGCACAGGATGGCAGCCCACCACACAACCCCTAGGACATCATGGCAACCAAAACT |
| | GAAACGTTGATCCAACGCTACGGCCGCAAAGCCGCAAACGTCCTCGCCGACAGGTCTATACCCGCATCCTGGCTAGCAAAAC |
| | AACTCACCCAAGCCGGATACCCCATCTCCGCCACCGTCATCAAAGACTACCGCCGCAAACAAGCCAACACCACTCCACAAAA |
| | GGAAGAGGATACCCGATGATAGACAACATAGACCGGCTCCTCACCCAGCTAGCCAACCACGACAACGCCATCGACACTATC |
| | GACGACATTCTCGCCAATGGTACTGTACGCCGCACACGCATCTCCGAATGGACCTTACCGAACGGAGAAACAGGCCGATCAA |
| | TCCAAAAAATTATTGACCACCAACCCCCATCAGACCCCTACCCTATAGACTAACTCGTCGATAAACTAGCCGAATGGACACC |
| | CCCAAAACCAGCCGACAACACTCGCACCGACTACAGCACTGCAGCCTTCGTCATCGGGGCGGGAGACTTCCAAGTCGGCAAA |
| | GGCATCCCCGGAGGAGAAACATCACACTTCGCCGACGACTATTTGCACTCCCTCACCGCCGCAAAACACTACTGGCAACAGG |
| | CAGGCAAACCCGAACGAGTCCACATCGCCTTCCTCGGCGACATGATCGAAGGATACGTGTCACAAGGAGGCAACAACGCCT |
| | GGCGCACACAAACCCCCTTGACGGAACAAATCCGGCTCACACGCATGGACATGATGCAACTCATCCACATGTTCGACCACTG |
| | CACCAACCTTACGGTCACCTCCATCCCCGGCAACCACGGAGAAGCCGTACGATTCGGCAAAGGCGTCACCACCAACGATGAT |
| | TCAATCGACGTCGACTGCTGCCGCGCCATCGCAGATGCCTACCAACTCACCAACAACTACCCCAACCTCCACATTTATTTCCC |
| | CCAACGCGACGAAATGACCACCACCGTCAACGTAGCCGGGGCTACCATCCTGCACGCCCACGGACACCAATGGCGCAACAA |
| | CCGACACTACGAATGGTGGCGCGGCCAAGAATTCCACAACGGCACCGTATCCCACATTCTCATGGCCGGACACAGGCACCAC |
| | CTCGAAATATCCGAGCAAGGACAACGCACATTCATACAATGCCCATCCATGGAAGGAGAATCCGCCTGGTACCGGCACAAG |
| | ACAGGCACCACCGGCAACCCCGGACTCGTGTGCTACACTATCAACCATAAAACACCAAACAACTACCAGATAGCCAGATAA |
| | AAACCATGAGTAGACGACCAACCAAAGCAGACCTAGCCACCACCGCATCGTGGGGATGGGCAACCCCCCACCACCAGCGCC |
| | AGCTACACAAAGCCTGCACCAACACGGCACGCCACTACCCCGCAATCAGCGTAGACGACCTCTACCAAGACTCCCTACTATA |
| | TATTGCGGTGCGGGAACAATACCACAACCTAGACAACAAACACTACACCAAAATGTGCTACAGGGTAGCCAAACGGCTAGC |
| | CAACAAAACCATACAACACCTAGACCAGCCGAAACCTTTACCCGATATCATTCATCTAGCCGACAACCAAACCAGCAACTAA |
| | GGAGAATCATAATGGTCACCACCATCCTCGACGACGGCACCCAAACCACCAGGCTACAAACAGTAGGCGCCACCACCACCG |
| | CCATCATCACCAACACAGAAACCCCCGAAACCATCACCGCCAAATACACCATCAGTAAAGACGGCACAGCCACCTACAGCAT |
| | CAGCGGAAACACCTATTTGGGAGACCACCAACACATTATCAAACTCATGTACGACTACTGCCACTGCGTCGGACGATTCGAC |
| | GCCACCAGCCAGCCAGACAACCTCGACAACCTGTTCCAGGGGTGACACATGACCCGAACCTACACCACCGCCGACATCACCC |
| | AAGCCGCCCAATGGATCTGGAACGGAGGCCCATGGAAACCCTCCGTCGAACCAGGAATGCCACCACCACCAACCGCCCCCC |
| | AACACCACGGCAACAACATCGTCTCCATGATCGATTTGCAGCTAGCCATCGACGACTAC |
| | SEQ ID NO.: 17; >5146 len: 10856 cycle: O |
| | GTCTTAAACTTCAATATAATAACCTTTAAATCTTAAAGCTTATAGGTATAATAATATAATATAACTATATAAGTATTAAAGCT |
| | TATAAGTTATAAAAGTTTTAGAAGAGCTAAGAGGTTAACTTCTTTACTTCTCTTCTCTCTTTGGTTCTTTCTCTCTTCTCTTCT |
| | TTTCTTCATCAGGGGAGAAGAGGAACCTTTACCATCAGCGCCGATGACCTTTGCCGTGTGTCTCGTGTACCACCGGTCGCACGC |
| | TCCCGGTTTGCACACTCCCCACACTCTGACACCCGTGTCCCTTTCAGGCTTAGCGTGTTCGGCTGAAGGCGTACGGCGTGTCA |
| | CGCTTAAACCCTTAACACCAGGTAAGACTTAAAGTGTATATTATAAGTAGAAGACTTTAAAACCTTAAGGTGTTCCCGCTTAG |
| | CCTGTGTCCTTTAACGCCAGGCGTCAAGCACTAAGCTGTGAAACGCGAACACCCATCCACCCCCATTTTTCTTTCGTGTCCTTC |
| | TCTTTTTGACACAGCTGGGGGGCGATGTGATCTTTCTCACAGGCCAGGGGGTAGTGGAGAAAACAAACACCCCCTCAAACGA |
| | ACAAAACAGCCCCCAGAATCGAATGACAGGGCAAGGGCAAGGTATTCATACCCCCAGACGATTCCAGGCCGTTACAAGAGC |
| | AAACAAGACCCGTACAGGGCTAGGCGAGGATCGGGGACGTGATGGCACACACCAACCGCACCGCATCATCCGCACACCGAC |
| | GCTGGCGGCAACGACTCATCACCCAAGCCCGACAGCAAGGCCAAACCGAATGCCCACTCTGCGGAGCCCAGATAGCCTGGG |
| | GCACACACGACCTACCAACCAGCCCCGAAGCCGACCACATCACACCCGTCAGCCTCGGGGGACTCAACCCCCTCGACAACGG |
| | GCAAATCATCTGCAGAACATGCAACAGAAGCAAAGGCAATCGCAGTGATCCAAACATTAGTTTCCAACAACAAACCACAAA |
| | AACGTTGATTCCATGGTGAAAAACCCGCCAACCCCCACCGGGGACACCCCCTGCACACCCGTGCAAAACCTCGTACGGCTTA |
| | GTGAAATACCTCCCTTTTGTGGATTTGCCTGTTTGTCGACTTTTTGTGTTGGTGGTGAGTGTTGTGCAGCCTGAGCTTCCTGAT |
| | AGTCGTGAGTGGTGAGGGGAGACGCGTCGTTGGTGGTGTGTGTGGGGTGAGGATAGTCGCGCGCAGTACGTGTCTGATGAGG |
| | AGTGGCTGTTTCTCATGGATGCTGCGGTGATTCATGATGTGGTGTGGCGTGAGGGTCGCGCTGATTTGGTGGCTTCGCTTCGT |
| | GCTCATGCGAAGGCGTTTATGGGCATGTTGGATCGTTATTCGGTTGATGTGGTGTCTGGTGGCCGTGGTGGGGGTTCTGCGGT |
| | GGCGATGATTGACCGGTATCGGAAGCGTAAAGGGGCCTAATGTCGAGCGTTGTTGGTTCTCAGGTTCCTCGTCACCGGGTGG |
| | CTGCGGCGTATTCGGTGTCTGCTGGCGGTGATGCTTGGGAGTTGGGTCGTGCGTATGGGTTGACGCCTGATCCGTGGCAGCAG |
| | CAGGTGTTGGATGATTGGCTGGCTGTCGGTAGCAATGGCAGGCTTGCTTCGGGTGTGTGTGGGGTGTTTGTGCCTCGCCAGAA |
| | TGGCAAGAATGCTATTTTGGAGGTTGTGGAGTTGTTTAAGGCGACTATTCAGGGTCGCCGTATTTTGCATACGGCTCACGAGT |
| | TGAAGTCGGCTCGTAAGGCGTTTATGCGGTTGAGGTCGTTTTTTGAGAATGAGCGGCAGTTTCCTGACTTGTATCGTATGGTG |
| | AAGTCGATTCGTGCAACGAATGGCCAGGAGGCTATTGTGTTGCATCATCCGGATTGTGCCACGTTTGAGCGTAAGTGTGGTTG |
| | CCCGGGTTGGGGTTCGGTGGAGTTTGTGGCTCGTAGCCGTGGTTCTGCTCGCGGGTTTACGGTTGATGATTTGGTGTGTGATG |
| | AGGCGCAGGAGTTGTCGGATGAGCAGTTGGAGGCGTTGCTTCCTACCGTGAGCGCTGCCCCGTCTGGTGATCCGCAGCAGAT |
| | TTTTTTGGGTACGCCTCCTGGGCCGTTGGCTGACGGTAGCGTGGTGTTGCGTCTTCGTGGGCAGGCTTTGTCGGGTGGTAAAA |
| | GGTTTGCGTGGACGGAGTTTTCGATTCCTGACGAGTCTGATCCGGATGATGTGTCGCGGCAGTGGCGGAAGTTGGCGGGGGA |
| | TACGAATCCGGCGTTGGGTCGCCGCCTGAATTTCGGGACCGTGTCGGATGAGCATGAGTCGATGTCTGCTGCCGGGTTTGCTC |
| | GGGAGCGGCTTGGCTGGTGGGATCGTGGCCAGTCTGCTGCGTCTGTGATTCCGGCGGATAAGTGGTCCCAGTCTGCGGTGGA |
| | TGAGGCTAGTTTGGTTGGCGGCAAGGTGTTTGGTGTCTCGTTTTCTCGTTCGGGGGATCGTGTCGCGTTGGCGGGTGCTGGTC |
| | GGACTGATGCTGGGGTTCATGTTGAGGTGATTGATGGGCTGTCGGGGACGATTGTTGATGGTGTGGGCCGGTTGGCTGATTG |
| | GTTGGCGGTTCGTTGGGGTGATACTGACCGGATCATGGTTGCCGGGTCTGGTGCGGTGTTGTTGCAGAAGGCGTTGACGGATC |
| | GTGGTGTTCCGGGCCGTGGCGTGGTGGTTGCCGATACTGGGGTGTATGTGGAGGCGTGTCAGGCGTTTTTGGAGGGTGTCAG |
| | GTCGGGTGTGATCAGTCATCCTCGTGCTGATTCTCGCCGTGACATGTTGGATATTGCTGTGAGGTCGGCTGTGCAGAAGAAGA |
| | AAGGCTCTGCGTGGGGTTGGGGTTCCTCGTTTAAGGATGGTTCTGAGGTTCCTTTGGAGGCTGTGTCTTTGGCGTTTATGGGG |
| | GCTAAACAGGCTAGGCGTGGCCGTCGTGATCGTGGTGGTAGGAAGCGGGTGTCTGTGGTATGAACTCGGATGAGTTGGCTTG |
| | GATTGAGGGCATGTTTGATCGGATTCAAAGGTTGTCTTCGTGGCATTGCCGTATTGAGGGCTACTATGAGGGTTCGAATCGGG |
| | TGCGTGATTTGGGGGTGGCCATTCCTCCGGAGTTGCAGCGTGTGCAGACTGTGGTGTCGTGGCCTGGTATAGCCGTGGATGCT |
| | TTGGAGGAGCGTCTGGATTGGCTTGGCTGGACTAACGGTGACGGCTACGGCCTGGATGGTGTGTATGCGGCGAATCGGCTTG |
| | CTACGGCGTCGTGTGATGTGCATTTGGATGCGCTGATTTTTGGGTTGTCGTTTGTGGCTGTTATTCCCCAAGGCGATGGGTCGG |
| | TGTTGGTTCGTCCGCAGTCACCCAAGAACTGTACAGGCCGGTTTTCGGCTGACGGGTCTCGTCTGGATGCTGGCCTTGTGGTG |
| | CAGCAGACGTGTGATCCTGAGGTTGTTGAGGCGGAGTTGTTGTTGCCTGATGTGTTTGTTCAGGTGGAGCGGCGAGGTAGCC |
| | GTGAGTGGGTTGAGGTGGATCGTATACCGAATGTGCTTGGGGCGGTTCCGTTGGTGCCGATTGTGAATCGGCGTAGGACGTC |
| | GCGTATTGATGGGCGTTCGGAGATTACGAGGTCTATTAGGGCTTACACGGATGGGGCTGTTCGCACACTGTTGGGGCAGTCT |
| | GTGAATCGTGACTTCTATGCGTATCCTCAGCGTTGGGTGACGGGTGTTTCGGCTGACGAGTTTTCGCAGCCTGGCTGGGTGTT |
| | GTCTATGGCTTCTGTGTGGGCTGTGGATAAGGATGATGATGGGGACACTCCGAATGTGGGGTCGTTTCCGGTTAATTCTCCGA |
| | CTCCGTATTCGGATCAGATGCGCTTGTTGGCGCAGTTGACTGCGGGTGAGGCTGCTGTTCAGGAGCGCTATTTCGGGTTTATT |
| | ACGTCTAATCCGCCTTCGGGTGAGGCTTTGGCTGCTGAGGAGTCGCGGCTTGTGAAGCGGGCTGAGCGGCGTCAGACGTCGT |
| | TTGGTCAGGGCTGGCTGTCGGTTGGTTTCCTGGCTGCCAAGTCGCTTGATTCTTCCGTTAACGAAGCTGATTTTTTTGGTGATG |
| | TTGGTTTGCGGTGGCGTGATGCATCAACACCTACTCGGGCGGCTACAGCGGACGCTGTGACGAAGCTTGTTGGTGCCGGTATT |
| | CTTCCTGCGGATTCTCGGACGGTGTTGGAGATGTTGGGTTTGGATGATGTGCAGGTTGAGGCTGTGATGCGTCATCGTGCCGA |
| | ATCTTCGGATCCGTTGGCTGCGCTTGCTGGGGCTATATCGCGTCAGACGAACGAGGTTTGATAGGCGATGGCTTCGGGGGTTG |
| | AGGCGAGGCTTGCTGCTGCCGGGTATCAGCGTGAGGCGATTCGTTTTGCCGGGAAGTATGCGGGCTATTATGCTGAGCTTGGT |
| | CGTTTGTGGCATTCCGGGAAGATGACAGATGCGCAGTATGTGCGTTTGTGTGTGGAGTTGGAGCGTGCCGGCCATGATGGTTC |
| | GGCATCGTTGGCTGCCAGGTTTGTGTCGGATTTTCGCCGGTTGAATGGTGTGGATCCTGGTTTGATTGTGTATGACTAGTTTGA |
| | TGCTGCGGCGGCTTTGGCTAGGTCGTTTTCGACTATGAAGACTCTTGAGAGTGACCCGGATAGGGTGAATGATACGATTGATG |
| | TGATGGCTGCGGGTGTTGATCGGGCTGTTATGAATGCTGACCGTGACACGGTTGAGTGGTCTGCTGGTGCGCAGGGTAGGTC |
| | GTGGCGTCGGGTGACGGATGGTGATCCGTGTGCTTTTTGTGCCATGTTGGCTACGAGGTCGGATTATACGACTAAGGAAAGG |
| | GCGCTCACGACTGGTCATACTCGGCGTCATAAGCGTGGCGGTAAGCGTCCGCTGGGTTCGAGGTATCATGATCATTGTGGGT |
| | GTACGGTGGTTGAGGTTGTTGGGCGTTGGGAGCCCTCCTCTGCGGACACCGCATATCAGCGGGTTTATGAGAAGGCTCGTGA |
| | GTGGGTTGATGATCATGGGTTGCAGCAGTCGCCTGGCAATATTTTGAAGGCTATGCGTACTGTTGGCGACATGAGATGATGGT |
| | TTCCGGTTGTGTGCCGCCGGTTATCGGTGCGCAGGGTTGTCTCCCGCACGGGGGTCAACAATGTTGTGTTGTTTTCCGCAAGG |
| | AGTGTAGGTTAGGCTATGGCCGATCAGAGTGTTGAGGAACAGAATGTTGACAATGATGCTGTGGAGCCCGGAAAGGGTGAC |
| | GGCATTGTTGACACGGTGAAGGGCGATCAAGGCAAACAGGTTGAAGATCAGTCGAAGAGTGAAGGCGAGGGTAAATCGTCT |
| | GGGACTGATTGGAAGGCGGAGGCCCGTAAGTGGGAGTCTCGTGCTAAAAGTAATTTCGCCGAGTTGGAGAAGCTTCGCGCCT |
| | CGGATGGTGATGCGGGGTCTGTGATTGATGATCTTCGCCGCAAGAATGAGGAACTCGAAGACCGGATCAATGGGTTTGTTCT |
| | TGAGGGTGTGAAGCGTGCTGTTGCGGCCGAGTGTGGCTTGTCGGGTGATGCGGTCGCTTTCTTGCACGGTAGCGACCGTGAA |
| | GCGCTGGTGGAGTCTGCTAAGGCTTTGAAGGGTTTGATCGACCATAGTAGTGGTGGCGCGGGTGTGCGCCGGCTTGCGGGGA |
| | GTGCCCCCGTTGATGATGTTAAACGACGTGAGGGTGTCGCGTTTGTGGATGCTCTTGTCAATAATTCTAGGAGATGATTTGTG |
| | ATGGCTGACGATTTTCTTTCTGCAGGGAAGCTTGAGCTTCCTGGTTCTATGATTGGTGCGGTTCGTGACCGTGCTATCGATTCT |
| | GGCGTTTTGGCGAAGCTTTCGCCGGAGCAGCCGACTATTTTTGGCCCTGTTAAGGGTGCCGTGTTTTCGGGTGTTCCTCGCGC |
| | CAAGATTGTTGGTGAGGGTGAGGTTAAGCCTTCCGCTAGTGTTGATGTTTCGGCGTTTACTGCGCAGCCTATTAAGGTTGTGA |
| | CTCAGCAGCGTGTCTCGGACGAGTTTATGTGGGCTGATGCTGATTACCGTCTGGGTGTGCTTCAGGATCTGATTTCCCCGGCT |
| | CTTGGTGCTTCGATTGGTCGCGCTGTGGATCTGATTGCTTTCCACGGTATTGATCCTGCCACTGGGGAGCCTGCTGCGGCTGTC |
| | AAGGTGTCGCTGGATAAGACGAAGAAGACGGTTGATGCCACGGATTCTGCTACGACCGATCTTGTTAAGGCTGTCGGTCTTA |
| | TCGCTGGGGCCGGTTTGCAGATTCCTAACGGTGTTGCTTTGGATCCGGCGTTCTCGTTTGCCCTGTCTACTGAGGTGTATCCGA |
| | AGGGGTCTCCGCTTGCCGGCCAGCCTATGTATCCTGCAGCTGGGTTTGCTGGTTTGGATAATTGGCGCGGCTTTAATGTTGGT |
| | GCTTCTTCGACTGTTTCGGGTGCCCCGGAGATGTCGCCTGCCTCTGGTGTTAAGGCGATTGTTGGTGATTTCTCTCGTGTTCAT |
| | TGGGGTTTCCAGCGTAACTTCCCGATCGAGCTCATCGAGTATGGTGACCCGGATCAGACTGGGCGTGACCTGAAGGGCCATA |
| | ATGAGGTTATGGTTCGTGCCGAGGCTGTGCTGTATGTTGCGATTGAGTCGCTTGATTCGTTTGCCGTGGTGAAGGAGAAGGCT |
| | GCCCCGAAGCCTAATCCTCCGGCTGGTAACTGATTCATTTGTTGCGATAATGTTTATGCTGTGTGCAGGGGGTGGTGTTGATG |
| | GGTATCATTTGGAAGCCTGAGGATATTGAGCCTTTCGCCGATATTCCTAGAGAGAAGCTTGAGGCGATGATCGCCGATGTGG |
| | AGGCTGTGGCTATCAGTGTCGCCCCCTGTATCGCTAAACCGGATTTCAAATACAAGGATGCCGCTAAGGCTATTCTGCGCAGG |
| | GCTTTGTTGCGCTGGAATGATACTGGCGTGTCGGGCAGGTGCAGTATGAGTCTGCGGGTCCTTTCGCTCAGACTACACGGTCT |
| | AATACTCCCACGAATTTGTTGTGGCCTTCTGAGATTGCTGCGTTGAAGAAGTTGTGTGAGGGTGATGGTGGGGCTGGTAAAGC |
| | GTTCACTATTACACCGACCATGAGGAGTAGTGTGAATCACTCTGAGGTGTGTTCCACGGTGTGGGGTGAGGGTTGCTCGTGCG |
| | GGTCGAATATTAACGGCTACGCTGGCCCTTTGTGGGAGATATGATATGACCAGTTTTCCTTATGGTGAAACGGTTGTGATGAT |
| | TCAACCGACTGTTCGTGTCGATGATCTTGGCGACAAGGTTGAAGACTGGTCTAAGCCTGTCGAGACTGTGTACCATAATGTGG |
| | CCATCTATGCTTCCGTTTCGCAGGAGGATGAGGCGGCAGGCCGTGACTCGGATTATGAGCATTGGTCGATGCTTTTCAAGTCC |
| | CCTGTTGTGGGCGCTGATTATCGTTGCCGGTGGCGTATTCGGGGTGTTGTGTGAGAGGCTGACGGGTCTCCTATCGTGTGGCA |
| | TCATCCAATGTCTGGCTGGGATGCGGGCACGCAGATCAATGTGAAGCGTAAGAAGGGCTGATCGGTTGTGGCTCAGGATGTG |
| | AATGTGAAGCTGAACTTGTCGGGTATTCGTGAGGTGTTGAAGTCTCCTGGTGTGCAGGGCATGTTGGCTGAGCGTGGCGAGC |
| | GGGTTAGGCGTGCCGCATCGGCGAATGTGGGCGGTAATGCTTACGATAGGGCCCAGTATCGTGCCGGGTTATCATCGGAGGT |
| | GCAGGTTCACCGTGTTGAGGCTGTCGCTCGTATAGGCACCACATATAAGGGTGGGAAGCGTATTGAGGCGAAGCATGGCACG |
| | TTGGCGAGGTCGATTGGGGCTGCGTCGTGATCGTTTACGGTGATCCGCGTGTGTGGGCTAAACGCGTGCTCAAGGATGATGG |
| | CTGGCTGTCTGGGATACCGTGTACAGGGACGGTGCCTGAGGATTTCAGCGGTGACCTGGTCTGGTTGGCGTTGGATGGTGGTC |
| | CGCAGTTGCATGTTCGTGAGCAGGTGTTTTTGCGGGTGAACGTGTTTTCGGATACTCCGGATAGGGCTATGTCTTTGTCGCGT |
| | CGTGTTGAGGCTGTCCTTGTTGACGGGGTTGATGGTGGCCCGGTGGTGTACTGCAAACGGTCTACTGGCCCTGATTTGCTGGT |
| | TGATGGTGCACGTTTTGATGTGTATTCGCTTTTTGAGCTGATATGCAGGCCTGTCGAATCTGAGTAAACGTATTTGTTTTTGTT |
| | TTAATGTAATTGTTTGATATTTAATGGGGGTTGTGATGGCTGCAACACGTAAAGCGTCCAATGTTCGCTCTGCTGTTACTGGC |
| | GACGTTTATATTGGTGACGCGCACGCCGGTGACACTATTGATGGTGTTGGGAAGATTCCTGACGGTCTTACCGCTTTAGGGTA |
| | TCTGTCGGATGATGGGTTTAAGATTAAGCCGGAGCGTAAAACGGATGATTTGAAGGCGTGGCAGAATGCGGATGTTGTTCGC |
| | ACTGTTGCCACCGAGTCGTCTATCGAGAATTCTTTCCAGCTGATCGAGTCTAAGAAGGAGGTTATCGAGCTGTTTTGGCAGTC |
| | GAAGGTTACTGCCGGAGCCGATTCGGGTTCGTTCGATATTTCTCCTGGCGCCACCACCGGGGTTCATGCCCTGTTGATGGATA |
| | TTGTTGATGGCGATCAGGTTATTCGCTACTATTTCCCTGAGGTTGAGCTCATTGATCGTGACGAGATTAAGGGTAAGAATGGC |
| | GAAGTGTACGGGTATGGTGTGACGTTGAGGGCGTATCCTGCCCAGATTAATCATAAGGGTGATGCGGTGTCGGTTCGGGGGT |
| | GGATGACGGCTTTAAAAGCTGATACTCCTCCGACGCCTCCTCCTTCGCCGAAGCCGGATCCTAATCCGCCGTCTGAGAACTGA |
| | TACACGATTTTAGGGATTGTTGATAGATGAGTGACACTGGTTACACGTTGAAGATTGGTGACCGTAGCTGGGTGTTGGCGGAT |
| | GCGGAGGAGACGGCTCAGGCTGTTCCTGCCCGCGTGTTCCGTCGTGCCGCCAGGATTGCCCAGTCGGGGGAGTCTGCGGATT |
| | TCGCCCAGGTTGAGGTGATGTTTTCTATGTTGGAGGCTGCCGCCCCGGCTGACGCGGTGGAGGCCCTGGAGGGGCTTCCTATG |
| | GTTCGTGTTGCCGAGATTTTCCGTCAATGGATGGAATACAAGCCTGATGGTAAGGGTGCCTCGCTGGGGGAATAGTTTGGCTC |
| | CACGGCCTGATTGATGATTATCGTGGGGCCATCGAATACGATTTTCGCACTAAATTTGGTGTTTCTGTTTATAGTGTTGGTGGC |
| | CCGCAGATGTGTTGGGGTGAGGCTGTCCGGCTGGCTGGCGTGTTGTGTACCGATACGTCGAGCCAGTTGGCGGCCCACCTGA |
| | ATGGTTGGCAGCGCCCGTTTGAGTGGTGCGAGTGGGCTGTGCTGGACATGCTGGATCATTACAGGTCTGCTAATAGTGAGGG |
| | GCAGCCGGAGCCTGTGGCGAGGCCTACGGATGAACGTAGGGCCCGGTTTACGTCTGGGCAGGTGGACGATATTTTGGCGCGT |
| | GTTCGTGCCGGTGGCGGGGTGTCTCGCGAGATTAATATTATGGGGTGAATAGTGTATGTCTGGTGAGATTGCTTCCGCATATG |
| | TGTCGTTGTATACGAAGATGCCGGGTTTGAAGTCTGATGTTGGTAAACAGTTGTCGGGTGTGATGCCTGCTGAGGGTCAGCGT |
| | TCGGGTAGCCTGTTTGCTAAGGGTATGAAGTTGGCTTTGGGTGGCGCCGCAATGGTGGGTGCCATCAACGTGGCTAAGAAGG |
| | GTCTCAAGTCTATCTATGATGTGACTATTGGTGGCGGTATTGCTAGGGCGATGGCTATTGATGAGGCTCAGGCTAAACTGACT |
| | GGTTTGGGTCACACGTCGTCTGCCACGTCGTCGATTATGAATTCGGCTATTGAGGCTGTGACGGGTACGTCGTATGCGTTGGG |
| | TGATGCGGCTTCTACTGCGGCGGCGTTGTCTGCTTCGGGTGTGAAGTCTGGCGGGCAGATGACGGATGTGTTGAAGACTGTCG |
| | CGGATGTGTCTTATATTTCGGGTAAGTCGTTTCAGGATACGGGCGCTATTTTTACGTCTGTTATGGCTCGCGGTAAGTTGCAG |
| | GGCGATGACATGTTGCAGCTTACTATGGCGGGTGTTCCTGTGCTGTCTTTGCTTGCCAGGCAGACTGGTAAAACGTCTGCTGA |
| | GGTGTCGCAGATGGTGTCGAAGGGGCAGATTGATTTTGCCACGTTTGCGGCTGCGATGAAGCTTGGCATGGGTGGTGCTGCG |
| | CAGGCGTCTGGTCAGACGTTTGAGGGCGCTATGAAGAATGTTAAGGGCGCCCTGGGTTATCTTGGTGCCACGGCTATGGCGC |
| | CGTTTCTTAACGGGTTGCGGCAGATTTTTGTTGCGTTGAATCCGGTTATTAAGTCTGTTACGGATTCTGTGAAGCCGATGTTTG |
| | CGTCGGTGGATCAGGGGATTCAGCGGGTGATGCCGTCTATTTTGGCGTGGATTAACCGTATGCCGGGCATGATCACGAGAAT |
| | GAATGCACAGATGCGCGCCAAGGTGGAGCAGCTGAAGGGCATTTTTGCGAGAATGCATTTGCCTGTCCCTAAAGTGAATTTG |
| | GGTGCCATGTTTGCGGGTGGCACGGCTGTGTTTGGTATTGTTGCTGCGGGGGTTGGGAAGCTTGTTGCAGGGTTTGCCCCGCT |
| | GGCGGTGTCGTTGAAGAATCTGTTGCCGTCGTTTGGTGCGTTGAAGGGTGCCGCCGGGGGGCTTGGCGGCGTGTTTCGTGCCC |
| | TGGGGGGCCCTGTCGGTATTGTGATCGGCTCGTTTGCGGCCATGTTTGCCACTTACGCCCAGTTTCGCGCCCGGGTAATGCTG |
| | CTTGTGGCTGTGGGTGGCCAGGCT |
| | SEQ ID NO.: 18; >6957 len:8925 cycle: O |
| | GCACAGCTCGCCGACCTTCTGAAACAGTACAAGAAGATCATCACCAGTAACAGGGACGTGTCTGAGGCTTTCCGCACCAAAC |
| | GCAACAGCCTCACCCGATAAAAACCGACACAAACCAACAAAACCAAACATAAACAGTAAAAGGAAAACAGTTATGGGACTC |
| | GCAAACTACCGCAACAACAGCAACAGCACCTTCTTCAACCCCTCCCGAAACCAGGACGCCACCGCCATCGCCTTCAAAATCC |
| | GCGACGTGCAGCACAACACTGAAGGCTACGGCGGACAGGTCGCAGACCGCATCTACGCTGATGTCACAATCTTCCACACCCT |
| | AGACGATCTCAACAACAGCACCCCAGAAACCATCCCCAACGCCATTATTGAGAAAGCGCGCGGCAACAACGACCGGCCACA |
| | CTCCATGATCCGCGAACTCGAAACCTACCTGGGAGAAGAGCAGGCCTTCAAACTAGCCACTGTGCGCACCAAAAACGGGTTC |
| | AACGCCGTTATTCTCAAACCATTGGACGACGCCATCTACGATAAGGTTGCCGAATACGTAGACAAGCGCGATAATGGCCAGA |
| | TAGACGACACCACAGCCTCTACTGACATCGATATCGACTCCATCTGACCACCACTCTCCGCCCAAACCGTAACAGATAGATA |
| | AGGTTCCGATGCTCTCTCTCCAACGATCCTTCGAGAGAGCCTCCCAAACCGCAGCCGAGCTGCCCCGCATACCACAACTAGA |
| | ACCCCTCTACCGCAACCTGGACATGCACATCCACAAAGGGGATCTCATCATGATTGCGGGGCGCTCCGGCAGCCAAAAATCC |
| | GGGCTAGCCATGTTCATCACAGCGATGCTCAACCAGCCCGCCCTCTACATATCAGGGGACATGACACCCTGGGAGGCCTCCA |
| | CACGAATCATCTCACTCAACACCCAACACACCACCACACAGATACAACAAAACATCGACGACTACGGGCCAGAATACTATCG |
| | AGACAGCATCCACCACGGCCAACGCATCACATTCTCATTCCAGTCACCCATCACATGGACAGACATCACCATGGGATTGCAA |
| | GCCTACATGGAAATGTGGAACACCTTCCCACCACTCATTGTTATCGACAACCTGATGGACATTCAAGACTGCGAATCCGACTA |
| | CCAGGCACAGCAAGAAGCCATGCAATGGATCACAGCAGTGGGCAGGGATACTGGCTCCACCATTATTGTCACACACCATGCA |
| | ACCGACAAAACCGGCTCCGACATCGAACACCCGCCAGCACGGCGAGAAATCAAAAACGGACTCTCAGAAAAACCACAACTC |
| | ATCCTCGGAGTCTCATTGTATGGTGGCGAGGATAATGGCAACGGCCTATCGATACCGGCAGAGGCACGCATCGCAGTGCTAA |
| | AACAGCGCACCGGCAAATCCAGCCCCGACGGCACCCGATACGAGCGGCTACGAGCCTACCCCGAATACACATTCTTCGGGCC |
| | ACTCGCCGAAAAACATCCCTGGAACATGACCACAACACACAAAGGACTATGATGGCGACACAGCAGGCACGCAACCGGCGG |
| | GCCGGAGCCGAATGGGAAACACGATTATTGCACCAGCTACGCGACACCGGACACAATATCGAACGCCTCCACCCCAACGGT |
| | AAAGAGGATGAGGGCGACCTCATCCTCACAACCGGCAACAAAACCTACATTATCGAGGCGAAAGCCGGGCAACCACACCTA |
| | GCCCAATTCGTCAAAGAAGCAACCCTTGAGGCACGCAACTACGAAACACACCGAAACAAACAAAACAATTCCACCATCGGA |
| | CTCGTAGTAATGAAACAGCGCAACAAGCCATGGAGGGAAGCCTATGTGGTATCAACCCTCAACGAGCTCCTCTCACACCTCT |
| | GACACCTGCCGCCTCCTCGACACCTACCGGATACGGTACAACCCGTCCAGGAACGAGCAACACATCCTCTGCCCGCTCCACG |
| | ACGATCACCAGCCCTCCATGAGCATCAACCTCGACAAGGGCGTCTGGTACTGCCACACATGCGGTGTCGGAGGCGGACTTGC |
| | CAAGCTACAACAACGATTAGAAGAAGAAAACCCGAATGTACGACAGTATACGCCCATACAACATTGCGGAACGCCGCCGAA |
| | TCCAGAAAGCCTCGGCCCTCTACGAAACCCACCTCGAAGACATACTCGACCTGCTCTCAGCAAGAGGCATCAGCGAAGAAAC |
| | AGCCCGCTACCACCACCTTGGATACATCGACAATGACCCCATACCAGGCCACGAAAACTACAACCAGTGCATCACCATCCCA |
| | TACATGTACCCCTTTTTGGGCGGCCCAGCCGAAATACGAAAAATGCGTTTCCGCTGCTCACTCCCGCACGACTGCAAAACCCA |
| | CAACCACCCCAAATATTTGACCCCAGCCGGGGACACAGGCTCCATCTACAACATGGCCGCCATGGCCAACCCGGCATCCGAA |
| | ATGCACATTTGCGAAGGCGAATTCGACTCCATGATCCTCGAACAATGCGGATGGTCGGCCGTTGCCCTACCCGGCGCAACCT |
| | CGTGGCAAACTTTTTGGACCAAATTTTTTGAAGGCTATGACCACATCTACATATGGTCCGACCCAGACCCGGCAGGCGACCA |
| | GATGGCCCAAACCCTCCAGTCAGCACTCCCCCAAGCCACCCATGTGCCCCTCACCCTGGGGGATGTCACAGACACCTACCTTC |
| | AGGCCGGAAAAACAGGGTTGACACAAGCACTAGACACTGTGCTACAGTAAAACACGTCAACAACACGAAACCAGAAAGGTA |
| | AACTAAAACATCATGGATCCGCTCGACACGTGCCCCATCCCCAACCGGCGCAAGACCAGCCAAACAGTCAGGAGGCGTATCC |
| | GCCTCGCCATCTGTGCAGAAAAATGGGCTGATGGTGAAGACCCCACCTACATCATGCACACCTGGGGCACCACCTATGATGG |
| | GATGCGATCCATGATCCGCGCCAACCCCGATATTAAACCACCCGACGACATGGCAGAACGTTTGCACAAAGTATGCCGGGAA |
| | ACCTACCCCAAAAACCAGCCCAACAGGCACCGAAGCGGATGGGACGCTTACGAGAAAAACTACTACACCCACGAAATCCTC |
| | TTCCTGAACCAATTTAACATCCCAGCCCTCGAAATCCTCGACCGGCTAGACGTGTCATGGACAATGTGGAAACAAATCATCA |
| | CCGAAAACCATCTCACCCGGCTACAAGACGAAACATACAATGCGTGCCACTGGCACTATCTGAAACAGCAACACCCAGACTG |
| | GACCGACCAACAAATCACACAAGCCCAACACGCCGGGAACAACACCTTCAACCAGTTCATGCAAGACGAACAGCCGGCACT |
| | ATCGTGAGCATCTCGTTCAAACCCACAAAACGCAACCTTCAAACCATCCGAGACATCCTCACCCAGGCCACCAACCTCAACC |
| | CTGACGATCTGCCTGACGACACCCTCAACAACATCATCCAATACTGCTGGAACACCTTCACCGACAACAACCGGTACGCCGT |
| | CGCGGCACAATACTGGCGAGGCCAAAACCCACCCGACAGTGAACATCAGCGCATACTAGTCGGCTACTACAAAACCGCTAA |
| | ACAAGCCATGAACGCGGCCAAACAATTCCACTGGAACACCCGGCTACAACAACAATGGAAAACATGGATACTCCCAGTCCA |
| | CAACGGCACCGTATCAGAGCATTTCACCAACCAGAAAACACTCTTCGACACACCAACCAGCAACCAGGGTGACGCCGCACTG |
| | CCGGAGCATCTACAAAACGTCATGTGCGGCAAAACACTCAACCACACAGACGGAACCGTATCGTGGTGCACACGCAAACCA |
| | GGTCACGACGGCGACTGCCGCACAGGATGGCAACCCACCACACAACCGATAGGACACCATGGCAACCAAAACTGAAACACT |
| | CATCCAACGCTACGGTAACAAGGCTGCCGACGTGCTCGCCGACAAAACCATCCCCGCCTCATGGCTAGCAAAACAGCTCACC |
| | CAGGCAGGATACCCCATCTCCCCCACTGTTATTAAAGACTATCGCCACAAACAAGCCAACACCACCCCGCAAGAGGAGAATA |
| | TCCGATGATAGACAATATTGACCGGCTACTCACCCAGCTAGCCAACCACGACAACGCCATCGACACCATCGACGACAATCTC |
| | GCCAATGGTACTGTACGCCGCACACGCATCTCCGAATGGACACTCCCCAACGGAGAAACAGGCCGATCCGTACAAAAAATCA |
| | TCGACCACCAACCCGCAACAAACCCCTACCCTGTAGACGAACTCGTCGATAAACTAGCCGAATGGCCACCCCCCAAACAGTC |
| | CGACACCCGTAGCAGCGATGCGGCCTTCGTCATCGGGGCAGGCGACTTCCAAATCGGCAAAGGCATCCCCGGAGGGGAAAC |
| | AGCACACTTCGCCGACGACTACCTACACTCCCTCGCCTCAGCTAAACACTACTGGCAGCAAGCAGGCCAGCCGCAACGAGTC |
| | CACATAGCCTTCCTCGGCGACATGATCGAAGGATACGTGTCACAAGGAGGCAACAACGCCTGGCGCACACAAACACCCTTGA |
| | CGGAACAAATCAGGCTCACCCGTATGGCCATGATGCAACTCATCCACATGTTCGACCACTGCGCCAACGTCACCATCACATC |
| | CATCCCCGGCAACCACGGTGAAGCCGTGCGCTTCGGTAAAGGCGTCACCACCTACGACGACTCCTTCGATGTGGACTGCTGC |
| | CGGGCCATCGCAGAAGCCCACCAGCTCAACAACCAATACCCCAACCTTCACTTCCACTTCCCCAGCCGAGACGAAATGACCA |
| | CCACCGTTGATGTGGCCGGTACACAAATCCTGCACGCCCACGGACACCAATGGCGCAACAACCAACACTACGAATGGTGGCG |
| | CGGCCAAGAATTCCACAACGGCACCGTATCCCACATTCTCATGGCCGGACACAGGCACCACCTCGAAATATCCGAGCAAGGA |
| | CAACGCACATTCATACAATGCCCATCCATGGAAGGAGAATCCGTCTGGTACCGGCACAAGACAGGCACCACCGGCAACCCCG |
| | GACTCGTGTGCTACACAATCAACCATAAAACACCAAACAACTACCAGATAGCCAGATAAAAACCATGAGTAGACGACCAAC |
| | CAAAGTCCAACAAGCCACCACCGCCAACTGGGGGTGGGCAACCCACCACCACCAACACCAGCTACACAAAGCTTGCACCAA |
| | CATTGCCCGCCACTACCCGGCCGTCAACCCCGACGACCTGTACCAAGACTCTCTACTATATATTGCGGTACGCAACCAATACC |
| | ACCAACTCACAGGCAACCAGTGGACCCAAATGTGCTACCGTGTCGCCCAACGACTAGCAAACAAAACAGTCCACCACCTAGA |
| | CCTACCCAAACCCGTCCAAGAAATCACAGCCATAGCCGACAACCAAACCAGCAACTAAGGAGAACCACAATGGTCACAACC |
| | ATCCTCGACGACGGCACCCAAACCACCAGGCTACAAACCGTAGGCTCCACCACCACAGCCATCATCACCAACACCGAAACAC |
| | CCGAAACCATCACCACCAAATACACCATCAGCAAAGACGGCACAGCCACCTACAGTATCAGCGGCAACACCTATTTGGGAG |
| | ACCACCAACACATCATCAAACTCATGTACGACTACTGCCACTGCGTCGGACGCTTCGACACCACCAACACCAACAGCCCAGA |
| | CAACCTCGACAACCTGTTCAGGGGGTGACACATGAACCACACCTACACCACCGCAGATATTATTCAAGCAGCCCAATGGATC |
| | TGGAACGGCGGCCCATGGAAACCCTCAGTAGAACCCGGCATGCCACCCCCACCAACCGCCCCCCAACACCACGGCAACAAC |
| | ATCGTCTCCATGATCGATTTGCAGCTAGCCATCGACGACTACACCCTCACCTGCCAGCCATCCAAACAGCGAAAACATTTGGC |
| | ACGGTTGGCAGCATTCCGGGAAGTCTACGGGTATGACCAAACATATGCGTCGGCCGCCCAACGATTGGGTGTGACAAGACAA |
| | ACCGTGAAACAGTGGGCAGACCAAACACTCATCACCCTCACAGGATACGCAAACAGTAAATACTATCCAGACGGCAACGAC |
| | GACAGCACAGGGATGGGATAAAACCATGAACAACACAACCAATATCCCCTACACTGCCCTCAAAACAGCGGTACACCGAAT |
| | CATCCAACAACAGCCCACCAACATGCAACAACTAGAAAACATTGTTGACGGTGTCGAAAACCAGTACCGTGTACCCATCTCC |
| | CTCGATGATGTGAACCTCACCGTCAAAGAAGTCAGCCTCGACGATCTCGACATCGACCAGGACACGCTAGACGAGTGCAGCG |
| | AAATCCTGTGGGACTGCGACAGTGCAGGACACCCCACAAACAACAGCAACACCCATGGCATGCCAGACGACACACAGGCAA |
| | GCCATGAAGCCATAGACTGGCTCGCCAGAATCGCATACCAGGCAAAACTACTGCAAGCGGAGGCCGACGATATCATGCAGT |
| | CCATCCTCTACCACCGCGACAACCACAAAGGCGTTATCGGCCAGGATGTTCTGCACCAGGCCAACGAAACTATCTCTACCTG |
| | CCTCCACCTGTATCAGATGCTCGAAGACACCATCGACAGCAACGAATCATAGCCACACCGCATAAACAAAAAGAGTGCCCCA |
| | GCGGCAACCACCACACAATCGTGGCAGCACCGCTGGGGCACACACATATTCAATTATGCAACAGTAGACTCTACCGTGCCAA |
| | CCTCCGACTCGGCGGCACGCCTCGGCTCATAGCCACCAAAATCAGCATCATCCTTCGGCTCGATCATGCCCGGATCCGACACA |
| | TCCACCGAGTGCGGCTCAACCAAGCCCCCATCGTCGGGTGGAACAAGCCCAGCATCCACAGGCGTAGTTTTCGGCTTACCGG |
| | CCACAAACGACGGGCTACCAAACGAGGTAGCAACCGACAGCACCGCAGCCACCGTTGCTGTAATCAGGGCAGACTCCCACG |
| | GCAAACCGCGAAACGACTCCGCAGTATACGTGACACCCGCCGTCACACCCAACACAGCAACAAACGTTTGAATAAAAGTTTT |
| | CAGTGCACGCTCAAACAAGCCCAACCAAAACTGTTTACCCACAACAAACCACCATCACTTTTTCAAACCGTTGACAGTCGAC |
| | TCAAGCCTGTCAATACGGCTACGACACTCCAGCACGTAATACCAGACACTCCACAAAGCGTCTTTTGTGCGCCACAGCTTCCC |
| | CGTCACCGGATTCTTCACCCACGACAAAGCCTCCACACGGCGCGCCAAGTCACCATTCTGAACCTGAACCACACCCACATCG |
| | TGATGCAGCTTATTCACCGACTGGGCCACCTGCCCAGACAACTGTTTAATCTGATCATGTAACGCTTTCACATCAGCCACCGT |
| | TAACTCCTCACTCTCTCCACTGCCGCCATTGACGACGGCCATAAACCTGTCCCACGGAAACCACGGCCCCGGATCGTCATGAT |
| | CCGACTGATGCCACGCATCCGTCACATCCACATGCCCGCAAACCCCACGCCTGCCAGCCTTCAAATCGGCAGCCGACAGTTTC |
| | CTTTTCGGAACATTATATTTGTCACACAAACGTCTACACAGCATGGCAGCCTTCTCCACCGCAGGCCACACGCGAGGATCAAG |
| | CCACTGCTCCCGAGTGTAGGCGTGCCCCGGCACCCTAAACGAGGCGTGCGAACCCCCATCCGCGCAAATCTCGATACCTAAA |
| | CTATGCGGATTCGGCGGGGCATGCCACCCAATCGTAGACTCCGACAAGCACTGCACCGTCTCCCCAATATCGCAAACATAAT |
| | GCGCCGAACCACCAGACGATGGGGACGCGAAATAGTTTGCCGTGGACACCGCCCGCCCTTTACGGGAGGCAGACGGAAACC |
| | CCACATCCGGGCACGTCGCATGAATCACAACCCTGTTCACCGGACTATTCGAGCCGGCCGAGTGATGCGCTGCAGGAATAAA |
| | CCTCACCACACACCACCACCAAACACTACCATCACAGCCACTCCTTTCTATTTGTGGGATGATATAGTCACTATAGGCGACGG |
| | TTTCACACCCTGGCAGGCCACCGAGCCCGATATTGTAGAAGCCACACCGTCACTATATTTCACAACAAGGCGGCCCCCGGAA |
| | CAGTACACAGACCCCACCGAGCGCCCATCTTTACCGTCTTTACCGTCGAATCCGTTCACACCAGCGGGGCCACGCTCACCCCG |
| | TTCACCCTGCGCGCCTTGCGGGCCGGCAGGACCCGAAGGCCCCACATCGCCGCGCCCACCGACCGAACCATCCCGACCATCA |
| | GCGCCGTCAACGCCGTTCACACCATCAGCACCTGCACGACCCGGAACACCATCACGGCCATCCGACCCATTCGCGCCAGGCA |
| | ACCCGTCAGGACCTTTCACACCATTCAAACCGGGAGAACCCTGCGAACCAACAGGGCCAACCAGCCCCGCCGAACCATTAAC |
| | ACCATCCCGGCCGCGTACACCTGCAGGGCCTTGTGGGCCGCGTACACCTGCAGGGCCAGGCACACCCTGCACGCTACGCTCA |
| | ACACGCACAGCATCCACACACATGCCAGACTGGTGAAGACGCACCGACTCCTGCCCACCCGAAGCACACGCCTGCCGCACAC |
| | GGTTTGCCAACCCTTTAGCCGCTGTACCATTAGACTGGGCCCTCGCCTGCTCCGAATCCCGCTCAGAGGATACAGCCCCGAAA |
| | CGCAAAGCACCCCCGGCAACCACCGCCAACAACACAAGCGACAAAAACAGCAACACCAGTGAAAACTTCTCAAAATTGCGG |
| | CGCTGCCGCTTCTCCTCCTCCAACTCCCTCAACCCTACTCACCTCCACCATCAACAGTATCTTTCAAAAACTCGGGCACATCA |
| | SEQ ID NO.: 19; >7340 len: 11354 cycle: O |
| | CCTGCGANCACATGGATTTCGNGGGCCTNGCCGAAGCCTACCGGCAAANCGTGGANGCCGAANTCCACAAGCTNCGCCCCA |
| | ACACGTTCATCGCCACCGACGGCACCGTCTACAGCCATGACGAGTGGAAGCCACTCACCGGTGGGGAAGCCACGCAACTCTT |
| | CTGGAACGTGATCCGCATCAATGTTGGCCATCTACTCACCCTGTTCGCCCTATAAAACCCCCACCACACACAATGAGTCTAGA |
| | ATCGCCTAGAAATACCTGACCTATATAATCCTACCTAAGAGCGCTAAAAGTCTCTTGTACGCTAAATGCAGTAGCTTAAAACA |
| | CGAAAGAAGGACCAATCATGCAATGGACATGCCACAAGTGTGACACCACAATCATCGGATACCGACCAGAACACTGCACAA |
| | TCTGCCATGAAACATTTAATGGCACTCATGCCGGGGACATGCACCGGACAGGCGATCACGGGGTCAAGGAAGGCCCCAGTCG |
| | CCGCAGGTGCCTCACTGTGGACGAGATGGAGGCTAAGGGATGGCTCGTAACCGCCATGGATACTGGACGCAAGGTAGTGAC |
| | ATGTCACGCTTAAGTGACAAAACCCCAAGCCACACAGGAAACGCTCACACAAACTGAGCGACACCTTGACAGTGGTAGTAC |
| | AGTCTGGAAATATTAACCATGTCAGCAACGAACAACACCCCAGAAAGGGGAACACAGCCATGAACAAGAAAAAAGGCTACA |
| | CCATCGCAGGCGCCACTATCGCCATTATTGCCGCATGCTCTTTCCTGCCAGCACCCGACGACAACCCGCCACTCGCCTCACAG |
| | TCAGCCGCACAGGCCACCACAGCCAACACCACATGGACACCTGAAACCGCCCAGCAGCGCAAAGCCGAGAAAGCAGCCAAG |
| | CAGGCCGCAGCAGTCCGCTCCCTACAAGCAGAACAAGCCAAAACCCACAAGCAAGCCCAAACAAGGGGTGAAGAAACCGCC |
| | ACCGGACTCACCATGCTCACCGCTGCACACGCCTGCAACCGCAAAGCCGAACAACAGGCCGCAGCACACGGTGTCAACTGG |
| | AACGGCAACCCCGACATCGACCTCCAACTCCACAAAACCATCGGTAAAGACACCTTCTCCATCGTCTACGGTGCAACCATGA |
| | AACAGCCCGGCGCATCCAAACTCCCAGTCACCGTACACTGCCTCGCCACCGGAACAGAAGACCACCCGCACGTCACCGACCT |
| | CAACATCAACCCGCAACAGTAACCCGCCAAGGAGCATCCCCGCTATGCCTCTCCTCTCCCACTATGCTGTCACCACCGGACTC |
| | GCCGACACGGCACACATTATTCACCACACCGGCGGCACACTACGCACAGCCACCGATATTGCATCCCGCATCAACGCCCTCA |
| | ACCCAGACATTGATCTCGACCACGAAATCCACCAACTGTTATCTATCGAAACCGACCTGTACAACATTTATAAAACCATCAAC |
| | ACCATTCTTCAGGAGCAAGCATGAACACCCCCAACAACAACATTGAGCTACACAGCTACGAAACGTTCTTCACCAGCCTAGC |
| | CTGGATCCAAGGCGGCATCATCACATGGATGTACGCCACCGGCACCCCACACAAGGCAGCCCTCGCCATCATCGCCGCATGC |
| | GCCCTCACCACCCTCCTAGGCGCATCAACACTCACCAACAATCCCCGAGACACTAAATGATCACAACACCCATCCTCATCGCT |
| | GAAACCCTCGCCATCATTATTCTCGCCGTAGCACTAGCCCACAACAACAACCAGTAACCCGCACTTAAGGAGCACACACCCC |
| | ATGGATAAGCCAACCCGCATATACACCAACCCCGAAACCGGCGCCAAGAAAGAACTCAAACTCTGCAGGCTATCCCTCATCG |
| | AACCCGCCGCCTTGCACTCGCTCGGCGAAGTAGCCGGATATGGTGCCACCAAATACGGCGACAACAACTGGACCGGCGGATA |
| | CCCCTGGTGTCACAGCGTCGACGCCCTCTACAGGCACCTGCTATCATGGCAGCAAGGAAACAACCTCGATTATGAATCCGGG |
| | CTACCCCACCTGGCACACGCAGCCTGGCACTGCCTCGCACTCCTCGCATACCAGCAACACAATGCCGGGGTAGACACCCGCA |
| | ACCCATGGAACACCCACAAAGGCGACCAGTAATGCCTCTAGCACAATACCCGAAAACCATCCACCATCCAGGCCACATCTCC |
| | TACTCATCACTGTCACAGTGGGCCGAATGCGGAGAAAAATGGCGCCTATCCCACGGCTACCACACCCAACACCACACCTGGT |
| | ACGCCACCATCGCCGGCAGCGCCATACACCACATCACCGAACAATACGACCTACACCTGTACAACCCCACCGAATACTCTGC |
| | ACTACCAGACAAACTCTCATCCTTCAAAAACATTTTCGACACCCAAGTCGCCCTCGCCGAATCCGAGGGCACAGAAATCGAA |
| | CCCTCCGGCAGAATATGCAAAAACATGTGTGAGTCGGGCGGGCCACACAAGAAAGACTACGGCTGGTGGATGGTTTACGGC |
| | CCCACCTTTGTGGACCGCTGGAAAACATGGAGGCGCAACCACCCAGAATACATCACCGCAATCCTGGACGGTAAACCAGGCA |
| | TCGAATACCCGGTAGAAACCATCCTGAAAGATGACACAAAAATAGTCGGATATATTGACAGAATATTCACCAACACCGACAC |
| | CGGCGAAACCTTCATCCTCGACCTCAAAACCGGACGCCTACCCGCCGACAGTATGCAGCTGCACACATACCGATACATGCTC |
| | AACCAACACGGCAACGATGTGACAAAAGGCATGTTTTGGACGCCAGCCACCAGCCGCAACGACGACAAGTCCCCAACACAA |
| | GGCACATCCACCGAACTCTACGACCTTGACAACAACACCTACCGGCATGTATCATCCATGTACAGTCAAGCAATGAAAGGAA |
| | TCAGCGAAGGCATCTTCGTCCCACACGTCACAGCACTCTGCAAAGGCTGCCCCGTACAAGACGCCTGCTGGGCCGTCAACGG |
| | CAAAAACTCGTACAGATACCCGATAGAATCAACCGTACAGCCACCAGAAACAGACAATAAAGAAAAGAACACCAAATGACC |
| | GATAAAGACAAGATCGACAATGATCGACTCACGATCACACTCAAATACGGTGGCGACTATGCTGCCCCATGGGCCGTCATCC |
| | GCGGAGACACCGCAGAAGACATCAAACAATCCATTATTGATCTGCTAGGAGGATTGAAAGACGAAACCGTATCCAAAGACT |
| | GGGACCTCGCAACCCTCATCGCTAGCGCAGCAATCCCGCTTCAAGACCGATACGATAAAGCCGCCAAAGACTACGTCAACAA |
| | GATCGCCAACAAGGAAAACGACATCATCATCGACCGGATCAACAAAGCCACCAGCAAGGCACAGCTCGCCGACCTTCTGAA |
| | ACAGTACAAGAAGATCATCACCAGTAACAGGGACGTGTCTGAGGCTTTCCGCAGCAAACGCAACAGCCTCACCCGATAAAA |
| | ACCGACACAAACCAACAAAACCAAACATAAACAGTAAAAGGAAAACAGTTATGGGACTCGCAAACTACCGCAACAACAGCA |
| | ACAGCACCTTCTTCAACCCCTCCCGAAACCAGGACGCCACCGCCATCGCCTTCAAAGTCCACGATGTAGAACACAACACTGA |
| | AGGCTACGGTGGACAAACAGCAGACCGCATCTACGCTGATGTCACAATCTTCCACACCCTAGGCGATCTCAACAACGGCACC |
| | CCAGAAACCATCCCCAACGCTATTATTGAGAAAGCGCGCGGCAACAACGACCGCCCACACTCCATGATCCGCGATCTAGAAG |
| | CCTATCTTGGCGAGGAGCAGGCCTTCAAACTCGCCACCGTACGCACCAAAAACGGCTTCAACGCGGTAATCCTCAAACCCCT |
| | TGATGACGCCATCTACGATAAGGTTGCCGCATACGTAGACAAGCGTGATAACGGCCAGCTAGACGACACCACAGCCTCAACT |
| | GATGCTGATATCGACATCGACTCCATCTGACCACCAAAACACAACCAACCAATAGATAGATAAGGTCCCGATGCTCTCTCTC |
| | CAACGATCCTTCGAGAGAGCCTCCCAAACCGCAGCCGAGCTGCCCCGCATACCCCAGCTAGAACCCCTCTACCGCAACCAGG |
| | ACATGCACATCCACAAAGGGGATCTCACCATGATTGCGGGGCGCTCCGGCAGCCAAAAATCCGGGCTAGCCATGTTCATCAC |
| | AGCGATGCTCAACCAGCCCGCCCTCTACATATCAGGGGACATGACACCCTGGGAGGCCTCCACACGAATCATCTCACTCAAC |
| | ACCCAACACACCACCACACAGATACAACAAAACATCGACGACTACGGGCCAGAATACTATCGAGACAGCATCCACCACGGC |
| | CAACACATCACATTCTCATTCCAGTCACCCATCACATGGACCGACATCACCACGGAATTGCAAGCCTACATGGAAATGTGGA |
| | ACACCTTCCCACCCATCATTGTTATCGACAACCTGATGGACATCCAAGACTGCGAGAGTGACTATCAGGCACAGCAAGAAGC |
| | CATGCAATGGATCACAGCATTAGGTAGGGATACTGGCTCCACCATCATCGTCACCCACCACGCCACCGACAAAACCGGCTCC |
| | GACATCGAACACCCGCCCGCCCGGCGAGAAATCAAAAACGGCCTCTCCGAAAAACCACAACTCATCCTCGGGGTCTCATTGT |
| | ATGGTGGCGAGGATAACGGCAACGGCCTCACGATACCGGCAGAGGCTCGCATCGCAGTGCTAAAACAGCGCACCGGCAAAT |
| | CCAGTCCTGACGGCACCCGATATGAGCGGCTCAGAGCCTACCCCGAATACACATTCTTCGGCCCCCTGGTAGAAAAACAGCC |
| | ATGGAACATGACCACAACACACAAAGGACTCTAAAAAACTATGGCCACACAGCAGGCACGCAACCGGCGGGCCGGAGCCGA |
| | ATGGGAAACACGACTCCTCCACCAGCTACGCGACACCGGCCACGATATAGAACGCCTCCACCCCAACGGCCGCGAAGACGA |
| | AGGCGACCTCATCCTCACAACCGGCCACAAAACCTATGTGATCGAAGCCAAAGCGGGCCAGCCACACCTAGCCCAATTCGTG |
| | AAACAAGCCAGCCGGGAGGCACGCAACTACGAAACCCACCGAAACCGCGAAAACCAGTCCACCATCGGACTCGTCATCATG |
| | AAACAGCGCAACAAACCCTGGAGCGAAGCCTATGTGGTATCAACCCTCAACGAGCTCCTCCCACACCTCTGACACCTGCCGC |
| | CTCCTCGACCACTACCAGATACGCTACAACCCGTCCAAAAACGAGCAACACATCCTCTGCCCGCTCCACGACGACCACCAGC |
| | CCTCCATGAGCATCAACCTCGACAAGGGCGTCTGGTACTGCCACACATGCGGTATCGGAGGCGGACTCCACAAGCTACAGCA |
| | ACGACTAGAGAAAGAAAACCCGAATGTACGACAGCATACGCCCCTACAACATTGCGGAACGCCACCGAATCCAGAAAGCCT |
| | CGGCCCTCTACGAAACCCACCTCGAAAACATACTCGACCTGCTCTCAGCAAGAGGCATCAGCGAAGAAACAGCCCGCTACCA |
| | CCACCTTGGATACATCGACAATGACCCCATACCCGGCCACGAAAACTACAACCAGTGCATCACCATCCCATACATGTACCCC |
| | GTTTGGGGCGGCCCAGCCGAAATACGAAAAATGCGTTTCCGCTGCTCACTCCCGCACGACTGCAAAACCCACAACCACCCCA |
| | AATATTTGACACCGGCAGGGGACACAGGCTCCATCTACAACATGGCCGCCATGGCCAACCCGGCATCCGAAATGCACATTTG |
| | CGAAGGCGAATTCGACTCCATGATCCTCGAACAATGCGGATGGTCGGCCGTTGCCCTACCCGGCGCAACCTCGTGGCAAACT |
| | TTTTGGACCAAATTTTTTGAAGGCTATGACCACATCTACATCTGGTCCGACCCAGACAAGGCGGGCCACCAGATGGCGCAAA |
| | CCCTAACCCAAGCATTACCGCAAGCCACCCATGTGCCCCTCACCCTGGGGGATGTCACAGACACCTACCTGCAGGCCGGAAA |
| | AACAGGGTTGACACAAGCACTAGACACTGTGCTACAGTAAAACACGTCAACAACACGAAACCAGAAAGGTACACTAAAACA |
| | TCATGGATCCCCTCGACACGTGCCCAATCCCCAACCGGCGCAACACCAGCCAAGCTGCCAGGAGGCGCATCCGCCTCGCCAT |
| | CTGAGCAGAAAAATGGGCTGATGGCGAAGACCCCACCTACATCATGCGCACCTGGGGCACCACCTATGATGGGATGCGATCC |
| | ATGATCCGCGCCAACCCCGACATTAAACTACCCGACGACATGGCCAAACGGTTACACAAAATCTGCCGGGAAGCCTACCCCA |
| | AAAACCAGCCCAACAGGCACCGAAGCGGATGGGACCAATACGAGAAAAACTACTACACCCACGAAATCCTCTTCCTCGACC |
| | AGTTTAACATCCCAGCCCTCGAAATCCTCAACCGGCTCGACGTGTCATGGACCATGTGGAAACAAATCATCGAAGAAAACCA |
| | TCTCACACGGCTACAAAACGAAACCTACGATGCGTGCCACTGGTATTATCTGAAACAGCAACACCCCGACTGGACCGACCAG |
| | CAAATCACACAAGCCCAACACGCCGGGAACAACACCTTCAACCAGTTCATGCAAGACGAACAGCCGGCACTATCGTGAGCA |
| | TCTCGTTCAAACCCACAAAACGCAACCTTCAAACCATCCGAGACATCCTCAACCAGGCCACCAACCTCAACCCAGATGATCT |
| | GCCAGATGACACCCTCAACAACATCATCCAATACTGCTGGAACACCTTCACCGACGACAACCGGTACGCCGTCGCGGCACAA |
| | TACTGGCGAGGCCAAAACCCGCCCGACCAGCAACACCAGCGCATACTCGTCGGCTACTACAAAACCATAAAACAGGCAGAA |
| | AACGCCGCCAAACAATTCCACTGGAACACCCGACTACAACAACAATGGAAAACATGGATACTACCCGTACACAACGGAACC |
| | GTCTCCGAACATTTCACCAAACAAAAAACACTCTTCGACACACAAAACGATAACAACCCGGAGCAGCTACCACAGCATCTCC |
| | AAGAAGTCATGTGCGGCAAAACACTCCACCACACAGACGGCACCACAACATGGTGCACACGCAAACCAGGACACGACGGCG |
| | ACTGCCGCACAGGATGGCAGCCCACCACACAACCCCTAGGACATCATGGCAACCAAAACTGAAACGTTGATCCAACGCTACG |
| | GCCGCAAAGCCGCAAACGTCCTCGCCGACAGGTCTATACCCGCATCCTGGCTATCAAAACAACTCACCCAAGCCGGATACCC |
| | CATCTCCGCCAGGGTCATAAAAGACTACCGCCGAAAACAAGCCAACACCACTCCACAAAAGGAAGAGGATACCCGATGATA |
| | GACAACATAGACCGGCTCCTCACCCAGCTAGCCAACCACGACAACGAAGCAGACACCATCCGCGATGATCTCGCCAACGGA |
| | ACCGTACGCCGCACACGCATCTCCGAATGGACCCTACCCAACGGAGAAACAGGCCGAACCATACAAAAAATCATCGACCAC |
| | CAACCCGCAACCGACCCCTACCCTGTAGACGAACTCGTCGATAAACTAGCCGAATGGACACCCCCCAAACCCGAACAAGACA |
| | CACACACCAACTACAGCACTGCAGCCTTCGTCATCGGGGCAGGAGACTTCCAAATCGGCAAAGGCATCCCCGGCGGAGAAA |
| | CAGCACACTTCGCCGACAACTACCTACACACCCTCATCGTCGCAAAACACTACTGGCAACAAGCAGGCAAACCGCAACGAGT |
| | CCACATCGCATTCCTCGGCGACATGATCGAAGGATACGTGTCACAAGGAGGCAACAACGCCTGGCGCACACAAACACCCCTC |
| | ACCGAACAAATCCGGCTCACCCGCATGGCCATGATGCAACTCGTCCACATGTCCGACCACTGCGCCAACGTCACCATCACAT |
| | CCATCCCAGGCAACCACGGTGAAGCAGTACGATTCGGTAAAGGCGTCACCACCTACGACGACTCCTTCGATGTAGACTGCTG |
| | CCGCGCCATCGCAGAAGCCTACCAGCTCAACAACCAATATCCCAACCTACACTTCCACTTCCCCAGCCGAGACGAAATGACC |
| | ACCACCGTTGATGTGGCCGGCACACAAATCCTGCACGCCCACGGGCACCAATGGAAAACCGGCAAACAGTACGAATGGTGG |
| | CGCGGCCAAGAATTCCACAACGGCACCGTATCCCACATTCTCATGGCCGGACACAGGCACCACCTCGAAATATCCGAGCAAG |
| | GACAACGCACATTCATACAATGCCCATCCATGGAAGGAGAATCCGTCTGGTACCGGCACAAGACAGGCACCACCGGCAACC |
| | CCGGACTCGTGTGCTACACTATCCACCATAAAACACCAAACAACTACCAGATAGCCCGATAAAAGAGATGCCATGAGTAGAC |
| | GACCAACCAAAGTCCAACAAGCCACCACCGCCAACTGGGGGTGGGCAACCTCCCACCACCAACACCAGCTACACAAAGCCT |
| | GCACCAACATTGCGCGCCACTACCCGGCCGTCAACCCCGACGACCTGTACCAAGACTCTCTACTATATATTGCGGTACGCAAC |
| | CAATACCACCAACTCACAGGCAACCAGTGGACCCAAATGTGCTACCGTGTCGCCCAACGACTAGCAAACAAAACAGTCCACC |
| | ACCTAGACCTACCCAAACCCGTCCAAGAAATCACAGCCATAGCCGACAACCAAACCAGCAACTAAGGAGAACCACAATGGT |
| | CACAACCATCCTCGACGACGGCACCCAAACCACCAGGCTACAAACAGTAGGCGCCACCACCACCGCCATCATCACCAACACA |
| | GAAACCCCCGAAACCATCACCGCCAAATACACCATCAGTAAAGACGGCACAGCCACCTACAGCATCAGCGGAAACACCTAT |
| | TTGGGAGACCACCAACACATTATCAAACTCATGTACGACTACTGCCACTGTGTGGGACGATTCGACACCACCCGCACCAGCA |
| | ACCCAGACAACCTCGACAACCTATTCAGGGGATGACACATGAGCCGAACCTACACCACCGCCGACATCATCCAAGCCGCCCA |
| | ATGGATCTGGAACGGAGGCCCATGGAAACCCTCCGTCGAACCAGGAATGCCACCACCACCAACCGCCCCCCAACACCACGG |
| | CAACAACATCGTCTCCATGATCGATTTGCAGCTAGCCATCGACGACTACAACCTCACCTGCCAGCCATCCAAACAGCGAAAA |
| | CATTTGGCACGGTTGGCAGCATTCCGGGAAGTCTACGGGTACGACCAAACATATGCGTCGGCCGCCCAGAGACTCGGGGTGA |
| | CAAGACAGACTGTGAAACAGTGGGCAGACCAAACATTGATCACCCTCACAGGATACGCAAACAGTCGATACTACCAAGACG |
| | ACAGCGCAGGGATGGAATAAAACCATGAACAACACAACCAATATCCTCTACACTGCCCTAAAAACAGCAGTACACCGAATC |
| | ATCCAACAACAACCCACCAACATGCAACAGTTGGAAAACATTGTTGCCGGTATCGAAAACCAGTACCATGTACCCATCTCCC |
| | TCGATGATGTGAGCCTCACCGTCAAAGAAGTCAGCCTCGACGATCTCGACATCGACCAGGACACGCTAGACGAGTGCAGCGA |
| | AATCCTGTGGGACTGCGACAGTGCAGGACACCCCACAAACAACAGCAACACCCATGGCATGCCAGACGACACACAGGCAAG |
| | CCATGAAGCCATAGACTGGCTCGCCAGAATCGCATACCAGGCAAAACTACTGCAAGCGGAGGCCGACGATATCATGCAGTCC |
| | ATCCTCTACCACCGCGACAACCACAAAGGCGTTATCGGCCAGGATGTTCTAGACCAGGCCAACGATACTATCTCAGCCTGCC |
| | TCCACCTGGACCAGCTCATCGAAGACACACTAGACAACAACCTGTAAAACCCCTATAGACACAAAAAGAGTGCCCCAGCAG |
| | CAGCCACCACACAACCGTGGCAGCACCGCTGGGGCACACATCTATATTCACTTATGCAACAGTAGACTCTACCGTGCCAACC |
| | TCAGACTCGGCAGCACGCCGAGGCTCATAGCCACCAAGATCAGCATCATCTGCAGGCTCGATCATGCCAGGATCCGACACAT |
| | CCACCGAATGAGGCTCCACTATTCCGGGATCATCCGGTGGAACCAAACCCGCATCCAGCTGAGGCTTGCCGGGCTTGCCGGC |
| | CACAAACGACGGGTTACCAAACGATGTAGCCACCGACAAAACCGCAGCAACCGTAGCTGTGATCAGGGCCGACTCCCACGG |
| | CAAACCGCGAAACGACTCCGCCGTATAGGTGACACCCGCAGTCACCCCCAACACAGCAACAAACGTTTGCACAAAAGTCTTA |
| | GCCGACCGCTCCAGTAAACCTAACCAAAACTGTTTACCCACAACAAACCACCATCACTTTTTCAAACCGTTGACAGTCGACTC |
| | AAGCCTGTCAATACGGCTACGACACTCCAGCACGTAATACCAGACACTCCACAAAGCATCCTTAGTGCGCCACAGCTCCCCC |
| | GTCACAGGATTCTTCACCCACGACAAAGCCTCGACACGCTTACGCAAGTCACCATTCTGCACCTGAACCACACCAACATCGT |
| | GATGCAGCCGGTTCACCGACTGGGCCACCTGCCCAGACAACTGTTTAATCTGATCATGCAAGGCTTTCACATCAGCCACCGTT |
| | AACTCCTCACTACTTGAACCGCCGCCGTGGCCATTCACCACAGCCATAAATTTGTCCCACGGAAACCACGGCCCCGGATCATC |
| | GTGATCCGACTGATGCCACGCATCCGTCACATCAGTGTGCCCGCAGATGCCCCGCCTACCAGCCTTCAAATCGGATACGCTAA |
| | GCTTCCTTTTCGGAACACCATGCTTGTCACACAACTGCCGACACAGGATGGCAGCCTTCTCCACCGCAGGCCACACGCGAGG |
| | ATCAAGCCACTGCTCCCGAGTGTAAGCGTGCCCCGGCACCCTAAACGAGGCGTGCGAACCCCCATCCGCGCAAATCTCGATA |
| | CCTAAACTATGCGGATTCGGCGGGGCATGCCACCCAATCGTAGACTCCGACAAGCACTGCACCGTCTCCCCAATATCGCAAA |
| | CATAATGCGCCGAACCACCAGGCGATGGGACGCGAAATAGTTTGCCGGGGACACCGCCCGCCCTTTACG |
| | SEQ ID NO.: 20; >7471 len: 17114 cycle: O |
| | AACATCTGGTTGACAAGGATGCTGTTGAGTCCGGAAAGGATAACGGCATTGTTGATACAGTAAAAGACGATGGCGGGCAGG |
| | AGGTAGCCGACAATCAGTTGAAGAATGAAGGCGAGGGTAAATCGCCGGGGACTGATTGGAAGGCTGAGGCCCGTAAGAGGG |
| | AGTCTCGTGCTAAAAGTAATTTCGCCGAGTTGGAGAAGCTTCGCGCCTCGGATGGTGATGCGGGGTCTGTGATCGATGATCTT |
| | CGCCGCAAGAATGAGGAACTCGAAGACCGGATCAATGGGTTTGTTCTTGAGGGTGTGAAGCGTGCTGTTGCGGCCGAGTGTG |
| | GCTTGTCGGGTGATGCGGTCGCTTTCTTGCACGGTAGCGACCGTGAAGCGCTGGTGGAGTCTGCTAAGGCTTTGAAGGGTTTG |
| | ATCGACCATAGTAGTGGCTGCGCGGGTGTGCGCCGTCTTGCGGGGAGTGCCCCCGTTGATGATGTTAAACGACGTGAGGGTG |
| | TCGCGTTTGTGGATGCTCTTGTCAATAATTCTAGGAGATGATTTCTGATGGCTGACGATTTTCTTTCTGCAGGGAAGCTTGAGC |
| | TTCCTGGTTCTATGATTGGTGCGGTTCGTGACCGTGCTATCGATTCTGGTGTTTTGGCGAAGCTTTCGCCGGAGCAGCCGACTA |
| | TTTTTGGCCCTGTTAAGGGTGCCGTGTTTAGTGGTGTTCCTCGCGCTTAGATTGTTGGTGAGGGCGAGGTTAAGCCTTCCGCAT |
| | CGGTGGATGTTTCGGCGTTTACTGCTCAGCCTATCAAGGTTGTGACTCAGCAGCGTGTCTCGGACGAGTTTATGTGGGCTGAT |
| | GCTGATTACTGTCTGGGTGTGCTTCAGGATCTGATTTCCCCGGCTCTTGGTGCTTCGATTGGTCGCGCTGTTGATCTGATTGCT |
| | TTCCATGGTGTTGATCCGGCTACGGGTAAGCCTGCTGCTGCTGTCAAGGTGTCGCTTGATAAGACGACGAAGACGGTTGATGC |
| | CACGGATTCTGCTACGACCGATCTTGTTAAGGCTGTCGGCCTGATTGCGGGGGCTGGTTTGCAGGTTCCTAACGGTGTTGCTT |
| | TGGATCCGGCGTTCTCGTTTGCCCTGTCTACTGAGGTGTATCCGAAGGGGTCTCCGCTTGCCGGTCAGCCGATGTATCCTACC |
| | GCCGGGTTTGCTGGTTTGGATAATTGGCGTGGCCTGAATGTTGGTGCTTCTTCGACTGTTTCTGGTGCCCCTGAGATGTCGCCT |
| | GCCTCTGGTGTTAAGGCGATTGTTGGTGATTTCTCTCGTGTTCATTGGGGTTTCCAGCGTAACTTCCCGATCGAGCTTATCGAG |
| | TATGGTGACCCGGATCAGACTGGGCGTGACCTGAAGGGCCATAATGAGGTTATGGTTCGTGCCGAGGCTGTGCTGTATGTTG |
| | CGATTGAGTCGCTTGATTCGTTTGCCGTGGTGAAGGAGAAGGCTGCCCCGAAGCCTAATCCTCCGGCTGGAAACTGATTCATT |
| | TGTTGCGATAATGTTTATGCTGTGTGCAGGGGGTGGTGTTGATGGGTATCATTTTGAAGCCTGAGGATATTGAGCCTTTCGCC |
| | GATATTCCTAGAGAGAAGCTTGAGGCGATGATTGCCGATGTGGAGGCTGTAGCTGTCAGTGTCGCCCCCTGTATCGCTAAAC |
| | CGGATTTCAAATATAAGGATGCCGCTAAGGCTATTCTGCGCAGGGCCCTGTTGCGCTGGAATGATACCGGGGTTTCGGGTCA |
| | GGTGCAGTATGAGTCTGCGGGTCCTTTTGCTCAGACGACACGGTCTAATACTCCCACGAATTTGTTGTGGCCTTCTGAGATTG |
| | CTGCGTTGAAGAAGTTGTGTGAGGGTGATGGTGGGGCTGGTAAAGCGTTCACTATTACACCGACCATGAGGAGTAGTGTGAA |
| | TCATTCTGAGGTGTGTTCCACGGTGTGGGGTGAGGGTTGCTCGTGCGGGTCGAATATTAACGGCTACGCTGGCCCTTTGTGGG |
| | AGATACGATATGACCAGTTTTCCTTATGGTGAAACGGTTGTGATGCTTCAACCGACTGTTCGTGTCGATGATCTTGGTGACAA |
| | GGTGGAAGACTGGTCTAAGCCTGTCGAGACTGTGTACCATAACGTGGCCATCTATGCTTCCGTTTCGCAGGAGGATGAGGCT |
| | GCGGGGCGTAACTCTGACTATGAGTATTGGTCGATGCTTTTCAAGCAGTCTGTTGTTGGTGCTGATTATCGTTGCAGGTGGCG |
| | TATCCGGGGTGTTGTGTGGGAGGCTGACGGGTCTCCTATCGTGTGGCATCATCCAATGTCTGGCTGGGATGCGGGCACGCAG |
| | ATCAATGTGAAGCGTAAGAAGGGCTGATGGGTAGTGGCTCAGGATGTGAATGTGAAGCTGAACTTGCCGGGTATTCGTGAGG |
| | TGTTGAAGTCTTCTGGGGTGCAGGGCATGTTGGCTGAGCGTGGCGAGCGTGTCAAGCGTGCAGCCTCGGCGAATGTGGGCGG |
| | TAACGCTTTCGATAAGGCCCAATACCGTAATGGTTTGTCGTCAGAGGTGCAGGTTCACCGTGTTGAGGCTGTCGCTCGTATAG |
| | GCACCACATATAAGGGTGGGAAGCGTATTGAGGCGAAGCATGGCACGCTGGCTCGTTCGATTGGGGCTGCGTCGTGATCGTC |
| | TACGATGACCCCAGGAAGTGGGCTAAACGCGTGCTCAAGGATGATGGCTGGCTGTCTGGGATACCTTGTACTGGGACAGTGC |
| | CGGATAGTTTTGAGGGTGACCTTGTTTGGTTGGCTCTTGATGGTGGCCCGCAGTTGCATGTGCGTGAGCAGGTTTTTTTGCGG |
| | GTGAATGTGTTTTCTGATACGCCGGATCGGGCTATGTCTTTGGCGCGTCGTGTTGAGGCTGTTCTGGCTGACGGGGTTGATGG |
| | TGATCCGGTGGTGTTTTGTAGGCGTTCTACGGGTCCTGATCTTCTGGTTGATGGTGCACGTTTTGATGTGTATTCGCTTTTTGA |
| | GCTGGTGTGTCGGCCTGTCGAATCCGAGTAAGCGTATCGTTGTTTTTGTTGATTGATTTTTAGTTTGATTGTTTTTGGGGGTTA |
| | TGATGGCTGCAACACGTAAAGCGTCTAATGTTCGCTCTGCTGTTACGGGTGACGTCTATATTGGTAAAGCTCATGCCGGTGAC |
| | GCTATTGATGGTGTGAAGACGGTTCCTGAGGGGTTGACTGCTTTAGGGTATCTGTCTGATGACGGGTTTAAGATTAAGCCTGA |
| | GCGTAAAACGGATGATTTGAAGGCTTGGCAGAATGCGGATGTTGTTCGCACGGTGGCTACAGAGTCGTCTATCGGGATTTCTT |
| | TCCAGCTGATCGAGTCTAAGAAGGAGGTTATCGAGCTGTTTTGGCAGTCGAAGGTTACTGCCGGATCCGATTCGGGTTCGTTC |
| | GATATTTCTCCTGGTGCTACCACTGGCGTGCACGCTTTACTGATGGATATTGTGGATGGCGATCAGGTTATTCGCTACTATTTC |
| | CCTGAGGTTGAGTTGATCGATCGTGACGAGATTGAGGGTAAGAATGGCGAGGTGTATGGGTATGGTGTGACGTTGAAGGCGT |
| | ATCCTGCCCAGATTAATAAGAAGGGTGATGCGGTGTCTGGTCGGGGGTGGATGACGGCTTTAAAAGCTGATACCCCTCCGGT |
| | TCCTCCGAAGCCTCAGCCGAATCCGAATCCGCCGTCCGATAACTGATACACGATTTTAGGGGATTGTTGATAGATGAGTGAC |
| | GCTGGTTTCACATTGAAGATTGGTGACCGTAGCTGGGTGTTGGCGGATGCGGAGGAGACGGCTCAGGCTGTTCCTGCCCGCG |
| | TTTTCCGTCGTGCCGCCAGGATTGCCCAGTCTGGGGAGTCTGCGGATTTCGCCCAGGTTGAGGTGATGTTTTCCATGTTGGAG |
| | GCTGCCGCCCCGGCTGACGCGGTGGAGGCCCTGGAGGGGCTTCCTATGGTTCGTGTGGCGGAGGTTTTCCGTGAGTGGATGG |
| | AATACAAGCCTGAGGGTAAGGGTGCCTCTTTGGGGGAATAGTTTGGCTCCACGGCCTGATTGATGATTATCGTGGGGCCATC |
| | GAATACGATTTCCGCACTAAATTTGGTGTTTCTGTTTATAGTGTTGGTGGCCCGCAGATGTGTTGGGGTGAGGCTGTCCGGCT |
| | GGCTGGCGTGTTGTGTACCGATACGTCGAGCCAGTTGGCGGCCCACCTGAATGGTTAGCAGCGCCCGTTTGAGTGGTGCGAG |
| | TGGGCTGTGTTGGACATGCTGGATCATTACAGGTCTGCTAATAGTGAGGGGCAGCCGGAGCCTGTGGCGAGGCCTACGGATG |
| | AGCGTAGAGCCCGGTTTACGTCTGGGCAGGTGGACGATATTTTGGCGCGTGTTCGTGCCGGTGGCGGGGTGTCTCGCGAGAT |
| | TAATATTATGGGGTGAATAGTGTATGTCTGGTGAGATTGCTTCTGCATATGTGTCGTTGTATACGAAGATGCCCGGCCTTAAA |
| | AGTGATGTTGGTAAACAGCTTTCTGGGGTGATGCCTGCTGAGGGTCAGCGTTCGGGTAGTCTTTTTGCTAAGGGTATGAAGTT |
| | GGCGCTTGGTGGTGCCGCAATGGTGGGTGCCATCAATGTTGCCAAGAAGGGCCTCAGGTCTATCTATGATGTGACTATTGGTG |
| | GCGGTATAGCTAGGGCGATGGCTATTGATGAGGCTCAGGCTAAGTTGACTGGTTTGGGTCACACGTCTTCTGACACGTCTTCG |
| | ATTATGAATTCTGCTATTGAGGCTGTGACGGGTACGTCGTATGCGTTGGGTGATGCGGCGTCTACGGCTGCGGCGTTGTCTGC |
| | TTCGGGTGTGTAGTCTGGCGGGCAGATGACGGATGTGTTGAAGACTGTCGCGGTTGTGTCTTATATTTCGGGTAAGTCGTTTC |
| | AGGATACGGGCGCTATTTTTACGTCTGTGATGGCTCGCGGTAAGTTGCAGGGCGATGACATGTTGCAGCTTACGATGGCGGG |
| | TGTTCCTGTGCTGTCTCTGTTGGCTAGGCAGACGGGTAAAACGTCTGCTGAGGTGTCGCAGATGGTGTCGAAGGGGCAGATT |
| | GATTTTGCCACGTTTGCGGCTGCGATGAAGCTTGGCATGGGTGGTGCTGCGCGGGCGTCTGGTAAGACGTTTGAGGGCGCTAT |
| | GAAGAATGTTAAGGGCGCCCTGGGTTATCTTGGTGCTACGGCTATGGCGCCGTTTCTTAACGGGTTGCGGCAGATTTTTGTTG |
| | CGTTGAATCCGGTTGTCAAGTCGGTGACGGATTCTGTGAAGCCGATGTTTGCGTCGGTGGATCAGGGTATTCAGCGGATGATG |
| | CCGTCTATTTTGGCGTGGATTAACCGTATGCCGGGCATGATCACGAGAATGAATGCACAGATGCGCGCCAAGGTGGAGCAGC |
| | TGAAGGGCATTTTTGCAAGGTTGCATTTACCTGTCCCTAAAGTGAATTTGGGTGCCATGTTTGCTGGCGGCACCGCAGTGTTT |
| | GGTATTGTTGCTGCCGGTGTGGGGAAGCTTGTTGCAGGGTTTGCCCCGCTGGCGGTGGCGTTGAAGAATCTACTGCCGTCGTT |
| | TGGTGCTTTGAAGGGTGCCGCTGGCGGGCTTGGTGGCGTGTTTCGCGCTTTGGGTGGCCCTGTCGGTATTGTGATCGGCTTGT |
| | TTGCTGCCATGTTTGCCACTAACGCCCAGTTTCGTGCCGCGGTTATGCAGCTGGTTGGTGTGGTTGGTCAGGCTTTGGGCCAG |
| | ATTATGGCGGCTATTCAGCCGCTGTTTGGTTTGGTTGCCGGGGTTGTGGCACAGTTGGCGCCAGTGTTCGGCCAGATTATCGG |
| | TATGGTTGCGGGTTTGGCTGCCCAGCTGGTGCCTTTGATTAGTATGCTTGTCGCCCGGCTGGTTCCTGTGATCACGCAGATTAT |
| | TGGTGCGGTGACACAGGTTGCTGCAATGTTGTTGCCTGCGTTGATGCCGGTGTTGCAGGCGGATGTTGCTGTGATACGGCAGG |
| | TTGTTGCCGGTTTGGCTGTGGGGATCACCGGGAATGCCGGTTTGGCTTTGGATGCGATGTCGGGTGTGGCGGGTCGGCTGCCT |
| | GATGCGGTTGATGCCCGGTTTGGTGTGCGATCGTCGGTGGGCTCGTTTACCCCGTATGGCAGGTATCAGCGTGCTAAGGGTGA |
| | GAGTGTTGTGGTGAATGTGAATGGGCCTACTTATGGTGATCCTAACGAGTTTGCGAAGCGGGTTGAGCGGCAGCAGCGTGAC |
| | GCTTTGAACGCGTTGGCTTACGTGTGATTGGAGTGTTGTGCATGTTTATTCCTGACCCGTCTGATCGTTCTGGTTTGACTGTTA |
| | CCTGGTCTATGTTGCCATTGATTGGTAATGATCCGGAGCGTGTGCTTCATTTGACGGACTATACGGGTGCGTCTCCTGTCATGT |
| | TGTTGAATGATTCGTTGCGCGGCCTGGGTGTGCCCGAGGTGGAGCATTTTTCTCAAACTCATGTTGGGGTGCACGGCTCGGAG |
| | TGGCGCGGGTTTAATGTGAAGCCTCGCGAGGTGACATTACCTGTGTTGGTGTCGGGTGTTGACCCGGATCCGGTGGGCGGGTT |
| | TCGTGACGGTTTTTTGAAAGCCTATGACGAGTTGTGGTCTGCTTTTCCTCCGGGCGGGGAGGGGGAGTTGTCGGTGAAGACTC |
| | CTGCCGGTGTTGAGCGTGTGCTAAAATGCCGGTTTGATTCGGTGGATGACACGTTTACGGTGGATCCGGTTAATCGTGGCTAT |
| | GCGCGCTATCTGTTGCATTTGACAGCCTATGACCCGTTTTGGTATGGGGATGAGCAGAAGTTTCGTTTCAGTAACGCGAAGTT |
| | GCAGGATTGGTTGGGTGGCGGCCCTGTCGGCAAGGATGGCACGGCGTTTCCTGTGGTGTTAACACCGGGTGTTGGTTCAGGTT |
| | GGGATAATCTGTCTAATAAGGGTGATGTGCCTGCGTGGCCTGTGATTCGTGTCGAGGGCCCGTTGGAGTCGTGGTCTGTGCAG |
| | ATTGATGGTTTGCGTGTGTCTTCTGATTATCCTGTCGAGGAGTATGATTGGATCACTATTGATACGGATCCTCGCCAGCAGTC |
| | GGCGTTGTTGAACGGGTTTGAGGATGTGATGGATCGTTTGACAGAGTGGGAGTTTGCGCCTATCCCGCCTGGCGGTTCTAAGA |
| | GTGTGAATATTGAGATGGTTGGTTTGGGTGCCATTGTTGTGTCGGTGCAGTACAGGTTTTTGAGGGCTTGGTGAATAGTTGAT |
| | GGCTGGTCTTGTTCCGCATGTAACATTGTTTACGCCGGATTATCGCCGTGTGGCGCCTATCAATTTTTTTGAGTCGTTGAAATT |
| | GTCGTTGAAGTGGAATGGTTTGTCGACGCTGGAGTTGGTGGTGTCGGGTGATCATTCTAGGCTTGACGGGTTGACTAGGCCGG |
| | GTGCACGGCTGGTTGTTGATTATGGTGGTGGCCAGATTTTTTCTGGGCCTGTGCGTCGGGTTCATGGTGTGGGTCCGTGGCGT |
| | TCTTCGCGGGTGACTATCACGTGTGAGGATGATATCCGCCTGTTGTGGCGTATGCTGATGTGGCCTGTGAATTATCGTCCCGG |
| | TTTGGTGGGTTCGGAGTGGCGTGCTGACAGGGATTATGCCCACTATTCGGGTGCGGCTGAGTCGGTGGCTAAGCAGGTGTTG |
| | GGGGATAAGGCTTGGCGTTTTCCTCCGGGTTTGTTTATGAACGATGATGAGAGTCGTGGCCGCTATATTAAGGATTTTCAGGT |
| | GCGGTTTCACGTGTTTGCCGATAAACTGTTGCCTGTCCTGTCGTGGGCTCGGATGACTGTCACGGTGAACCAGTTTGAGAATG |
| | AGAAGTTTGATCAGCGTGGTTTAGTGTTTGATTGTGTGCCGGCGGTGACCCGGAAGCATGTGTTGACTGCCGAGTCTGGTTCG |
| | ATTGTGTCGTGGGAGTATGTGCGTGACGCCCCGAAGGCGACATCTGTGGTTGTGGGTGGCCGCGGCGAGGGCCGGGATCGGC |
| | TGTTTTGTGAGGATGTTGATTCGATGGCCGAGGATGACTGGTTTGATCGTGTCGAGGTGTTTAAGGATGCCCGTAACACGGAT |
| | TCTGAACATGTGCATCTTATTGATGAGGCTGAGCGGGTGTCAGGGGAGTCTGGGGCCACGTCGGGGTTTAAGATCGAGTTGG |
| | CGGAGTCGGATGTGTTGCGGTTTGGGCCCGGCAATCTGATGCCAGGGGATTTGATCTATGTGGATGTGGGCTCGGGTCCTATT |
| | GCGGAGATTGTGCGGCAGATTGATGTGGAGTGTGTATCGCCGGGTGACGGCTGGACGAAGGTGACTCCTATTGCGGGGGATT |
| | ATGAGGATAATCCGTCGGCGTTGCTAGCACGGCGTGTTGCTGGTTTGGCTGCGGGTGTGCGGTATTTGCAAAAATTCTAAAAA |
| | GATTAGGGGTTTGTTGTGGGTATTGTGTGTAAAGGGTTTGATGGTGTGTTGACCGAGTATGATTGGGCTCAAATGTCTGGTCT |
| | GATGGGTAATATGCCGTCTGTGAAGGGGCCGGACGATTTTCGTGTGGGTACTACTGTTCAGGGTGCCACGGTGTTGTGTGAG |
| | GTTTTTCCGGGGCAGGCTTGGGCTCACGGGGTGATGTGCACTTCTAGTAGTGTTGAGACGGTGACAGGGCAGCTGCCTGGCC |
| | CCGGTGAGACCCGATACGACTATGTGGTGTTGTCTCGGGATTGGGAGCAGAACACAGCCAAGTTGGAGATTGTTCAGGGTGG |
| | CCGTGCGGAGCGTGCCCGTGACGTGTTGAGGGCCGAGCCTGGCGTGTTCCATCAGCAACTGTTGGCTACCCTGGTGGTGTCGT |
| | CTAACGGGTTGCAGCAGCAGCTTGACAGGCGTGCTATAGCTGCCCGTGTGGCGTTTGGGGAGTCTGCTGCGTGTGACCCGAC |
| | CCCTGTGGAGGGTGACCGTGTGATGGTTCCTTCTGGGGCTGTGTGGGCTAACCATTCGGGCGAGTGGATGCTACTGTCTCCGA |
| | GGATTGAGACGGGCACTAAGCAGATCATGTTTGGCGGGTCTGCTGTGTATGCTTACACGATCCCGTTTGAGCGGCCGTTCGGT |
| | GCTCCGCCGGTTGTGGTGGCGTCTATGGCTACGGCGGCTGGGGGTACGACACAGATTGATGTGAAAGCCTACAATATTACTA |
| | ATAGTAATTTTAGTTTGGCTTTTATTACGAATGACGGGTCTAAGCCTTCTGGTGTGCCTGCGATAGCTAACTGGATTGCTGTCG |
| | GCGTGTGACCGGGCTGTTGTTGTGGCGGATGGTGTGATGTCGGGGGGCTGTGGTGTCGTGGTTTACTCCTGCACTGGTGGCCT |
| | CTATCTGTACCGCATTGGCCACGGTTTTGGGTTCGGTTCAGGCTGCCACATCTAAATCTCGGAAGCGTTTGCGGCGGCTGTCG |
| | GCTCAGGTAGATGCGATGGAAGAGTATACGTGGTGTGTGCGGCGCGAGGTGCGCCAGTTTAACGCCCGGCTTCCTGACAGGG |
| | TTGATCCGATTGTTTTGCCTGAGTTGCCTAGTTTCCTGTCGGACACGTTGGGGGGTGAATGATGAGGGAGTTGGAGGAGGAG |
| | AAGCGGCAGCGCCGCAATTTTGAGAAGTTTTCACTGGTGTTGCTGTTTTTGTCGCTTGTGTTGTTGGCGGTGGTTGCCGGGGG |
| | TGCTTTGCGTTTCGGGGCTGTATCCTCTGAGCGGGATTCGGAGCAGGCGAGGGCCCAGTCTAATGGTACAGCGGCTAAAGGG |
| | TTGGCTAGCAGTGTGAAGCGGGTGTGTGCTTCGGGTGGGCGGGAGTCGGTGCGTCTTCACCAGTCTGGTTTGTGTGTGGATGC |
| | TGTGCGTGTTGAGCGTAGCGTGCAGGGTGTGCCGGGTCCTGCCGGTGAGCGCGGCCCGCAAGGGCCTGCAGGTGCTGATGGG |
| | CGTGATGGTGTTAATGGTTCGGCTGGGCTGGTTGGCCCTGTTGGTCCGCAGGGTTCTCCTGGTTTGAATGGTGTGAAAGGTCC |
| | TGACGGGTTGCCTGGTGTGAATGGGTCGGATGGCCGTGATGGTGTTCCAGGCCGTGCAGGTGCTGACGGTGTGAACGGCGTT |
| | GATGGCACTGCTGGTCGGGATGGTTCGGCCGGTGAGCGCGGTGATGTGGGCCCTTCAGGTCCTGCCGGCCCGCAAGGTGCAC |
| | AGGGTGAACGGGGTGAGCGCGGCCCCGCCGGTGCGAACGGATCCGATGGTAAAGATGGTAAGGATGGCCGTTCTGTTGCGT |
| | CTGTGTACTGTTCCGGGGGCCGCCTTGTTGTGAAATATAGTGACGGTGTGGCTTCTACAATATCGGGCTCGGTGGCCTGCCAG |
| | GGTGTGAAACCGTCGCCTATAGTGACTATATCATCCCACAAATAGAAAGGAGTGGCTGTGATGGTAGTGTTTGGTGGTGGTG |
| | TGTGGTGAGGTTTATTCCTGCAGCACATCACTCGGCCGGCTCGAATAGTCCGGTGAACAGGGTTGTGATTCATGCGACGTGCC |
| | CGGATGTGGGGTTTCCGTCTGCCTCCCGTAAAGGGCGGGCGGTGTCCACGGCAAACTATTTCGTGTCCCCATCGTCTGGTGGT |
| | TCGGCGCATTATGTTTGCGATATTGGGGAGACGGTGCAGTGCTTGTCGGAGTCTACGATTGGGTGGCATGCCCCGCCGAATCC |
| | GCATAGTTTGGGTATAGAGGTTTGCGCAGATGGCGGTTCGCACGCCTCGTTCCGGGTGCCAGGGCATGCTTACACTCGTGAGC |
| | AGTGGCTGGATCCTCGGGTGTGGCCCGCGGTGGAAAGAGCGGCGGTGCTGTGCCGGCAGTTGTGTGACAAGCATGGTGTTCC |
| | GAAGAGGAAACTGTCTGTGGCTGATTTGAAGGCTGGCAGGCGGGGCATCTGCGGGCACACTGATGTGACGGATGCGTGGCAT |
| | CAGTCGGATCATGACGATCCGGGGCCGTGGTTTCCGTGGGACAGGTTTATGGCCGTCGTGAACGGCGGCAGTGGAGAGAGTG |
| | AGGAGTTAACGGTGGCTGATGTGAAAGCCTTGCGTGATCAGATTAAACAATTGTCTGCTCAGCTTACTGGTTCGGTGAATAA |
| | GCTGCATCACGATGTTGGTGTGGTTCAGGTGCAGAATGGTGACTTGCGTAAGCGTGTCGAGGCTTTGTCGTGGGTGAAGAAT |
| | CCTGTGACGGGGAAGCTGTGGCGCACTAAGGATGCTTTGTGGAGTGTCTGGTATTACGTGTTGGAGTGTCGTAGCCGTATTGA |
| | CAGGCTCGAGTCTGCTGTTAACGGTTTGAAAAAGTAATGGTGGTTTGTTGTGGGTAAACAGTTTTGGTTAGGTTTACTGGAGC |
| | GGGCGGCTAAGACTTTTGTGCAAACGTTTGTTGCGGTGTTGGGGGTGACGGCGGGTGTCACGTATACTGCGGAGTCGTTTCGC |
| | GGTTTGCCGTGGGAGTCTGCCCTGATAACAGCAACGGTGGCTGCGGTGTTGTCGGTTGCTACATCGTTTGGTAGCCCGTCGTT |
| | TGTGGCTGGGAAACCTAAAACCACGGTTGTGGATGCGGGTTTGGTTCCGCCCGATGACGGGGGCATGGTTGAGCCGCACATG |
| | GGGGATGTGTCGGATCCTGGCATGATTGAGCCTGCAGATGATGTGTATGGTTTTGCCGGCTATGTGCCTCGGCGTGCAGCCGA |
| | GTCTGAGGTTGGCACGGTAGAGTCTACTGTTGCATAATTGAATATGTGTGTGCCCCAGCGGTGCTGCCACGATCGTGTGGTGG |
| | CTGCTGCTGGGGCACTATTTTTGTGTCTATAGTATTCTATGATTCGTTATTGTTGATGGTTTCCTCAAGCATCTGGTGCAGGTG |
| | GAGGCAGGCTGAGATAGTATCGTTGGCCTGGTCTAGAACATCCTGGCCGATAACGCCTTCGTGGTTGTCGCGGTGGTAGAGG |
| | ATGGACTGCATGATATCGTCGGCCTCCGCTTGCAGTAGTTTTGCCTGGTATGCGATTCTGGCGAGCCAGTCTATGGCTTCATG |
| | GCTTGCCTGTGTGTCGTCCGGCATGCCATGGGTGTTGCTGTTGTTTGTGGGGTGTCCTGCACTGTCGCAGTCCCACAGGATTTC |
| | GCTGCACTCGTCTAGCGTGTCCTGGTCGATGTCGAGATCGTCGAGGCTGACTCCTTTGACGGTGAGGTTCACATCATCGAGGG |
| | AGATGGGTACATGGTACTGGTTTTCGATACCGGCAACAATGTTTTCCAACTGTTGCATGTTGGTGGGTTGTTGTTGGACGATA |
| | CGGTGTACTACTGTTTTGAGGGCAGTGTAGGGGATATTGGTTGTGTTGTCCATCGTGTTATGCCATTCCTTCGTTATCGTCTGG |
| | CATGTAGTATGTGCTGTTTGCGTACTCGGATAACGTCATCAGTGTTTGGTCTGCCCACTGTTTCACGGTTTGTCTTGTCACCCC |
| | GAGTCGTTGGGCGGCCGACGCATATGTTTGGTCATACCCGTATACTTCACGGAAGGCTGCCAGGCGGGCTAGCCGTTTGCGCT |
| | GTTTGGAGGGTTCACATGTGAGGGTGTAGTCGTCTATCGCTAATTGTAGGTCGATCATGGTGGCAATGTTGTTGCCGTGGTGT |
| | TGTGGCGCGGTTGGTGGTGGTGGCATGCCCGGCTCCACACTGGGTTTCCATGGGCCGCCGTTCCAGATCCATTGGGCGGCTTG |
| | GATGATGTCGGCGGTGGTGTAGGTTCGGGTCATGTGTCACCCCTGGAACAGGTTGTCGAGGTTGTCTGGCTGGCTGGTGGTGT |
| | CGAATCGTCCGACGCAGTGGCAGTAGTCGTACATGAGTTTGATGATGTGTTGGTGGTCTCCCAAATAGGTGTTGCCGCTGATA |
| | CTGTAGGTGGCTGTGCCGTCTTTGCTGATGGTGTATTTGGCGGTGATGGTTTCGGGGTTTTCTGTGTTGGTGATGATTGCTGTG |
| | GTGGTGGCGCCTACTGTTTGGAGTATGGTGGTTTGGGTTCCGTCGTCGAGGATGGTTGTGACCATTGTGGTTCTCCTTAGTTGC |
| | TGGTTTGGTTGTCGGCTATGGCTGTGATTTCTTGGACGGGTTTGGGTAGGTCTAGGTGGTGGACTGTTTTGTTTGCTAGTCGTT |
| | GGGCGACACGGTAGCACATTTGGGTCCACTGGTTGCCTGTGAGTTGGTGGTATTGGTTGCGTACCGCAATATATAGTAGAGA |
| | GTCTCGGTACAGGTCGTCTGGGNTGACAGCCGGGTAGCGGCGGGCNATATTGGTGCAGGCTTTGTGTAGCTGGTGTTGGTGG |
| | TGGGGGGTTGCCCATTCCCAATCAGCTGTGGTGGCTAGGTCTGCTTTTGTTGGTCGTCTACTCATGGTTTTTATCTGGCTATCT |
| | GGTAGTTGTTTGGTGTTTTGTTGTTGATAGTGTAGCACACGAGTCCGGGGTTGCCGTTGGTGCCCGTCTTGTGCAGGTACCAG |
| | ACGGATTCTCCTTCCATGGATGGGCGTTGGATGAAGGTGCGTTGTCCTTGCTCGGATATTTCGAGGTGGTGCCGGTGTCCGGC |
| | CATGAGTATATGGGATACGGTGCCGTTGTGGAATTCTTGGCCGCGCCACCATTCGTAGTGTTGGTTGTTGCGCCATTGGTGTC |
| | CGTGGGCGTGCAGGATGGTAGCCCCGGCTACGTTGACGGTGGTGGTCATTTCGTCTCGGCTGGGGAAGTGGAAGTGAAGGTT |
| | GGGGTATTGGTTGTTGAGCTGGTAGGCTTCTGCGATGGCCCGGCAGCAGTCCACTTCGAAACTATCGTCGTAGGTGGTGACTC |
| | CTTTACCGAAGCGCACGGCTTCTCCGTGGTTGCCGGGGATGGATGTGACGGTCACATTTTGGCAGTGGTCGAATTGGTGGATG |
| | AGTTGCATCATGGCCATGCGGGTGAGCCTGATTTGTTCCGTCAAGGGTGTTTGTGTGCGCCAGGCGTTGTTGCCGCCTTGTGA |
| | CACGTATCCTTCGATCATGTCGCCGAGGAAGGCTATGTGGACTCGTTGCGGCTGGCCTGCTTGCTGCCAGTAGTGTTTAGCTG |
| | AGGCGAGGGAGTGTAGGTAGTCGTCGGCGAAGTGTGCTGTTTCTCCTCCGGGGATGCCTTTGCCGATTTGGAAGTCGCCTGCC |
| | CCGATGACGAAGGCCGCATCGCTGCTACGGGTGTCGGACTGTTTGGGGGGTGTCCATTCGGCTAGTTTATCGACGAGTTCGTC |
| | TACAGGGTAGGGGTCGGTTGCGGGTTGGTGGTCGATGATTTTTTGTATGGATCGGCCTGTTTCTCCGTTGGGTAGGGTCCATT |
| | CGGAGATGCGTGTGCGGCGTACGGTTCCGTTGGCGAGATCATCGCGGATGGTGTCTGCTTCGTTGTCGTGGTTGGCTAGCTGG |
| | GTGAGGAGCCGGTCTATGTTGTCTATCATCGGGTATCCTCTTCCTTTTGTGGAGTGGTGTTGGCTTGTTTGCGGCGATAGTCTT |
| | TGATGACGGTGGCGGAGATGGGGTATCCGGCTTGGGTGAGTTGTTTTGCTAGCCAGGATGCGGGTATAGACCTGTCGGCGAG |
| | GACGTTTGCGGCTTTGCGGCCGTAGCGTTGGATCAACGTTTCAGTTTTGGTTGCCATGATGTCCTAGGGGTTGTGTGGTGGGC |
| | TGCCATCCTGTGCGGCAGTCGCCGTCGTGTCCTGGTTTGCGTGTGCACCACGATGTTGTTCCGTCTGTGTGGTTGAGTGTTTTG |
| | CCGCACATGACGTTTTGTAGATGCTCCGGCAGCTCGCTATTGCTATCGTCTTGCTCGTCTAGCAAAGTTTTTTGTTGGGTGAAA |
| | AACTCGGACACGGTGCCGTTGTGGACTGGGAGTGTCCATGTTTTCCATTGTTGTTGTAGTCGGGTGTTCCAGTGGAATTGTTT |
| | GGCGGCGTTTTCTGCCTGTTTTATGGTTTTGTAGTAGCCGACGAGTATGCGCTGGTGTTGCTGGTCGGGCGGGTTTTGGCCTCG |
| | CCAGTATTGTGCCGCGACGGCGTACCGGTTGTTGTCGGTGAAGGTGTTCCAGCAGTATTGGATGATGTTGTTGAGGGTGTCAT |
| | CTGGCAGATCGTCTGGGTTGAGGTTGGTGGCCTGGGTGAGGATGTCTCGGATGGTTTGAAGGTTGCGTTTTGTGGGTTTGAAC |
| | GAGATGCTCACGATAGTGCCGGCTGTTCGTCTTGCATGAACTGGTTGAAGGTGTTGTTCCCGGCGTGTTGGGCTTGTGTGATT |
| | TGCTGGTCGGTCCAGTCGGGGTGTTGCTGTTTCAGATAATACCAGTGGCTCGCATTGTAGGTTTCGTTTTGTAGCCGTGTGAG |
| | ATGGTTTTCTTCGATGATTTGTTTCCACATGGTCCATGACACGTCGAGCCGGTTGAGGATTTCGAGGGCTGGGATGTTAAACT |
| | GGTCGAGGAAGAGGATTTCGTGGGTGTAGTAGTTTTTCTCGTAGGCGTCCCATCCGCTTCGGTGCCTGTTGGGCTGGTTTTGG |
| | GGTAGGCTTCCCGGCAGATTTTGTGTAACCGTTTGGCCATGTCGTCGGGTAGTTTAATGTCGGGGTTGGCGCGGATCATGGAT |
| | CGCATCCCATCATAGGTGGTGCCCCAGGTGTGCATGATGTAGGTGGGGTCTTCGCCATCAGCCCATTTCTCTGCACAGATGGC |
| | GAGGCGGATGCGCCTCCTGGCTGTTTGGCTGGTGTTGCGCCGGTTGGGGATGGGGCACGTGTCGAGGGGATCCATGATGTTTT |
| | AGTTTACCTTTCTGGTTTCGTGTTGTTGACGTGTTTTACTGTAGCACAGTGTCTAGTGCTTGTGTCAACCCTGTTTTTCCGGCCT |
| | GCAGGTAGGTGTCTGTGACATCCCCGACAGTGAGGGGCACATGGGTGGCTTGCGGTAATGCTTGGGTTAGGGTTTGGGCCAT |
| | CTTGTCTCCCGCGGGGTCTGGGTCGGACCAGATGTAGATGTGGTCATAGCCTACAAAAAATTTGGTCCAAAAAGTTTGCCAC |
| | GAGGTTGCGCCGGGTAGGGCAACGGCCGACCATCCGCATTGTTCGAGGATCATGGAGTCGAATTCGCCTTCGCAAATGTGCA |
| | TTTCGGATGCCGGGTTGGCCATGGCGGCCATGTTGTAGATGGAGCCTGTGTCTCCTGCCGGGGTTAGGTATTTGGGGTGGTTG |
| | TGGGTTTTGCAGTCGTGCTGGAGTGAGCAGCGGAAACGCATTTTTCTTATTTCGGCTGGGCCGCCCCAAACGGGGTACATGTA |
| | TGGGTTGGTGATGCGCTGGTTGTAGTTTTCGTGGCCGGGTATGGGGTCATTGTCGATGTATCCAAGGTGGTGGTAGCAGGCTG |
| | TTTCTTCGCTGATGCCTCTTGCTGAGAGCAGGTCGAGTATGTTTTCGAGGTGGGTTTCGTAGAGGGCCGAGGCTTTCTGGATT |
| | CGGCGGCGTTCCGCAATGTTGTATGGGCGTATACTGTCGTACATTCGGGTTTTCTTCTTCTAATCGTTGTTGTAGCTTGGCAAG |
| | TCCGCCTCCGACACCGCATGTGTGGCAGTACCAGACGCCCTTGTCGAGGTTGATGCTCATGGAGGGCTGGTGGTCGTCGTGG |
| | AACGGGCAGAGGATGTGTTGCTCGTTCTTGGATGGGTTGTACCGTATGTGGTAGGTGTCGAGGAGGCGGCAGGTGTCAGAGG |
| | TGTGGGAGGAGTTCTGTGAGGGTTGATACCACATAGGCTTCGCTCCATGGCTTGTTGCGCTGTTTCATGACGACGAGGCCGAT |
| | GGTGGACTGGTTTTCGCGGTTTCGGTGGGTTTCGTAGTTGCGTGCCTCAAGGGTTGCTTCTTTGACGAATTGGGCGAGGTGTG |
| | GCTGTCCTGCTTTCGCTTCAATCACGTATGTGTGGTTTTTGGTTTTGAGGATGAGGTCGCCTTCGTCTTCGCGGCCGTTGAGGT |
| | GGAGGCGTTCTATATCGTGGCCGGTGTCGCGTAGCTGGTGCAATAATCGTGTTTCCCATTCGGCGCCTGCCCGCCGGTTTCTT |
| | GATTGTTGTGAAGCCATCATAGTCCTTTGTGTGTTGTGGTCATGTTCCATGGCTGTTTTTCGGCGAGGGGTCCGAAGAATGTGT |
| | ATTCGGGGTAGGCTCGTAGCCGCTCGTATCGGGTGCCGTCGGGGCTTGACTGTCCGGTGCGCTGTTTGAGTACGGCGATGCGT |
| | GCCTCTGCCGGTATCGTGAGGCCGTTGCCATTATCCTCGCCACCATACAATGAGACTCCGAGGATGAGTTGTGGTTTTTCGGA |
| | GAGCCCGTTTTTGATTTCCCGCCTAGCCGGGGGGTGTTCGATGTCGGC |

Provided are nonwoven polymer fabrics for use in a personal care product. The nonwoven polymer fabric comprises a phage combination attached to a polymer fabric, wherein the phage combination comprises one or more sets of phages that infect one or more bacterial species. The one or more bacterial species are selected from a population of microorganisms comprising Staphylococcus aureus, Staphylococcus argenteus, Streptococcus pyogenes, Streptococcus pneumoniae, Escherichia coli, Enterococcus faecalis, Cutibacterium acnes, Corynebacterium tuberculostearicum, or combinations thereof. The personal care product, for example, is a facemask, a surgical facemask, a sanitary pad, a tampon, a baby diaper, or adult incontinence products.

In some embodiments, the personal care product is a surgical facemask with an inner sheet made from nonwoven fabrics such as polypropylene, a rayon/polyester blend, or polycaprolactone (PCL). Facemasks abrade the face and increase moisture and temperature on the skin. Bacteria including Staphylococcus aureus, Staphylococcus argenteus and Cutibacterium acnes proliferate on the interior surface of facemasks as well as on the surface of the skin. Proliferating bacteria and irritated, moist skin promotes the development of folliculitis, called maskne, a portmanteau of mask and acne. To reduce the proliferation of S. aureus/argenteus and C. acnes, phages that infect those species are attached to the nonwoven fabric by the following method. The surface of the nonwoven fabric is activated by imparting a positive charge via surface plasma treatment or corona discharge treatment. Phages are attached to the activated surface of the nonwoven fabric by spraying, misting, printing, or immersing the fabric in a low-salt buffer (salts at concentration less than 50 mM) containing at least 109 PFU/mL (PFU=plaque forming units). The naturally negatively charged capsids of the phages attach to the charged, activated surface of the nonwoven fabric, leaving the positively charged phage tail prone to infect and kill bacteria that contact the surface of the nonwoven fabric incorporated into the facemask (See FIG. 1). Alternatively, phages may be covalently bonded to the nonwoven polypropylene or rayon/polyester fabric via surface activation chemistry such as EDC/NHS chemistry or acidic surface activation that enables amine groups on amino acid side-chains on the phage capsid to covalently bond to activated carboxyl groups on the polymer surface.

In some embodiments, the nonwoven polymer fabric is used in a diaper for adult incontinence with a topsheet made from nonwoven fabrics such as curled-fiber cellulose fiber, polyester fiber, polyethylene fiber, nylon fiber, polypropylene fiber, polylactic acid fiber, polyhydroxyalkanoate fiber, polyethylene terephthalate fiber, polypropylene carbonate fiber, or mixtures of the different fibers. When urine is absorbed by the diaper and held against the skin, it increases moisture and pH, reduces the natural moisture barrier of the skin, causes redness, broken skin, and sometimes bleeding. Bacteria such as Enterococcus species and Escherichia coli proliferate and exacerbate incontinence associated dermatitis (IAD). To reduce proliferation of bacteria associated with IAD, phages that target the Enterococcus species and E. coli will be attached to the nonwoven polymer topsheet of the diaper in a similar fashion as in the case of the surgical facemask, activating the surface by imparting a charge to bind to the negatively charged phage capsid or creating active sites for covalently bonding the phage capsid.

The nonwoven polymer fabric can be used in a baby diaper with a topsheet made from nonwoven fabrics such as polyester fiber, polyethylene fiber, polypropylene fiber, rayon/viscose fiber, polylactic acid fiber, acrylics, olefins, polyhydroxyalkanoate fiber, polyethylene terephthalate fiber, polypropylene carbonate fiber, or mixtures of the different fibers. Diaper dermatitis (DD) in babies is strongly associated with Candida albicans, Staphylococcus aureus/argenteus, and Enterococcus species. S. aureus/argenteus in particular produces an exfoliative toxin that can exacerbate DD and shows a strong statistical correlation with DD. Phage that infect S. aureus/argenteus and Enterococcus species can be attached to the nonwoven polymer topsheet of the diaper in a similar fashion to the surgical facemask.

The nonwoven polymer fabric can be used in a sanitary pad with a topsheet made from nonwoven fabrics such as polypropylene fiber, polyethylene fiber, polyethylene/polypropylene fiber, polylactic acid fiber, polyhydroxyalkanoate fiber, polyethylene terephthalate fiber, polypropylene carbonate fiber, or mixtures of the different fibers. Pad rash is an unpleasant but non-life-threatening complication of wearing sanitary pads. The sensitive skin around the vagina becomes inflamed due to friction from the pad, increased moisture, pH, and temperature. Skin occlusion also generally promotes the proliferation of bacteria on the surface of the skin that can contribute to pad rash. Less often, but more critically, sanitary pads can promote the growth of Staphylococcus aureus, Staphylococcus argenteus or Streptococcus species, which enter the vagina and can reach the bloodstream. If they reach the bloodstream, they may cause Device-Associated Menstrual Toxic Shock Syndrome, which is life-threatening. Phage that infect S. aureus/argenteus and Streptococcus species can be attached to the nonwoven polymer topsheet of the sanitary pad in a similar fashion to the surgical facemask. The phage-functionalized topsheet prevents proliferation of S. aureus/argenteus and Streptococcus species in the pad and on the surface of the skin, thus reducing the occurrence of pad rash and Device-Associated Menstrual Toxic Shock Syndrome.

In some embodiments, the nonwoven polymer fabric is used in a tampon with a topsheet made from nonwoven fabrics such as polypropylene fiber, polyhydroxyalkanoate fiber, polyethylene fiber, polyethylene terephthalate fiber, polylactic acid fiber, polypropylene carbonate fiber, or mixtures of the different fibers. Tampons can promote the growth of Staphylococcus aureus, Staphylococcus argenteus or Streptococcus species when not changed after 4-8 hours. The bacteria can reach the bloodstream. If they reach the bloodstream, they may cause Device-Associated Menstrual Toxic Shock Syndrome, which is life-threatening. Phage that infect S. aureus, S. argenteus and Streptococcus species can be attached to the nonwoven polymer topsheet of the sanitary pad in a similar fashion to the surgical facemask. The phage-functionalized topsheet prevents proliferation of S. aureus/argenteus and Streptococcus species in the pad and on the surface of the skin, thus reducing the occurrence of Device-Associated Menstrual Toxic Shock Syndrome.

Phage isolates used in formulating phage-functional activated polymer fabric (e.g., nonwoven fabric) surfaces are provided in Table 1.

### 6.2.1. Formulation

Bacteriophage formulations have been described in WO2022198091A1, which is incorporated by reference herein in its entirety. The bacteriophage formulations or mixtures may comprise at least one bacteriophage capable of lysing a microorganism (e.g., bacteria) or inhibiting growth of a microorganism, e.g., a microorganism found in a biological sample (e.g., skin, gut, mouth) that is obtained from a subject, and may be useful, in some instances, in treating a health condition (e.g., skin condition or disorder). A method may comprise determining or identifying a microorganism in the sample (e.g., identifying a host bacterium) and generating a bacteriophage mixture or formulation comprising at least one bacteriophage capable of infecting the microorganism. The bacteriophage mixture or formulation may be administered to the subject from which the sample was obtained. Accordingly, the methods provided herein may be useful in generating personalized formulations or mixtures to treat individual health conditions. In some instances, the bacteriophage mixture or formulation comprises at least two different bacteriophages, which may have the same or different host ranges. Such a combination of bacteriophages with a designated host range may aid in prevention of bacterial or microbial resistance.

The present disclosure also provides formulations for incorporating a bacteriophage or bacteriophage mixture, e.g., to a personal care product. A formulation may comprise a cosmetic formulation of skincare formulation. The formulation may comprise any useful ingredients or compositions, such as an excipient that is configured to stabilize the one or more bacteriophages (e.g., maintain a percentage of viability of the one or more bacteriophages for a duration of time, e.g., at least one week). The excipient may comprise a substance for bulking up a solid, liquid, or gel formulation comprising the one or more bacteriophages. In some cases, the substance may confer a therapeutic enhancement to the one or more bacteriophages, e.g., by enhancing solubility, decreasing or increasing dissolution, enhancing stability, increasing penetration into the skin or portion thereof (e.g., the stratum corneum), increasing bacteriophage activity, etc. In some instances, the excipient may comprise one or more liposomes or lipophilic moieties (e.g., micelles, vesicles), which may encapsulate the one or more bacteriophages. The excipient or a substance of the excipient may be used to change a property of the composition, such as the viscosity. The substance may be used to change a property of the therapeutic agent, e.g., bioavailability, absorption, hydrophilicity, hydrophobicity, pharmacokinetics, etc. The excipient may comprise a binding agent, anti-adherent agent, a coating, a disintegrant, a glidant (e.g., silica gel, talc, magnesium carbonate), a lubricant, a preservative, a sorbent, a sweetener, a vehicle, or a combination thereof. For instance, the excipient may comprise a powder, a mineral, a metal, a sugar (e.g. saccharide or polysaccharide), a sugar alcohol, a naturally occurring polymer (e.g., cellulose, methylcellulose) synthetic polymer (e.g., polyethylene glycol or polyvinylpyrrolidone), an alcohol, a thickening agent, a starch, a macromolecule (e.g., lipid, protein, carbohydrate, nucleic acid molecule), etc.

The formulation may comprise additional components for treating a skin condition (e.g., aging, acne). For example, the formulation may comprise one or more anti-aging ingredients, including but not limited to: retinoids (vitamin A derivatives), niacinamide (vitamin B3), ascorbic acid (vitamin C), skin-active peptides, proteins or peptides (e.g., collagen, hyaluronic acid, or derivatives thereof), plant growth factors, e.g., kinetin, ubiquinone (coenzyme Q10), etc. Alternatively or in addition to, the formulation may comprise one or more anti-inflammatory agents, e.g., anti-histamines, salicylates, and the like. In some instances, the formulation may comprise one or more anti-acne agents, such as benzoyl peroxide, salicylic acid, probiotics, antibiotics, antifungals, etc.

In some embodiments, the composition contains one or more bacteriophages formulated in a mixture. In some embodiments, the composition is formulated in a liquid, semi-liquid, solid, semi-solid, or powder form. In some embodiments, the composition is formulated for attaching the phages to surface-active nonwoven fabrics and is integrated into disposable personal care products. In some embodiments, the composition is formulated in a liquid for applying the composition onto surface-active non-woven fabrics.

In some embodiments, the composition contains one or more bacteriophages formulated in a culture medium (e.g., a cocktail of bacteriophages).

Polymer fabrics with phage combination as described herein alone or incorporated on personal care products may be maintained, stored, or shipped in a culture having a pH value between 1-14. Suitable pH values are about pH 1, pH 2, pH 3, pH 4, pH 5, pH 6, pH 7, pH 8, pH 9, pH 10, pH 11, pH 12, pH 13, or pH 14. As an example, the pH value may be an acidic pH or lower than pH 7 (e.g., pH 6.9, pH 6.5, pH 6, pH 5, pH 4, pH 3, pH 2, pH 1). As another example, the pH value may be an alkaline pH or higher than pH 7 (e.g., pH 7.1, pH 7.5, pH 8, pH 9, pH 10, pH 11, pH 12, pH 13, or pH 14). As yet another example, the pH value may be close to neutral (e.g., about pH 7, about pH 6-pH 8).

Polymer fabrics attached with phage combination as described herein or personal care products incorporated with the phage combination or the phage cocktail can be packaged, stored, or shipped at about 4° C. to about 100° C., about 10° C. to about 80° C., about 15° C. to about 60° C., about 20° C. to about 40° C., or about 25° C. to about 35° C. In some embodiments, the personal care product incorporated with the bacteriophages or the bacteriophage cocktail is packaged, stored, or shipped at a temperature no more than 100° C., 90° C., 80° C., 70° C., 60° C., 50° C., 40° C., 30° C., 20° C., 10° C., or 4° C. In some embodiments, the personal care product incorporated with the bacteriophages or the bacteriophage cocktail is packaged, stored, or shipped at room temperature.

### 6.2.2. Polymer Fabrics

In some embodiments, the polymer fabric is a polymer matrix. As used herein, the term “polymer matrix” means a polymer which undergoes a physicochemical change in response to a compound produced or released by the infection of a microorganism by a phage. The polymer may be degradable or non-degradable. Suitable polymers include, but are not limited to, polylactic acid, polycaprolactones, PHA, and PHB. The polymer matrix may comprise one polymer, or a combination of polymers.

In some embodiments, the polymer fabric is a polymer matrix and comprises a composite of polymers. In some embodiments, the polymer matrix comprises two polymer layers. In some embodiments, the polymer matrix comprises a degradable polymer and/or a non-degradable polymer. The polymer matrix can be patterned at the nano-, micro- or millimeter-scale, or a combination thereof. Such patterning confers a distinctive appearance to the polymer matrix which may comprise a pure polymer or a polymer blended with a non-degradable polymer or non-polymeric material. In some embodiments, the top layer comprises a degradable polymer, and the bottom layer comprises a porous polymer.

In some embodiments, the polymer matrix is a free-standing film of polymer, a blend of polymers, or a composite material. In some embodiments, the polymer matrix is coated onto a carrier layer in the form of a film or a coating.

In some embodiments, the polymer fabric is nonwoven fabric. As used herein, the term “nonwoven fabric” refers to a fabric-like material made from staple fibre (short) and long fibres (continuous long), bonded together by chemical, mechanical, heat or solvent treatment. Nonwoven fabrics are engineered fabrics that may be single-use, have a limited life, or be very durable. Nonwoven fabrics provide specific functions such as absorbency, liquid repellence, resilience, stretch, softness, strength, flame retardancy, washability, cushioning, thermal insulation, acoustic insulation, filtration, use as a bacterial barrier and sterility. Nonwoven fabrics can be used alone or as components of personal care products such as facemask, sanitary pad, tampon, baby diaper, or adult incontinence products.

In some embodiments, the nonwoven fabric is treated with plasma surface treatment to create surface-active nonwoven fabrics. For instance, phages can be covalently or noncovalently attached to surface-active nonwoven fabrics using methods described herein and disclosed in U.S. Pat. No. 9,277,751; Wang et al., Immobilization of active bacteriophages on polyhydroxyalkanoate surfaces. ACS Appl. Mater. Interfaces, 216, 8, 2, 1128-1138, each of which is incorporated by reference herein in its entirety.

In some embodiments, phages are noncovalently attached to surface-active nonwoven fabrics integrated into disposable personal care products. The phages are attached so that the capsid adsorbs to the surface of the nonwoven fabric via electrostatic charge and the tail is exposed away from the surface of the fabric. In some embodiments, misting is used to attach phages to surface-active nonwoven fabrics.

In some embodiments, phages are immobilized to the polymer fabric (e.g., nonwoven fabric) by chemical bonding, physical adsorption, bulk mixing, or a combination thereof. One or more phages may be immobilized to the polymer fabric to enable killing or suppressing growth of multiple microorganism strains. Methods for immobilizing phages to polymer matrix are described in U.S. Pat. No. 9,921,219, which is incorporated by reference herein in its entirety. Suitable methods for immobilization include, but are not limited to, biotinylation of the phage capsid for biotin-streptavidin affinity bonding (Gervais et al., 2007; Edgar et al., 2006; Smelyanski et al., 2011); direct covalent bonding using n-hydroxysulfosuccinimide and 1-ethyl-3-(3-dimethylaminopropyl) carbodiimide as linkers (Hermanson, 2008); and plasma-treated surface bonding (Pearson et al., 2013), each of which is incorporated by reference herein in its entirety.

### 6.2.3. Personal Care Products

In some embodiments, phage combinations are attached to a personal care product that directly contacts or has prolonged contact with a subject's skin. Non-limiting examples of personal care products are facemasks, sanitary pads, tampons, baby diapers, adult incontinence products, cosmetics, cosmetic facial pads or wipes, fingernail wipes, or patient bathing hygiene textile.

As described herein, the personal care product can be a facemask incorporated with the phage combination. The facemask can be a surgical or a non-surgical facemask. Such a facemask typically has an inner sheet made from nonwoven fabrics such as polypropylene, a rayon/polyester blend, or polycaprolactone (PCL). Facemasks abrade the face and increase moisture and temperature on the skin. Bacteria including Staphylococcus aureus, Staphylococcus argenteus and Cutibacterium acnes proliferate on the interior surface of facemasks as well as on the surface of the skin. Proliferating bacteria and irritated, moist skin promotes the development of folliculitis, called maskne, a portmanteau of mask and acne. To reduce the proliferation of S. aureus, S. argenteus and C. acnes, phages that infect those species are attached to the nonwoven fabric by methods as described herein, for example in Example 4.

The personal care product can be a diaper for adult incontinence incorporated with the phage combination. The diaper may have a top sheet made from nonwoven fabrics such as curled-fiber cellulose fiber, polyester fiber, polyethylene fiber, nylon fiber, polypropylene fiber, polylactic acid fiber, polyhydroxyalkanoate fiber, polyethylene terephthalate fiber, polypropylene carbonate fiber, or mixtures of the different fibers. When urine is absorbed by the diaper and held against the skin, it increases moisture and pH, reduces the natural moisture barrier of the skin, causes redness, broken skin, and sometimes bleeding. Bacteria such as Enterococcus species and Escherichia coli proliferate and exacerbate incontinence associated dermatitis (IAD). To reduce proliferation of bacteria associated with IAD, phages that target Enterococcus species and E. coli may be attached to the nonwoven polymer top sheet of the diaper in a similar fashion as in the case of the surgical facemask, activating the surface by imparting a charge to bind to the negatively charged phage capsid or creating active sites for covalently bonding the phage capsid.

As another example, the personal care product can be a baby diaper incorporated with the phage combination. The diaper may have a top sheet or outward facing surface made from nonwoven fabrics such as polyester fiber, polyethylene fiber, polypropylene fiber, rayon/viscose fiber, polylactic acid fiber, acrylics, olefins, polyhydroxyalkanoate fiber, polyethylene terephthalate fiber, polypropylene carbonate fiber, or mixtures of the different fibers. Diaper dermatitis (DD) in babies is strongly associated with Candida albicans, Staphylococcus aureus, S. argenteus, and Enterococcus species. S. aureus, S. argenteus in particular produces an exfoliative toxin that can exacerbate DD and shows a strong statistical correlation with DD. Phage that infect S. aureus, S. argenteus and Enterococcus species can be attached to the nonwoven polymer top sheet of the diaper in a similar fashion to the surgical facemask.

The personal care product can be a feminine care product such as a sanitary pad incorporated with the phage combination. The sanitary pad may have a top sheet or outer-facing surface made from nonwoven fabrics such as polypropylene fiber, polyethylene fiber, polyethylene/polypropylene fiber, polylactic acid fiber, polyhydroxyalkanoate fiber, polyethylene terephthalate fiber, polypropylene carbonate fiber, or mixtures of the different fibers. Pad rash is an unpleasant but non-life-threatening complication of wearing sanitary pads. The sensitive skin around the vagina becomes inflamed due to friction from the pad, increased moisture, pH, and temperature. Skin occlusion also generally promotes the proliferation of bacteria on the surface of the skin that can contribute to pad rash. Less often, but more critically, sanitary pads can promote the growth of Staphylococcus aureus, S. argenteus or Streptococcus species, which enter the vagina and can reach the bloodstream. If they reach the bloodstream, they may cause Device-Associated Menstrual Toxic Shock Syndrome, which is life-threatening. Phage that infect S. aureus, S. argenteus and Streptococcus species can be attached to the nonwoven polymer topsheet of the sanitary pad in a similar fashion to the surgical facemask. The phage-functionalized topsheet prevents proliferation of S. aureus, S. argenteus and Streptococcus species in the pad and on the surface of the skin, thus reducing the occurrence of pad rash and Device-Associated Menstrual Toxic Shock Syndrome.

Another example of a personal care product is a tampon incorporated with the phage combination. The tampon may have a top sheet or outer surface made from nonwoven fabrics such as polypropylene fiber, polyhydroxyalkanoate fiber, polyethylene fiber, polyethylene terephthalate fiber, polylactic acid fiber, polypropylene carbonate fiber, or mixtures of the different fibers. Tampons can promote the growth of Staphylococcus aureus, S. argenteus or Streptococcus species when not changed after a period of time (e.g., about 4-8 hours). The bacteria can reach the bloodstream. If they reach the bloodstream, they may cause Device-Associated Menstrual Toxic Shock Syndrome, which is life-threatening. Phage that infect S. aureus/argenteus and Streptococcus species can be attached to the nonwoven polymer topsheet of the sanitary pad in a similar fashion to the surgical facemask. The phage-functionalized topsheet prevents proliferation of S. aureus, S. argenteus and Streptococcus species in the pad and on the surface of the skin, thus reducing the occurrence of Device-Associated Menstrual Toxic Shock Syndrome.

### 6.3. Methods

### 6.3.1. Identification of Bacteriophage Combination

Selection of bacteriophages: Bacteriophages may be selected for inclusion in a mixture or formulation based on any useful characteristic. Characteristics that may be used for selection of bacteriophages, include, in non-limiting examples, non-lysogeny or non-integration in a host genome, host range (e.g., specific to a host organism with minimal off target binding), and stability in a given formulation. In some instances, combinations of bacteriophages may be selected for a designated microbiome type or host range (e.g., for targeting a given bacterial strain such as C. acne), which in combination may aid in preventing bacterial resistance.

Accordingly, in a second aspect, disclosed herein are methods for selecting the phages to target the bacteria most commonly associated with contact dermatitis. In certain embodiments, contact dermatitis is skin presentation of one or more of diaper rash, maskne, sanitary pad rash, or incontinence-associated rash.

In some embodiments, the method further comprises screening skin samples from a subject or a population of subjects to identify bacteria commonly present and are associated with contact dermatitis. Bacteriophage assays are as described in WO2022198091A1, which is incorporated by reference herein in its entirety.

Bacteriophage assays: Screening of the samples may be performed to determine one or more agents (e.g., small molecules, bacteriophages, or biological molecules, e.g., proteins, peptides, lipids, carbohydrates, metabolites, or combinations thereof) capable of inhibiting growth or otherwise killing (e.g., via lysing) of one or more microorganisms of the samples. In some examples, the one or more agents comprises one or more bacteriophages. For example, the one or more bacteriophages may be naturally occurring, recombinant, or synthetic bacteriophages that are capable of lysing a type of bacteria present on the skin sample. In such an example, the screening of the samples may comprise: obtaining one or more microorganisms from the sample, optionally culturing the one or more microorganisms, and exposing the one or more microorganisms to a plurality of bacteriophages to generate a microorganism-bacteriophage mixture. The growth, proliferation, or death of the microorganisms may be monitored over a duration of time, and the microorganisms may be collected, stored, and/or sequenced. Sequencing (e.g., shotgun metagenomic sequencing) may yield information on the bacteriophages that are present in or on the microorganisms and that may be capable of killing or inhibiting growth of the one or more microorganisms. Accordingly, such screening, e.g., the combination of culturing microorganisms with bacteriophages and sequencing the mixture, may be useful in determining combinations of bacteriophages to remove particular microorganism (e.g., bacterial) strains.

In some instances, the screening may comprise sequencing one or more microorganisms isolated from a sample. For example, the one or more microorganisms isolated from the sample may be (i) cultured to generate a microorganism library and (ii) sequenced (e.g., via shotgun metagenomic sequencing) to generate a library of sequence reads comprising sequences from the microorganisms present within the sample. Additionally, the cultured microorganisms may be further exposed to one or more bacteriophages, and then isolated and sequenced, as described above. The sequences corresponding to the one or more bacteriophages may be stored in a bacteriophage library (Table 1). Alternatively or in addition to, the one or more bacteriophages may be stored and further screened, e.g., using a bacteriophage host range assay, to determine bacterial strains which may be susceptible to the one or more bacteriophages.

Bacteriophage source: The one or more bacteriophages may be synthetic or naturally occurring. The one or more bacteriophages may be isolated or extracted from an environmental source, including, but not limited to: sewage, soil suspensions, ocean sediment, terrestrial sub surfaces, etc. In such instances, further processing of the environmental source may be performed to purify or enrich for the bacteriophages.

Bacteriophage host range assay: A bacteriophage host range assay may be used to determine a host or suite of host cells (e.g., bacterial cells) that are susceptible to a given bacteriophage in vitro. In some instances, the bacteriophage host range assay is performed using the one or more microorganisms isolated from the sample, or a plurality of samples, and one or more bacteriophages present in an environmental sample. For example, bacteria from one or more skin samples may be collected and optionally, a portion of the collected bacteria may be sequenced for identification of the bacteria type (e.g., bacterial species or variant). The collected bacteria may be cultured (e.g., on agar plates), distributed on a phage host range assay dish, and contacted with viral fractions from an environmental source, which may comprise one or more bacteriophages (e.g., lytic bacteriophages). Optionally, the collected bacteria may be pre-sorted or processed, such that only one strain type or class of strain types is present per dish of the phage host range assay. The bacteria-bacteriophage mixtures on the phage host range assay may optionally be cultured. The bacteriophages that grow on or within the collected and cultured bacteria and/or lyse or prevent growth of the cultured bacteria on the assay may be isolated, optionally propagated, and stored as part of a bacteriophage library. Alternatively or in addition to, the bacteriophages may be sequenced. Such sequencing data may then be used to associate each of the isolated bacteriophages with a particular host organism. Accordingly, such an assay may be useful in determining the identities and genomic sequences of bacteriophages that are capable of removing one or more microorganisms from a sample. As the identities of the one or more microorganisms are also known (e.g., from sequencing), the host range of each bacteriophage may be determined. Accordingly, for a given target bacterial strain, an appropriate bacteriophage may be selected for inclusion in a formulation designed to lyse the targeted bacterial strain.

### 6.3.2. Attaching Bacteriophage Combination to Polymer Fabric

Also disclosed herein are methods for attaching phages to polymer fabric that is incorporated into disposable personal care products. In some embodiments, the polymer fabric is nonwoven fabric.

The surface of the polymer fabric (e.g., nonwoven fabric) is activated by imparting a positive charge via surface plasma treatment or corona discharge treatment. Phages are attached to the activated surface of the polymer fabric (e.g., nonwoven fabric) by spraying, misting, or printing. In certain embodiments, the phages are attached to the activated surface of the polymer fabric by misting. The polymer fabric (e.g., nonwoven fabric) may be immersed in a low-salt buffer (salts at concentration less than 50 mM) containing at least 109 PFU/mL (PFU=plaque forming units). Without being bound to any theory, the naturally negatively charged capsids of the phages attach to the charged, activated surface of the nonwoven fabric, leaving the positively charged phage tail prone to infect and kill bacteria that contact the surface of the polymer fabric (e.g., nonwoven fabric) incorporated into the facemask. Alternatively, phages may be covalently bonded to the nonwoven polypropylene or rayon/polyester fabric via surface activation chemistry such as EDC/NHS chemistry or acidic surface activation that enables amine groups on amino acid side-chains on the phage capsid to covalently bond to activated carboxyl groups on the polymer surface.

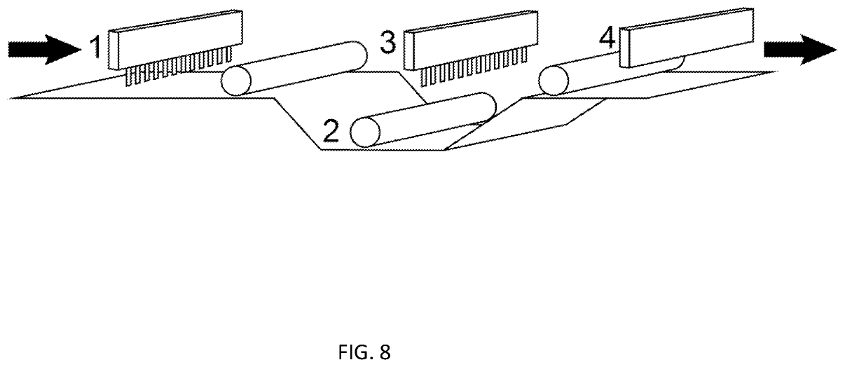

An exemplary method for attaching bacteriophage combination to a polymer fabric is shown in FIG. 8, it describes a processes enabling the treatment of nonwoven fabrics of diverse composition to be impregnated with phages of diverse morphology and host specificity after plasma treatment of the nonwoven fabric inline during a manufacturing process as follows: (1) The nonwoven fabric is plasma-treated (either encased or open-air plasma treatment) in-line in an automated manufacturing process; (2) Surface-active nonwoven fabric can be impregnated on both surfaces using a dip tray (also called an impregnation tank or dip tank); and (3) The nonwoven fabric is gently dried with an air dryer at temperatures below 50° C. that do not denature phage virions

### 6.3.3. Subject

The term “subject,” as used herein, generally refers to an animal, such as a mammal (e.g., human) or avian (e.g., bird), or other organism, such as a plant. For example, the subject can be a vertebrate, a mammal, a rodent (e.g., a mouse), a primate, a simian or a human.

Animals may include, but are not limited to, farm animals, sport animals, and pets. A subject can be a healthy or asymptomatic individual, an individual that has or is suspected of having a disease (e.g., skin conditions, cancer), or a pre-disposition to the disease, an infection, a health condition, and/or an individual that is in need of therapy or suspected of needing therapy. A subject can be a human. A subject can be a patient.

In some embodiments, the subject has acne. In some embodiments, the subject has one or more skin conditions such as but are not limited to acne, diaper rash, maskne, sanitary pad rash, and incontinence-associated rash, redness, eczema, rosacea, aged skin, or skin infection. The subject may have or at risk (e.g., more susceptible or prone to be having the condition) of having one or more skin conditions caused by or associated with a particular microbiome subtype. The subject may have one or more microbial variants detected in or living on the subject's skin.

In accordance with the provided methods of treatment, the bacteriophage mixtures or formulations may be used to treat a skin condition which may be caused by or associated with a particular microbiome subtype (e.g., a particular microorganism, such as a bacterial strain, or combination of microorganisms). For example, a formulation described herein may be used to treat acne, inflammation, redness, eczema, rosacea, enlarged hair follicle pore size, rough skin texture, increased trans-epidermal water loss, skin dehydration, skin discoloration (e.g., hyperpigmentation), disproportionate elasticity of the skin or portion thereof (stratum corneum, dermis, etc.). The formulation may comprise one or more bacteriophages that are capable of lysing or inhibiting growth of one or more microorganisms that are associated with a skin condition, e.g., Cutibacterium acnes or Staphylococcus aureus, which may be associated with acne.

As described herein, the skin samples may comprise one or more microorganisms associated with a skin condition (e.g., acne, eczema, redness, etc.). Accordingly, the bacteriophage mixtures and formulations may be targeted for any of the species or variants of skin microorganisms which may be associated with the skin condition. For instance, one or more bacteriophages may target a bacteria, such as a bacteria from the Actinobacteria, Firmicutes, Proteobacteria, Bacteroidetes phyla. The one or more microorganisms may comprise, in some examples, a Cutibacterium bacterium (e.g., C. acnes, C. namnatense, C. avidum), a Staphylococcus bacterium (e.g., S. aureus, S. epidermidis, S. warneri, S. pyogenes, S. mitis), a Corynebacterium bacterium, an Acinelobacler bacterium (e.g A. johnsonii), a Pseudomonas bacteria (e.g., P. aeruginosa), other bacteria, or combinations thereof. In some instances, the one or more bacteriophages may be targeted for a fungal microorganism, including, but not limited to: yeasts, such as Candida albicans, Rhodotorula rubra, Torulopsis and Trichosporon cutaneum, dermatophytes such as Microsporum gypseum, and Trichophyton rubrum, and nondermatophyte fungi such as Rhizopus stolonifer, Trichosporon cutaneum, Fusarium, Scopulariopsis brevicaulis, Curvularia, Alternaria alternata, Paecilomyces, Aspergillus flavus and Penicillium. The one or more bacteriophages may target a combination of microorganisms; such a combination may be determined, for example, using a host range assay, as described elsewhere herein or in WO2022198091A, which is incorporated by reference herein in its entirety.

### 6.3.4. Kits

Also provided herein are kits for incorporating phage combination attached to a synthetic nonwoven polymer fabric in a disposable personal care product used in close contact with the skin. Such a kit may comprise a phage combination non-covalently attached to a polymer fabric (e.g., synthetic nonwoven polymer fabric). The kit may comprise phage combination in a culture for attaching the phage combination onto a polymer fabric (e.g., synthetic nonwoven polymer fabric). Such a kit may comprise instructions for attaching the phage combination onto the polymer fabric. The kit may comprise, for instance, reagents, such as stabilization agents, buffers, preservatives, fixatives, or pH balancing reagents. The kit may additionally comprise instructions for applying the personal care product incorporated with the polymer fabric, storage, transportation, or treatment.

### 6.4. Treatment Regimens

In the methods provided herein, the terms “treatment”, “treating”, and the like are used herein to generally mean obtaining a desired pharmacologic and/or physiologic effect. In certain embodiments, the effect is therapeutic in terms of a partial or complete response of the microorganism growth which includes inhibiting growth or preventing regrowth of the microorganism, and/or reducing or eliminating one or more symptoms associated with contact dermatitis.

### 6.4.1. Combination Administration

In some embodiments of the provided methods for treating one or more skin conditions, the phage combination is administered to a subject in combination with one or more skin care products or medications. For instance, the phage combination attached to a polymer fabric (e.g., nonwoven fabric) may be used in combination with a skin lotion, cream, toner, cleanser, and/or wipe. In another example, the phage combination attached to a polymer fabric (e.g., nonwoven fabric) may be used in combination with a medical procedure, e.g., a surgery. In such example, polymer fabric (e.g., a mesh wound dressing) containing the phage combination may be applied onto a wound, a cut, or a surgery site to prevent bacterial growth or infection.

### 6.4.1.1 Administration of Bacteriophage

In various embodiments, the phage combination attached to a polymer fabric (e.g., nonwoven fabric) is incorporated into a personal care product for administration to a subject. In some cases, the phage combination attached to a polymer fabric (e.g., nonwoven fabric) is administered to a subject by directly applying the polymer fabric onto the skin of the subject.

In some embodiments, the phage combination is administered to the subject by the subject, a physician, a health care professional, and/or a caretaker.

In certain embodiments, the phage combination is administered once, twice, three times, four times, or more every day. In certain embodiments, the phage combination is administered once every day. In certain embodiments, the personal care product incorporated with the phage combination is administered once every two days. In certain embodiments, the personal care product incorporated with the phage combination is administered once every three days. In certain embodiments, the personal care product incorporated with the phage combination is administered once every four days. In certain embodiments, the personal care product incorporated with the phage combination is administered once every five days. In certain embodiments, the personal care product incorporated with the phage combination is administered once every six days. In certain embodiments, the personal care product incorporated with the phage combination is administered once every week. In certain embodiments, the personal care product incorporated with the phage combination is administered once every two weeks. In certain embodiments, the personal care product incorporated with the phage combination is administered once every three weeks. In certain embodiments, the personal care product incorporated with the phage combination is administered once every month. In certain embodiments, the personal care product incorporated with the phage combination is administered as needed.

In some embodiments, the personal care product incorporated with the phage combination is administered to a subject until the subject's skin condition (e.g., contact dermatitis) is partially or fully eliminated. In some embodiments, the personal care product incorporated with the phage combination is administered to the subject reduces one or more symptoms of contact dermatitis or skin infection, and/or reduces body odor.

### 7. EXAMPLES

### 7.1. Example 1: Composition of Multiple Phages that Infect Bacteria on the Surface of the Skin that are Associated with Contact Dermatitis

In this example, phages capable of lysing or killing one or more bacteria including Staphylococcus aureus, Staphylococcus argenteus, Streptococcus pyogenes, Streptococcus pneumoniae, Escherichia coli, Enterococcus faecalis, Cutibacterium acnes, and Corynebacterium tuberculostearicum are identified. The combination of multiple phages that target or infect the bacteria most commonly associated with contact dermatitis are attached to plasma surface-treated synthetic nonwoven polymer fabrics including polylactic acid (PLA), polyester (PE), acrylics, olefins, polyhydroxyalkanoates (PHA) such as polyhydroxybutyrate (PHB), polyethylene terephthalate (PET), polypropylene (PP) and polypropylene carbonate (PPC). The surface-treated nonwoven fabric is incorporated in a disposable personal care product that is in close contact with the skin, such as a facemask, sanitary pad, tampon, baby diaper, adult incontinence products, or cosmetic facial pads or wipes, fingernail wipes, or patient bathing hygiene textiles.

### 7.2. Example 2: Selecting the Phages to Target the Bacteria Most Commonly Associated with Contact Dermatitis

In this example, phages that target or infect the bacteria described in Example 1 are selected.

Those bacterial strains are cultivated. Phages infecting those strains are isolated from environmental samples. The environmental samples are enriched for phages by adding enrichment broth and broth culture to the sample and incubating overnight. The enriched phage population is separated from the broth culture by sterile filtration. Phages are isolated from the enrichment broth filtrate by the classical top-agar plaque-plate method. Phage isolates are amplified on the target bacterial strain. DNA extractions from the isolates are sequenced to screen the phage genomes for genes related to antibiotic resistance, toxicity, lysogeny, or transduction. Screened phages are tested for host range and combined to maximize host range. Sets of phages that infect the same host species are tested in pairs for preventing bacterial regrowth in broth culture. Pairs that prevent bacterial regrowth are selected. The pairs selected may be pairs that do not occur together in nature. They may be selected to be complementary in their action for presenting bacterial regrowth. Particular subunits of the genomes of the selected phage pairs are selected for a composition, where these are selected to prevent antibiotic resistance or have various other beneficial properties, such as inhibiting bacterial growth, infecting and/or killing bacteria, and preventing bacterial regrowth.

### 7.3. Example 3: Attaching Phages to Nonwoven Fabrics that are Incorporated into Disposable Personal Care Products

In this example, phages that target or infect the bacteria described in Example 1 are attached to a nonwoven polymer fabric. Phages are concentrated from bacterial growth broth and resuspended in a low-salt phage buffer via TFF diafiltration. Plasma surface treatment is applied to the nonwoven fabric and the phage buffer is misted onto the surface-treated nonwoven fabric and dried at room temperature. The nonwoven fabric is incorporated into the disposable personal care product.

### 7.4. Example 4: Preparing Activated Polymer Fabric for Phage Combination Attachment

In this example, nonwoven fabric is used. The surface of the nonwoven fabric is activated by imparting a positive charge via surface plasma treatment or corona discharge treatment. Phages are attached to the activated surface of the nonwoven fabric by spraying, misting, printing, or immersing the fabric in a low-salt buffer (salts at concentration less than 50 mM) containing at least 109 PFU/mL (PFU=plaque forming units). The naturally negatively charged capsids of the phages attach to the charged, activated surface of the nonwoven fabric, leaving the positively charged phage tail prone to infect and kill bacteria that contact the surface of the nonwoven fabric incorporated into the facemask.

FIG. 1 illustrates the use of a composition comprising phages as described herein for use in a personal care product. A subject's skin harbors a variety of bacterial species living in or on the skin surface (1) commensally. Phage targets a specific type of bacteria (2) by recognizing the phage receptor (3) presented on a target host bacterial species. It is noted that the nontargeted skin microbial commensal (4) remains uninterrupted. The phage (5) presents a negatively charged capsid (6), which is capable of binding to the positively charged (7) and activated polymer fabric surface (8). The polymer fabric is a nonwoven fiber. The phage (5) is a myovirus tailed phage. The phage (9) is a podovirus tailed phage, a distinct phage morphotype from a myovirus tailed phage. Upon phage attachment, the polymer fabric (e.g., surface activated nonwoven-fiber) (8) is functionalized with the immobilized phage (5) or (9).

Alternatively, phages may be covalently bonded to the nonwoven polypropylene or rayon/polyester fabric via surface activation chemistry such as EDC/NHS chemistry or acidic surface activation that enables amine groups on amino acid side-chains on the phage capsid to covalently bond to activated carboxyl groups on the polymer surface.

In some cases, more than one type of phage (e.g., a myovirus and a podovirus, or a phage that infects S. aureus and a phage that infects E. coli) is attached on a fabric, such as PET or PHB, to target more than one type of bacteria present that may be present in the area on which the fabric is placed or in contact. Prior to this, it was not known whether phages such as myoviruses or podoviruses or both might be able to be attached to nonwoven fabrics, such as PET and PHB, and whether there might be challenges that prevent the attachment of, for example, both types of phages to the same piece of nonwoven fabric or a mixture of phages to a nonwoven fabric. In addition, using phage treatments on personal care products is not simple as such products may include one or more chemicals or treatments, or various fabric features that affect phage attachment or use.

### 7.5. Example 5: Attaching Phage to PET Nonwoven Fabric and Inhibiting Bacterial Growth

In this example, nonwoven fabric was used. Phages were attached to the surface of the PET nonwoven fabric and inhibition of bacterial growth was measured.

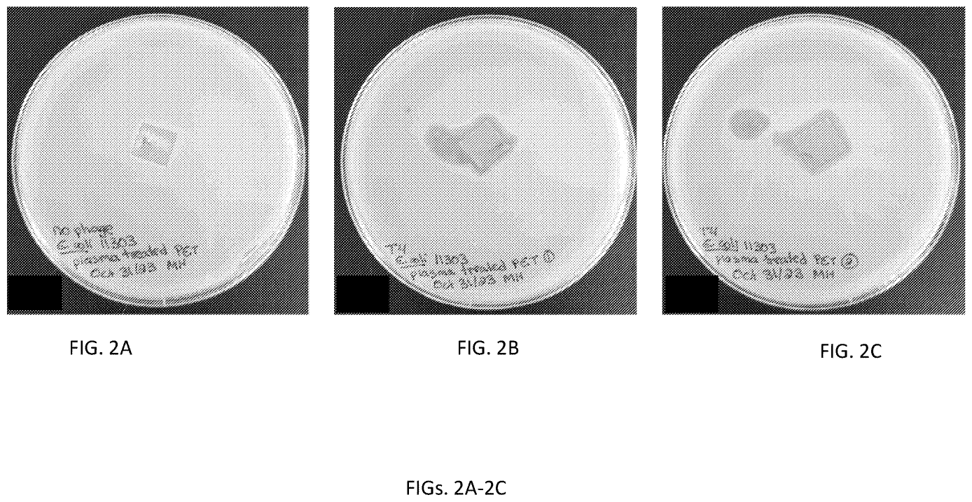

FIGS. 2A-2C show phage attached to PET nonwoven fabric and was able to inhibit E. coli growth. As shown in FIG. 2A, a 1 cm² plasma-treated PET nonwoven fabric square without phage attached to it was applied to an agar plate with a lawn of Escherichia coli strain 11303. The sharp demarcation of the fabric square indicates that plasma-treated PET alone inhibited E. coli growth. However, no halo effect due to phage infection was measured.

FIG. 2B shows a 1 cm² plasma-treated PET nonwoven fabric square with E. coli myovirus T4 attached to it and was applied to an agar plate with a lawn of Escherichia coli strain 11303. There were clear halos extending from the PET square. These halos were plaques- or lacunae-formed by phage infection of E. coli. The data demonstrate phage were successfully attached to the plasma-treated PET nonwoven fabric square.

FIG. 2C is a replicate of FIG. 2B showing repeatable phage attachment to plasma-treated PET nonwoven fabric squares.

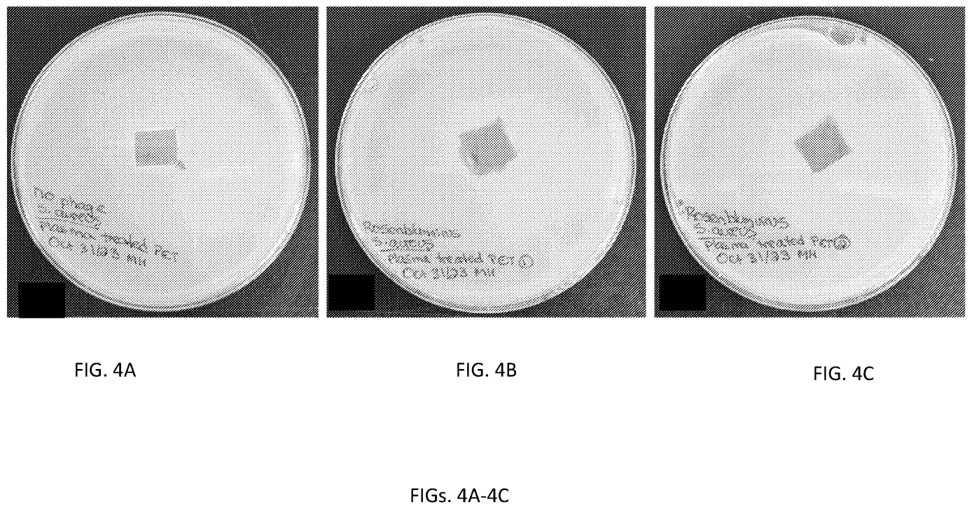

FIGS. 4A-4C show phage attached to PET nonwoven fabric and was able to inhibit Staphylococcus aureus growth. FIG. 4A shows a 1 cm² plasma-treated PET nonwoven fabric square without phage attached to it and was applied to an agar plate with a lawn of Staphylococcus aureus strain 100311. The sharp demarcation of the fabric square indicates that plasma-treated PET alone inhibited S. aureus growth. However, no halo effect due to phage infection was measured.

FIG. 4B shows an 1 cm² plasma-treated PET nonwoven fabric square with S. aureus podovirus Rosenblumvirus P00474 attached to it was applied to an agar plate with a lawn of Staphylococcus aureus strain 100311. There were clear halos extending from the PET square. These halos were plaques- or lacunae-formed by phage infection of S. aureus. E. coli myovirus T4 was successfully attached to the plasma-treated PET nonwoven fabric square and inhibited E. coli growth on a parallel solid plane such as an agar plate, in support of FIG.

FIG. 4C is a replicate of FIG. 4B showing repeatable phage attachment to plasma-treated PET nonwoven fabric squares.

In summary, the data demonstrate that, for the first time, phage was able to attach and retain on PET nonwoven fabric polymers and successfully inhibited multiple different bacterial growth.

### 7.6. Example 6: Attaching Phage to PHB Nonwoven Fabric and Inhibiting Bacterial Growth

In this example, nonwoven fabric was used. Phages were attached to the surface of the PHB nonwoven fabric and inhibition of bacterial growth.

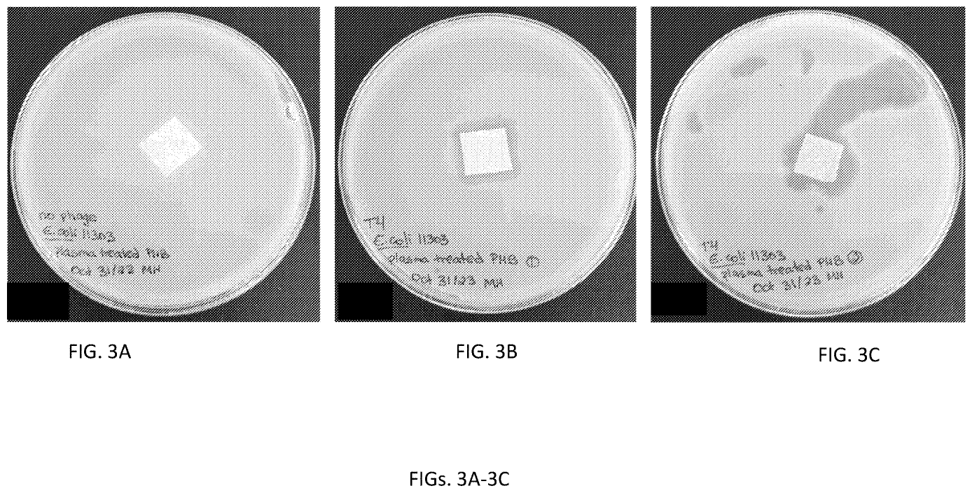

FIGS. 3A-3C show phage attached to PHB nonwoven fabric and was able to inhibit E. coli growth. As shown in FIG. 3A, a 1 cm² plasma-treated PHB nonwoven fabric square without phage attached to it and was applied to an agar plate with a lawn of Escherichia coli strain 11303. No growth inhibition of E. coli due to phage infection was measured.

FIG. 3B shows a 1 cm² plasma-treated PHB nonwoven fabric square with E. coli myovirus T4 attached to it and was applied to an agar plate with a lawn of Escherichia coli strain 11303. There were clear halos extending from the PHB square. These halos were plaques- or lacunae-formed by phage infection of E. coli. The data demonstrate that phage were successfully attached to the plasma-treated PHB nonwoven fabric square.

FIG. 3C is a replicate of FIG. 3B showing repeatable phage attachment to plasma-treated PHB nonwoven fabric squares.

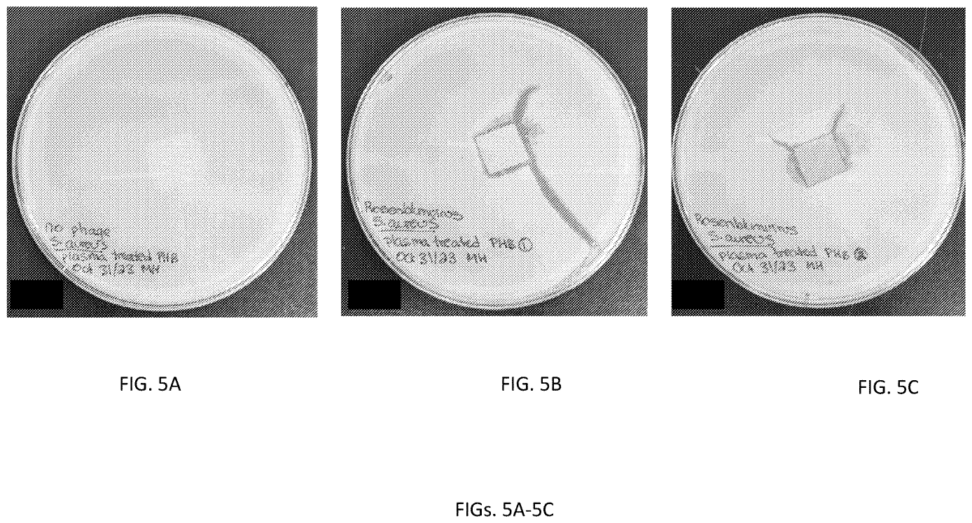

FIGS. 5A-5C show phage attached to PHB nonwoven fabric and was able to inhibit Staphylococcus aureus growth. As shown in FIG. 5A, a 1 cm² plasma-treated PHB nonwoven fabric square without phage attached to it and was applied to an agar plate with a lawn of Staphylococcus aureus strain 100311. No growth inhibition of S. aureus due to phage infection was measured.

FIG. 5B shows a 1 cm² plasma-treated PHB nonwoven fabric square with S. aureus podovirus Rosenblumvirus P00474 attached to it is applied to an agar plate with a lawn of Staphylococcus aureus strain 100311. There were clear halos extending from the PHB square. These halos were plaques- or lacunae-formed by phage infection of S. aureus. S. aureus podoviruses were successfully attached to the plasma-treated PHB nonwoven fabric square and inhibited S. aureus growth on a parallel solid plane such as an agar plate, in support of FIG. 1.

FIG. 5C is a replicate of B showing repeatable phage attachment to plasma-treated PHB nonwoven fabric squares.

In summary, the data demonstrate that phage was able to attach and retain on PHB nonwoven fabric polymers and successfully inhibited multiple different bacterial growth.

### 7.7. Example 7: Effect of T4 Myovirus Phage Attached to Nonwoven Fibers on Bacterial Growth

In this example, nonwoven fabric was used. T4 myovirus phages were attached to the surface of the PET or PHB nonwoven fabric and bacterial growth was monitored.

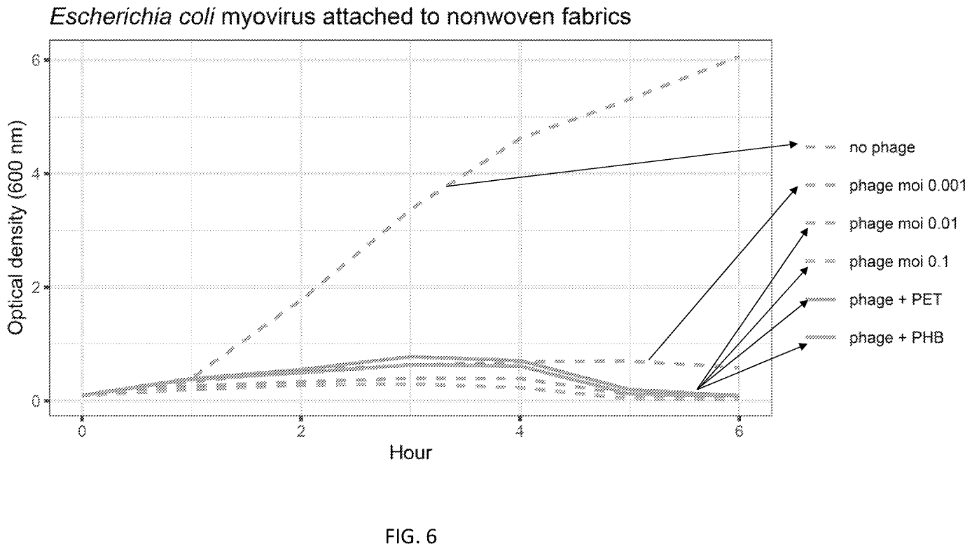

FIG. 6 shows dynamic growth curves of Escherichia coli strain 11303 showing the effect of T4 myovirus phage attached to plasma-treated PET and PHB nonwoven fibers. Dashed lines show E. coli growth curves with 1) no phage infection, 2) phage infection at a multiplicity of infection (m.o.i.) of 0.001, 3) phage infection at m.o.i. of 0.01, and 4) phage infection at m.o.i. of 0.1. These were compared with E. coli growth curves plotted by solid lines showing 1-cm2 plasma-treated nonwoven fabric squares of either PET or PHB with phage attached to them. Phages attached to surfaces such as PET or PHB were not as readily available for bacterial infection as phages added in suspension to a bacterial culture. Nonetheless, the phages attached to PET or PHB nonwoven fabric squares inhibited E. coli growth better than a phage suspension at an m.o.i. of 0.001 and less efficiently than—though comparable to—a phage suspension at an m.o.i. of 0.1. E. coli myovirus phage T4 was successfully attached to diverse types of plasma-treated nonwoven fabrics and inhibited E. coli growth.

### 7.8. Example 8: Effect of Podovirus Phage Attached to Nonwoven Fibers on Bacterial Growth

In this example, nonwoven fabric was used. S. aureus podovirus Rosenblumvirus P00474 phages were attached to the surface of PET or PHB nonwoven fabric and bacterial growth was monitored.

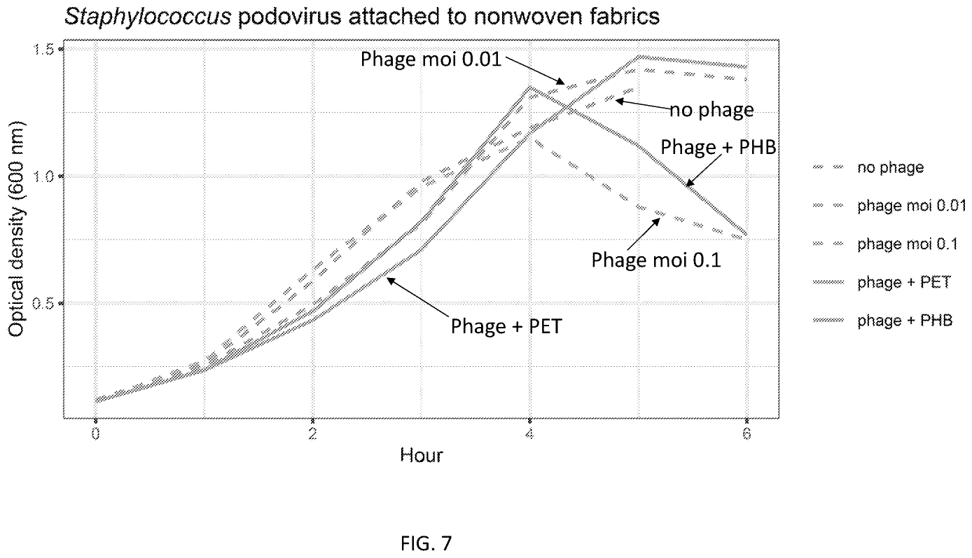

FIG. 7 shows Dynamic growth curves of Staphylococcus aureus strain 100311 showing the effect of S. aureus podovirus Rosenblumvirus PHP00474 attached to plasma-treated PET and PHB nonwoven fibers. Dashed lines showed S. aureus growth curves with 1) no phage infection, 2) phage infection at m.o.i. of 0.01, and 4) phage infection at m.o.i. of 0.1. These were compared with S. aureus growth curves plotted by solid lines showing 1-cm² plasma-treated nonwoven fabric squares of either PET or PHB with phage attached to them. Phages attached to surfaces such as PET or PHB were not as readily available for bacterial infection as phages added in suspension to a bacterial culture. Nonetheless, the phages attached to PET nonwoven fabric squares inhibited S. aureus growth similarly to a phage suspension at an m.o.i. of 0.01. Phages attached to PHB nonwoven fabric squares inhibited S. aureus growth similarly to a phage suspension at an m.o.i. of 0.1. S. aureus podoviruses were successfully attached to diverse types of plasma-treated nonwoven fabrics and can inhibit S. aureus growth.

### 7.9. Example 9: Effect of Siphovirus Phage Attached to Nonwoven Fibers on Bacterial Growth

In this example, nonwoven fabric is used. Siphovirus phages are attached to the surface of the PET or PHB nonwoven fabric and bacterial growth is monitored.

Growth of siphovirus infecting bacteria in four conditions are tested: 1) no phage infection, 2) phage infection at m.o.i. of 0.01, and 4) phage infection at m.o.i. of 0.1. Phages attached to surfaces such as PET or PHB nonwoven fabric squares inhibit siphovirus infecting bacteria growth. Siphovirus are successfully attached to diverse types of plasma-treated nonwoven fabrics and can inhibit targeted bacterial growth.

### 7.10. Example 10: Surface Modification of Nonwoven Fabrics

In this example, nonwoven fabrics were used. PET or PHB fabric squares of 2 cm×2 cm were treated with oxygen plasma. Plasma treatment was performed through Reactive Ion Etching (μEtch) in a cylindrical chamber 30 cm in diameter. A uniform glow discharge was created within a copper coil wrapped onto the chamber and connected to a radiofrequency (RF) generator. Commercially available oxygen gas (99.993%, Praxair, Edmonton, Canada) was input into the chamber. Samples of PET or PHB polymer fabric were treated in plasma at a floating potential for 1 min under a RF power of 100 W, an oxygen concentration of 25%, and a vacuum of 100 mTorr.

### 7.11. Example 11: Phage Immobilization on Nonwoven Fabrics

In this example, nonwoven fabrics were used. Adsorption on Untreated and Plasma Treated (PT) Fabrics. Attachment methods were performed in PBS buffer at pH 7.5. Oxygen plasma-treated and untreated PET or PHB fabrics of dimensions 2 cm×2 cm were immersed in 125-mL shake flasks containing 20 mL of a myovirus or podovirus suspension (2× 108 pfu/mL) and mixed at 4° C. and 60 rpm for 2 h. After exposure to phages, the films were washed seven times in PBS buffer to remove any unbound or loosely attached phages. All washing steps were performed at room temperature.

### 7.12. Example 12: Manufacturing of Surface Activated Nonwoven Fabrics

In this example, nonwoven fabrics were used.

This example illustrates enablement of inline phage treatment of nonwoven fabrics during manufacturing. As shown in FIG. 8, the processes described enables the treatment of nonwoven fabrics of diverse composition to be impregnated with phages of diverse morphology and host specificity after plasma treatment of the nonwoven fabric inline during a manufacturing process as follows: (1) The nonwoven fabric is plasma-treated (either encased or open-air plasma treatment) in-line in an automated manufacturing process; (2) Surface-active nonwoven fabric can be impregnated on both surfaces using a dip tray (also called an impregnation tank or dip tank); and (3) The nonwoven fabric is gently dried with an air dryer at temperatures below 50° C. that do not denature phage virions.

Alternatively, the process is as follows: (1) The nonwoven fabric is plasma-treated (either encased or open-air plasma treatment) in-line in an automated manufacturing process; (2) Alternatively, nonwoven fabric can be impregnated on one surface alone using a spray-on or misting apparatus; and (3) The nonwoven fabric is gently dried with an air dryer at temperatures below 50° C. that do not denature phage virions.

### 8. EQUIVALENTS AND INCORPORATION BY REFERENCE

While the invention has been particularly shown and described with reference to a preferred embodiment and various alternate embodiments, it will be understood by persons skilled in the relevant art that various changes in form and details can be made therein without departing from the spirit and scope of the invention.

All references, issued patents and patent applications cited within the body of the instant specification are hereby incorporated by reference in their entirety, for all purposes.

What is claimed is:

1. A composition comprising a phage combination attached to a polymeric fabric,
wherein the phage combination comprises one or more sets of phages that infect one or more bacterial species,
wherein the phages are selected from Myoviridae, Podoviridae and Siphoviridae, and
wherein the polymeric fabric is selected from at least one of a polyhydroxybutyrate (PHB) or a polyethylene terephthalate (PET) nonwoven fabric.

2. The composition of claim 1, wherein the phages are of the genus Pahexavirus or Rosenblumvirus.

3. The composition of claim 1 or claim 2, wherein the phages are tailed viruses.

4. The composition of any one of claims 1-3, wherein the phages are at least one of Myoviridae or Podoviridae.

5. The composition of claim 4, wherein the phages are T4 myoviruses.

6. The composition of claim 5, wherein the phages are podoviruses.

7. The composition of any one of claims 1-6, wherein the one or more sets of phages infect at least one common target bacterial species.

8. The composition of any one of claims 1-7, wherein each set of phages comprises at least two, three, four, five, or more phages selected from one or more phage libraries,
wherein the one or more phage libraries comprise phages from one or more genera.

9. The composition of any one of claims 1-8, wherein the phage combination is noncovalently or covalently attached to the polymeric fabric.

10. The composition of claim 9, wherein the polymeric fabric is synthetic nonwoven fabric.

11. The composition of any one of claims 1-10, wherein the phage combination is deposited onto at least one material that forms at least a portion of a personal care product.

12. The composition of claim 11, wherein the personal care product is selected from a group consisting of facemasks, sanitary pads, tampons, baby diapers, adult incontinence products, or cosmetic facial pads or wipes, fingernail wipes, or patient bathing hygiene textiles.

13. The composition of any one of claims 1-12, wherein the bacterial species is commonly associated with contact dermatitis.

14. The composition of claim 13, wherein the bacterial species is selected from a skin microbiome biobank.

15. The composition of claim 14, wherein the bacterial species is from a skin microbiome isolated from a biological sample of a subject.

16. The composition of any one of claims 1-15, wherein the one or more bacterial species are selected from a population of microorganisms comprising Staphylococcus aureus, Staphylococcus argenteus, Streptococcus pyogenes, Streptococcus pneumoniae, Escherichia coli, Enterococcus faecalis, Cutibacterium acnes, Corynebacterium tuberculostearicum, or combinations thereof.

17. The composition of any one of claims 1-16, wherein the phage is an Escherichia coli infecting phage.

18. The composition of claims 1-16, wherein the phages are Staphylococcus aureus infecting phages.

19. The composition of any one of claims 1-18, wherein the polymeric fabric further comprises a fabric is selected from the group consisting of a polylactic acid (PLA), polyester (PE), polycaprolactone (PCL), acrylics, olefins, polyhydroxyalkanoates (PHA), polyhydroxybutyrate (PHB), polyethylene terephthalate (PET), polypropylene (PP), and polypropylene carbonate (PPC) nonwoven fabric.

20. A composition comprising a phage combination attached to a polymeric fabric,
wherein the phage combination comprises one or more sets of Caudoviricetes phages, and
wherein the polymeric fabric is a polyethylene terephthalate (PET) nonwoven fabric.

21. A composition comprising a phage combination attached to a polymeric fabric,
wherein the phage combination comprises at least Staphylococcus aureus infecting phages, and
wherein the polymeric fabric is selected from at least one of a polyhydroxybutyrate (PHB) or a polyethylene terephthalate (PET) nonwoven fabric.

22. A composition comprising a phage combination attached to a polymeric fabric,
wherein the phage combination comprises at least Escherichia coli infecting phages, and
wherein the polymeric fabric is selected from at least one of a polyhydroxybutyrate (PHB) or a polyethylene terephthalate (PET) nonwoven fabric.

23. A composition comprising a phage combination attached to a polymeric fabric,
wherein the phage combination comprises one or more sets of Caudoviricetes phages, and
wherein the polymeric fabric is selected from the group consisting of a polylactic acid (PLA), polyester (PE), polycaprolactone (PCL), acrylics, olefins, polyhydroxyalkanoates (PHA), polyhydroxybutyrate (PHB), polyethylene terephthalate (PET), polypropylene (PP), and polypropylene carbonate (PPC) nonwoven fabric.

24. A composition comprising a phage combination attached to a polymeric fabric, wherein the phage combination comprises one or more sets of phages that infect one or more bacterial species.

25. The composition of any one of claims 20-24, wherein the phages are of the genus Pahexavirus or Rosenblumvirus.

26. The composition of any one of claims 20-25, wherein the phages are selected from a Myoviridae, Podoviridae and Siphoviridae.

27. The composition of any one of claims 20-26, wherein the phages are tailed viruses.

28. The composition of any one of claims 20-27, wherein the phages are at least one of Myoviridae or Podoviridae.

29. The composition of claim 28, wherein the phages are T4 myoviruses.

30. The composition of claim 28, wherein the phages are podoviruses.

31. The composition of any one of claims 20-30, wherein the one or more sets of phages infect at least one common target bacterial species.

32. The composition of any one of claims 20-31, wherein each set of phages comprises at least two, three, four, five, or more phages selected from one or more phage libraries,
wherein the one or more phage libraries comprise phages from one or more genera.

33. The composition of any one of claims 20-32, wherein the phage combination is noncovalently or covalently attached to the polymeric fabric.

34. The composition of claim 24, wherein the polymeric fabric is synthetic nonwoven fabric.

35. The composition of any one of claims 20-34, wherein the phage combination is deposited onto at least one material that forms at least a portion of a personal care product that contacts human skin.

36. The composition of claim 35, wherein the personal care product is selected from a group consisting of facemasks, sanitary pads, tampons, baby diapers, adult incontinence products, or cosmetic facial pads or wipes, fingernail wipes, or patient bathing hygiene textiles.

37. The composition of any one of claims 20-36, wherein the bacterial species is commonly associated with contact dermatitis.

38. The composition of claim 37, wherein the bacterial species is selected from a skin microbiome biobank.

39. The composition of claim 37, wherein the bacterial species is from a skin microbiome isolated from a biological sample of a subject.

40. The composition of any one of claims 20 and 23-39, wherein the one or more bacterial species are selected from a population of microorganisms comprising Staphylococcus aureus, Staphylococcus argenteus, Streptococcus pyogenes, Streptococcus pneumoniae, Escherichia coli, Enterococcus faecalis, Cutibacterium acnes, Corynebacterium tuberculostearicum, or combinations thereof.

41. The composition of any one of claims 20 and 21-40, wherein the phage combination further comprises at least Escherichia coli infecting phages.

42. The composition of claims 20 and 22-40, wherein the phage combination further comprises at least Staphylococcus aureus infecting phages.

43. The composition of any one of claims 20-42, wherein the polymeric fabric is selected from the group consisting of a polylactic acid (PLA), polyester (PE), polycaprolactone (PCL), acrylics, olefins, polyhydroxyalkanoates (PHA), polyhydroxybutyrate (PHB), polyethylene terephthalate (PET), polypropylene (PP), and polypropylene carbonate (PPC) nonwoven fabric.

44. A method of selecting the phage combination of any one of claims 1-43.

45. A method of attaching the phage combination of any one of claims 1-43 to a nonwoven polymeric fabric.

46. The method of claim 45, wherein attaching the phage combination is achieved by permeating the phage capsid onto the surface of the nonwoven polymeric fabric.

47. The method of claim 46, wherein the phage capsid is absorbed to the surface of the polymeric fabric via electrostatic charge.

48. The method of claim 47, wherein the tail is exposed away from the fabric.

49. The method of any one of claims 44-48, wherein attaching the phage combination is achieved by misting, chemical bonding, physical adsorption, bulk mixing, plasma-treated surface bonding, or a combination thereof.

50. The method of any one of claims 44-49, wherein attaching the phage combination is achieved by plasma-treated surface bonding.

51. A method of preparing a personal care product, the method comprising
(a) contacting a nonwoven polymeric fabric with a composition comprising a phage combination,
wherein the phage combination comprises one or more sets of phages of Caudoviricetes phages that infect one or more bacterial species, and
wherein the nonwoven polymeric fabric is selected from at least one of a polyhydroxybutyrate (PHB) or a polyethylene terephthalate (PET) nonwoven fabric.

52. A method of preparing a personal care product, the method comprising
(a) contacting a nonwoven polymeric fabric with a plasma composition to generate a plasma-treated nonwoven fabric,
wherein the nonwoven polymeric fabric is selected from at least one of a polyhydroxybutyrate (PHB) or a polyethylene terephthalate (PET) nonwoven fabric;
(b) immobilizing a combination of phages comprising at least one or more sets of Caudoviricetes phages onto the nonwoven polymeric fabric,
wherein the phage combination comprises one or more sets of phages that infect one or more bacterial species; and
(c) incorporating the nonwoven polymeric fabric into the personal care product.

53. A nonwoven polymeric fabric comprising a phage combination attached to the polymeric fabric,
wherein the phage combination comprises one or more sets of Caudoviricetes phages that infect one or more bacterial species, and
wherein the polymeric fabric is selected from a polylactic acid (PLA), polyester (PE), acrylics, olefins, polyhydroxyalkanoates (PHA), polyhydroxybutyrate (PHB), polyethylene terephthalate (PET), polypropylene (PP) and polypropylene carbonate (PPC) nonwoven fabric.

54. Manufacturing of a personal care product for reducing contact dermatitis, reducing skin infection and/or reducing body odor, the personal care product comprising a nonwoven polymeric fabric,
wherein the nonwoven polymeric fabric comprises a phage combination attached to the nonwoven fabric selected from at least one of a polyhydroxybutyrate (PHB) or a polyethylene terephthalate (PET) nonwoven fabric, and
wherein the phage combination comprises one or more sets of Caudoviricetes phages that infect one or more bacterial species.

55. The personal care product of claim 54, wherein the personal care product is selected from a group consisting of facemasks, sanitary pads, tampons, baby diapers, adult incontinence products, or cosmetic facial pads or wipes, fingernail wipes, or patient bathing hygiene textiles.

56. The phage of the composition of any one of claims 1-43, the phage of the method of any one of claims 44-52, or the phage of the nonwoven polymeric fabric of claim 52, or the phage of the personal care product of claims 53 and 54, wherein the phage comprises a nucleic acid sequence having at least 80%, 85%, 90%, 95%, 96%, 97%, 98%, or 99% identity with any one of SEQ ID NOs.: 1-132.

57. The phage of the composition of any one of claims 1-43, the phage of the method of any one of claims 44-52, or the phage of the nonwoven polymeric fabric of claim 52, or the phage of the personal care product of claims 53 and 54, wherein the phage comprises a nucleic acid sequence having at least 95% identity with any one of SEQ ID NOs.: 1-132.

58. The phage of the composition of any one of claims 1-43, the phage of the method of any one of claims 44-52, or the phage of the nonwoven polymeric fabric of claim 52, or the phage of the personal care product of claims 53 and 54, wherein the phage comprises a nucleic acid sequence having a sequence of any one of SEQ ID NOs.: 1-132.
